(19) United States
(12) Patent Application Publication
(10) Pub. No.: US 2026/0364188 A1
(43) Pub. Date: Nov. 19, 2026

(54) BISECTING-GLYCAN BRIDGED CONJUGATION FOR PRODUCING GLYCOPROTEIN CONJUGATES

(71) Applicant: Merck Sharp & Dohme LLC, Rahway, NJ (US)

(72) Inventors: Yen-Pang HSU, Saugus, MA (US); Sampat L. INGALE, Lexington, MA (US)

(73) Assignee: Merck Sharp & Dohme LLC, Rahway, NJ (US)

(21) Appl. No.: 19/125,526

(22) PCT Filed: Nov. 2, 2023
(86) PCT No.: PCT/US2023/078425

§ 371 Date: Apr. 29, 2025

Related U.S. Application Data
(60) Provisional application No. 63/423,282, filed on Nov. 7, 2022.
(60) Provisional application No. 63/509,684, filed on Jun. 22, 2023.

(51) Int. Cl. A61K 47/68; C07K 16/32

(52) U.S. Cl. CPC A61K 47/68031; A61K 47/6849; A61K 47/6855; A61K 47/6889; C07K 16/32; C07K 2317/92

(57) ABSTRACT

A bisecting glycan-bridged conjugation strategy that enables site-specific conjugation of glycoproteins without the need for chemical synthesis of oligosaccharide and amino acid engineering is disclosed. Application of this method is demonstrated by conjugation of human and mouse antibodies with a cytotoxin. This strategy preserves the Fc effector function of the antibodies and provides the ability to remodel glycans to fine-tune the immunogenicity and pharmacokinetic properties of antibody-drug conjugates through glycoengineering.

### CROSS-REFERENCE TO RELATED APPLICATIONS

This application claims the benefit of U.S. Provisional Patent Application Ser. No. 63/423,282 filed Nov. 7, 2022 and 63/509,684 filed Jun. 22, 2023, the entire contents of which are incorporated by reference herein.

### BACKGROUND OF THE INVENTION

### (1) Field of the Invention

The present invention relates to a bisecting glycan-bridged conjugation strategy that enables site-specific conjugation of payloads to antibodies and other glycoproteins without the need for chemical synthesis of oligosaccharide and amino acid engineering.

### (2) Description of Related Art

Antibody-drug-conjugates (ADCs) present a promising and rapidly growing class of therapeutics for disease treatments (Tong et al., Molecules 26, 5847 (2021); Leung et al., Antibodies (Basel) 9, doi:10.3390/antib9010002 (2020)). The success of ADCs against various oncology indications has stimulated their rapid growth in other applications, such as infectious disease and protein degradation (Lehar et al., Nature 527, 323-328 (2015); McPherson & Hobson, Meth. Mol. Biol. 2078, 23-36 (2020); Banik et al., Nature 584, 291-297 (2020)). ADCs are versatile molecules composed of a monoclonal antibody covalently connected to small molecules. The antibody endows high binding specificity to the target, an improved lifetime in vivo, and the ability to trigger effector-mediated immune responses to clear the pathogenic targets. The payload molecules provide cytotoxicity with mechanisms of action to kill or impair functions of the targets. In concert, these functionalities are imperative in creating effective therapeutics with improved safety and pharmacokinetic properties.

Conjugation through the existing lysine residues (e.g., by amine-succinimidyl ester) is one of the most commonly used methods for ADC development (Alves, Antib. Ther. 2, 33-39 (2019); Chari et al., Angew. Chemie Int. Ed. 53, 3796-3827 (2014)). This method generates ADCs with varying numbers of payloads from 2 to 8n, presenting an easy-to-use strategy to create high-DAR (drug-to-antibody ratio) ADCs. However, the exact number of payloads and their conjugation sites are not controllable. This might result in compromised antibody binding affinity to the targets, or loss of efficacy caused by, for instance, steric effects. Contemporary trends in ADC development have steered toward site-specific conjugation strategies because they result in high product homogeneity and more controllable molecular properties. A remarkable example is the incorporation of cysteine residues into the antibody framework to enable site-specific cysteine-maleimide conjugation (Junutula et al., Nature Biotechnology 26, 925-932 (2008); Junutula et al., J. Immunol. Methods 332, 41-52 (2008); Shinmi et al., Bioconjugate Chemistry 27, 1324-1331 (2016)). Many more examples encompassing enzymatic and chemical site-specific conjugation strategies are reported in the literature (Beerli et al., PLOS ONE 10, e0131177 (2015); Chen et al., Scientific Reports 6, 31899 (2016)). However, the need to modify the antibody framework to create conjugation sites could impede ADC development by altering the antibody's integrity, in addition to the predisposed challenges of engineering itself (Junutula et al., J. Immunol. Methods 332, 41-52 (2008)).

To address this issue, new strategies that possess site-specificity without the need for antibody engineering have been developed, such as affinity-assisted lysine conjugation (Kishimoto et al., Bioconjugate Chem. 30, 698-702 (2019); Dennler et al., Bioconjugate Chem. 25, 569-578 (2014)). The antibody N-glycan presents another great opportunity for this purpose because (1) antibody glycosylation sites are highly conserved and (2) the difference between glycan and peptide chemistry (i.e., glycosidic bonds versus amide bonds) enables several bio-orthogonal reactions to modify glycan structures (Agnew et al., Anal. Bioanal. Chem. 413, 4989-5001 (2021); Li et al., Angew. Chem. Int. Ed. Engl. 53, 7179-7182 (2014); Okeley et al., Bioconjugate Chem. 24, 1650-1655 (2013); Zhou et al., Bioconjugate Chem. 25, 510-520 (2014); Zhu et al., MAbs 6, 1190-1200 (2014)). To conjugate a drug through glycans, researchers first have to overcome the structural heterogeneity of glycans (Higel et al., Eur. J. Pharm. Biopharm. 100, 94-100 (2016)). For example, Immunoglobulin G1 (IgG1), the major antibody class for ADCs development, has conserved N-glycans at Asn297 in the Fc domain with over 25 glycoforms being identified (Pucić et al., Mol. Cell. Proteomics 10, doi:10.1074/mcp.M111.010090 (2011)). Endoglycosidases have been used to trim the glycan structures down to the conserved fucose-N-acetylglucosamine (GlcNAc) dipeptide to enable enzymatic or chemical installation of coupling handles, such as azide (Angew. Chem. Int. Ed. Engl. 53, 7179-7182 (2014); Walls et al., Nature Structural & Molecular Biology 23, 899-905 (2016); van Geel et al., Bioconjugate Chem. 26, 2233-2242 (2015)). However, the removal of glycan structures might affect the stability of the Fc domain and the antibody binding affinity to Fc gamma receptors (FcγRs), resulting in altered immunogenicity and life-time (Krapp et al., J. Mol. Biol. 325, 979-989 (2003); Mimura et al., Mol. Immunol. 37, 697-706 (2000); Mimura et al., Protein Cell 9, 47-62 (2018); Goetze et al., Glycobiology 21, 949-959 (2011); Chiang et al., Curr. Opin. Struct. Biol. 40, 104-111 (2016)). While this issue can be addressed by the re-installation of synthetic azido-glycans using glycosynthases, the difficulties of glycan synthesis still make this approach challenging for many applications (Li et al., Angew. Chem. Int. Ed. Engl. 53, 7179-7182 (2014); Huang et al., J. Amer. Chem. Soc. 134, 12308-12318 (2012)).

### BRIEF SUMMARY OF THE INVENTION

The present invention provides a conjugation strategy that enables efficient, gene editing-free conjugation with site-specificity for generating glycoprotein conjugates and ADCs in particular. The present invention comprises linking N-azido acetylglucosamine (azido-GlcNAc or GlcNAz) to the α1,4 linked mannose of the trimannose core of one or more IgG N-glycans, each azido-GlcNAc conjugated to the asparagine residue of an N-glycosylation site having the consensus amino acid sequence Asparagine-X-serine/threonine, wherein X is any amino acid except for proline, using human N-acetylglucosaminyltransferase III (GnT-III or MGAT3), wherein the azido-GlcNAc may then serve as an attachment point for azide-alkyne click chemistry conjugation to alkyne-bearing payloads (See Narasimhan, J. Biol. Chem. 257, 10235-10242 (1982) and Horisawa, Frontiers in Physiology 5, doi: 10.3389/fphys.2014.00457 (2014) for azide-alkyne click chemistry).

Utility of the present invention is demonstrated herein by conjugating an alkyne-bearing payload to human and mouse antibodies that recognize particular antigens found on the surface of cancer cells. As demonstrated by the binding affinity assays disclosed herein, this conjugation strategy is highly compatible with IgG functionalities, including target binding and Fc receptor interactions (Bai et al., Biochem. Pharmacol. 39, 1941-1949 (1990)). Moreover, in contrast to N-glycan conjugation schemes disclosed in the prior art, conjugation through the bisecting position of the N-glycan preserves the opportunity to remodel the N-glycan antenna structures of the bisected N-glycan to be terminated with galactose and/or α2,3 or α2,6 sialic acid residues, which are known to mediate various effector functions of antibodies, creating possibilities to fine-tune the ADC immune responses.

The conjugation strategy of the present invention enables the production of ADCs having a DAR of up to two when the only N-glycosylation sites on the antibody are located at the Asn at position 297 (according to Eu numbering) of the heavy chain constant domain but may be greater than two if there are additional naturally occurring N-glycosylation sites located in the V_{H} and/or V_{L} domains. In addition, by engineering additional non-native N-glycan sites into the antibody, either in the constant domains or the variable domains, ADCs with DARs of up to 4, 6, 8, 10, 12, 14, 16, 18, or 20 may be obtained (See for example, U.S. Pat. No. 11,332,544 for engineered non-native N-glycan sites in antibodies).

The present invention provides a glycoprotein comprising protein conjugated to at least one bisected N-glycan in which the nonreducing end of the β1,4 linked mannose residue of the trimannosyl core (M3) of the bisected N-glycan is linked to the reducing end of an azido-N-acetyl glucosamine (6-azido-GlcNAc or GlcNAz) residue in a β1,4 linkage, wherein M3 is represented by the formula comprising Manα1-3(Manα1-6)Man⊕1-4GlcNAcβ1-4GlcNAcβ1-Asn; wherein the 6-azido-GlcNAc and GlcNAz is represented by the formula comprising

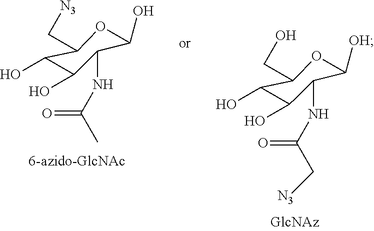 [SMILES: CC(=O)NC1C(O)OC(CN=[N+]=[N-])C(O)C1O ; [N-]=[N+]=NCC(=O)NC1C(O)OC(CO)C(O)C1O]

and, wherein the nonreducing end of at least one of the α1,6-linked or α1,3-linked mannose residues is linked to the reducing end of an N-acetyl glucosamine (GlcNAc) residue by a β1,2 linkage.

In a further embodiment, the nonreducing end of the GlcNAc residue is linked to the reducing end of a galactose residue by a β1,4 linkage. In a further embodiment, the nonreducing end of the galactose residue is linked to the reducing end of a sialic acid residue by an α2,6 linkage. In a further embodiment, the nonreducing end of the galactose residue is linked to the reducing end of a sialic acid residue by an α2,3 linkage. In a further embodiment, the nonreducing end of the α1,6-linked mannose residue is linked to the reducing end of a first GlcNAc residue by a β1,2 linkage and the nonreducing end of the α1,3-linked mannose residue is linked to the reducing end of a second GlcNAc residue by a β1,2 linkage. In a further embodiment, (i) the nonreducing end of the first GlcNAc residue is linked to the reducing end of a galactose residue by a β1,4 linkage; (ii) the nonreducing end of the second GlcNAc residue is linked to the reducing end of a galactose residue by a β1,4 linkage; or (iii) the nonreducing end of the first GlcNAc residue is linked to the reducing end of a first galactose residue by a β1,4 linkage and the nonreducing end of the second GlcNAc residue is linked to the reducing end of a second galactose residue by a β1,4 linkage. In a further embodiment, the nonreducing end of the galactose residue of (i) or (ii) or the nonreducing end of the first and second galactose residues of (iii) each linked to the reducing end of a sialic acid residue by an α2,3 linkage.

In a further embodiment, the nonreducing end of the galactose residue of (i) or (ii) or the nonreducing end of the first and second galactose residues of (iii) each linked to the reducing end of a sialic acid residue by an α2,6 linkage.

In a further embodiment, the azido group of the azido-GlcNAc or GlcNAz is conjugated to a payload comprising an alkyne function group in a triazole linkage.

In a further embodiment, the payload is a therapeutic agent, a diagnostic agent, radionuclide, or protecting group.

In a further embodiment, the therapeutic agent is a cytotoxic agent, an anti-inflammatory agent, a peptide, an immune agonist, or a nucleic acid molecule or nucleic acid analog.

In a further embodiment, the anti-inflammatory agent is a glucocorticoid receptor agonist. In a further embodiment, the anti-inflammatory agent is Cortisol, cortisone acetate, beclometasone, prednisone, prednisolone, methylprednisolone, betamethasone, trimcinolone, budesonide, dexamethasone, fluticasone, or mometasone.

In a further embodiment, the cytotoxic agent is selected from the group consisting of taxanes, anthracyclines, camptothecins, epothilones, mytomycins, combretastatins, vinca alkaloids, maytansinoids, enediynes, duocarmycins, tubulysins, amatoxins, bleomycins, dolastatins and auristatins, pyrrolobenzodiazepine dimers, indolinobenzodiazepine dimers, and radioisotopes.

In a further embodiment, the protein comprises the fragment crystallizable (Fc) domain of an antibody. In a further embodiment, the protein is an antibody having a heavy chain and a light chain.

The present invention provides an antibody conjugated to at least one bisected N-glycan in which the nonreducing end of the β1,4 linked mannose residue of the trimannosyl core (M3) of the bisected N-glycan is linked to the reducing end of an azido-N-acetyl glucosamine (6-azido-GlcNAc or GlcNAz) residue in a β1,4 linkage, wherein M3 is represented by the formula comprising Manα1-3(Manα1-6)Manβ1-4GlcNAcβ1-4GlcNAcβ1-Asn; wherein the 6-azido-GlcNAc is represented by the formula comprising

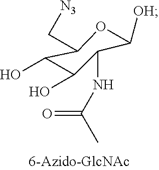 [SMILES: CC(=O)NC1C(O)OC(CN=[N+]=[N-])C(O)C1O]

and GlcNAz is represented by the formula comprising

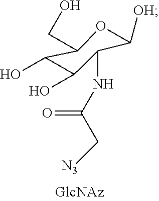 [SMILES: [N-]=[N+]=NCC(=O)NC1C(O)OC(CO)C(O)C1O]

wherein the nonreducing end of at least one of the α1,6-linked or α1,3-linked mannose residues is linked to the reducing end of an N-acetyl glucosamine (GlcNAc) residue by a β1,2 linkage.

In a further embodiment, the nonreducing end of the GlcNAc residue is linked to the reducing end of a galactose residue by a β1,4 linkage. In a further embodiment, the nonreducing end of the galactose residue is linked to the reducing end of a sialic acid residue by an α2,6 linkage. In a further embodiment, the nonreducing end of the galactose residue is linked to the reducing end of a sialic acid residue by an α2,3 linkage. In a further embodiment, the nonreducing end of the α1,6-linked mannose residue is linked to the reducing end of a first GlcNAc residue by a β1,2 linkage and the nonreducing end of the α1,3-linked mannose residue is linked to the reducing end of a second GlcNAc residue by a β1,2 linkage. In a further embodiment, (i) the nonreducing end of the first GlcNAc residue is linked to the reducing end of a galactose residue by a β1,4 linkage; (ii) the nonreducing end of the second GlcNAc residue is linked to the reducing end of a galactose residue by a β1,4 linkage; or (iii) the nonreducing end of the first GlcNAc residue is linked to the reducing end of a first galactose residue by a β1,4 linkage and the nonreducing end of the second GlcNAc residue is linked to the reducing end of a second galactose residue by a β1,4 linkage. In a further embodiment, the nonreducing end of the galactose residue of (i) or (ii) or the nonreducing end of the first and second galactose residues of (iii) each linked to the reducing end of a sialic acid residue by an α2,3 linkage.

In a further embodiment, the nonreducing end of the galactose residue of (i) or (ii) or the nonreducing end of the first and second galactose residues of (iii) each linked to the reducing end of a sialic acid residue by an α2,6 linkage.

In a further embodiment, the azido group of the 6-azido-GlcNAc or GlcNAz is conjugated to a payload comprising an alkyne function group in a triazole linkage.

In a further embodiment, the payload is a therapeutic agent, a diagnostic agent, radionuclide, or protecting group.

In a further embodiment, the therapeutic agent is a cytotoxic agent, an anti-inflammatory agent, a peptide, an immune agonist, or a nucleic acid molecule or nucleic acid analog.

In a further embodiment, the anti-inflammatory agent is a glucocorticoid receptor agonist. In a further embodiment, the anti-inflammatory agent is Cortisol, cortisone acetate, beclometasone, prednisone, prednisolone, methylprednisolone, betamethasone, trimcinolone, budesonide, dexamethasone, fluticasone, or mometasone.

In a further embodiment, the cytotoxic agent is selected from the group consisting of taxanes, anthracyclines, camptothecins, epothilones, mytomycins, combretastatins, vinca alkaloids, maytansinoids, enediynes, duocarmycins, tubulysins, amatoxins, bleomycins, dolastatins and auristatins, pyrrolobenzodiazepine dimers, indolinobenzodiazepine dimers, and radioisotopes.

In a further embodiment, the bisected N-glycan is conjugated to an asparagine residue at position 297 of the constant domain, wherein the position is according to Eu numbering. In a further embodiment, the bisected N-glycan is conjugated to an asparagine residue of a N-glycosylation site comprising the amino acid sequence asparagine-X-serine/threonine wherein X is any amino acid except proline.

The present invention further provides an antibody comprising the structure:

Ab-[B]ₙ

wherein Ab is an antibody; B is a bisected N-glycan wherein the bisecting sugar residue of the bisected N-glycan is a 6-azido-GlcNAc or GlcNAz residue having an azide functional group; and, n is an integer from 1-20.

In a further embodiment, the bisected N-glycan B comprises a structure selected from the group consisting of A2B-azide; FA2B-azide; A2B[3]G1-azide; FA2 [3]BG1-azide; A2B[6]G1-azide; FA2B[6]G1-azide; A2BG2-azide; FA2BG2-azide; A2B[3]G1S1(6)-azide; FA2B[3]G1S1(6)-azide; A2B[6]G1S1(6)-azide; FA2B[6]G1S1(6)-azide; A2BG2S2(6)-azide; FA2BG2S2(6)-azide; A2B[3]G1S1(3)-azide; FA2B[3]G1S1(3)-azide; A2B[6]G1S1(3)-azide; FA2B[6]G1S1(3)-azide; A2BG2S2(3)-azide; FA2BG2S2(3)-azide; A1B[3]-azide; FA1B[3]-azide; A1B[3]G1-azide; FA1B[3]G1-azide; A1B[3]G1S1(6)-azide; A1B[3]G1S1(3)-azide; FA1B[3]G1S1(6)-azide; and, FA1B[3]G1S1(3)-azide; wherein the azide refers to the azide of 6-azido-GlcNAc or GlcNAz.

The present invention further provides a composition comprising a plurality of antibodies and a pharmaceutically acceptable carrier, wherein each antibody independently comprises the structure:

Ab-[B]ₙ

wherein Ab is an antibody; B is a bisected N-glycan wherein the bisecting sugar residue of the bisected N-glycan is provided by a 6-azido-GlcNAc or GlcNAz residue having an azide functional group; and, n is an integer from 1-20.

In a further embodiment, the bisected N-glycan B comprises a structure selected from the group consisting of A2B-azide; FA2B-azide; A2B[3]G1-azide; FA2 [3]BG1-azide; A2B[6]G1-azide; FA2B[6]G1-azide; A2BG2-azide; FA2BG2-azide; A2B[3]G1S1(6)-azide; FA2B[3]G1S1(6)-azide; A2B[6]G1S1(6)-azide; FA2B[6]G1S1(6)-azide; A2BG2S2(6)-azide; FA2BG2S2(6)-azide; A2B[3]G1S1(3)-azide; FA2B[3]G1S1(3)-azide; A2B[6]G1S1(3)-azide; FA2B[6]G1S1(3)-azide; A2BG2S2(3)-azide; FA2BG2S2(3)-azide; A1B[3]-azide; FA1B[3]-azide; A1B[3]G1-azide; FA1B[3]G1-azide; A1B[3]G1S1(6)-azide; A1B[3]G1S1(3)-azide; FA1B[3]G1S1(6)-azide; and, FA1B[3]G1S1(3)-azide; wherein the azide refers to the azide of 6-azido-GlcNAc or GlcNAz.

In particular embodiments of the composition, the composition comprises predominantly one or two bisected N-glycan B structures. In further embodiments, the predominant one or two bisected N-glycan B structures comprise greater than 50 mole %, 60 mole %, 70 mole %, 80 mole %, 90 mole %, 95 mole %, 96 mole %, 97 mole %, 98 mole %, 99 mole %, or 100 mole % of the total N-glycans of the composition.

The present invention further provides an antibody conjugate comprising the structure:

Ab-[B-T-L-Pay]ₙ

wherein Ab is an antibody; B is a bisected N-glycan wherein the bisecting sugar residue of the bisected N-glycan is a 6-azido-GlcNAc or GlcNAz residue; L is a linker element comprising an alkyne functional group or a bond; Pay is a payload, which comprises an alkyne functional group when L is a bond; T is a triazole linkage formed between the linkage of the azide functional group and the alkyne functional group; and, n is an integer from 1-20.

In a further embodiment, L is a self-immolative linker. In a further embodiment, L has the formula L¹-L², wherein each of L¹ and L² is independently a linker element or a bond. In a further embodiment, L comprises a distal end comprising an alkyne containing group linked to the azide of the 6-azido-GlcNAc or GlcNAz residue of the bisected N-glycan B in a triazole linkage T and a proximal end linked to Pay.

In a further embodiment, the alkyne group is a strained cyclooctyne. In a further embodiment, the strained cyclooctyne is DBCO.

In a further embodiment, the bisected N-glycan B comprises a structure selected from the group consisting of A2B-azide; FA2B-azide; A2B[3]G1-azide; FA2 [3]BG1-azide; A2B[6]G1-azide; FA2B[6]G1-azide; A2BG2-azide; FA2BG2-azide; A2B[3]G1S1(6)-azide; FA2B[3]G1S1(6)-azide; A2B[6]G1S1(6)-azide; FA2B[6]G1S1(6)-azide; A2BG2S2(6)-azide; FA2BG2S2(6)-azide; A2B[3]G1S1(3)-azide; FA2B[3]G1S1(3)-azide; A2B[6]G1S1(3)-azide; FA2B[6]G1S1(3)-azide; A2BG2S2(3)-azide; FA2BG2S2(3)-azide; A1B[3]-azide; FA1B[3]-azide; A1B[3]G1-azide; FA1B[3]G1-azide; A1B[3]G1S1(6)-azide; A1B[3]G1S1(3)-azide; FA1B[3]G1S1(6)-azide; and, FA1B[3]G1S1(3)-azide, wherein the azide refers to the azide of 6-azido-GlcNAc or GlcNAz and which reacts with the alkyne to form the triazole linkage T.

In a further embodiment, wherein the bisecting sugar residue is azido-GlcNAc and the triazole linkage has the structure

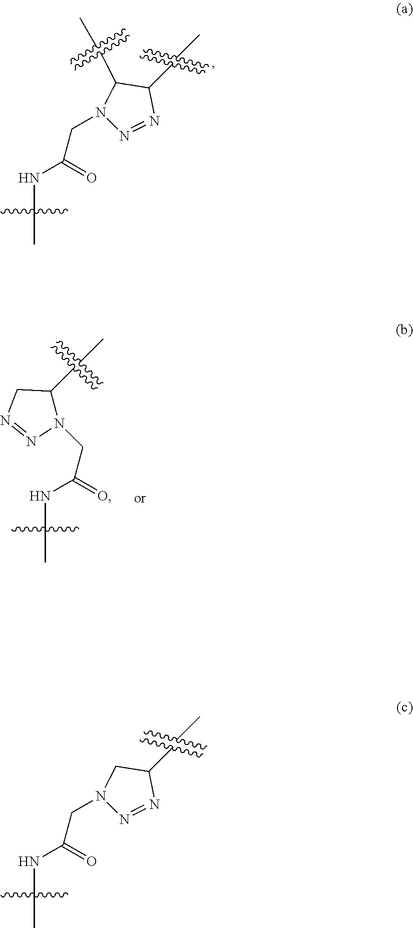

wherein the wavy line identifies the bond between the amine group of the GlcNAz residue and the C2 carbon of the glucose residue comprising the GlcNAz residue, and the double wavy lines identify bonds between the alkyne functional group of L or Pay when L is a bond; or, the bisecting sugar residue is 6-azido-GlcNAc and the triazole linkage has the structure

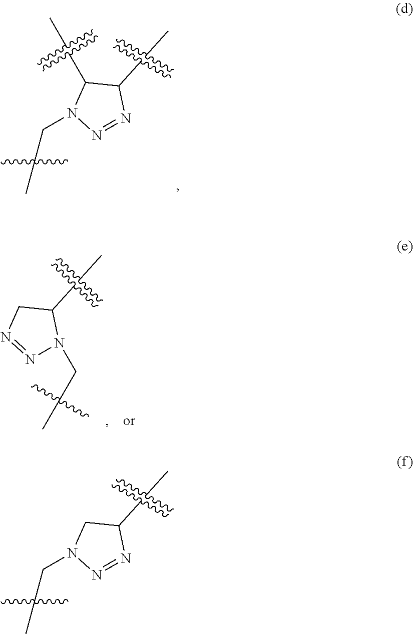

wherein the wavy line identifies the bond between the C5 and C6 carbons of the glucose residue comprising the 6-azido-GlcNAc residue and the double wavy lines identify bonds between the alkyne functional group of L or Pay when L is a bond.

In a further embodiment, the payload is a therapeutic agent, a diagnostic agent, radionuclide, or protecting group.

In a further embodiment, the therapeutic agent is a cytotoxic agent, an anti-inflammatory agent, a peptide, or a nucleic acid molecule or nucleic acid analog.

In a further embodiment, the anti-inflammatory agent is a glucocorticoid receptor agonist. In a further embodiment, the anti-inflammatory agent is Cortisol, cortisone acetate, beclometasone, prednisone, prednisolone, methylprednisolone, betamethasone, trimcinolone, budesonide, dexamethasone, fluticasone, or mometasone.

In a further embodiment, the cytotoxic agent is selected from the group consisting of taxanes, anthracyclines, camptothecins, epothilones, mytomycins, combretastatins, vinca alkaloids, maytansinoids, enediynes, duocarmycins, tubulysins, amatoxins, bleomycins, dolastatins and auristatins, pyrrolobenzodiazepine dimers, indolinobenzodiazepine dimers, and radioisotopes.

The present invention further provides a composition comprising a plurality of ADCs and a pharmaceutically acceptable carrier, wherein each ADC independently comprises the structure:

Ab-[B-T-L-Pay]ₙ

wherein Ab is an antibody; B is a bisected N-glycan wherein the bisecting sugar residue of the bisected N-glycan is a 6-azido-GlcNAc or GlcNAz residue having an azide functional group; L is a linker element comprising an alkyne functional group or a bond; Pay is a payload, which comprises an alkyne functional group when L is a bond; T is a triazole linkage formed between the linkage of the azide functional group and the alkyne functional group; and, n is an integer from 1-20.

In a further embodiment, L is a self-immolative linker. In a further embodiment, L has the formula L¹-L², wherein each of L¹ and L² is independently a linker element or a bond.

In a further embodiment, L comprises a distal end comprising an alkyne containing group linked to the azide of the 6-azido-GlcNAc or GlcNAz residue of bisected N-glycan B in triazole linkage T and a proximal end linked to Pay.

In a further embodiment, the alkyne group is a strained cyclooctyne. In a further embodiment, the strained cyclooctyne is DBCO.

In a further embodiment, the bisected N-glycan B comprises a structure selected from the group consisting of A2B-azide; FA2B-azide; A2B[3]G1-azide; FA2 [3]BG1-azide; A2B[6]G1-azide; FA2B[6]G1-azide; A2BG2-azide; FA2BG2-azide; A2B[3]G1S1(6)-azide; FA2B[3]G1S1(6)-azide; A2B[6]G1S1(6)-azide; FA2B[6]G1S1(6)-azide; A2BG2S2(6)-azide; FA2BG2S2(6)-azide; A2B[3]G1S1(3)-azide; FA2B[3]G1S1(3)-azide; A2B[6]G1S1(3)-azide; FA2B[6]G1S1(3)-azide; A2BG2S2(3)-azide; FA2BG2S2(3)-azide; A1B[3]-azide; FA1B[3]-azide; A1B[3]G1-azide; FA1B[3]G1-azide; A1B[3]G1S1(6)-azide; A1B[3]G1S1(3)-azide; FA1B[3]G1S1(6)-azide; and, FA1B[3]G1S1(3)-azide, wherein the azide refers to the azide of 6-azido-GlcNAc or GlcNAz and which reacts with the alkyne to form the triazole linkage T.

In particular embodiments of the composition, the composition comprises predominantly one or two bisected N-glycan B structures. In further embodiments, the predominant one or two bisected N-glycan B structures comprise greater than 50 mole %, 60 mole %, 70 mole %, 80 mole %, 90 mole %, 95 mole %, 96 mole %, 97 mole %, 98 mole %, 99 mole %, or 100 mole % of the total N-glycans of the composition.

In a further embodiment, wherein the bisecting sugar residue is azido-GlcNAc and the triazole linkage has the structure

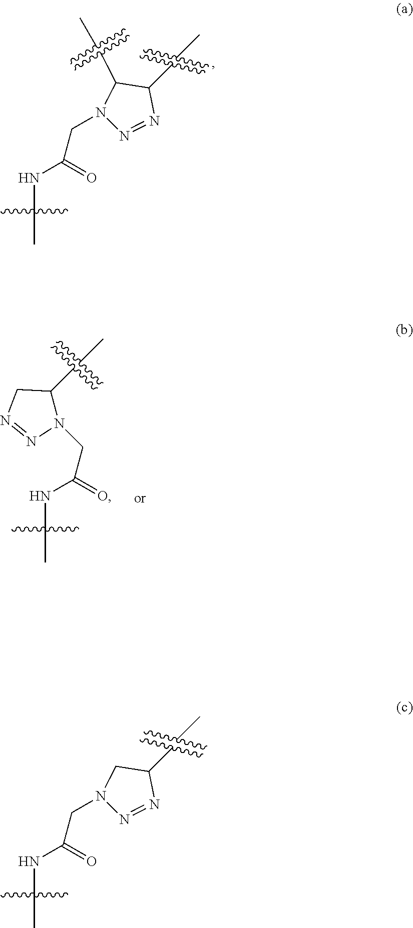

wherein the wavy line identifies the bond between the amine group of the GlcNAz residue and the C2 carbon of the glucose residue comprising the GlcNAz residue and the double wavy lines identify bonds between the alkyne functional group of L or Pay when L is a bond; or, the bisecting sugar residue is 6-azido-GlcNAc and the triazole linkage has the structure

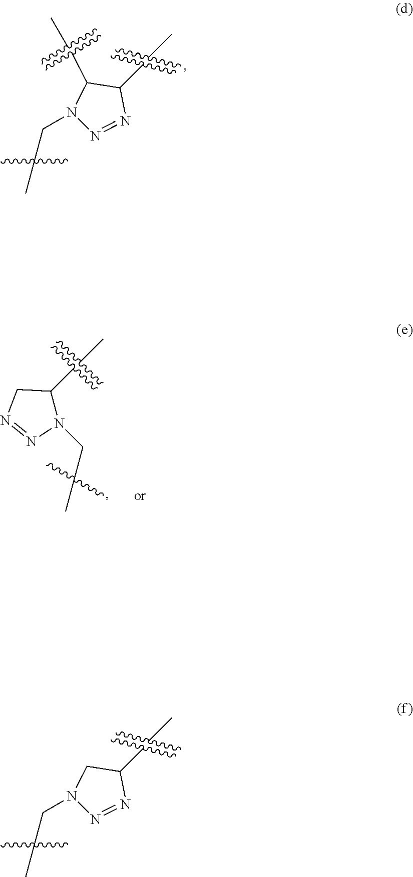

wherein the wavy line identifies the bond between the C5 and C6 carbons of the glucose residue comprising the 6-azido-GlcNAc residue and the double wavy lines identify bonds between the alkyne functional group of L or Pay when L is a bond.

In a further embodiment, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B-azide or FA2B-azide or both A2B-azide and FA2B-azide, wherein the azide is part of the triazole linkage T.

In a further embodiment, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[3]G1-azide or FA2B[3]G1-azide or both A2B[3]G1-azide and FA2B[3]G1-azide, wherein the azide is part of the triazole linkage T.

In a further embodiment, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[6]G1-azide or FA2B[6]G1-azide or both A2B[6]G1-azide and FA2B[6]G1-azide, wherein the azide is part of the triazole linkage T.

In a further embodiment, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2BG2S2-azide or FA2BG2S2-azide or both A2BG2-azide and FA2BG2-azide, wherein the azide is part of the triazole linkage T.

In a further embodiment, the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[3]G1S1(6 or 3)-azide or FA2B[3]G1S1(6 or 3)-azide or both A2B[3]G1S1(6 or 3)-azide and FA2B[3]G1S1(6 or 3)-azide, wherein the azide is part of the triazole linkage T.

In a further embodiment, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[6]G1S1(6 or 3)-azide or FA2B[6]G21S1(6 or 3)-azide or both A2B[6]G1S1(6 or 3)-azide and FA2B[6]G1S1(6 or 3)-azide, wherein the azide is part of the triazole linkage T.

In a further embodiment, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2BG2S2(6 or 3)-azide or FA2BG2S2(6 or 3)-azide or both A2BG2S2(6 or 3)-azide and FA2BG2S2(6 or 3)-azide, wherein the azide is part of the triazole linkage T.

In a further embodiment, the payload is a therapeutic agent, a diagnostic agent, radionuclide, or protecting group.

In a further embodiment, the therapeutic agent is a cytotoxic agent, an anti-inflammatory agent, a peptide, an immune agonist, or a nucleic acid molecule or nucleic acid analog.

In a further embodiment, the anti-inflammatory agent is a glucocorticoid receptor agonist. In a further embodiment, the anti-inflammatory agent is Cortisol, cortisone acetate, beclometasone, prednisone, prednisolone, methylprednisolone, betamethasone, trimcinolone, budesonide, dexamethasone, fluticasone, or mometasone.

In a further embodiment, the cytotoxic agent is selected from the group consisting of taxanes, anthracyclines, camptothecins, epothilones, mytomycins, combretastatins, vinca alkaloids, maytansinoids, enediynes, duocarmycins, tubulysins, amatoxins, bleomycins, dolastatins and auristatins, pyrrolobenzodiazepine dimers, indolinobenzodiazepine dimers, and radioisotopes.

The present invention further provides for the use of an antibody conjugate comprising the structure:

Ab-[B-T-L-Pay]ₙ

wherein Ab is an antibody; B is a bisected N-glycan wherein the bisecting sugar residue of the bisected N-glycan is a 6-azido-GlcNAc or GlcNAz residue having an azide functional group; L is a linker element comprising an alkyne functional group or a bond; Pay is a payload, which comprises an alkyne functional group when L is a bond; T is a triazole linkage formed between the linkage of the azide functional group and the alkyne functional group; and, n is an integer from 1-20 for the manufacture of a medicament for treatment of a disease or disorder in a subject. In particular embodiments, the disease or disorder is cancer.

In a further embodiment, L is a self-immolative linker. In a further embodiment, L has the formula L¹-L², wherein each of L¹ and L² is independently a linker element or a bond. In a further embodiment, L comprises a distal end comprising an alkyne containing group linked to the azide of the 6-azido-GlcNAc or GlcNAz residue of bisected N-glycan B in triazole linkage T and a proximal end linked to Pay.

In a further embodiment, the alkyne group is a strained cyclooctyne. In a further embodiment, the strained cyclooctyne is DBCO.

In a further embodiment, the bisected N-glycan B comprises a structure selected from the group consisting of A2B-azide; FA2B-azide; A2B[3]G1-azide; FA2 [3]BG1-azide; A2B[6]G1-azide; FA2B[6]G1-azide; A2BG2-azide; FA2BG2-azide; A2B[3]G1S1(6)-azide; FA2B[3]G1S1(6)-azide; A2B[6]G1S1(6)-azide; FA2B[6]G1S1(6)-azide; A2BG2S2(6)-azide; FA2BG2S2(6)-azide; A2B[3]G1S1(3)-azide; FA2B[3]G1S1(3)-azide; A2B[6]G1S1(3)-azide; FA2B[6]G1S1(3)-azide; A2BG2S2(3)-azide; FA2BG2S2(3)-azide; A1B[3]-azide; FA1B[3]-azide; A1B[3]G1-azide; FA1B[3]G1-azide; A1B[3]G1S1(6)-azide; A1B[3]G1S1(3)-azide; FA1B[3]G1S1(6)-azide; and, FA1B[3]G1S1(3)-azide, wherein the azide refers to the azide of 6-azido-GlcNAc or GlcNAz and which reacts with the alkyne to form the triazole linkage T.

In a further embodiment, wherein the bisecting sugar residue is azido-GlcNAc and the triazole linkage has the structure

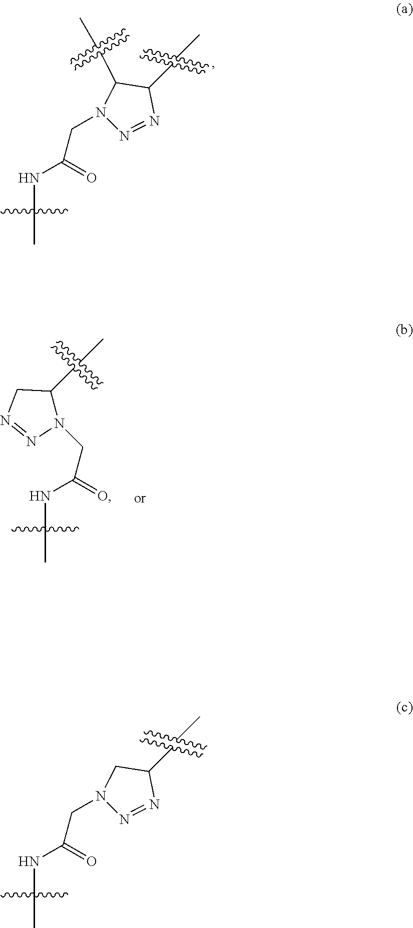

wherein the wavy line identifies the bond between the amine group of the GlcNAz residue and the C2 carbon of the glucose residue comprising the GlcNAz residue and the double wavy lines identify bonds between the alkyne functional group of L or Pay when L is a bond; or, the bisecting sugar residue is 6-azido-GlcNAc and the triazole linkage has the structure

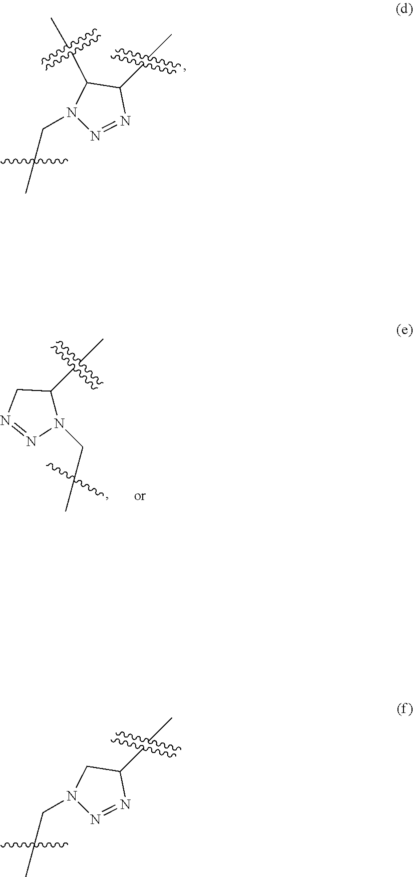

wherein the wavy line identifies the bond between the C5 and C6 carbons of the glucose residue comprising the 6-azido-GlcNAc residue and the double wavy lines identify bonds between the alkyne functional group of L or Pay when L is a bond.

In a further embodiment, the payload is a therapeutic agent, a diagnostic agent, radionuclide, or protecting group.

In a further embodiment, the therapeutic agent is a cytotoxic agent, an anti-inflammatory agent, a peptide, an immune agonist, or a nucleic acid molecule or nucleic acid analog.

In a further embodiment, the anti-inflammatory agent is a glucocorticoid receptor agonist. In a further embodiment, the anti-inflammatory agent is Cortisol, cortisone acetate, beclometasone, prednisone, prednisolone, methylprednisolone, betamethasone, trimcinolone, budesonide, dexamethasone, fluticasone, or mometasone.

In a further embodiment, the cytotoxic agent is selected from the group consisting of taxanes, anthracyclines, camptothecins, epothilones, mytomycins, combretastatins, vinca alkaloids, maytansinoids, enediynes, duocarmycins, tubulysins, amatoxins, bleomycins, dolastatins and auristatins, pyrrolobenzodiazepine dimers, indolinobenzodiazepine dimers, and radioisotopes.

The present invention further provides an antibody conjugate comprising the structure:

Ab-[B-T-L-Pay]ₙ

wherein Ab is an antibody; B is a bisected N-glycan wherein the bisecting sugar residue of the bisected N-glycan is a 6-azido-GlcNAc or GlcNAz residue having an azide functional group; L is a linker element comprising an alkyne functional group or a bond; Pay is a payload, which comprises an alkyne functional group when L is a bond; T is a triazole linkage formed between the linkage of the azide functional group and the alkyne functional group; and, n is an integer from 1-20 for treatment of a disease or disorder. In particular embodiments, the disease or disorder is cancer.

In a further embodiment, L is a self-immolative linker. In a further embodiment, L has the formula L¹-L², wherein each of L¹ and L² is independently a linker element or a bond. In a further embodiment, L comprises a distal end comprising an alkyne containing group linked to the azide of the 6-azido-GlcNAc or GlcNAz residue of bisected N-glycan B in triazole linkage T and a proximal end linked to Pay.

In a further embodiment, the alkyne group is a strained cyclooctyne. In a further embodiment, the strained cyclooctyne is DBCO.

In a further embodiment, the bisected N-glycan B comprises a structure selected from the group consisting of A2B-azide; FA2B-azide; A2B[3]G1-azide; FA2 [3]BG1-azide; A2B[6]G1-azide; FA2B[6]G1-azide; A2BG2-azide; FA2BG2-azide; A2B[3]G1S1(6)-azide; FA2B[3]G1S1(6)-azide; A2B[6]G1S1(6)-azide; FA2B[6]G1S1(6)-azide; A2BG2S2(6)-azide; FA2BG2S2(6)-azide; A2B[3]G1S1(3)-azide; FA2B[3]G1S1(3)-azide; A2B[6]G1S1(3)-azide; FA2B[6]G1S1(3)-azide; A2BG2S2(3)-azide; FA2BG2S2(3)-azide; A1B[3]-azide; FA1B[3]-azide; A1B[3]G1-azide; FA1B[3]G1-azide; A1B[3]G1S1(6)-azide; A1B[3]G1S1(3)-azide; FA1B[3]G1S1(6)-azide; and, FA1B[3]G1S1(3)-azide, wherein the azide refers to the azide of 6-azido-GlcNAc or GlcNAz and which reacts with the alkyne to form the triazole linkage T.

In a further embodiment, wherein the bisecting sugar residue is azido-GlcNAc and the triazole linkage has the structure

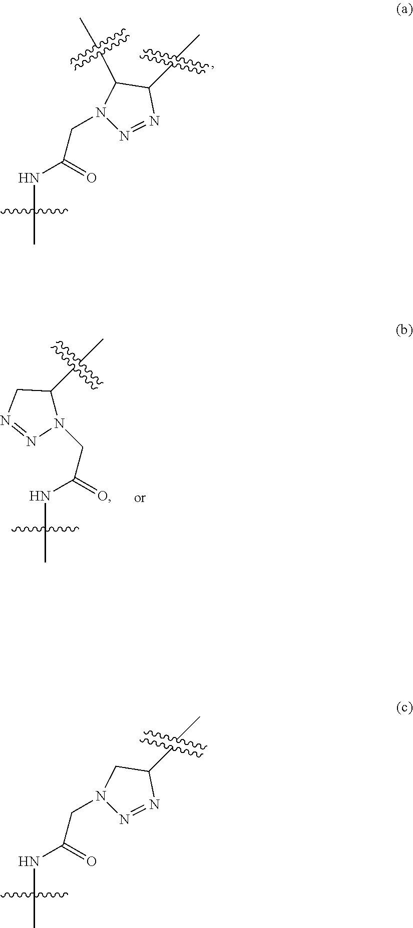

wherein the wavy line identifies the bond between the amine group of the GlcNAz residue and the C2 carbon of the glucose residue comprising the GlcNAz residue and the double wavy lines identify bonds between the alkyne functional group of L or Pay when L is a bond; or, the bisecting sugar residue is 6-azido-GlcNAc and the triazole linkage has the structure

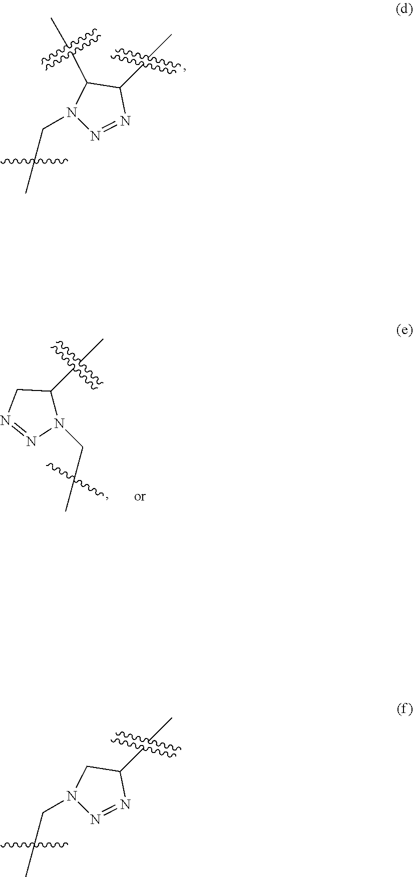

wherein the wavy line identifies the bond between the C5 and C6 carbons of the glucose residue comprising the 6-azido-GlcNAc residue and the double wavy lines identify bonds between the alkyne functional group of L or Pay when L is a bond.

In a further embodiment, the payload is a therapeutic agent, a diagnostic agent, radionuclide, or protecting group.

In a further embodiment, the therapeutic agent is a cytotoxic agent, an anti-inflammatory agent, a peptide, an immune agonist, or a nucleic acid molecule or nucleic acid analog.

In a further embodiment, the anti-inflammatory agent is a glucocorticoid receptor agonist. In a further embodiment, the anti-inflammatory agent is Cortisol, cortisone acetate, beclometasone, prednisone, prednisolone, methylprednisolone, betamethasone, trimcinolone, budesonide, dexamethasone, fluticasone, or mometasone.

In a further embodiment, the cytotoxic agent is selected from the group consisting of taxanes, anthracyclines, camptothecins, epothilones, mytomycins, combretastatins, vinca alkaloids, maytansinoids, enediynes, duocarmycins, tubulysins, amatoxins, bleomycins, dolastatins and auristatins, pyrrolobenzodiazepine dimers, indolinobenzodiazepine dimers, and radioisotopes.

The present invention further provides a method for treating a disease or disorder in a subject comprising:
administering a therapeutically effective amount of an antibody conjugate comprising the structure:

Ab-[B-T-L-Pay]ₙ

wherein Ab is an antibody; B is a bisected N-glycan wherein the bisecting sugar residue of the bisected N-glycan is a 6-azido-GlcNAc or GlcNAz residue having an azide functional group; L is a linker element comprising an alkyne functional group or a bond; Pay is a payload, which comprises an alkyne functional group when L is a bond; T is a triazole linkage formed between the linkage of the azide functional group and the alkyne functional group; and, n is an integer from 1-20. In particular embodiments, the disease or disorder is cancer.

In a further embodiment, L is a self-immolative linker. In a further embodiment, L has the formula L¹-L², wherein each of L¹ and L² is independently a linker element or a bond. In a further embodiment, L comprises a distal end comprising an alkyne containing group linked to the azide of the 6-azido-GlcNAc or GlcNAz residue of bisected N-glycan B in triazole linkage T and a proximal end linked to Pay.

In a further embodiment, the alkyne group is a strained cyclooctyne. In a further embodiment, the strained cyclooctyne is DBCO.

In a further embodiment, the bisected N-glycan B comprises a structure selected from the group consisting of A2B-azide; FA2B-azide; A2B[3]G1-azide; FA2 [3]BG1-azide; A2B[6]G1-azide; FA2B[6]G1-azide; A2BG2-azide; FA2BG2-azide; A2B[3]G1S1(6)-azide; FA2B[3]G1S1(6)-azide; A2B[6]G1S1(6)-azide; FA2B[6]G1S1(6)-azide; A2BG2S2(6)-azide; FA2BG2S2(6)-azide; A2B[3]G1S1(3)-azide; FA2B[3]G1S1(3)-azide; A2B[6]G1S1(3)-azide; FA2B[6]G1S1(3)-azide; A2BG2S2(3)-azide; FA2BG2S2(3)-azide; A1B[3]-azide; FA1B[3]-azide; A1B[3]G1-azide; FA1B[3]G1-azide; A1B[3]G1S1(6)-azide; A1B[3]G1S1(3)-azide; FA1B[3]G1S1(6)-azide; and, FA1B[3]G1S1(3)-azide, wherein the azide refers to the azide of 6-azido-GlcNAc or GlcNAz and which reacts with the alkyne to form the triazole linkage T.

In a further embodiment, wherein the bisecting sugar residue is azido-GlcNAc and the triazole linkage has the structure

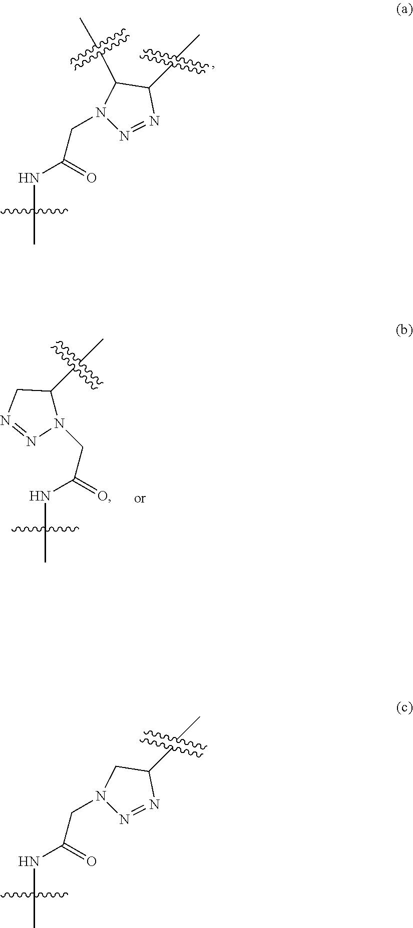

wherein the wavy line identifies the bond between the amine group of the GlcNAz residue and the C2 carbon of the glucose residue comprising the GlcNAz residue and the double wavy lines identify bonds between the alkyne functional group of L or Pay when L is a bond; or, the bisecting sugar residue is 6-azido-GlcNAc and the triazole linkage has the structure

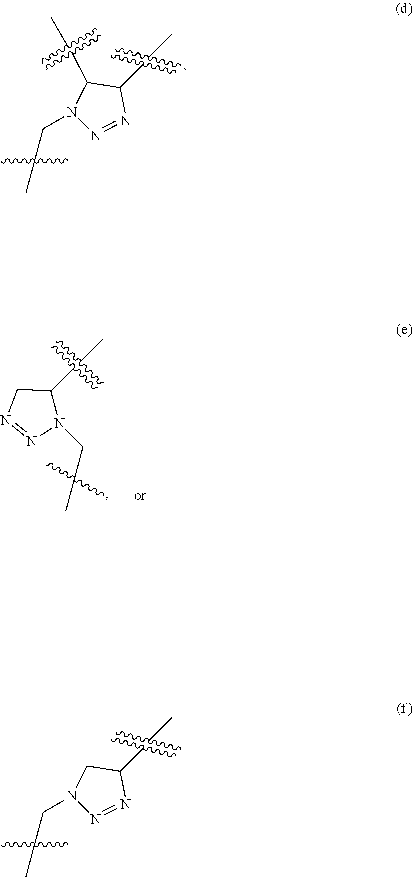

wherein the wavy line identifies the bond between the C5 and C6 carbons of the glucose residue comprising the 6-azido-GlcNAc residue and the double wavy lines identify bonds between the alkyne functional group of L or Pay when L is a bond.

In a further embodiment, the payload is a therapeutic agent, a diagnostic agent, radionuclide, or protecting group.

In a further embodiment, the therapeutic agent is a cytotoxic agent, an anti-inflammatory agent, a peptide, an immune agonist, or a nucleic acid molecule or nucleic acid analog.

In a further embodiment, the anti-inflammatory agent is a glucocorticoid receptor agonist. In a further embodiment, the anti-inflammatory agent is Cortisol, cortisone acetate, beclometasone, prednisone, prednisolone, methylprednisolone, betamethasone, trimcinolone, budesonide, dexamethasone, fluticasone, or mometasone.

In a further embodiment, the cytotoxic agent is selected from the group consisting of taxanes, anthracyclines, camptothecins, epothilones, mytomycins, combretastatins, vinca alkaloids, maytansinoids, enediynes, duocarmycins, tubulysins, amatoxins, bleomycins, dolastatins and auristatins, pyrrolobenzodiazepine dimers, indolinobenzodiazepine dimers, and radioisotopes.

The present invention further provides use of an antibody comprising the structure:

Ab-[B]ₙ

wherein Ab is an antibody; B is a bisected N-glycan wherein the bisecting sugar residue of the bisected N-glycan is a 6-azido-GlcNAc or GlcNAz residue having an azide functional group; and, n is an integer from 1-20 for the manufacture of a medicament for treatment of a disease or disorder. In particular embodiments, the disease or disorder is cancer.

In a further embodiment, the bisected N-glycan B comprises a structure selected from the group consisting of A2B-azide; FA2B-azide; A2B[3]G1-azide; FA2 [3]BG1-azide; A2B[6]G1-azide; FA2B[6]G1-azide; A2BG2-azide; FA2BG2-azide; A2B[3]G1S1(6)-azide; FA2B[3]G1S1(6)-azide; A2B[6]G1S1(6)-azide; FA2B[6]G1S1(6)-azide; A2BG2S2(6)-azide; FA2BG2S2(6)-azide; A2B[3]G1S1(3)-azide; FA2B[3]G1S1(3)-azide; A2B[6]G1S1(3)-azide; FA2B[6]G1S1(3)-azide; A2BG2S2(3)-azide; FA2BG2S2(3)-azide; A1B[3]-azide; FA1B[3]-azide; A1B[3]G1-azide; FA1B[3]G1-azide; A1B[3]G1S1(6)-azide; A1B[3]G1S1(3)-azide; FA1B[3]G1S1(6)-azide; and, FA1B[3]G1S1(3)-azide, wherein the azide refers to the azide of 6-azido-GlcNAc or GlcNAz.

The present invention further provides an antibody comprising the structure:

Ab-[B]ₙ

wherein Ab is an antibody; B is a bisected N-glycan wherein the bisecting sugar residue of the bisected N-glycan is a 6-azido-GlcNAc or GlcNAz having an azide functional group; and, n is an integer from 1-20 for the manufacture of a medicament for treatment of a disease or disorder. In particular embodiments, the disease or disorder is cancer.

In a further embodiment, the bisected N-glycan B comprises a structure selected from the group consisting of A2B-azide; FA2B-azide; A2B[3]G1-azide; FA2 [3]BG1-azide; A2B[6]G1-azide; FA2B[6]G1-azide; A2BG2-azide; FA2BG2-azide; A2B[3]G1S1(6)-azide; FA2B[3]G1S1(6)-azide; A2B[6]G1S1(6)-azide; FA2B[6]G1S1(6)-azide; A2BG2S2(6)-azide; FA2BG2S2(6)-azide; A2B[3]G1S1(3)-azide; FA2B[3]G1S1(3)-azide; A2B[6]G1S1(3)-azide; FA2B[6]G1S1(3)-azide; A2BG2S2(3)-azide; FA2BG2S2(3)-azide; A1B[3]-azide; FA1B[3]-azide; A1B[3]G1-azide; FA1B[3]G1-azide; A1B[3]G1S1(6)-azide; A1B[3]G1S1(3)-azide; FA1B[3]G1S1(6)-azide; and, FA1B[3]G1S1(3)-azide, wherein the azide refers to the azide of 6-azido-GlcNAc or GlcNAz.

The present invention further provides an antibody comprising the structure:

Ab-[B]ₙ

wherein Ab is an antibody; B is a bisected N-glycan wherein the bisecting sugar residue is azido-GlcNAc or GlcNAz having an azide functional group; and, n is an integer from 1-20 for the manufacture of an antibody conjugate.

In a further embodiment, the bisected N-glycan B comprises a structure selected from the group consisting of A2B-azide; FA2B-azide; A2B[3]G1-azide; FA2 [3]BG1-azide; A2B[6]G1-azide; FA2B[6]G1-azide; A2BG2-azide; FA2BG2-azide; A2B[3]G1S1(6)-azide; FA2B[3]G1S1(6)-azide; A2B[6]G1S1(6)-azide; FA2B[6]G1S1(6)-azide; A2BG2S2(6)-azide; FA2BG2S2(6)-azide; A2B[3]G1S1(3)-azide; FA2B[3]G1S1(3)-azide; A2B[6]G1S1(3)-azide; FA2B[6]G1S1(3)-azide; A2BG2S2(3)-azide; FA2BG2S2(3)-azide; A1B[3]-azide; FA1B[3]-azide; A1B[3]G1-azide; FA1B[3]G1-azide; A1B[3]G1S1(6)-azide; A1B[3]G1S1(3)-azide; FA1B[3]G1S1(6)-azide; and, FA1B[3]G1S1(3)-azide, wherein the azide refers to the azide of 6-azido-GlcNAc or GlcNAz.

The present invention provides a method for making a composition of antibodies comprising the structure:

Ab-[B]ₙ

wherein Ab is an antibody; B is a bisected N-glycan wherein the bisecting sugar residue of the bisected N-glycan is a 6-azido-GlcNAc or GlcNAz residue having an azide functional group; and, n is an integer from 1-20, comprising the steps of:
(a) providing an aqueous composition of N-glycosylated antibodies comprising a plurality of complex N-glycans;
(b) reacting the N-glycosylated antibodies with a neuraminidase and a galactosidase to produce N-glycosylated antibodies comprising predominantly GlcNAc terminated N-glycans;
(c) reacting the GlcNAc-terminated N-glycans with human N-acetylglucosaminyltransferase III (GnT-III or MGAT3) in the presence of 6-azido-GlcNAc or GlcNAz to produce antibodies comprising the formula Ab-[B]ₙ.

In a further step, the antibodies from step (c) are isolated to provide a composition comprising the antibodies comprising structure Ab-[B]ₙ.

In a further embodiment, the antibodies comprising structure Ab-[B]ₙ are reacted with one or more fucosidases to produce a composition comprising afucosylated antibodies comprising structure Ab-[B]ₙ.

In a particular embodiments, the bisected N-glycan B comprises predominantly a structure selected from the group consisting of A2B-azide and FA2B-azide, wherein the azide refers to the azide of 6-azido-GlcNAc or GlcNAz.

In particular embodiments, the antibodies in step (c) are reacted with a β1-4 galactosyltransferase in the presence of UDP-galactose to produce galactose-terminated bisected N-glycans comprising predominantly a structure selected from the group consisting of A2B[3]G1-azide; FA2B[3]G1-azide; A2B[6]G1-azide; FA2B[6]G1-azide; A2BG2-azide; and, FA2BG2-azide, wherein the azide refers to the azide of 6-azido-GlcNAc or GlcNAz.

In a further embodiment, the galactose-terminated bisected N-glycans are reacted with (i) α2-6-sialyltransferase in the presence of CMP-sialic acid to produce sialic acid terminated N-glycans comprising predominantly a structure selected from the group consisting of A2B[3]G1S1(6)-azide; FA2B[3]G1S1(6)-azide; A2B[6]G1S1(6)-azide; FA2B[6]G1S1(6)-azide; A2BG2S2(6)-azide; and, FA2BG2S2(6)-azide or (ii) α2-3-sialyltransferase in the presence of CMP-sialic acid to produce sialic acid terminated N-glycans comprising predominantly a structure selected from the group consisting of A2B[3]G1S1(3)-azide; FA2B[3]G1S1(3)-azide; A2B[6]G1S1(3)-azide; FA2B[6]G1S1(3)-azide; A2BG2S2(3)-azide; and, FA2BG2S2(3)-azide, wherein the azide refers to the azide of 6-azido-GlcNAc or GlcNAz.

In a further embodiment, the antibodies in step (b) are reacted with one or more N-acetyl-glucosaminidases to produce antibodies comprising paucimannose N-glycans, which are then reacted with GnT-I to produce antibodies comprising N-glycans comprising the structure A1[3] and FA1[3] and the resulting antibodies are reacted with GnT-III to produce bisected N-glycan B comprising a structure selected from the group consisting of A1B[3]-azide and FA1B[3]-azide, wherein the azide refers to the azide of 6-azido-GlcNAc or GlcNAz.

In particular embodiments, the antibodies in step (c) are further reacted with a β1-4 galactosyltransferase in the presence of UDP-galactose to produce galactose-terminated bisected N-glycans comprising predominantly a structure selected from the group consisting of A1B[3]-azide; FA1B[3]-azide; A1B[3]G1-azide; and FA1B[3]G1-azide, wherein the azide refers to the azide of 6-azido-GlcNAc or GlcNAz.

In a further embodiment, the galactose-terminated bisected N-glycans are reacted with (i) α2-6-sialyltransferase in the presence of CMP-sialic acid to produce sialic acid terminated N-glycans comprising predominantly a structure selected from the group consisting of A1B[3]G1S1(6)-azide and FA1B[3]G1S1(6)-azide; or (ii) α2-3-sialyltransferase in the presence of CMP-sialic acid to produce sialic acid terminated N-glycans comprising predominantly a structure selected from the group consisting of A1B[3]G1S1(6)-azide and FA1B[3]G1S1(6)-azide, wherein the azide refers to the azide of 6-azido-GlcNAc or GlcNAz.

The present invention further provides a method for producing an antibody conjugate comprising:
(a) providing an antibody comprising the structure Ab-[B]ₙ
   wherein Ab is an antibody; B is a bisected N-glycan wherein the bisecting sugar residue of the bisected N-glycan is a 6-azido-GlcNAc or GlcNAz residue having an azide functional group; and, n is an integer from 1-20; and
(b) conjugating the alkyne functional group of L-Pay or Pay, when L is a bond, to the azide functional group to produce an antibody conjugate comprising the structure Ab-[B-T-L-Pay]ₙ.

In a further embodiment of the method, the bisected N-glycan B comprises a structure selected from the group consisting of A2B-azide; FA2B-azide; A2B[3]G1-azide; FA2 [3]BG1-azide; A2B[6]G1-azide; FA2B[6]G1-azide; A2BG2-azide; FA2BG2-azide; A2B[3]G1S1(6)-azide; FA2B[3]G1S1(6)-azide; A2B[6]G1S1(6)-azide; FA2B[6]G1S1(6)-azide; A2BG2S2(6)-azide; FA2BG2S2(6)-azide; A2B[3]G1S1(3)-azide; FA2B[3]G1S1(3)-azide; A2B[6]G1S1(3)-azide; FA2B[6]G1S1(3)-azide; A2BG2S2(3)-azide; FA2BG2S2(3)-azide; A1B[3]-azide; FA1B[3]-azide; A1B[3]G1-azide; FA1B[3]G1-azide; A1B[3]G1S1(6)-azide; A1B[3]G1S1(3)-azide; FA1B[3]G1S1(6)-azide; and, FA1B[3]G1S1(3)-azide, wherein the azide refers to the azide of 6-azido-GlcNAc or GlcNAz, and which forms a triazole with an alkyne.

The present invention further provides an antibody comprising one or more N-glycans selected from the group consisting of A2B-azide; FA2B-azide; A2B[3]G1-azide; FA2 [3]BG1-azide; A2B[6]G1-azide; FA2B[6]G1-azide; A2BG2-azide; FA2BG2-azide; A2B[3]G1S1(6)-azide; FA2B[3]G1S1(6)-azide; A2B[6]G1S1(6)-azide; FA2B[6]G1S1(6)-azide; A2BG2S2(6)-azide; FA2BG2S2(6)-azide; A2B[3]G1S1(3)-azide; FA2B[3]G1S1(3)-azide; A2B[6]G1S1(3)-azide; FA2B[6]G1S1(3)-azide; A2BG2S2(3)-azide; FA2BG2S2(3)-azide; A1B[3]-azide; FA1B[3]-azide; A1B[3]G1-azide; FA1B[3]G1-Azide; A1B[3]G1S1(6)-azide; A1B[3]G1S1(3)-azide; FA1B[3]G1S1(6)-azide; and, FA1B[3]G1S1(3)-azide, wherein the azide refers to the azide of 6-azido-GlcNAc or GlcNAz.

The present invention further provides an antibody comprising one or more N-glycans selected from the group consisting of A2B-azide; FA2B-azide; A2B[3]G1-azide; FA2 [3]BG1-azide; A2B[6]G1-azide; FA2B[6]G1-azide; A2BG2-azide; FA2BG2-azide; A2B[3]G1S1(6)-azide; FA2B[3]G1S1(6)-azide; A2B[6]G1S1(6)-azide; FA2B[6]G1S1(6)-azide; A2BG2S2(6)-azide; FA2BG2S2(6)-azide; A2B[3]G1S1(3)-azide; FA2B[3]G1S1(3)-azide; A2B[6]G1S1(3)-azide; FA2B[6]G1S1(3)-azide; A2BG2S2(3)-azide; FA2BG2S2(3)-azide; A1B[3]-azide; FA1B[3]-azide; A1B[3]G1-azide; FA1B[3]G1-Azide; A1B[3]G1S1(6)-azide; A1B[3]G1S1(3)-azide; FA1B[3]G1S1(6)-azide; and, FA1B[3]G1S1(3)-azide, wherein the azide refers to the azide of 6-azido-GlcNAc.

The present invention further provides an antibody comprising one or more N-glycans selected from the group consisting of A2B-azide; FA2B-azide; A2B[3]G1-azide; FA2 [3]BG1-azide; A2B[6]G1-azide; FA2B[6]G1-azide; A2BG2-azide; FA2BG2-azide; A2B[3]G1S1(6)-azide; FA2B[3]G1S1(6)-azide; A2B[6]G1S1(6)-azide; FA2B[6]G1S1(6)-azide; A2BG2S2(6)-azide; FA2BG2S2(6)-azide; A2B[3]G1S1(3)-azide; FA2B[3]G1S1(3)-azide; A2B[6]G1S1(3)-azide; FA2B[6]G1S1(3)-azide; A2BG2S2(3)-azide; FA2BG2S2(3)-azide; A1B[3]-azide; FA1B[3]-azide; A1B[3]G1-azide; FA1B[3]G1-Azide; A1B[3]G1S1(6)-azide; A1B[3]G1S1(3)-azide; FA1B[3]G1S1(6)-azide; and, FA1B[3]G1S1(3)-azide, wherein the azide refers to the azide of GlcNAz.

The present invention further provides an antibody-drug conjugate (ADC) comprising an antibody disclosed herein conjugated to a payload comprising an alkyne group. In a further embodiment, the payload is a cytotoxic agent, an anti-inflammatory agent, a radionuclide, or diagnostic agent.

### BRIEF DESCRIPTION OF THE DRAWINGS

FIG. 1A shows a scheme for converting FA2 to FA2B using N-acetylglucosaminyltransferase III (GnT-III) in the presence of UDP-azido-GlcNAc (N₃-UDP-▪).

FIG. 1B shows installing azido-GlcNAc (N₃-UDP-▪) to antibody N-glycans through the activity of N-acetylglucosaminyltransferase III (GnT-III). Shown is dose-dependent treatment of GnT-III on intact human serum IgGs with FA2 and FA2B glycoforms to introduce the bisecting azido-GlcNAc residue thereon. The bottom chromatogram shows the control experiment using UDP-GlcNAc as the saccharide donor, instead of UDP-azido-GlcNAc. The substrate serum IgG was treated with neuraminidase and galactosidase to enrich the substrate glycan species (FA2) for GnT-III reaction. Concentration indicates GnT-III-to-IgG equivalent (2 hours reaction).

FIG. 1C shows a comparison of GnT-III activity between different UDP-GlcNAc derivative substrates.

FIG. 2 shows glycan profile analysis using liquid chromatography. Upper panel: glycan standards isolated from human serum. Oxford notation is used for glycan nomenclature. Bottom panel: native anti-HER2 mouse IgG2a glycan profile. Glycan mass was confirmed by a tandem mass spectrometer (data available on inquiry).

FIGS. 3A-3D show payload conjugation through the N-glycan bisecting site.

FIG. 3A shows an exemplary scheme of the bisecting glycan conjugation strategy. The N-glycans of an IgG comprising fucose are harmonized to FA2. GnT-III is then used to install an azido-GlcNAc to the bisecting position of IgG N-glycans. A payload comprising a dibenzocyclooctyne (DBCO) group is conjugated to the azide group of the azido-GlcNAc. The resulting conjugate comprises the payload linked to the bisecting N-glycan in a triazole linkage. The oval shape represents the payload.

FIG. 3B shows an exemplary payload comprising the cytotoxin MMAE linked to a DBCO group via PEG4 linker.

FIG. 3C shows deconvoluted mass of the mouse IgG substrate before and after monomethylauristatin E (MMAE) conjugation to the exemplary payload of FIG. 3B. Data shows the heavy chains (HC) only for simplicity. No conjugation was observed on the light chains. The oval shape represents the payload.

FIG. 3D shows LC-MS analysis on isolated substrate N-glycans confirmed the formation of the FA2-azido glycoform after glycan remodeling. N-glycans are labeled with RapiFluor-MS for improved signal-to-noise ratio in the analysis.

FIG. 4A-4C shows a linker cleavage study using Cathepsin B and Endoglycosidase S (Endo S).

FIG. 4A shows that mass spectrometry analysis indicated that Cathepsin B cleaves the Valine-Citruline-p-aminobenzylalcohol (Val-Cit-PAB) linker (ii); while Endo S cleaves the β1-4 GlcNAc-GlcNAc linkage (iii). In-line fragmentation of the MMAE/linker was observed (i) in the analysis.

FIG. 4B shows formulas of MMAE and linker with (FIG. 4B-1, FIG. 4B-2, and FIG. 4B-3) and without (FIG. 4B-3, FIG. 4B-4, and FIG. 4B-5) the cleavable Val-Cit-PABC peptides conjugated to the FA2B-azide N-glycans of an antibody. Dashed lines indicated the cleavage sites observed in the MS analysis.

FIG. 4C shows quantitative analysis of ADC recovery from rat plasma after incubation for 3 days. Day 0: the samples were frozen immediately after the ADCs were added to plasma.

FIGS. 5A-5B shows synthesis of human IgG-MMAE ADC using bisecting-glycan bridged conjugation to FA2B-azide.

FIG. 5A shows LC-MS analysis of isolated N-glycans from anti-HER2 human IgG1 (Trastuzumab biosimilar) and its glycoengineering into FA2B-azide and A2B-azide glycoforms.

FIG. 5B shows MMAE conjugation was confirmed by deconvoluted IgG mass. Data shows the heavy chains (HC) only for simplicity. No conjugation was observed on the light chains. The oval shape represents the payload.

FIGS. 6A-6D show payload conjugation through bisecting glycans is biocompatible. Isotype hIgG1 refers to a generic human IgG isotype standard; hIgG1 refers to anti-HER2 human IgG1 (Trastuzumab biosimilar); and hIgG1-MMAE refers to Trastuzumab biosimilar conjugated to MMAE via the bisecting N-glycan FA2B-azide.

FIG. 6A shows a comparison of HER2 binding ability before and after MMAE conjugation to FA2B-azide using biolayer interferometry (BLI). Star marks: show that isotype hIgG1 has no detectable binding signal in the tested concentration range.

FIG. 6B shows flow cytometry assay confirmed the binding of hIgG1-MMAE to SKBR3 surface. A: cells without secondary antibody staining; B: cells without hIgG1-MMAE treatment; C: cells treated with both hIgG1-MMAE and secondary antibody. The hIgG1-MMAE was used as the primary antibody to detect HER2 and an anti-human IgG antibody was used as the secondary antibody.

FIG. 6C shows hIgG1-MMAE internalization study using SKBR3 cells and Incucyte® ADC internalization assay (Sartorius AG).

FIG. 6D shows a comparison of FcγRs binding ability before and after MMAE conjugation using BLI.

FIG. 6E shows cell viability studies demonstrate target-specific toxicity of the hIgG-MMAE compared to ADCs synthesized by using lysine conjugation or engineered cysteine conjugation (positive controls). ADCs made of isotype human IgG1 were used as the negative controls. FA2B-azide structure is shown.

FIG. 6F shows a comparison of HER2-expressing cell killing activity between hIgG-MMAE conjugated to FA2B-azide or FA2BG2-azide.

FIGS. 7A-7C shows glycoengineering of ADCs and its impact on FcγR interactions.

FIG. 7A shows a scheme of glycoengineering methods to control ADC glycoforms.

FIG. 7B-1 shows chromatographic analysis of engineered N-glycans.

FIG. 7B-2 Mass spectrometry analysis of the reduced human IgG1 Trastuzumab biosimilar with engineered N-glycoforms and the corresponding MMAE ADCs. HC stands for heavy chain. The oval shape represents the payload.

FIG. 7C shows FcγR binding response assay using BLI reveals the regulatory effects of ADC glycoforms. The binding assay was triplicated. Curve width indicates the standard deviation at each time point.

FIG. 8 shows optimization of GnT-III reactions to install azido-GlcNAc on intact human serum IgG. Serum IgG with FA2 glycoform was used as the substrate. The conversion ratio was calculated based on the consumption of the FA2 glycan species in the population. N.D.=not detectable. The figure presents each data point and the mean value.

FIG. 9 shows GnT-III substrate selectivity on serum IgG with different glycoforms. GnT-III only functions on N-glycans having a terminal GlcNAc residue. The figure shows the mean value and standard deviation (N=3). The transformation of N-glycan structures was carried out by successive enzymatic remodeling as reported in Hsu, Y.-P. et al. Successive remodeling of IgG glycans using a solid-phase enzymatic platform. Communications Biology 5, 328 (2022).

FIG. 10 shows synthesis of mouse IgG-MMAE ADC. Panel A, shows a table summarizing the observed mass changes of mouse IgG heavy chain (HC) after glycan harmonization, azido-GlcNAc introduction, and MMAE conjugation reactions. Refer to FIG. 3C for the mass spectra. Panel B, shows that a change in hydrophilicity was observed after MMAE conjugation, as indicated by hydrophilic interaction chromatography. Panel C, shows that size-exclusion chromatography did not show any significant change in protein size after MMAE conjugation, indicating no aggregation or degradation after the reaction.

FIG. 11 shows synthesis of human IgG-MMAE ADC. Panel A, the table summarizes the observed mass changes of human IgG heavy chain (HC) after glycan harmonization, azido-GlcNAc introduction, and MMAE conjugation reactions. Refer to FIG. 5B for the mass spectra. Panel B shows a change in hydrophilicity was observed after MMAE conjugation, as indicated by hydrophilic interaction chromatography. Panel C shows that size-exclusion chromatography did not show any significant change in protein size after MMAE conjugation.

FIG. 12A shows a Western Blot analysis of human IgG1-MMAE ADC binding ability to human HER2. The ADC was used as the primary antibody to detect HER2, and an anti-human IgG antibody was used as the secondary antibody.

FIG. 12B shows cell images were collected from the Incucyte® ADC internalization assay. FabFluoris a pH-sensitive probe that turns red color after internalization into cell lysosome.

FIG. 13 shows a comparison of HER2 and FcγRs binding ability before and after MMAE conjugation to the mouse IgG2a substrate using BLI. Star mark: no detectable binding signal in the tested concentration range.

FIG. 14 shows that melting temperature (Tm) and aggregation temperature (Tagg) assays showed no detectable destabilizing effect after the payload conjugation. Each data point and the mean value are presented.

FIG. 15A, FIG. 15B, FIG. 15C, FIG. 15D and FIG. 15E show various biantennary and bisected N-glycans structures drawn following the recommendations of the Symbol Nomenclature for Glycans (SNFG), National Institutes of Health (NIH), National Library of Medicine, National Center for Biotechnology Information, Bethesda, MD and may be accessed at www.ncbi.nlm.nih.gov/glycans/snfg.html. The names of the structures correspond to the Oxford Notation Names in Table 1. The β1-N linkage shown is the linkage between the reducing end of the chitobiose core (GlcNAcβ1-4GlcNAc) to the sidechain amino group of Asn that comprises the N-glycosylation site Asn-X-Thr/Ser wherein X is any amino acid except Pro.

FIG. 16A, FIG. 16B, and FIG. 16C show exemplary azido-GlcNAc bisected N-glycan structures of the present invention drawn following the recommendations of the SNFG. The β1-N linkage shown is the linkage between the reducing end of the chitobiose core to the sidechain amino group of Asn that comprises the N-glycosylation site Asn-X-Thr/Ser wherein X is any amino acid except Pro.

FIG. 17 and FIG. 17-1 show a representative structure of N-glycans comprising an A2B-azide core linked to the Asn residue of Ab or an FA2B-azide core linked to the Asn residue of Ab. As shown, n is an integer from 1 to 20 and Ab is antibody.

FIG. 18 and FIG. 18-1 show a representative structure of N-glycans comprising an A2B-triazole-L-payload core linked to the Asn residue of Ab or an FA2B-triazole-L-payload core linked to the Asn residue of Ab. As shown, n is an integer from 1 to 20, Pay is payload, L is a linker or a bond and Ab is antibody.

FIG. 19 and FIG. 19-1 show a representative structure of N-glycans comprising an A1B[3]-azide core linked to the Asn residue of Ab or FA1B[3]-azide core linked to the Asn residue of Ab. As shown, n is an integer from 1 to 20 and Ab is antibody.

FIG. 20 and FIG. 20-1 show a representative structure of N-glycans comprising an A1B[3]-triazole-payload core linked to the Asn residue of Ab or an FA1B[3]-triazole-L-payload core linked to the Asn residue of Ab. As shown, n is an integer from 1 to 20, Pay is payload, L is linker or a bond, and Ab is antibody.

### DETAILED DESCRIPTION OF THE INVENTION

### Definitions

As used herein, the terms “N-glycan” and “glycoform” are used interchangeably and refer to an N-linked oligosaccharide, for example, one that is attached by an asparagine-N-acetylglucosamine linkage to an asparagine residue of a polypeptide. N-linked glycoproteins contain an N-acetylglucosamine residue linked to the amide nitrogen of an asparagine residue in the protein. The predominant sugars found on glycoproteins are glucose (Glc), galactose (Gal), mannose (Man), fucose (Fuc), N-acetylgalactosamine (GalNAc), N-acetylglucosamine (GlcNAc) and sialic acid (Sia). Sialic acids are a class of α-keto acid sugars with a nine-carbon backbone. The most common member of this group is N-acetylneuraminic acid (Neu5Ac or NANA) found in animals and some prokaryotes. The processing of the sugar groups occurs co-translationally in the lumen of the endoplasmic reticulum (ER) and continues post-translationally in the Golgi apparatus for N-linked glycoproteins.

N-glycans have a common pentasaccharide core of Man₃GlcNAc₂ comprising a mannose linked at its reducing end to the nonreducing end of a chitobiose core (GlcNAcβ1-4GlcNAc) in a β1,4 linkage and one mannose residue linked to the β1,4 linked mannose in an α1,3 linkage and the other mannose linked to the β1,4-linked mannose in an α1,6 linkage, represented by the structure showing the Man₃GlcNAc₂ linked to an asparagine residue comprising an N-glycosylation site in a glycoprotein

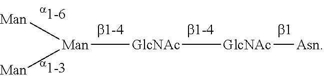

The GlcNAc residue at the reducing end may also be linked to a fucose residue in an α1,6 linkage. Usually, N-glycan structures are presented with the nonreducing end to the left and the reducing end to the right or the nonreducing end at the top and the reducing end at the bottom. The reducing end of the N-glycan is the end that is attached to the Asn residue comprising the glycosylation site on the protein. N-glycans differ with respect to the number of branches (antennae) comprising peripheral sugars (e.g., GlcNAc, galactose, fucose and sialic acid) that are added to the Man₃GlcNAc₂ (“Man₃”) core structure, which is also referred to as the “trimannose core”, the “pentasaccharide core”, the trimannosyl core”, or the “paucimannose core”.

N-glycans are classified according to their branched constituents (e.g., high mannose, complex, or hybrid). A “high mannose” type N-glycan comprises five or more mannose residues. A “complex” type N-glycan typically has at least one GlcNAc residue attached in a β1,2 linkage to the nonreducing end of the mannose residue at the nonreducing end of the 1,3 mannose arm and at least one GlcNAc residue attached in a β1,2 linkage to the nonreducing end of the mannose residue at the nonreducing end of the 1,6 mannose arm of the trimannose core. Complex N-glycans may further include galactose (“Gal”) or N-acetylgalactosamine (“GalNAc”) residues that are optionally further linked to sialic acid (“Sia”) or Sia derivatives (e.g., “NANA” or “NeuAc”, where “Neu” refers to neuraminic acid and “Ac” refers to acetyl). Complex N-glycans may also have intrachain substitutions comprising “bisecting” GlcNAc residue and core fucose (“Fuc”) residue. Complex N-glycans may also have multiple antennae on the “trimannose core,” often referred to as “multiantennary N-glycans.” A “hybrid” N-glycan comprises at least one GlcNAc attached in a β1,2 linkage to the nonreducing end of the mannose residue at the nonreducing end of the 1,3 mannose arm of the trimannose core and zero or more mannoses attached to the nonreducing end of the mannose on the nonreducing end of the 1,6 mannose arm of the trimannose core. The GlcNAc residue may then be attached to a galactose residue and the galactose residue may be attached to a sialic acid residues. The various N-glycans are also referred to as “glycoforms.”

With respect to complex N-glycans, the terms “G-2”, “G-1”, “G0”, “G1”, “G2”, “A1”, and “A2” mean the following. “G-2” refers to an N-glycan structure that can be characterized as Man₃GlcNAc₂; the term “G-1” refers to an N-glycan structure that can be characterized as GlcNAcMan₃GlcNAc₂; the term “G0” refers to an N-glycan structure that can be characterized as GlcNAc₂Man₃GlcNAc₂; the term “G1” refers to an N-glycan structure that can be characterized as GalGlcNAc₂Man₃GlcNAc₂; the term “G2” refers to an N-glycan structure that can be characterized as Gal₂GlcNAc₂Man₃GlcNAc₂; the term “A1” refers to an N-glycan structure that can be characterized as SiaGal₂GlcNAc₂Man₃GlcNAc₂; and, the term “A2” refers to an N-glycan structure that can be characterized as Sia₂Gal₂GlcNAc₂Man₃GlcNAc₂. Unless otherwise indicated, the terms “G-2”, “G-1”, “G0”, “G1”, “G2”, “A1”, and “A2” refer to N-glycan species that lack fucose attached to the GlcNAc residue at the reducing end of the N-glycan. When the term includes an “F”, the “F” indicates that the N-glycan species contains a fucose residue on the GlcNAc residue at the reducing end of the N-glycan. For example, G0F, G1F, G2F, A1F, and A2F all indicate that the N-glycan further includes a fucose residue attached to the GlcNAc residue at the reducing end of the N-glycan. Lower eukaryotes such as yeast and filamentous fungi do not normally produce N-glycans that produce fucose.

Complex N-glycans include biantennary N-glycans, bisected N-glycans, and multiantennary N-glycans. With respect to a biantennary N-glycan, the N-glycan comprises a 1,6 mannose arm and a 1,3 mannose arm in which the 1,6 mannose arm and the 1,3 mannose arm each comprises one GlcNAc residue linked in a β1,2 linkage to the mannose residue at the non-reducing end of the arm. Each GlcNAc residue may be independently further attached to a galactose residue and each galactose residue may be independently further attached to a sialic acid residue. Thus, biantennary N-glycans may include N-glycans having the short-hand formula GlcNAc₂Man₃GlcNAc₂, Gal₍₁₋₂₎GlcNAc₂Man₃GlcNAc₂, or Sia₍₁₋₂₎Gal₍₁₋₂₎GlcNAc₂Man₃GlcNAc₂. The term “(1-2)” refers to 1 or 2 sugar residues.

With respect to multiantennary N-glycans, the term “multiantennary N-glycan” refers to a biantennary N-glycan that further comprises (i) a GlcNAc residue attached in a β1,4 linkage to the nonreducing end of the mannose residue comprising the non-reducing end of the 1,6 arm or the 1,3 arm of the N-glycan or (ii) a GlcNAc residue is attached in a β1,4 linkage to the nonreducing end of the mannose residue comprising the non-reducing end of the 1,6 arm and a GlcNAc residue is attached in a β1,4 linkage to the nonreducing end of the mannose residue comprising the non-reducing end of the 1,3 arm of the N-glycan. Each GlcNAc residue may be independently further attached to a galactose residue and the galactose residue may be independently further attached to a sialic acid residue. Thus, multiantennary N-glycans can be characterized by the short-hand formulas GlcNAc₍₃₋₄₎Man₃GlcNAc₂, Gal₍₁₋₄₎GlcNAc₍₃₋₄₎Man₃GlcNAc₂, or Sia₍₁₋₄₎Gal₍₁₋₄₎GlcNAc₍₃₋₄₎Man₃GlcNAc₂. The term “(1-4)” refers to 1, 2, 3, or 4 residues and the term “(3-4)” refers to 3 or 4 sugar residues.

With respect to bisected N-glycans, the term “bisected N-glycan” refers to N-glycans in which the reducing end of a GlcNAc residue is linked in a β1,4 linkage to the nonreducing end of the central mannose residue at the nonreducing end of the trimannose core. A bisected N-glycan may be characterized by the formula GlcNAc₃Man₃GlcNAc₂ wherein each mannose residue is linked at its non-reducing end to a GlcNAc residue. In contrast, when a multiantennary N-glycan is characterized as GlcNAc₃Man₃GlcNAc₂, the short-hand formula indicates that two GlcNAc residues are linked to the mannose residue at the non-reducing end of one of the two arms of the N-glycan and one GlcNAc residue is linked to the mannose residue at the non-reducing end of the other arm of the N-glycan. While the GlcNAc residues at the end of the 1,3 and 1,6 arms of the bisected N-glycan may each be further linked to a galactose residue, which may be further linked to a sialic acid residue, the bisecting GlcNAc residue is not further extended.

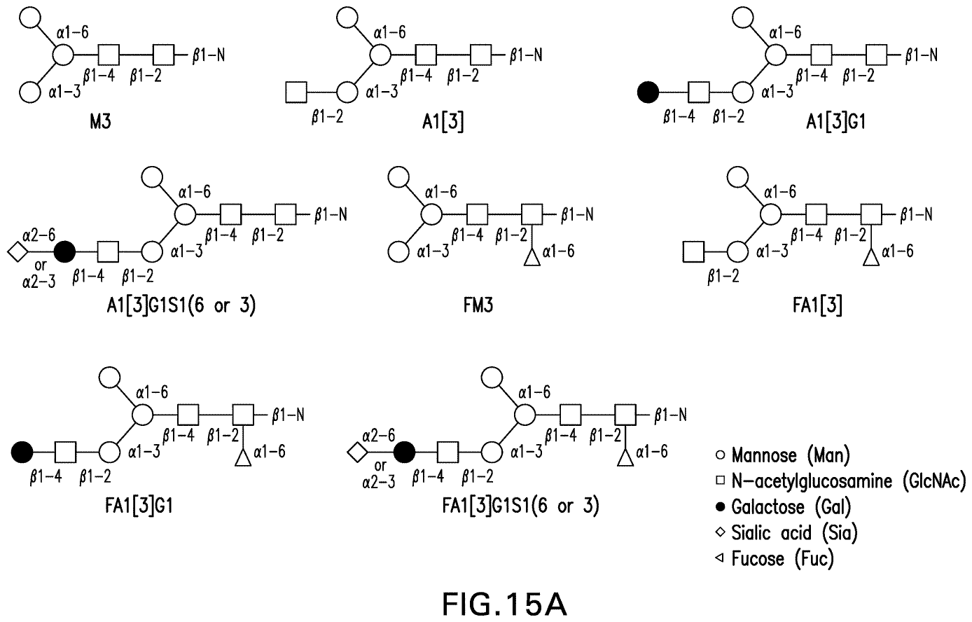

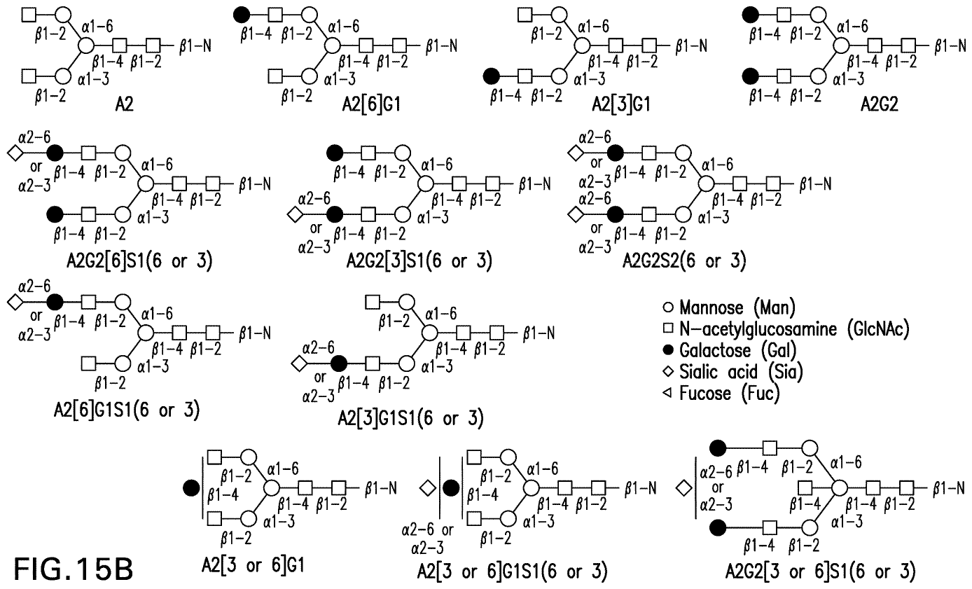

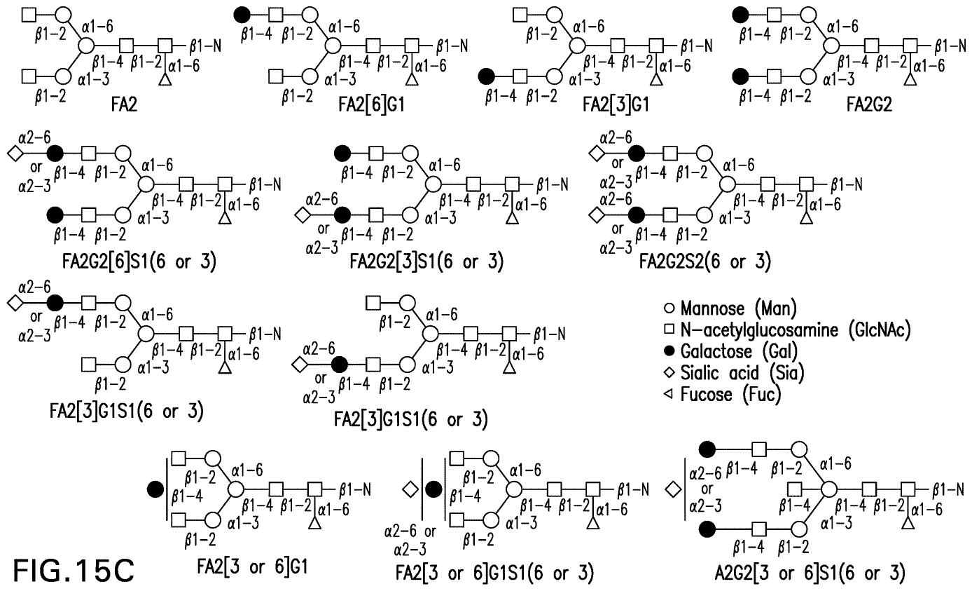

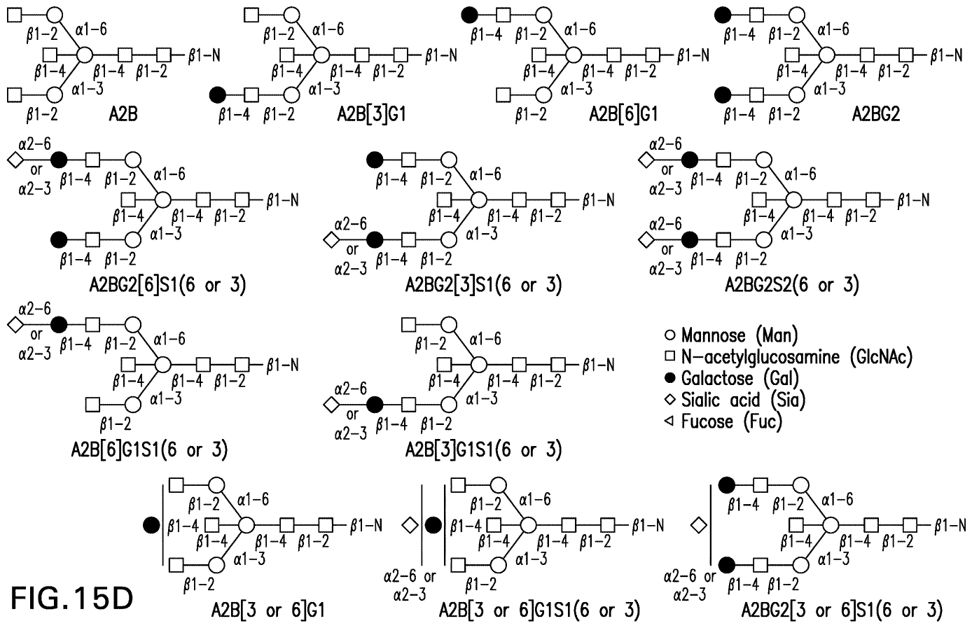

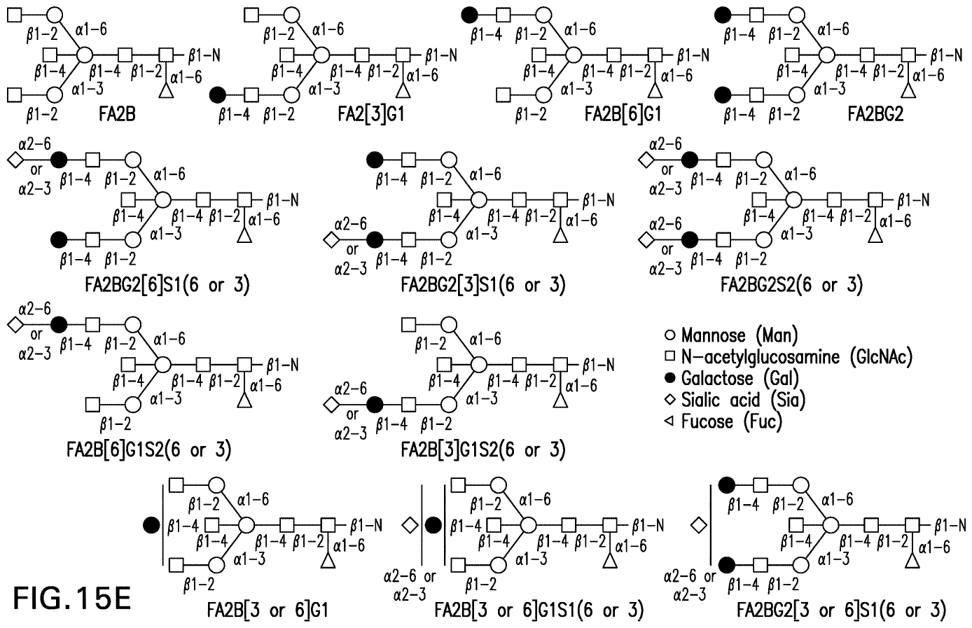

Table 1 provides a list of representative biantennary and bisected N-glycans, which includes the linkage to the Asn residue in the N-glycosylation site Asn-X-Ser/Thr, wherein X may be any amino acid other than proline. FIG. 15A, FIG. 15B, FIG. 15C, and FIG. 15D shows these various biantennary and bisected N-glycans structures drawn following the recommendations of the Symbol Nomenclature for Glycans (SNFG), National Institutes of Health (NIH), National Library of Medicine, National Center for Biotechnology Information, Bethesda, MD and may be access at www.ncbi.nlm.nih.gov/glycans/snfg.html.

TABLE 1

| | | Short- | |
|---|---|---|---|
| | Oxford | hand | |
| No. | Notation Name | Name | Formula |
| 1 | M3 | G-2 | Manα1-3(Manα1-6)Manβ1-4GlcNAcβ1-4GlcNAcβ1- |
| | | | Asn |
| 2 | A1[3] | G-1 | GlcNAcβ1-2Manα1-3(Manα1-6)Manβ1-4GlcNAcβ1- |
| | | | 4GlcNAcβ1-Asn |
| 3 | A1B[3] | G-1B | GlcNAcβ1-2Manα1-3(Manα1-6)(GlcNAcβ1-4)Manβ1- |
| | | | 4GlcNAcβ1-4GlcNAcβ1-Asn |
| 4 | A1[6] | G-1 | Manα1-3(GlcNAcβ1-2Manα1-6)Manβ1-4GlcNAcβ1- |
| | | | 4GlcNAcβ1-Asn |
| 5 | A1B[6] | G-1B | Manα1-3(GlcNAcβ1-2Manα1-6)(GlcNAcβ1-4)Manβ1- |
| | | | 4GlcNAcB1-4GlcNAcβ1-Asn |
| 6 | A2 | G0 | GlcNAcβ1-2Manα1-3(GlcNAcβ1-2Manα1-6)Manβ1- |
| | | | 4GlcNAcβ1-4GlcNAcβ1-Asn |
| 7 | A2B | G0B | GlcNAcβ1-2Manα1-3(GlcNAcβ1-2Manα1-6)(GlcNAcβ1-4) |
| | | | Manβ1-4GlcNAcβ1-4GlcNAcβ1-Asn |
| 8 | A1[3]G1 | G-1G1 | Galβ1-4GlcNAcβ1-2Manα1-3(Manα1-6)Manβ1- |
| | | | 4GlcNAcβ1-4GlcNAcB1-Asn |
| 9 | A1B[3]G1 | G-1B | Galβ1-4GlcNAcB1-2Manα1-3(Manα1-6)(GlcNAcβ1-4) |
| | | | Manβ1-4GlcNAcβ1-4GlcNAcβ1-Asn |
| 10 | A1[6]G1 | G-1G1 | Manα1-3(Galβ1-4GlcNAcβ1-2Manα1-6)Manβ1- |
| | | | 4GlcNAcβ1-4GlcNAcβ1-Asn |
| 11 | A1B[6]G1 | G-1BG1 | Manα1-3(Galβ1-4GlcNAcβ1-2Manα1-6)(GlcNAcβ1-4) |
| | | | Manβ1-4GlcNAcβ1-4GlcNAcβ1-Asn |
| 12 | A2[3]G1 | G1 | Galβ1-4GlcNAcβ1-2Manα1-3(GlcNAcβ1-2Manα1-6) |
| | | | Manβ1-4GlcNAcβ1-4GlcNAcβ1-Asn |
| 13 | A2B[3]G1 | G1B | Galβ1-4GlcNAcβ1-2Manα1-3(GlcNAcβ1-2Manα1-6) |
| | | | (GlcNAcβ1-4)Manβ1-4GlcNAcβ1-4GlcNAcβ1-Asn |
| 14 | A2[6]G1 | G1 | GlcNAcB1-2Manα1-3(Galβ1-4GlcNAcB1-2Manα1-6) |
| | | | ManB1-4GlcNAcB1-4GlcNAcβ1-Asn |
| 15 | A2B[6]G1 | G1B | GlcNAcB1-2Manα1-3(Galβ1-4GlcNAcB1-2Manα1-6) |
| | | | (GlcNAcβ1-4)Manβ1-4GlcNAcB1-4GlcNAcB1-Asn |
| 16 | A2G2 | G2 | Galβ1-4GlcNAcβ1-2Manα1-3(Galβ1-4GlcNAcβ1- |
| | | | 2Manα1-6)Manβ1-4GlcNAcB1-4GlcNAcβ1-Asn |
| 17 | A2BG2 | G2B | Galβ1-4GlcNAcβ1-2Manα1-3(Galβ1-4GlcNAcβ1- |
| | | | 2Manα1-6)(GlcNAcβ1-4)Manβ1-4GlcNAcβ1- |
| | | | 4GlcNAcβ1-Asn |
| 18 | A1[3]G1S1 | G-1A1 | Siaα2-3Galβ1-4GlcNAcβ1-2Manα1-3(Manα1-6)Manβ1- |
| | | | 4GlcNAcβ1-4GlcNAcβ1-Asn |
| 19 | A1B[3]G1S1 | G-1BA1 | Siaα2-3Galβ1-4GlcNAcβ1-2Manα1-3(Manα1-6) |
| | | | (GlcNAcβ1-4)Manβ1-4GlcNAcβ1-4GlcNAcβ1-Asn |
| 20 | A1[6]G1S1 | G-1A1 | Manα1-3(Siaα2-3Galβ1-4GlcNAcβ1-2Manα1-6)Manβ1- |
| | | | 4GlcNAcβ1-4GlcNAcβ1-Asn |
| 21 | A1B[6]G1S1 | G-1BA1 | Manα1-3(Siaα2-3Galβ1-4GlcNAcβ1-2Manα1-6) |
| | | | (GlcNAcβ1-4)Manβ1-4GlcNAcβ1-4GlcNAcβ1-Asn |
| 22 | A2[3]G1S1 | A1 | Siaα2-3Galβ1-4GlcNAcβ1-2Manα1-3(GlcNAcβ1- |
| | | | 2Manα1-6)Manβ1-4GlcNAcβ1-4GlcNAcβ1-Asn |
| 23 | A2B[3]G1S1 | A1B | Siaα2-3Galβ1-4GlcNAcβ1-2Manα1-3(GlcNAcβ1- |
| | | | 2Manα1-6)(GlcNAcβ1-4)Manβ1-4GlcNAcβ1- |
| | | | 4GlcNAcβ1-Asn |
| 24 | A2[6]G1S1 | A1 | GlcNAcβ1-2Manα1-3(Siaα2-3Galβ1-4GlcNAcβ1- |
| | | | 2Manα1-6)Manβ1-4GlcNAcβ1-4GlcNAcβ1-Asn |
| 25 | A2B[6]G1S1 | A1B | GlcNAcβ1-2Manα1-3(Siaα2-3Galβ1-4GlcNAcβ1- |
| | | | 2Manα1-6)(GlcNAcβ1-4)Manβ1-4GlcNAcβ1- |
| | | | 4GlcNAcβ1-Asn |
| 26 | A2G2[3]S1 | A1 | Siaα2-3Galβ1-4GlcNAcβ1-2Manα1-3(Galβ1- |
| | | | 4GlcNAcβ1-2Manα1-6)Manβ1-4GlcNAcβ1- |
| | | | 4GlcNAcβ1-Asn |
| 27 | A2BG2[3]S1 | A1B | Siaα2-3Galβ1-4GlcNAcβ1-2Manα1-3(Galβ1- |
| | | | 4GlcNAcβ1-2Manα1-6)(GlcNAcβ1-4)Manβ1- |
| | | | 4GlcNAcβ1-4GlcNAcβ1-Asn |
| 28 | A2G2[6]S1 | A1 | Galβ1-4GlcNAcβ1-2Manα1-3(Siaα2-3Galβ1- |
| | | | 4GlcNAcβ1-2Manα1-6)Manβ1-4GlcNAcβ1- |
| | | | 4GlcNAcβ1-Asn |
| 29 | A2BG2[6]S1 | A1B | Galβ1-4GlcNAcβ1-2Manα1-3(Siaα2-3Galβ1- |
| | | | 4GlcNAcβ1-2Manα1-6)(GlcNAcβ1-4)Manβ1- |
| | | | 4GlcNAcβ1-4GlcNAcβ1-Asn |
| 30 | A2G2S2 | A2 | Siaα2-3Galβ1-4GlcNAcβ1-2Manα1-3(Siaα2-3Galβ1- |
| | | | 4GlcNAcβ1-2Manα1-6)Manβ1-4GlcNAcβ1- |
| | | | 4GlcNAcβ1-Asn |
| 31 | A2BG2S2 | A2B | Siaα2-3Galβ1-4GlcNAcβ1-2Manα1-3(Siaα2-3Galβ1- |
| | | | 4GlcNAcβ1-2Manα1-6)(GlcNAcβ1-4)Manβ1- |
| | | | 4GlcNAcβ1-4GlcNAcβ1-Asn |
| 32 | A1[3]G1S1(6) | G-1A1 | Siaα2-6Galβ1-4GlcNAcβ1-2Manα1-3(Manα1-6)Manβ1- |
| | | | 4GlcNAcβ1-4GlcNAcβ1-Asn |
| 33 | A1B[3]G1S1(6) | G-1BA1 | Siaα2-6Galβ1-4GlcNAcβ1-2Manα1-3(Manα1-6) |
| | | | (GlcNAcβ1-4)Manβ1-4GlcNAcβ1-4GlcNAcβ1-Asn |
| 34 | A1[6]G1S1(6) | G-1A1 | Manα1-3(Siaα2-6Galβ1-4GlcNAcβ1-2Manα1-6)Manβ1- |
| | | | 4GlcNAcβ1-4GlcNAcβ1-Asn |
| 35 | A1B[6]G1S1(6) | G-1BA1 | Manα1-3(Siaα2-6Galβ1-4GlcNAcβ1-2Manα1-6) |
| | | | (GlcNAcβ1-4)Manβ1-4GlcNAcβ1-4GlcNAcβ1-Asn |
| 36 | A2[3]G1S1(6) | A1 | Siaα2-6Galβ1-4GlcNAcβ1-2Manα1-3(GlcNAcβ1- |
| | | | 2Manα1-6)Manβ1-4GlcNAcβ1-4GlcNAcβ1-Asn |
| 37 | A2B[3]G1S1(6) | A1B | Siaα2-6Galβ1-4GlcNAcβ1-2Manα1-3(GlcNAcβ1- |
| | | | 2Manα1-6)(GlcNAcβ1-4)Manβ1-4GlcNAcβ1- |
| | | | 4GlcNAcβ1-Asn |
| 38 | A2[6]G1S1(6) | A1 | GlcNAcβ1-2Manα1-3(Siaα2-6Galβ1-4GlcNAcβ1- |
| | | | 2Manα1-6)Manβ1-4GlcNAcβ1-4GlcNAcβ1-Asn |
| 39 | A2B[6]G1S1(6) | A1B | GlcNAcβ1-2Manα1-3(Siaα2-6Galβ1-4GlcNAcβ1- |
| | | | 2Manα1-6)(GlcNAcβ1-4)Manβ1-4GlcNAcβ1- |
| | | | 4GlcNAcβ1-Asn |
| 40 | A2G2[3]S1(6) | A1 | Siaα2-6Galβ1-4GlcNAcβ1-2Manα1-3(Galβ1- |
| | | | 4GlcNAcβ1-2Manα1-6)Manβ1-4GlcNAcβ1- |
| | | | 4GlcNAcβ1-Asn |
| 41 | A2BG2[3]S1(6) | A1B | Siaα2-6Galβ1-4GlcNAcβ1-2Manα1-3(Galβ1- |
| | | | 4GlcNAcβ1-2Manα1-6)(GlcNAcβ1-4)Manβ1- |
| | | | 4GlcNAcβ1-4GlcNAcβ1-Asn |
| 42 | A2G2[6]S1(6) | A1 | Galβ1-4GlcNAcβ1-2Manα1-3(Siaα2-6Galβ1- |
| | | | 4GlcNAcβ1-2Manα1-6)Manβ1-4GlcNAcβ1- |
| | | | 4GlcNAcβ1-Asn |
| 43 | A2BG2[6]S1(6) | A1B | Galβ1-4GlcNAcβ1-2Manα1-3(Siaα2-6Galβ1- |
| | | | 4GlcNAcβ1-2Manα1-6)(GlcNAcβ1-4)Manβ1- |
| | | | 4GlcNAcβ1-4GlcNAcβ1-Asn |
| 44 | A2G2S2(6) | A2 | Siaα2-6Galβ1-4GlcNAcβ1-2Manα1-3(Siaα2-6Galβ1- |
| | | | 4GlcNAcβ1-2Manα1-6)Manβ1-4GlcNAcβ1- |
| | | | 4GlcNAcβ1-Asn |
| 45 | A2BG2S2(6) | A2B | Siaα2-6Galβ1-4GlcNAcβ1-2Manα1-3(Siaα2-6Galβ1- |
| | | | 4GlcNAcβ1-2Manα1-6)(GlcNAcβ1-4)Manβ1- |
| | | | 4GlcNAcβ1-4GlcNAcβ1-Asn |
| 46 | FA1[3] | G-1F | GlcNAcβ1-2Manα1-3(Manα1-6)Manβ1-4GlcNAcβ1- |
| | | | 4(Fucα1,6)GlcNAcβ1-Asn |
| 47 | FA1B[3] | G-1BF | GlcNAcβ1-2Manα1-3(Manα1-6)(GlcNAcβ1-4)Manβ1- |
| | | | 4GlcNAcβ1-4(Fucα1,6)GlcNAcβ1-Asn |
| 48 | FA1[6] | G-1F | Manα1-3(GlcNAcβ1-2Manα1-6)Manβ1-4GlcNAcβ1- |
| | | | 4(Fucα1,6)GlcNAcβ1-Asn |
| 49 | FA1B[6] | G-1BF | Manα1-3(GlcNAcβ1-2Manα1-6)(GlcNAcβ1-4)Manβ1- |
| | | | 4GlcNAcβ1-4(Fucα1,6)GlcNAcβ1-Asn |
| 50 | FA2 | G0F | GlcNAcβ1-2Manα1-3(GlcNAcβ1-2Manα1-6)Manβ1- |
| | | | 4GlcNAcβ1-4(Fucα1,6)GlcNAcβ1-Asn |
| 51 | FA2B | G0BF | GlcNAcβ1-2Manα1-3(GlcNAcβ1-2Manα1-6)(GlcNAcβ1-4) |
| | | | Manβ1-4GlcNAcβ1-4(Fucα1,6)GlcNAcβ1-Asn |
| 52 | FA1[3]G1 | G-1G1F | Galβ1-4GlcNAcβ1-2Manα1-3(Manα1-6)Manβ1- |
| | | | 4GlcNAcβ1-4(Fucα1,6)GlcNAcβ1-Asn |
| 53 | FA1B[3]G1 | G-1G1BF | Galβ1-4GlcNAcβ1-2Manα1-3(Manα1-6)(GlcNAcβ1-4) |
| | | | Manβ1-4GlcNAcβ1-4(Fucα1,6)GlcNAcβ1-Asn |
| 54 | FA1[6]G1 | G-1G1F | Manα1-3(Galβ1-4GlcNAcβ1-2Manα1-6)Manβ1- |
| | | | 4GlcNAcβ1-4GlcNAcβ1-Asn |
| 55 | FA1B[6]G1 | G-1G1BF | Manα1-3(Galβ1-4GlcNAcβ1-2Manα1-6)(GlcNAcβ1-4) |
| | | | Manβ1-4GlcNAcβ1-4(Fucα1,6)GlcNAcβ1-Asn |
| 56 | FA2[3]G1 | G1F | Galβ1-4GlcNAcβ1-2Manα1-3(GlcNAcβ1-2Manα1-6) |
| | | | Manβ1-4GlcNAcβ1-4GlcNAcβ1-Asn |
| 57 | FA2B[3]G1 | G1F | Galβ1-4GlcNAcβ1-2Manα1-3(GlcNAcβ1-2Manα1-6) |
| | | | (GlcNAcβ1-4)Manβ1-4GlcNAcβ1-4GlcNAcβ1-Asn |
| 58 | FA2[6]G1 | G1F | GlcNAcβ1-2Manα1-3(Galβ1-4GlcNAcβ1-2Manα1-6) |
| | | | Manβ1-4GlcNAcβ1-4GlcNAcβ1-Asn |
| 59 | FA2B[6]G1 | G1F | GlcNAcβ1-2Manα1-3(Galβ1-4GlcNAcβ1-2Manα1-6) |
| | | | (GlcNAcβ1-4)Manβ1-4GlcNAcβ1-4GlcNAcβ1-Asn |
| 60 | FA2G2 | G2F | Galβ1-4GlcNAcβ1-2Manα1-3(Galβ1-4GlcNAcβ1- |
| | | | 2Manα1-6)Manβ1-4GlcNAcβ1-4(Fucα1,6)GlcNAcβ1- |
| | | | Asn |
| 61 | FA2BG2 | G2BF | Galβ1-4GlcNAcβ1-2Manα1-3(Galβ1-4GlcNAcβ1- |
| | | | 2Manα1-6)(GlcNAcβ1-4)Manβ1-4GlcNAcβ1- |
| | | | 4GlcNAcβ1-Asn |
| 62 | FA1[3]G1S1(3) | G-1A1 | Siaα2-3Galβ1-4GlcNAcβ1-2Manα1-3(Manα1-6)Manβ1- |
| | | | 4GlcNAcβ1-4(Fucα1,6)GlcNAcβ1-Asn |
| 63 | FA1B[3]G1S1(3) | G-1BA1 | Siaα2-3Galβ1-4GlcNAcβ1-2Manα1-3(Manα1-6) |
| | | | (GlcNAcβ1-4)Manβ1-4GlcNAcβ1- |
| | | | 4(Fucα1,6)GlcNAcβ1-Asn |
| 64 | FA1[6]G1S1(3) | G-1A1 | Manα1-3(Siaα2-3Galβ1-4GlcNAcβ1-2Manα1-6)Manβ1- |
| | | | 4GlcNAcβ1-4(Fucα1,6)GlcNAcβ1-Asn |
| 65 | FA1B[6]G1S1(3) | G-1BA1 | Manα1-3(Siaα2-3Galβ1-4GlcNAcβ1-2Manα1-6) |
| | | | (GlcNAcβ1-4)Manβ1-4GlcNAcβ1- |
| | | | 4(Fucα1,6)GlcNAcβ1-Asn |
| 66 | FA2[3]G1S1(3) | A1 | Siaα2-3Galβ1-4GlcNAcβ1-2Manα1-3(GlcNAcβ1- |
| | | | 2Manα1-6)Manβ1-4GlcNAcβ1-4(Fucα1,6)GlcNAcβ1- |
| | | | Asn |
| 67 | FA2B[3]G1S1(3) | A1B | Siaα2-3Galβ1-4GlcNAcβ1-2Manα1-3(GlcNAcβ1- |
| | | | 2Manα1-6)(GlcNAcβ1-4)Manβ1-4(Fucα1,6)GlcNAcβ1- |
| | | | 4GlcNAcβ1-Asn |
| 68 | FA2[6]G1S1(3) | A1 | GlcNAcβ1-2Manα1-3(Siaα2-3Galβ1-4GlcNAcβ1- |
| | | | 2Manα1-6)Manβ1-4GlcNAcβ1-4(Fucα1,6)GlcNAcβ1- |
| | | | Asn |
| 69 | FA2B[6]G1S1(3) | A1B | GlcNAcβ1-2Manα1-3(Siaα2-3Galβ1-4GlcNAcβ1- |
| | | | 2Manα1-6)(GlcNAcβ1-4)Manβ1-4GlcNAcβ1- |
| | | | 4(Fucα1,6)GlcNAcβ1-Asn |
| 70 | FA2G2[3]S1(3) | A1 | Siaα2-3Galβ1-4GlcNAcβ1-2Manα1-3(Galβ1- |
| | | | 4GlcNAcβ1-2Manα1-6)Manβ1-4GlcNAcβ1- |
| | | | 4(Fucα1,6)GlcNAcβ1-Asn |
| 71 | FA2BG2[3]S1(3) | A1B | Siaα2-3Galβ1-4GlcNAcβ1-2Manα1-3(Galβ1- |
| | | | 4GlcNAcβ1-2Manα1-6)(GlcNAcβ1-4)Manβ1- |
| | | | 4(Fucα1,6)GlcNAcβ1-4GlcNAcβ1-Asn |
| 72 | FA2G2S2(3) | A2 | Siaα2-3Galβ1-4GlcNAcβ1-2Manα1-3(Siaα2-3Galβ1- |
| | | | 4GlcNAcβ1-2Manα1-6)Manβ1-4GlcNAcβ1- |
| | | | 4(Fucα1,6)GlcNAcβ1-Asn |
| 73 | FA2BG2S2(3) | A2B | Siaα2-3Galβ1-4GlcNAcβ1-2Manα1-3(Siaα2-3Galβ1- |
| | | | 4GlcNAcβ1-2Manα1-6)(GlcNAcβ1-4)Manβ1- |
| | | | 4GlcNAcβ1-4(Fucα1,6)GlcNAcβ1-Asn |
| 74 | FA2G2[6]S1(3) | A1 | Galβ1-4GlcNAcβ1-2Manα1-3(Siaα2-3Galβ1- |
| | | | 4GlcNAcβ1-2Manα1-6)Manβ1-4GlcNAcβ1- |
| | | | 4(Fucα1,6)GlcNAcβ1-Asn |
| 75 | FA2BG2[6]S1(3) | A1B | Galβ1-4GlcNAcβ1-2Manα1-3(Siaα2-3Galβ1- |
| | | | 4GlcNAcβ1-2Manα1-6)(GlcNAcβ1-4)Manβ1- |
| | | | 4GlcNAcβ1-4(Fucα1,6)GlcNAcβ1-Asn |
| 76 | FA1[3]G1S1(6) | G-1A1 | Siaα2-6Galβ1-4GlcNAcβ1-2Manα1-3(Manα1-6)Manβ1- |
| | | | 4GlcNAcβ1-4(Fucα1,6)GlcNAcβ1-Asn |
| 77 | FA1B[3]G1S1(6) | G-1BA1 | Siaα2-6Galβ1-4GlcNAcβ1-2Manα1-3(Manα1-6) |
| | | | (GlcNAcβ1-4)Manβ1-4GlcNAcβ1- |
| | | | 4(Fucα1,6)GlcNAcβ1-Asn |
| 78 | FA1[6]G1S1(6) | G-1A1 | Manα1-3(Siaα2-6Galβ1-4GlcNAcβ1-2Manα1-6)Manβ1- |
| | | | 4GlcNAcβ1-4(Fucα1,6)GlcNAcβ1-Asn |
| 79 | FA1B[6]G1S1(6) | G-1BA1 | Manα1-3(Siaα2-6Galβ1-4GlcNAcβ1-2Manα1- |
| | | | 6)(GlcNAcβ1-4)Manβ1-4GlcNAcβ1- |
| | | | 4(Fucα1,6)GlcNAcβ1-Asn |
| 80 | FA2[3]G1S1(6) | A1 | Siaα2-6Galβ1-4GlcNAcβ1-2Manα1-3(GlcNAcβ1- |
| | | | 2Manα1-6)Manβ1-4GlcNAcβ1-4(Fucα1,6)GlcNAcβ1- |
| | | | Asn |
| 81 | FA2B[3]G1S1(6) | A1B | Siaα2-6Galβ1-4GlcNAcβ1-2Manα1-3(GlcNAcβ1- |
| | | | 2Manα1-6)(GlcNAcβ1-4)Manβ1-4GlcNAcβ1- |
| | | | 4(Fucα1,6)GlcNAcβ1-Asn |
| 82 | FA2[6]G1S1(6) | A1 | GlcNAcβ1-2Manα1-3(Siaα2-6Galβ1-4GlcNAcβ1- |
| | | | 2Manα1-6)Manβ1-4GlcNAcβ1-4(Fucα1,6)GlcNAcβ1- |
| | | | Asn |
| 83 | FA2B[6]G1S1(6) | A1B | GlcNAcβ1-2Manα1-3(Siaα2-6Galβ1-4GlcNAcβ1- |
| | | | 2Manα1-6)(GlcNAcβ1-4)Manβ1-4GlcNAcβ1- |
| | | | 4(Fucα1,6)GlcNAcβ1-Asn |
| 84 | FA2G2[3]S1(6) | A1 | Siaα2-6Galβ1-4GlcNAcβ1-2Manα1-3(Galβ1- |
| | | | 4GlcNAcβ1-2Manα1-6)Manβ1-4GlcNAcβ1- |
| | | | 4(Fucα1,6)GlcNAcβ1-Asn |
| 85 | FA2BG2[3]S1(6) | A1B | Siaα2-6Galβ1-4GlcNAcβ1-2Manα1-3(Galβ1- |
| | | | 4GlcNAcβ1-2Manα1-6)(GlcNAcβ1-4)Manβ1- |
| | | | 4GlcNAcβ1-4(Fucα1,6)GlcNAcβ1-Asn |
| 86 | FA2G2[6]S1(6) | A1 | Galβ1-4GlcNAcβ1-2Manα1-3(Siaα2-6Galβ1- |
| | | | 4GlcNAcβ1-2Manα1-6)Manβ1-4GlcNAcβ1- |
| | | | 4(Fucα1,6)GlcNAcβ1-Asn |
| 87 | FA2BG2[6]S1(6) | A1B | Galβ1-4GlcNAcβ1-2Manα1-3(Siaα2-6Galβ1- |
| | | | 4GlcNAcβ1-2Manα1-6)(GlcNAcβ1-4)Manβ1- |
| | | | 4GlcNAcβ1-4(Fucα1,6)GlcNAcβ1-Asn |
| 88 | FA2G2S2(6) | A2 | Siaα2-6Galβ1-4GlcNAcβ1-2Manα1-3(Siaα2-6Galβ1- |
| | | | 4GlcNAcβ1-2Manα1-6)Manβ1-4GlcNAcβ1- |
| | | | 4(Fucα1,6)GlcNAcβ1-Asn |
| 89 | FA2BG2S2(6) | A2B | Siaα2-6Galβ1-4GlcNAcβ1-2Manα1-3(Siaα2-6Galβ1- |
| | | | 4GlcNAcβ1-2Manα1-6)(GlcNAcβ1-4)Manβ1- |
| | | | 4GlcNAcβ1-4(Fucα1,6)GlcNAcβ1-Asn |

The [3] and[6] indicate the mannose arm of the N-glycan to which additional sugars may be linked.
The (3) indicates the sialic acid is linked to the galactose in an α2-3 linkage and the (6) indicates the sialic acid is linked to the galactose in an α2-6 linkage. The Asn residue is within the N-glycosylation consensus sequence Asn-Xaa-Ser/Thr, wherein Xaa is any amino acid other than Pro. The carboxamide group of the Asn residue is linked to the GlcNAc residue at the reducing end of the N-glycan in a β1 linkage.

Abbreviations used herein are of common usage in the art, see, e.g., abbreviations of sugars, above. Other common abbreviations include “PNGase”, or “glycanase” or “glucosidase” which all refer to peptide N-glycosidase F (EC 3.2.2.18).

As used herein, the terms “N-azidoacetylglucosamine”, “N₃-GlcNAc”, “azido-GlcNAc”, or “GlcNAc-N₃” refers to an N-acetylglucosamine comprising an azide group linked to the terminal carbon of the acetyl group thereof or to the C6 carbon thereof.

As used herein, the term “GlcNAz” refers to an N-acetylglucosamine comprising an azide group linked to the terminal carbon of its acetyl group and may be represented by the following formula

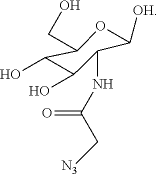 [SMILES: [N-]=[N+]=NCC(=O)NC1C(O)OC(CO)C(O)C1O]

As used herein, the term “GlcNAz residue” refers to the GlcNAz linked to the β1-4-linked mannose residue of the trimannose core and may be represented by the following formula

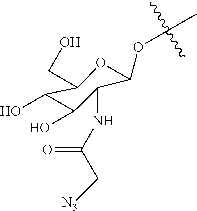 [SMILES: *OC1OC(CO)C(O)C(O)C1NC(=O)CN=[N+]=[N-]]

wherein the wavy line is a bond to the C4 carbon of the β1-4-linked mannose residue of the trimannose core. The term further includes embodiments wherein the azide group is also linked to an alkyne of a payload or linker-payload.

As used herein, the term “6-azido-6-deoxy-N-acetyl-glucosamine” or “6-azido-GlcNAc” refers to a GlcNAc residue comprising an azido group linked to the C6 carbon and may be represented by the formula

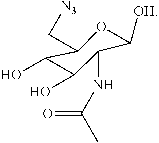 [SMILES: CC(=O)NC1C(O)OC(CN=[N+]=[N-])C(O)C1O]

As used herein, the term “6-azido-GlcNAc residue” refers to the GlcNAz linked to the β1-4-linked mannose residue of the trimannose core and may be represented by the following formula

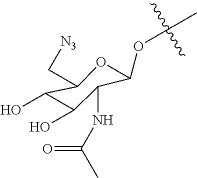 [SMILES: *OC1OC(CN=[N+]=[N-])C(O)C(O)C1NC(C)=O]

wherein the wavy line is a bond to the C4 carbon of the β1-4-linked mannose residue of the trimannose core. The term further includes embodiments wherein the azide group is also linked to an alkyne of a payload or linker-payload.

As used herein, the term “glycoprotein” refers to any protein having one or more N-glycans attached thereto. Thus, the term refers both to proteins that are generally recognized in the art as a glycoprotein and to proteins which have been genetically engineered to contain one or more N-linked glycosylation sites.

As used herein, a “humanized glycoprotein” or a “human-like glycoprotein” refers alternatively to a protein having attached thereto complex or hybrid N-glycans.

The term “recombinant host cell” (“expression host cell”, “expression host system”, “expression system” or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism. Preferred host cells are yeasts and fungi.

When referring to “mole percent” of an N-glycan present in a preparation of a glycoprotein, the term means the molar percent of a particular N-glycan present in the pool of N-linked oligosaccharides released when the protein preparation is treated with PNGase and then quantified by a method that is not affected by glycoform composition, (for instance, labeling a PNGase released glycan pool with a fluorescent tag such as 2-aminobenzamide and then separating by high performance liquid chromatography or capillary electrophoresis and then quantifying glycans by fluorescence intensity). For example, 50 mole percent Galβ1-4GlcNAcβ1-2Manα1-3(Manα1-6)Manβ1-4GlcNAcβ1-4 (Fucα1,6) GlcNAc means that 50 percent of the released glycans are Galβ1-4GlcNAcβ1-2Manα1-3(Manα1-6)Manβ1-4GlcNAcβ1-4 (Fucα1,6) GlcNAc and the remaining 50 percent are comprised of other N-linked oligosaccharides. In embodiments, the mole percent of a particular glycan in a preparation of glycoprotein will be between 20% and 100%, preferably above 25%, 30%, 35%, 40% or 45%, more preferably above 50%, 55%, 60%, 65% or 70% and most preferably above 75%, 80% 85%, 90% or 95%.

The term “operably linked” expression control sequences refers to a linkage in which the expression control sequence is contiguous with the gene of interest to control the gene of interest, as well as expression control sequences that act in trans or at a distance to control the gene of interest.

The term “expression control sequence” or “regulatory sequences” are used interchangeably and as used herein refer to polynucleotide sequences which are necessary to affect the expression of coding sequences to which they are operably linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “transfect”, transfection”, “transfecting” and the like refer to the introduction of a heterologous nucleic acid into eukaryote cells, both higher and lower eukaryote cells. Historically, the term “transformation” has been used to describe the introduction of a nucleic acid into a yeast or fungal cell; however, herein the term “transfection” is used to refer to the introduction of a nucleic acid into any eukaryote cell, including yeast and fungal cells.

The term “eukaryotic” refers to a nucleated cell or organism, and includes insect cells, plant cells, mammalian cells, animal cells and lower eukaryotic cells.

The term “lower eukaryotic cells” includes yeast and filamentous fungi. Yeast and filamentous fungi include, but are not limited to Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus sp., Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa.

As used herein, the term “antibody” or “immunoglobulin” as used herein refers to a glycoprotein comprising at least two heavy chains (HCs) and two light chains (LCs) inter-connected by disulfide bonds. Each HC is comprised of a heavy chain variable region or domain (V_{H}) and a heavy chain constant region or domain. Each light chain is comprised of an LC variable region or domain (V_{L}) and a LC constant domain. In certain naturally occurring IgG, IgD and IgA antibodies, the heavy chain constant region is comprised of three domains, CH1, CH2 and CH3. In general, the basic antibody structural unit for antibodies is a Y-shaped tetramer comprising two HC/LC pairs (2H). Each tetramer includes two identical pairs of polypeptide chains, each pair having one LC (about 25 kDa) and HC chain (about 50-70 kDa) (H+L). Each HC: LC pair comprises one V_{H}: one V_{L} pair. The one V_{H}: one V_{L} pair may be referred to by the term “Fab”. Thus, each antibody tetramer comprises two Fabs, one per each arm of the Y-shaped antibody.

The LC constant domain is comprised of one domain, CL. The human V_{H} includes seven family members: V_{H}1, V_{H}2, V_{H}3, V_{H}4, V_{H}5, V_{H}6, and V_{H}7; and the human V_{L} includes 16 family members: V_{κ}1, V_{κ}2, V_{κ}3, V_{κ}4, V_{κ}5, V_{κ}6, V_{λ}1, V_{λ}2, V_{λ}3, V_{λ}4, V_{λ}5, V_{λ}6, V_{λ}7, V_{λ}8, V_{λ}9, and V_{λ}10. Each of these family members can be further divided into particular subtypes. The V_{H} and V_{L} can be further subdivided into regions of hypervariability, termed complementarity determining region (CDR) areas, interspersed with regions that are more conserved, termed framework regions (FR). Each V_{H} and V_{L} is composed of three CDR regions and four FR regions, arranged from amino-terminus to carboxy-terminus in the following order: FR1, CDR1, FR2, CDR2, FR3, CDR3, FR4. Numbering of the amino acids in a VH may be determined using the Kabat numbering scheme. See Béranger, et al., Ed. Ginetoux, Correspondence between the IMGT unique numbering for C-DOMAIN, the IMGT exon numbering, the Eu and Kabat numberings: Human IGHG, Created: 17 May 2001, Version: Aug. 6, 2016, which is accessible at www.imgt.org/IMGTScientificChart/Numbering/Hu_IGHGnber.html).

The constant regions of the antibodies may mediate the binding of the immunoglobulin to host tissues or factors, including various cells of the immune system (e.g., effector cells) and the first component (C1q) of the classical complement system. Typically, the numbering of the amino acids in the heavy chain constant domain begins with number 118, which is in accordance with the Eu numbering scheme. The Eu numbering scheme is based upon the amino acid sequence of human IgG1 (Eu), which has a constant domain that begins at amino acid position 118 of the amino acid sequence of the IgG1 described in Edelman et al., Proc. Natl. Acad. Sci. USA. 63:78-85 (1969), and is shown for the IgG1, IgG2, IgG3, and IgG4 constant domains in Béranger et al., op. cit.

The variable regions of the heavy and light chains contain a binding domain comprising the CDRs that interacts with an antigen. A number of methods are available in the art for defining CDR sequences of antibody variable domains (see Dondelinger et al., Frontiers in Immunol. 9: Article 2278 (2018)). The common numbering schemes include the following.
Kabat numbering scheme is based on sequence variability and is the most commonly used (See Kabat et al. Sequences of Proteins of Immunological Interest, 5th Ed. Public Health Service, National Institutes of Health, Bethesda, Md. (1991) (defining the CDR regions of an antibody by sequence);
Chothia numbering scheme is based on the location of the structural loop region (See Chothia & Lesk, J. Mol. Biol. 196:901-917 (1987); Al-Lazikani et al., J. Mol. Biol. 273:927-948 (1997));
AbM numbering scheme is a compromise between the two used by Oxford Molecular's AbM antibody modelling software (see Karu et al., ILAR Journal 37:132-141 (1995);
Contact numbering scheme is based on an analysis of the available complex crystal structures (See www.bioinf.org.uk: Prof. Andrew C. R. Martin's Group; Abhinandan & Martin, Mol. Immunol. 45:3832-3839 (2008)).
IMGT (ImMunoGeneTics) numbering scheme is a standardized numbering system for all the protein sequences of the immunoglobulin superfamily, including variable domains from antibody light and heavy chains as well as T cell receptor chains from different species and counts residues continuously from 1 to 128 based on the germ-line V sequence alignment (see Giudicelli et al., Nucleic Acids Res. 25:206-11 (1997); Lefranc, Immunol Today 18:509 (1997); Lefranc et al., Dev Comp Immunol. 27:55-77 (2003)).

The following general rules disclosed in www.bioinf.org.uk: Prof. Andrew C. R. Martin's Group and reproduced in Table 2 below may be used to define the CDRs in an antibody sequence that includes those amino acids that specifically interact with the amino acids comprising the epitope in the antigen to which the antibody binds. There are rare examples where these generally constant features do not occur; however, the Cys residues are the most conserved feature.

TABLE 2

| Loop | Kabat | AbM | Chothia¹ | Contact² | IMGT |
|---|---|---|---|---|---|
| L1 | L24--L34 | L24--L34 | L24--L34 | L30--L36 | L27--L32 |
| L2 | L50--L56 | L50--L56 | L50--L56 | L46--L55 | L50--L52 |
| L3 | L89--L97 | L89--L97 | L89--L97 | L89--L96 | L89--L97 |
| H1 | H31--H35B | H26--H35B | H26--H32.34 | H30--H35B | H26--H35B |
| | (Kabat Numbering)³ | | | | |
| H1 | H31--H35 | H26--H35 | H26--H32 | H30--H35 | H26--H33 |
| | (Chothia Numbering) | | | | |
| H2 | H50--H65 | H50--H58 | H52--H56 | H47--H58 | H51--H56 |
| H3 | H95--H102 | H95--H102 | H95--H102 | H93--H101 | H93--H102 |

¹Some of these numbering schemes (particularly for Chothia loops) vary depending on the individual publication examined.
²Any of the numbering schemes can be used for these CDR definitions, except the Contact numbering scheme uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering scheme places the insertions at H35A and H35B.)
If neither H35A nor H35B is present, the loop ends at H32
If only H35A is present, the loop ends at H33
If both H35A and H35B are present, the loop ends at H34

The entire nucleotide sequence of the heavy chain and light chain variable regions are commonly numbered according to Kabat while the three CDRs within the variable region may be defined according to any one of the aforementioned numbering schemes.

As used herein, the term “isolated” antibodies or antigen-binding fragments thereof are at least partially free of other biological molecules from the cells or cell cultures in which they are produced. Such biological molecules include nucleic acids, proteins, lipids, carbohydrates, or other material such as cellular debris and growth medium. An isolated antibody or antigen-binding fragment may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antibodies or fragments.

As used herein, the term “monoclonal antibody” refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains that are often specific for different epitopes. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler et al., Nature 256:495 (1975) or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991), and Marks et al., J. Mol. Biol. 222:581-597 (1991), for example. See also Presta, J. Allergy Clin. Immunol. 116:731 (2005).

As used herein, the term “Fc domain”, “Fc region”, or “Fc” as used herein is the crystallizable fragment domain or region obtained from an antibody that comprises the CH2 and CH3 domains of an antibody, which defines the C-terminal region of an immunoglobulin heavy chain, including, for example, native sequence Fc regions, recombinant Fc regions, and variant Fc regions. Typically, amino acids in the Fc domain are numbered according to the Eu numbering convention (See Edelmann et al., Biochem. 63: 78-85 (1969)). Although the boundaries of the Fc region of an immunoglobulin heavy chain might vary, the human IgG heavy chain Fc region is often defined to stretch from an amino acid residue at position Cys226, or from Pro230, to the carboxyl-terminus thereof.

The heavy and light chains of antibodies are typically expressed as a precursor protein comprising a leader or signal peptide sequence at the N-terminus, which is removed following translation to provide the mature heavy and light chains. The mature heavy chain and light chain N-terminal amino acid residues are usually independently a Gln or Glu residue. Either amino acid may spontaneously cyclize in vitro to form pyroglutamate, which may occur for substantially all antibodies in a composition. Accordingly, a composition of intact antibodies may comprise antibody populations in which all N-terminal residues are pyroglutamate, antibody populations in which all N-terminal residues are Glu or Gln, and antibody populations having a mixture of antibodies in which the N-terminal residue is Glu, Gln, or pyroglutamate. Thus, the term “antibody” or “immunoglobulin” or “VH” or “VL” or “HC” or “LC” include variants that comprise N-terminal pyroglutamate.

The C-terminal lysine of the heavy chain (residue 447 of the Fc domain according to the EU numbering system) may be removed, for example, during production or purification of the antibody, or by recombinantly engineering the nucleic acid encoding a heavy chain of the antibody to not include the codon for the K447 lysine. Accordingly, a composition of intact antibodies may comprise antibody populations with all K447 residues removed, antibody populations with no K447 residues removed, and antibody populations having a mixture of antibodies with and without the K447 residue. In particular embodiments of a heavy chain lacking the K447 residue, the heavy chain further has the G446 residue removed as well. Thus, the term “antibody” or “immunoglobulin” or “HC” or “LC” include variants that lack a C-terminal K447 residue or G446 K447 residues.

As used herein, the term “antibody” or “immunoglobulin” or “HC” or “LC” include a composition of intact antibodies that comprises antibody populations in which all N-terminal residues are pyroglutamate, antibody populations in which all N-terminal residues are Glu or Gln, and antibody populations having a mixture of antibodies in which the N-terminal residue is Glu, Gln, or pyroglutamate and may further comprise antibody populations with all K447 or G446 K447 residues removed, antibody populations with no K447 residues removed, and antibody populations having a mixture of antibodies with and without the K447 or G446 K447 residue.

As used herein, the term “antigen” as used herein refers to any foreign substance which induces an immune response in the body.

The term “fragments” within the scope of the terms “antibody” or “immunoglobulin” include those produced by digestion with various proteases, those produced by chemical cleavage and/or chemical dissociation and those produced recombinantly, so long as the fragment remains capable of specific binding to a target molecule. Among such fragments are Fc, Fab, Fab′, Fv, F(ab′)2, and single chain Fv (scFv) fragments. Hereinafter, the term “immunoglobulin” also includes the term “fragments” as well.

Immunoglobulins further include immunoglobulins or fragments that have been modified in sequence but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (See, for example, Intracellular Antibodies: Research and Disease Applications, (Marasco, ed., Springer-Verlag New York, Inc., 1998).

The interaction of antibodies and antibody-antigen complexes with cells of the immune system and the variety of responses, including antibody-dependent cell-mediated cytotoxicity (ADCC) and complement-dependent cytotoxicity (CDC), clearance of immunocomplexes (phagocytosis), antibody production by B cells and IgG serum half-life are defined respectively in the following: Daeron et al., Annu. Rev. Immunol. 15: 203-234 (1997); Ward and Ghetie, Therapeutic Immunol. 2:77-94 (1995); Cox and Greenberg, Semin. Immunol. 13: 339-345 (2001); Heyman, Immunol. Lett. 88:157-161 (2003); and Ravetch, Curr. Opin. Immunol. 9: 121-125 (1997).

The term “catalytic antibody” refers to immunoglobulin molecules that are capable of catalyzing a biochemical reaction. Catalytic antibodies are well known in the art and have been described in U.S. Pat. Nos. 7,205,136; 4,888,281; 5,037,750 to Schochetman et al., U.S. Pat. Nos. 5,733,757; 5,985,626; and U.S. Pat. No. 6,368,839 to Barbas, III et al. (the disclosures of which are all incorporated herein by reference).

The interaction of antibodies and antibody-antigen complexes with cells of the immune system and the variety of responses, including antibody-dependent cell-mediated cytotoxicity (ADCC) and complement-dependent cytotoxicity (CDC), clearance of immunocomplexes (phagocytosis), antibody production by B cells and IgG serum half-life are defined respectively in the following: Daeron et al., Annu. Rev. Immunol. 15: 203-234 (1997); Ward and Ghetie, Therapeutic Immunol. 2:77-94 (1995); Cox and Greenberg, Semin. Immunol. 13: 339-345 (2001); Heyman, Immunol. Lett. 88:157-161 (2003); and Ravetch, Curr. Opin. Immunol. 9:121-125 (1997).

As used herein, the term “consisting essentially of” will be understood to imply the inclusion of a stated integer or group of integers; while excluding modifications or other integers which would materially affect or alter the stated integer. With respect to species of N-glycans, the term “consisting essentially of” a stated N-glycan will be understood to include the N-glycan whether or not that N-glycan is fucosylated at the N-acetylglucosamine (GlcNAc) which is directly linked to the asparagine residue of the glycoprotein.

As used herein, the term “predominant” or “predominantly” used with respect to the production of N-glycans refers to a structure which represents the major peak detected by ultrahigh pressure liquid chromatography with ultraviolet (UPLC-UV) absorbance measurement. Glycan masses may be confirmed with UPLC interfaced to electrospray quadrupole time-of-flight mass spectrometry (ESI-QTOF-MS). For example, if a composition consists of species A at 40 mole percent, species B at 35 mole percent and species C at 25 mole percent, the composition comprises predominantly species A, and species B would be the next most predominant species. Some host cells may produce compositions comprising neutral N-glycans and charged N-glycans such as mannosylphosphate. Thus, as used herein, “predominant N-glycan” means that of the total plurality of neutral N-glycans in the composition, the predominant N-glycan is of a particular structure.

As used herein, the term “essentially free of” a particular sugar residue, such as fucose, or galactose and the like, is used to indicate that the glycoprotein composition is substantially devoid of N-glycans which contain such residues. Expressed in terms of purity, essentially free means that the amount of N-glycan structures containing such sugar residues does not exceed 10%, and preferably is below 5%, more preferably below 1%, most preferably below 0.5%, wherein the percentages are by weight or by mole percent. Thus, substantially all of the N-glycan structures in a glycoprotein composition according to the present invention are free of, for example, fucose, or galactose, or both.

As used herein, a glycoprotein composition “lacks” or “is lacking” a particular sugar residue, such as fucose or galactose, when no detectable amount of such sugar residue is present on the N-glycan structures at any time. For example, in preferred embodiments of the present invention, the glycoprotein compositions are produced by lower eukaryotic organisms, as defined above, including yeast (for example, Pichia sp.; Saccharomyces sp.; Kluyveromyces sp.; Aspergillus sp.), and will “lack fucose,” because the cells of these organisms do not have the enzymes needed to produce fucosylated N-glycan structures. Thus, the term “essentially free of fucose” encompasses the term “lacking fucose.” However, a composition may be “essentially free of fucose” even if the composition at one time contained fucosylated N-glycan structures or contains limited, but detectable amounts of fucosylated N-glycan structures as described above.

As used herein, the term “multiplicity” refers to a large number of the same or substantially same molecules, proteins, enzymes, species, etc. For example, a multiplicity of glycoproteins refers to a population of glycoproteins in which the glycoproteins have the same amino acid sequence and the same N-glycan structure. For example, a multiplicity of glycoproteins refers to a population of glycoproteins wherein the glycoproteins have substantially the same amino acid sequence and the N-glycans have substantially the same structure.

As used herein, the term “plurality” refers to a large number of a variety of species of molecules, proteins, enzymes, species, etc. For example, a plurality of glycoproteins refers to (i) a population of glycoproteins in which the glycoproteins have substantially the same amino acid sequence and more than one N-glycan structure, e.g., N-glycans comprising a mixture of Sia₍₁₋₂₎Gal₍₁₋₂₎GlcNAc₍₁₋₂₎ Man₃GlcNAc₂, Gal₍₁₋₂₎GlcNAc₍₁₋₂₎Man₃GlcNAc₂, and GlcNAc₍₁₋₂₎Man₃GlcNAc₂ structures, or (ii) a population of glycoproteins in which the glycoproteins comprise more than one amino acid sequence and a single N-glycan structure or more than one N-glycan structure.

As used herein, the term “sequentially” refers to forming or following in a logical order or sequence. For example, a glycoprotein may be reacted with a neuraminidase, a galactosidase, and a GlcNAcase in that order to produce a paucimannose N-glycan.

As used herein, the term “remodel” or “remodeling” refers to changing the structure of an N-glycan having a particular glycoform to another glycoform, by removing and/or adding sugar residues to the N-glycan. The term further includes changing the structures of the N-glycans of a population of glycoproteins.

As used herein, the term “glycosylation enzymes” refers to exoglycosidases, endoglycosidases, and glycosyltransferases, and includes both native and naturally occurring enzymes and recombinant variants thereof that comprise at least the catalytic activity of the glycosylation enzyme.

As used herein, the term “exoglycosidases” are glycoside hydrolase enzymes (EC 3.2) that cleave the glycosidic linkage of a terminal monosaccharide in an oligosaccharide or polysaccharide. Exoglycosidases include neuraminidases, galactosidases, N-acetylglucosaminidases, and fucosidases.

As used herein, “endoglycosidases” are glycoside hydrolase enzymes (EC 3.2) that cleave the glycosidic linkage between the two N-acetylglucosamine (GlcNAc) residues of the chitobiose core, but which recognize and cleave different types of N-linked glycans. For example, endoglycosidase H cleaves within the chitobiose core of high mannose and some hybrid oligosaccharides from N-linked glycoproteins, endoglycosidase S is highly specific for removing N-linked glycans from the heavy chain of native IgG, and endoglycosidase D cleaves paucimannose N-linked glycans.

As used herein, “glycosyltransferases” are enzymes (EC 2.4) that catalyze the transfer of saccharide moieties from an activated nucleotide sugar (also known as the “glycosyl donor”) to a nucleophilic glycosyl acceptor molecule, the nucleophile of which can be oxygen-, carbon-, nitrogen-, or sulfur-based. Examples of glycosyltransferases include but are not limited to N-acetylglucosaminyltransferase (GnT) I, which transfers GlcNAc from UDP-GlcNAc to the α1,3-linked terminal mannose (Man) residue on paucimannose N-glycans; GnTII, which transfers GlcNAc from UDP-GlcNAc to the α1,6-linked terminal mannose of GlcNAcMan₃GlcNAc₂ N-glycans in a β1,2 linkage; GnTIII, which transfers GlcNAc from UDP-GlcNAc to the α1,4-linked central mannose of GlcNAcMan₃GlcNAc₂ N-glycans in a β1,4 linkage; β1,4-galactosyltransferase (GalT), which transfers galactose (Gal) from UDP-galactose to the terminal GlcNAc residues of N-glycans in a β1,4 linkage; α2,3-sialyltransferase (α2,3-sialylT) or α2,6-sialyltransferase (α2,6-sialyIT), which transfers sialic acid from CMP-sialic acid to the terminal galactose residues of N-glycans in an α2,6 linkage; α2,3-sialyltransferase (α2,3-sialylT), which transfers sialic acid from CMP-sialic acid to the terminal galactose residues of N-glycans in an α2-3 linkage; and, α1,6-fucosyltransferase (FUT8) transfers fucose from GDP-fucose to the GlcNAc residue at the reducing end of the chitobiose core in an α1,6 linkage.

As used herein, the term “effector moiety” comprises agents (e.g., proteins, nucleic acids, lipids, carbohydrates, glycopeptides, and fragments thereof) with biological or other functional activity. For example, the effector moiety may be a therapeutic agent, a diagnostic agent, or a functional element such as a cleavable linker. As used herein, a payload that is directly or indirectly conjugated to the antibody may comprise an effector moiety.

### Introduction

The development of antibody-based drugs has grown significantly in the past decade (Lu et al., J. Biomed. Sci. 27, 1 (2020)). Conjugating an antibody to a small molecule drug increases target-specificity and lifetime in the bloodstream, resulting in improved safety and pharmacokinetic properties. Various designs of antibody conjugates have been developed, including antibody-drug conjugates (ADC), extracellular drug conjugates (EDCs), and antibody subunit conjugates, such as Fab- or Fc-fusions (Czajkowsky et al., EMBO Mol. Med. 4, 1015-1028 (2012); Lu, D. et al., J. Immunol. Meth. 267, 213-226 (2002)). Conjugation strategy plays a critical role to enable the fusion of the desired properties from both the antibody and the small molecule payload.

The present invention provides a new antibody conjugation strategy comprising modifying the N-glycans of the antibody to comprise bisected N-glycans wherein the bisecting sugar of the N-glycan is an azidoacetylglucosamine (6-azido-GlcNAc or GlcNAz) residue and conjugating payloads to the bisecting azidoacetylglucosamine residue. The azido group allows for azide-alkyne click chemistry conjugation of the antibody to payloads comprising an alkyne group suitable for conjugation. The strategy enables site-specific conjugation with high biocompatibility to the antibody and does not require genetic editing. The Examples herein show that this strategy can be applied to human IgG1 and mouse IgG2a and may be extrapolated to other IgG subclasses, for example, the IgG4 subclass. Remarkably, payload conjugation through the bisecting N-azidoacetylglucosamine residue preserves the opportunity to remodel the structure of the bi-antennary arms of the bisected N-glycan to have different glycoforms, for example, for the N-glycans to be GlcNAc terminated, galactose terminated, sialic acid terminated, fucosylated, afucosylated, or the like. The Examples herein show that several of these glycoforms result in ADCs with different binding affinities to FcγRs. These results show that the present invention enable the production of ADCs that can further be modified to have particular binding affinities for FcγRs by modifying the glycoforms of the bisected N-glycans conjugated to the payload.

The Examples show that N-glycans can serve as a regulatory handle to optimize the immunogenicity and pharmacokinetics of antibody conjugates. Together, conjugation through the bisecting IgG N-glycan presents a versatile strategy for developing antibody conjugates with site-specificity. The highly conserved core structure of N-glycans enables this strategy to translate to glycoproteins other than antibodies for biotherapeutic development. Thus, the present invention provides a method for making glycoprotein conjugates in general and antibody-drug conjugates in particular.

### Bisected N-Glycans Comprising a Bisecting N-Azidoacetylglucosamine Residue

Glycoproteins expressed in mammalian cell culture may comprise one or more N-glycans and in a composition of such glycoproteins, the glycoforms present may be heterogenous. For example, each Asn at position 297 (Eu numbering) of the Fc domain of an antibody expressed in mammalian cell culture is conjugated to an N-glycan having a particular glycoform. Additional glycoforms may be present in antibodies comprising one or naturally occurring N-glycosylation sites in the VH and/or VL domains and further still in antibodies engineered to comprise one or more non-naturally occurring N-glycosylation sites. In a composition, the particular glycoform varies from antibody to antibody, which results in a composition comprising a heterogenous mixture N-glycan glycoforms. To provide a composition of glycoproteins having a more homogenous population of N-glycans comprising predominantly FA2 N-glycans, the glycoproteins are purified from the cell culture and then treated in vitro with one or more neuraminidases and one or more galactosidases to produce a composition of glycoproteins in which the predominant N-glycan is the bi-antennary FA2 N-glycan represented by the formula GlcNAcβ1-2Manα1-3 (GlcNAcβ1-2Manα1-6)Manβ1-4GlcNAcβ1-4 (Fucα1-6) GlcNAcβ1-Asn or the formula

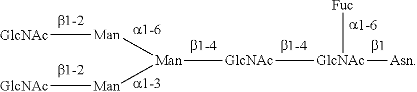

Alternatively, the glycoproteins may be expressed in a yeast host cell that has been genetically modified to produce glycoproteins comprising mammalian or human-like N-glycans. Yeast such as Pichia pastoris and Saccharomyces cerevisiae do not produce complex N-glycans because they lack GnT-I, GnT-II, various mannosidases, galactosyltransferases, fucosyltransferases, and sialyltransferases. Instead, the yeast N-glycosylation pathway comprises initiating α1,6-mannosyltransaferase and a number of other mannosyltransferases, which together result in the production of high mannose N-glycans, which are not found in mammalian cells.

However, the N-glycosylation pathway in yeast may be modified by deleting the gene encoding the initiating α1,6-mannosyltransaferase and introducing into the yeast cell those glycosyltransferases needed to produce N-glycans having a particular glycoforms. For example, yeast such as Pichia pastoris and Saccharomyces cerevisiae comprising a deletion of the gene encoding the initiating α1,6-mannosyltransaferase and the introduction of nucleic acid molecules encoding α1,2-mannosidase, GnT-I, mannosidase II, and GnT-II, and optionally UDP-GlcNAc transporters results in a yeast host cell that is capable of producing glycoproteins comprising predominantly the A2 biantennary N-glycan. (See for example, U.S. Pat. No. 7,326,681). Further including α1,6-fucosyltransferase and UDP-fucose transporters, results in a yeast host cell that is capable of producing glycoproteins comprising predominantly the FA2 biantennary N-glycan shown above.

The glycoproteins comprising the FA2 biantennary N-glycans may be reacted in vitro with a GnT-III in the presence of UDP-azido-GlcNAc (UDP-GlcNAz or UDP-6-azido-GlcNAc) to produce a composition of N-glycosylated glycoproteins in which the predominant N-glycan is a bisected FA2B-azide N-glycan represented by the formula (i) GlcNAcβ1-2Manα1-3 (GlcNAcβ1-2Manα1-6) (Azido-GlcNAcβ1-4)Manβ1-4GlcNAcβ1-4 (Fucα1-6) GlcNAcβ1-Asn or

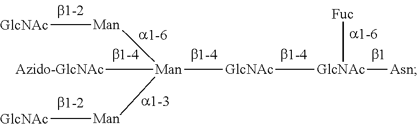

wherein Azido-GlcNAc is GlcNAz or 6-azido-GlcNAc.

The glycoproteins comprising predominantly FA2B-azide N-glycans may be used for conjugating to a payload as disclosed herein. However, in further embodiments, it may be desirable to conjugate payloads to glycoproteins comprising galactosylated N-glycans or sialylated N-glycans.

In further embodiments, glycoproteins comprising FA2B-azide N-glycans are reacted in vitro with a β1-4 galactosyltransferase (GalT) in the presence of UDP-glucose to produce a composition of N-glycosylated glycoproteins in which the predominant N-glycan is a bisected FA2BG2-azide N-glycan represented by the formula Galβ1-4GlcNAcβ1-2Manα1-3(Galβ1-4GlcNAcβ1-2Manα1-6)(GlcNAcβ1-4)Manβ1-4GlcNAcβ1-4 (Fucα1-6) GlcNAcβ1-Asn or

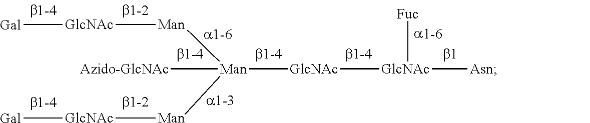

wherein Azido-GlcNAc is GlcNAz or 6-azido-GlcNAc. The glycoproteins comprising predominantly FA2BG2-azide N-glycans may be used for conjugating to a payload as disclosed herein.

In particular embodiments, the predominant N-glycans are a mixture of bisected FA2BG2-azide, bisected FA2B[3]G1-azide, and bisected FA2B[6]G1-azide N-glycans wherein the bisected FA2B[3]G1-azide N-glycan is represented by the formula Galβ1-4GlcNAcβ1-2Manα1-3 (GlcNAcβ1-2Manα1-6)(GlcNAzβ1-4)Manβ1-4GlcNAcβ1-4 (Fucα1-6) GlcNAcβ1-Asn or the formula

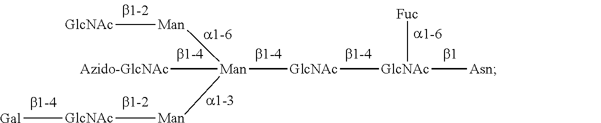

wherein Azido-GlcNAc is GlcNAz or 6-azido-GlcNAc; and the FA2B[6]G1-azide N-glycan is represented by the formula GlcNAcβ1-2Manα1-3(Galβ1-4GlcNAcβ1-2Manα1-6)(GlcNAzβ1-4)Manβ1-4GlcNAcβ1-4(Fucα1-6)GlcNAcβ1-Asn or the formula

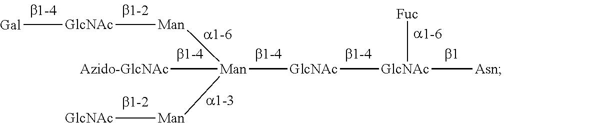

wherein Azido-GlcNAc is GlcNAz or 6-azido-GlcNAc. The glycoproteins comprising these predominant glycoforms may be used for conjugating to a payload as disclosed herein.

In further embodiments, glycoproteins comprising FA2BG2-azide N-glycans are reacted with an α2-6 sialyltransferase (SiaT) in the presence of CMP-sialic acid to produce a composition of N-glycosylated glycoproteins in which the predominant N-glycan is a bisected FA2BG2S2(6)-azide N-glycan represented by the formula Siaα2-6Galβ1-4GlcNAcβ1-2Manα1-3(Siaα2-6Galβ1-4GlcNAcβ1-2Manα1-6)(GlcNAzβ1-4)Manβ1-4GlcNAcβ1-4(Fucα1-6)GlcNAcβ1-Asn or the formula

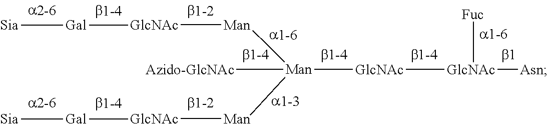

wherein Azido-GlcNAc is GlcNAz or 6-azido-GlcNAc. The glycoproteins comprising predominantly FA2BG2S2(6)-azide N-glycans may be used for conjugating to a payload as disclosed herein.

In particular embodiments, the predominant N-glycans are a mixture of bisected FA2BG2S2(6)-azide, bisected FA2BG2[3]S1(6)-azide, and bisected FA2BG2[6]S1(6)-azide N-glycans wherein the bisected FA2BG2[3]S1(6)-azide N-glycan is represented by the formula Siaα2-6Galβ1-4GlcNAcβ1-2Manα1-3(Galβ1-4GlcNAcβ1-2Manα1-6)(GlcNAzβ1-4)Manβ1-4GlcNAcβ1-4 (Fucα1-6)GlcNAcβ1-Asn or the formula

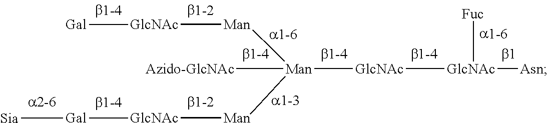

wherein Azido-GlcNAc is GlcNAz or 6-azido-GlcNAc; and the FA2BG2[6]S1(6)-azide N-glycan is represented by the formula Galβ1-4GlcNAcβ1-2Manα1-3(Siaα2-6Galβ1-4GlcNAcβ1-2Manα1-6)(GlcNAzB1-4)Manβ1-4GlcNAcβ1-4 (Fucα1-6)GlcNAcβ1-Asn or the formula

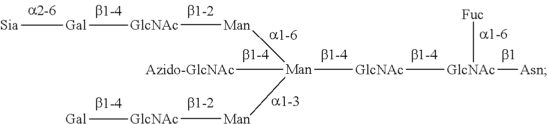

wherein Azido-GlcNAc is GlcNAz or 6-azido-GlcNAc. The glycoproteins comprising these predominant glycoforms may be used for conjugating to a payload as disclosed herein.

In further embodiments, glycoproteins comprising FA2BG2-azide N-glycans are reacted with an α2-3 sialyltransferase (SiaT) in the presence of CMP-sialic acid to produce a composition of N-glycosylated glycoproteins in which the predominant N-glycan is a bisected FA2BG2S2(3)-azide N-glycan represented by the formula Siaα2-3Galβ1-4GlcNAcβ1-2Manα1-3(Siaα2-3Galβ1-4GlcNAcβ1-2Manα1-6)(GlcNAzB1-4)Manβ1-4GlcNAcβ1-4(Fucα1-6)GlcNAcβ1-Asn or the formula

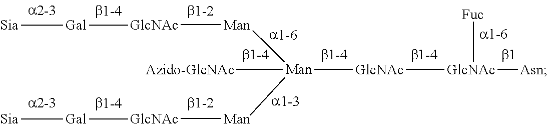

wherein Azido-GlcNAc is GlcNAz or 6-azido-GlcNAc. The glycoproteins comprising predominantly FA2BG2S2(3)-azide N-glycans may be used for conjugating to a payload as disclosed herein.

In particular embodiments, the predominant N-glycans are a mixture of bisected FA2BG2S2(3)-azide, bisected FA2BG2[3]S1(3)-azide, and bisected FA2BG2[6]S1(3)-azide N-glycans wherein the bisected FA2BG2[3]S1(3)-azide N-glycan is represented by the formula Siaα2-3Galβ1-4GlcNAcβ1-2Manα1-3(Galβ1-4GlcNAcβ1-2Manα1-6)(GlcNAzβ1-4)Manβ1-4GlcNAcβ1-4(Fucα1-6)GlcNAcβ1-Asn or the formula

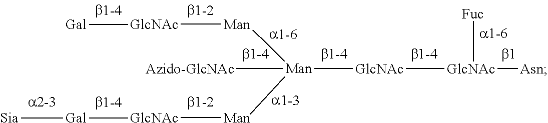

wherein Azido-GlcNAc is GlcNAz or 6-azido-GlcNAc; and the FA2BG2[6]S1(3)-azide N-glycan is represented by the formula Galβ1-4GlcNAcβ1-2Manα1-3(Siaα2-3Galβ1-4GlcNAcβ1-2Manα1-6)(GlcNAzB1-4)Manβ1-4GlcNAcβ1-4 (Fucα1-6)GlcNAcβ1-Asn or the formula

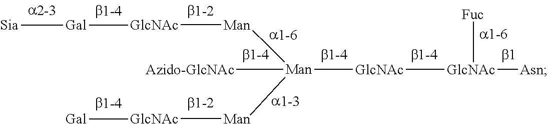

wherein Azido-GlcNAc is GlcNAz or 6-azido-GlcNAc. The glycoproteins comprising the above predominant glycoforms may be used for conjugating to a payload directly or indirectly comprising an alkyne group for forming a triazole linkage with the azido group of the bisecting GlcNAz as disclosed herein. In further embodiments, the glycoprotein may be an antibody, a Fab, scFv, or a fusion protein comprising an Fc domain of an antibody fused or conjugated to a heterologous protein.

The classic antibody ADCC response is mediated by natural killer (NK) cells following the binding of the FcγRIIIa to the Fc region of antibody molecules. This binding triggers the NK cells to release cytokines and cytolytic agents that eventually kill the target cell. The ADCC activity is highly affected by the Fc N-glycan. In recombinant IgG therapeutics produced in Chinese hamster ovary (CHO) cells, the Fc N-glycans are heterogeneous biantennary complex type with a fucose residue attached to the core position. These N-glycans contain little to no sialic acid with zero galactose residues (A2), one galactose residue (A2[3 or 6]G1) or two galactose residues (A2G2). In the study by Shields et al., humanized IgG1 glycoproteins expressed in CHO Lec13 cells demonstrated a 50-fold improvement in binding affinity to human FcγRIIIa compared to the same glycoproteins produced in wild type CHO cells (See Shields et al., Lack of fucose on human IgG1 N-linked oligosaccharide improves binding to human Fcγ RIII and antibody-dependent cellular toxicity. J Biol Chem. 277: 26733-40 (2002)).

In particular embodiments, the glycoproteins may be expressed in a mammalian cell culture in which the mammalian cells thereof have been genetically engineered to produce glycoproteins comprising afucosylated N-glycans. For example, deleting the FUT8 gene in Chinese hamster ovary (CHO) cells produces a mutant CHO cell that produces afucosylated glycoproteins (Yamane-Ohnuki et al., Establishment of FUT8 knockout Chinese hamster ovary cells: an ideal host cell line for producing completely defucosylated antibodies with enhanced antibody-dependent cellular cytotoxicity. Biotechnol Bioeng. 87: 614-22 (2004) and Malphettes et al., Highly efficient deletion of FUT8 in CHO cell lines using zinc-finger nucleases yields cells that produce completely nonfucosylated antibodies. Biotechnol Bioeng. 106: 774-83 (2010)).

To provide glycoproteins having more homogenous N-glycans comprising predominantly A2 N-glycans, the glycoproteins are purified from the cell culture and then treated in vitro with one or more neuraminidases and one or more galactosidases to produce a composition of glycoproteins in which the predominant N-glycan is the bi-antennary afucosylated A2 N-glycan represented by the formula GlcNAcβ1-2Manα1-3(GlcNAcβ1-2Manα1-6)Manβ1-4GlcNAcβ1-4GlcNAcβ1-Asn or the formula

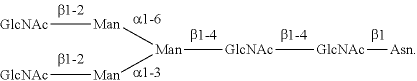

Alternatively, the glycoproteins may be expressed in a yeast host cell that has been genetically modified to produce glycoproteins comprising mammalian or human-like N-glycans. The N-glycosylation pathway in yeast may be modified by deleting the gene encoding the initiating α1,6-mannosyltransaferase and introducing into the yeast cell those glycosyltransferases needed to produce N-glycans having a particular glycoforms. For example, yeast such as Pichia pastoris and Saccharomyces cerevisiae comprising a deletion of the gene encoding the initiating α1,6-mannosyltransaferase and the introduction of nucleic acid molecules encoding α1,2-mannosidase, GnT-I, mannosidase II, and GnT-II and optionally UDP-GlcNAc transporters results in a yeast host cell that is capable of producing glycoproteins comprising predominantly afucosylated A2 N-glycans comprising the structure above. (See for example, U.S. Pat. No. 7,326,681).

The glycoproteins comprising the biantennary GlcNAc₂Man₃GlcNAc₂ N-glycans are reacted with a GnT-III in the presence of UDP-azide-GlcNAc to produce a composition of N-glycosylated glycoproteins in which the predominant N-glycan is a bisected A2B-azide N-glycan represented by the formula GlcNAcβ1-2Manα1-3(GlcNAcβ1-2Manα1-6)(GlcNAzβ1-4)Manβ1-4GlcNAcβ1-4GlcNAcβ1-Asn or the formula

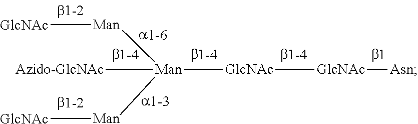

wherein Azido-GlcNAc is GlcNAz or 6-azido-GlcNAc.

The glycoproteins comprising predominantly A2B-azide N-glycans may be used for conjugating to a payload as disclosed herein. However, in further embodiments, it may be desirable to conjugate payloads to glycoproteins comprising galactosylated N-glycans of sialylated N-glycans.

In further embodiments, glycoproteins comprising A2B-azide N-glycans are reacted with a β1-4 galactosyltransferase (GalT) in the presence of UDP-glucose to produce a composition of N-glycosylated glycoproteins in which the predominant N-glycan is a bisected A2BG2-azide N-glycan represented by the formula Galβ1-4GlcNAcβ1-2Manα1-3(Galβ1-4GlcNAcβ1-2Manα1-6)(GlcNAzβ1-4)Manβ1-4GlcNAcβ1-4GlcNAcβ1-Asn or the formula

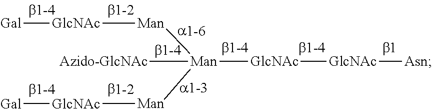

wherein Azido-GlcNAc is GlcNAz or 6-azido-GlcNAc. The glycoproteins comprising predominantly FA2BG2-azide N-glycans may be used for conjugating to a payload as disclosed herein.

In particular embodiments, the predominant N-glycans are a mixture of bisected A2BG2-azide, bisected A2B[3]G1-azide, and bisected A2B[6]G1-azide N-glycans wherein the bisected A2B[3]G1-azide N-glycan is represented by the formula Galβ1-4GlcNAcβ1-2Manα1-3(GlcNAcβ1-2Manα1-6)(GlcNAzβ1-4)ManB1-4GlcNAcβ1-4GlcNAcβ1-Asn or the formula

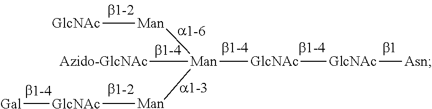

wherein Azido-GlcNAc is GlcNAz or 6-azido-GlcNAc; and the A2B[6]G1-azide N-glycan is represented by the formula GlcNAcβ1-2Manα1-3(Galβ1-4GlcNAcβ1-2Manα1-6)(GlcNAzβ1-4)Manβ1-4GlcNAcβ1-4GlcNAcβ1-Asn or the formula

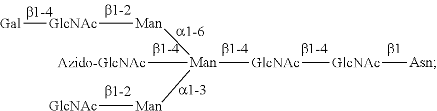

wherein Azido-GlcNAc is GlcNAz or 6-azido-GlcNAc. The glycoproteins comprising these predominant glycoforms may be used for conjugating to a payload as disclosed herein.

In further embodiments, antibodies comprising A2BG2-azide N-glycans are reacted with an α2-6 sialyltransferase (SiaT) in the presence of CMP-sialic acid to produce a composition of N-glycosylated glycoproteins in which the predominant N-glycan is a bisected A2BG2S2(6)-azide N-glycan represented by the formula Siaα2-6Galβ1-4GlcNAcβ1-2Manα1-3(Siaα2-6Galβ1-4GlcNAcβ1-2Manα1-6)(GlcNAzβ1-4)Manβ1-4GlcNAcβ1-4GlcNAcβ1-Asn or the formula

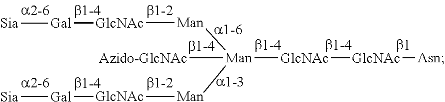

wherein Azido-GlcNAc is GlcNAz or 6-azido-GlcNAc. The glycoproteins comprising predominantly FA2BG2S2(6)-azide N-glycans may be used for conjugating to a payload as disclosed herein.

In particular embodiments, the predominant N-glycans are a mixture of bisected A2BG2S2(6)-azide, bisected A2BG2[3]S1(6)-azide, and bisected A2BG2[6]S1(6)-azide N-glycans wherein the bisected A2BG2[3]S1(6)-azide N-glycan is represented by the formula Siaα2-6Galβ1-4GlcNAcβ1-2Manα1-3(Galβ1-4GlcNAcβ1-2Manα1-6)(GlcNAzβ1-4)Manβ1-4GlcNAcβ1-4GlcNAcβ1-Asn or the formula

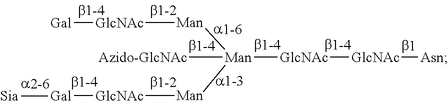

wherein Azido-GlcNAc is GlcNAz or 6-azido-GlcNAc; and the A2BG2[6]S1(6)-azide N-glycan is represented by the formula Galβ1-4GlcNAcβ1-2Manα1-3(Siaα2-6Galβ1-4GlcNAcβ1-2Manα1-6)(GlcNAzβ1-4)Manβ1-4GlcNAcβ1-4GlcNAcβ1-Asn or the formula

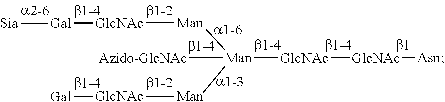

wherein Azido-GlcNAc is GlcNAz or 6-azido-GlcNAc. The glycoproteins comprising these predominant glycoforms may be used for conjugating to a payload as disclosed herein.

In further embodiments, glycoproteins comprising A2BG2-azide N-glycans are reacted with an α2-3 sialyltransferase (SiaT) in the presence of CMP-sialic acid to produce a composition of N-glycosylated glycoproteins in which the predominant N-glycan is a bisected A2BG2S2(3)-azide N-glycan represented by the formula Siaα2-3Galβ1-4GlcNAcβ1-2Manα1-3(Siaα2-3Galβ1-4GlcNAcβ1-2Manα1-6)(GlcNAzβ1-4)Manβ1-4GlcNAcβ1-4GlcNAcβ1-Asn or the formula

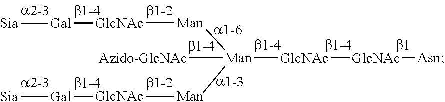

wherein Azido-GlcNAc is GlcNAz or 6-azido-GlcNAc. The glycoproteins comprising predominantly FA2BG2S2(3)-azide N-glycans may be used for conjugating to a payload as disclosed herein.

In particular embodiments, the predominant N-glycans are a mixture of bisected A2BG2S2(3)-azide, bisected A2BG2[3]S1(3)-azide, and bisected A2BG2[6]S1(3)-azide N-glycans wherein the bisected A2BG2[3]S1(3)-azide N-glycan is represented by the formula Siaα2-3Galβ1-4GlcNAcβ1-2Manα1-3(Galβ1-4GlcNAcβ1-2Manα1-6)(GlcNAzβ1-4)Manβ1-4GlcNAcβ1-4GlcNAcβ1-Asn or the formula

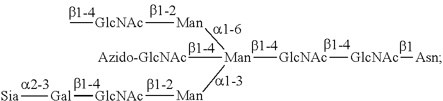

wherein Azido-GlcNAc is GlcNAz or 6-azido-GlcNAc; and the A2BG2[6]S1(3)-azide N-glycan is represented by the formula Galβ1-4GlcNAcβ1-2Manα1-3(Siaα2-3Galβ1-4GlcNAcβ1-2Manα1-6)(GlcNAzβ1-4)Manβ1-4GlcNAcβ1-4GlcNAcβ1-Asn or the formula

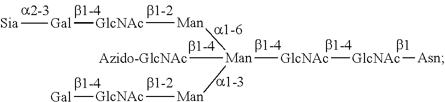

wherein Azido-GlcNAc is GlcNAz or 6-azido-GlcNAc. The glycoproteins comprising these predominant glycoforms may be used for conjugating to a payload as disclosed herein.

In particular embodiments of the aforementioned glycoproteins, the glycoprotein is an antibody, Fab, or scFv comprising at least one N-glycosylation site. In particular embodiments, the glycoprotein is a fusion protein comprising the Fc domain of an antibody fused to a heterologous protein or heterologous glycoprotein. In particular embodiments, the heterologous glycoprotein is a Fab or scFv comprising at least one N-glycosylation site.

### Engineering Non-Native N-Glycosylation Sites in Antibodies

Antibodies comprise an N-glycosylation site beginning with the Asn at position 297 (Eu numbering) of the constant domain of the heavy chain and may comprise one or more naturally occurring N-glycosylation sites located in the V_{H} and V_{L} domains. Since antibodies are tetramers comprising two heterodimers, each heterodimer comprising one light chain and one heavy chain, the maximum DAR achievable for an ADC comprising solely the N-glycosylation sites at position 297 is two.

To provide ADCs with DARs greater than two, the present invention further provides antibodies comprising one or more amino acid substitutions in the variable or constant domain of the antibody that generate one or more non-native N-glycosylation sites comprising the amino acid sequence Asn-X-Thr/Ser, wherein X is any amino acid except Pro, and wherein the N-glycan conjugated to each non-native N-glycosylation site comprises a bisected N-glycan in which the bisecting sugar of the bisected N-glycan is an azido-GlcNAc residue. In further embodiments, the azide group of the azido-GlcNAc residue of the bisecting N-glycan is further conjugated in a triazole linkage to an alkyne group of a payload. Antibodies may be engineered to comprise one to ten amino acid substitutions (or pairs of amino acid substitutions) in the heavy chain constant domain, which generate one to ten non-native N-glycosylation sites, the amino acid substitutions being selected from the group consisting of S134N, G161T, G161S, N203T, N203S, V363T, V363S, Q438N, S176N, A118N, S132N, K133N, A162N, T195N, K210T, Y391T, F423T, F423S, Y436T, Y436S, L193N, K392T, K392S, F423T, S176N/G178T, S176N/G178S, Q419N/N421T, Q419N/N421S, S191N/L193T, S191N/L193S, G194N/Q196T, and G194N/Q196S, wherein the position numbering is according to EU numbering.

In general, an ADC in which the N-glycosylation site is at position 297 (Eu numbering) will have a DAR of up to 2. By making amino acid substitutions at one or more of the specified positions ADCs may be constructed that may comprise a DAR of up to 4, 6, 8, 10, 12, 14, 15, 18, 20, or 22.

In particular embodiments, the engineered antibody or fragment thereof may comprise a human IgG1, IgG2, or IgG4 constant domain. In particular embodiments, the engineered antibody comprises a human IgG1 constant domain. In particular embodiments, the engineered antibody comprises a human IgG4 constant domain.

In an embodiment of the present invention, the engineered antibody comprises at least one or two amino acid substitutions (or a pair of substitutions) in the heavy chain constant domain selected from S134N, G161T and S134N/G161T. In another embodiment, the engineered antibody comprises at least amino acid substitutions in the heavy chain constant domain selected from G161T/S134T and G161S/S134T. In a particular embodiment, the antibody comprises an S134N amino acid substitution, which when combined with the native N297 N-glycosylation site results in an ADC that may comprise a DAR of up to 4. In a further embodiment, the antibody comprises a G161T amino acid substitution, which when combined with the native N297 N-glycosylation site results in an ADC that may comprise a DAR of up to 4. In a further embodiment, the antibody comprises an S134N amino acid substitution and a G1261T amino acid substitution, which when combined with the native N297 N-glycosylation site results in an ADC that may comprise a DAR of up to 6.

In another embodiment, the antibody possess one to two amino acid substitutions in the heavy chain variable framework domain which generate one to two non-native N-glycosylation sites, the amino acid substitutions being selected from the group consisting of Q105N and S113N, the position numbering is according to Kabat numbering. An antibody with a Q105N amino acid substitution when combined with the native N297 N-glycosylation site results in an ADC that may comprise a DAR of up to 4. An antibody with a S113N amino acid substitution when combined with the native N297 N-glycosylation site results in an ADC that may comprise a DAR of up to 4. An antibody with a Q105N amino acid substitution and an S113N amino acid substitution when combined with the native N297 N-glycosylation site results in an ADC that may comprise a DAR of up to 4.

Glycoproteins other than antibodies may be modified to comprise one or more non-native N-glycosylation sites comprising the amino acid sequence Asn-Xaa-Thr/Ser, wherein X is any amino acid except Pro, and wherein the N-glycan conjugated to each non-native N-glycosylation site comprises a bisected N-glycan in which the bisecting sugar of the bisected N-glycan is an azido-GlcNAc residue. In further embodiments, the azide group of the azido-GlcNAc residue of the bisecting N-glycan is further conjugated in a triazole linkage to an alkyne group of a payload. The position of the non-native N-glycosylation will depend on the particular glycoprotein that is modified.

### Payloads

### (a) Therapeutic Effector Moieties

In certain embodiments, the antibodies of the current disclosure are conjugated to a therapeutic effector moiety comprising a therapeutic agent, e.g., a drug moiety (or prodrug thereof) or radiolabeled compound. In one embodiment, the therapeutic agent is a cytotoxin.

Exemplary drug moieties include anti-inflammatory agents, anti-cancer agents, anti-infective agents (e.g., anti-fungal, antibacterial, anti-parasitic, anti-viral, etc.), and anesthetic therapeutic agents. In a further embodiment, the drug moiety is an anti-cancer agent.

Exemplary anti-cancer agents include, but are not limited to, cytostatics, enzyme inhibitors, gene regulators, cytotoxic nucleosides, tubulin binding agents, tubulin disrupters, tubulin inhibitors, topoisomerase inhibitors, proteasome inhibitors, hormones and hormone antagonists, anti-angiogenesis agents, and the like. Any such molecule may be conjugated to an antibody or other glycoprotein according to the present invention.

Exemplary cytostatic anti-cancer agents include alkylating agents such as the anthracycline family of drugs (e.g., adriamycin, carminomycin, cyclosporin-A, chloroquine, methopterin, mithramycin, porfiromycin, streptonigrin, porfiromycin, anthracenediones, and aziridines). Other cytostatic anti-cancer agents include DNA synthesis inhibitors (e.g., methotrexate and dichloromethotrexate, 3-amino-1,2,4-benzotriazine 1,4-dioxide, aminopterin, cytosine β-D-arabinofuranoside, 5-fluoro-5′-deoxyuridine, 5-fluorouracil, ganciclovir, hydroxyurea, actinomycin-D, and mitomycin C), DNA-intercalators or cross-linkers (e.g., bleomycin, carboplatin, carmustine, chlorambucil, cyclophosphamide, cis-diammineplatinum (II) dichloride (cisplatin), melphalan, mitoxantrone, and oxaliplatin), and DNA-RNA transcription regulators (e.g., actinomycin D, daunorubicin, doxorubicin, homoharringtonine, and idarubicin).

Other exemplary cytostatic agents that are compatible with the present disclosure include ansamycin benzoquinones, quinonoid derivatives (e.g., quinolones, genistein, bactacyclin), busulfan, ifosfamide, mechlorethamine, triaziquone, diaziquone, carbazilquinone, indoloquinone EO9, diaziridinyl-benzoquinone methyl DZQ, triethylenephosphoramide, and nitrosourea compounds (e.g., carmustine, lomustine, semustine).

Exemplary cytotoxic nucleoside anti-cancer agents include, but are not limited to: adenosine arabinoside, cytarabine, cytosine arabinoside, 5-fluorouracil, fludarabine, floxuridine, ftorafur, and 6-mercaptopurine. Exemplary anti-cancer tubulin binding agents include, but are not limited to: taxoids (e.g., paclitaxel, docetaxel, taxane), nocodazole, rhizoxin, dolastatins (e.g., Dolastatin-10, -11, or -15), colchicine and colchicinoids (e.g., ZD6126), combretastatins (e.g., Combretastatin A-4, AVE-6032), and vinca alkaloids (e.g., vinblastine, vincristine, vindesine, and vinorelbine (navelbine)). Exemplary anti-cancer hormones and hormone antagonists include, but are not limited to: corticosteroids (e.g., prednisone), progestins (e.g., hydroxyprogesterone or medroprogesterone), estrogens, (e.g., diethylstilbestrol), antiestrogens (e.g., tamoxifen), androgens (e.g., testosterone), aromatase inhibitors (e.g., aminogluthetimide), 17-(allylamino)-17-demethoxygeldanamycin, 4-amino-1,8-naphthalimide, apigenin, brefeldin A, cimetidine, dichloromethylene-diphosphonic acid, leuprolide (leuprorelin), luteinizing hormone-releasing hormone, pifithrin-a, rapamycin, sex hormone-binding globulin, and thapsigargin. Exemplary anti-cancer, anti-angiogenesis compounds include, but are not limited to: Angiostatin K1-3, DL-a-difluoromethyl-ornithine, endostatin, fumagillin, genistein, minocycline, staurosporine, and (±)-thalidomide.

Exemplary anti-cancer enzyme inhibitors include, but are not limited to: S(+)-camptothecin, curcumin, (−)-deguelin, 5,6-diCHlorobenz-imidazole 1-β-D-ribofuranoside, etoposide, formestane, fostriecin, hispidin, 2-imino-1-imidazolidineacetic acid (cyclocreatine), mevinolin, trichostatin A, tyrphostin AG 34, and tyrphostin AG 879.

Exemplary anti-cancer gene regulators include, but are not limited to: 5-aza-2′-deoxycytidine, 5-azacytidine, cholecalciferol (vitamin D3), 4-hydroxytamoxifen, melatonin, mifepristone, raloxifene, trans-retinal (vitamin A aldehydes), retinoic acid, vitamin A acid, 9-cis-retinoic acid, 13-cis-retinoic acid, retinol (vitamin A), tamoxifen, and troglitazone.

Other classes of anti-cancer agents include, but are not limited to: the pteridine family of drugs, diynenes, and the podophyllotoxins. Particularly useful members of those classes include, for example, methopterin, podophyllotoxin, or podophyllotoxin derivatives such as etoposide or etoposide phosphate, leurosidine, vindesine, leurosine and the like.

Still other anti-cancer agents that are compatible with the teachings herein include auristatins (e.g., auristatin E and monomethylauristan E), geldanamycin, calicheamicin, gramicidin D, maytansanoids (e.g., maytansine), neocarzinostatin, topotecan, taxanes, cytochalasin B, ethidium bromide, emetine, tenoposide, colchicin, dihydroxy anthracindione, mitoxantrone, procaine, tetracaine, lidocaine, propranolol, puromycin, and analogs or homologs thereof.

Still other anti-cancer agents that are compatible with the teachings herein include tomaymycin derivatives, maytansine derivatives, cryptophycine derivatives, anthracycline derivatives, bisphosphonate derivatives, leptomycin derivatives, streptonigrin derivatives, auristatine derivatives, and duocarmycin derivatives.

Another class of compatible anti-cancer agents that may be used as drug moieties are radiosensitizing drugs that may be effectively directed to tumor or immunoreactive cells. Such drug moieties enhance the sensitivity to ionizing radiation, thereby increasing the efficacy of radiotherapy. Not to be limited by theory, an antibody modified with a radiosensitizing drug moiety and internalized by the tumor cell would deliver the radiosensitizer nearer the nucleus where radiosensitization would be maximal. Antibodies which lose the radiosensitizer moiety would be cleared quickly from the blood, localizing the remaining radiosensitization agent in the target tumor and providing minimal uptake in normal tissues. After clearance from the blood, adjunct radiotherapy could be administered by external beam radiation directed specifically to the tumor, radioactivity directly implanted in the tumor, or systemic radioimmunotherapy with the same modified antibody.

In one embodiment, the therapeutic agent comprises radionuclides or radiolabels with high-energy ionizing radiation that are capable of causing multiple strand breaks in nuclear DNA, leading to cell death. Exemplary high-energy radionuclides include: ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re. These isotopes typically produce high energy α- or β-particles which have a short path length. Such radionuclides kill cells to which they are in close proximity, for example neoplastic cells to which the conjugate has attached or has entered. They have little or no effect on non-localized cells and are essentially non-immunogenic. Alternatively, high-energy isotopes may be generated by thermal irradiation of an otherwise stable isotope, for example as in boron neutron-capture therapy (Guan et al., Homogeneous immunoconjugates for boron neutron-capture therapy: Design, synthesis, and preliminary characterization. Proc. Natl. Acad. Sci. USA 95: 13206-10, (1998)).

In one embodiment, the therapeutic agent is selected from MMAE, MMAF, and PEGS-Dol10. In further embodiments, the therapeutic agent is DXd, DM4, SN-38, camptothecin, or PNU.

Exemplary cytotoxic agents may be selected from the group consisting of taxanes, anthracyclines, camptothecins, epothilones, mytomycins, combretastatins, vinca alkaloids, maytansinoids, enediynes, duocarmycins, tubulysins, amatoxins, bleomycins, dolastatins and auristatins, pyrrolobenzodiazepine dimers, indolinobenzodiazepine dimers, and radioisotopes.

Exemplary anti-inflammatory agents include glucocorticoid receptor agonists, which in further embodiments include cortisol, cortisone acetate, beclometasone, prednisone, prednisolone, methylprednisolone, betamethasone, trimcinolone, budesonide, dexamethasone, fluticasone, or mometasone.

In certain embodiments, the effector moiety contains more than one therapeutic agent. These multiple therapeutic agents can be the same or different.

### b) Diagnostic Effector Moieties

In certain embodiments, the antibodies of the current disclosure are conjugated to a payload wherein the payload is a diagnostic effector moiety comprising a diagnostic agent. In one embodiment, the diagnostic agent is a detectable small molecule label e.g., biotin, fluorophores, chromophores, spin resonance probes, or radiolabels. Exemplary fluorophores include fluorescent dyes (e.g., fluorescein, rhodamine, and the like) and other luminescent molecules (e.g., luminal). A fluorophore may be environmentally-sensitive such that its fluorescence changes if it is located close to one or more residues in the modified binding polypeptide that undergo structural changes upon binding a substrate (e.g., dansyl probes). Exemplary radiolabels include small molecules containing atoms with one or more low sensitivity nuclei (¹³C, ¹⁵N, ²H, ¹²⁵I, ¹²⁴I, ¹²³I, ⁹⁹Tc, ⁴³K, ⁵²Fe, ⁶⁴Cu, ⁶⁸Ga, ¹¹¹In, and the like). The radionuclide can be, e.g., a gamma, photon, or positron-emitting radionuclide with a half-life suitable to permit activity or detection after the elapsed time between administration and localization to the imaging site.

In one embodiment, the diagnostic agent is a polypeptide. Exemplary diagnostic polypeptides include enzymes with fluorogenic or chromogenic activity, e.g., the ability to cleave a substrate which forms a fluorophore or chromophore as a product (i.e., reporter proteins such as luciferase). Other diagnostic proteins may have intrinsic fluorogenic or chromogenic activity (e.g., green, red, and yellow fluorescent bioluminescent aequorin proteins from bioluminescent marine organisms) or they may comprise a protein containing one or more low-energy radioactive nuclei (¹³C, ¹⁵N, ²H, ¹²⁵I, ¹²⁴I, ¹²³I, ⁹⁹Tc, ⁴³K, ⁵²Fe, ⁶⁴Cu, ⁶⁸Ga, ¹¹¹In, and the like).

With respect to the use of radiolabeled conjugates in conjunction with the present disclosure, antibodies of the current disclosure may be directly labeled (such as through iodination) or may be labeled indirectly through the use of a chelating agent. As used herein, the phrases “indirect labeling” and “indirect labeling approach” both mean that a chelating agent is covalently attached to a binding polypeptide and at least one radionuclide is associated with the chelating agent. Such chelating agents are typically referred to as bifunctional chelating agents as they bind both the polypeptide and the radioisotope. Exemplary chelating agents comprise 1-isothiocycmatobenzyl-3-methyldiothelene triaminepentaacetic acid (“MX-DTPA”) and cyclohexyl diethylenetriamine pentaacetic acid (“CHX-DTPA”) derivatives. Other chelating agents comprise P-DOTA and EDTA derivatives. Exemplary radionuclides for indirect labeling include ¹¹¹In and ⁹⁰Y. Most imaging studies utilize 5 mCi ¹¹¹In-labeled antibody, because this dose is both safe and has increased imaging efficiency compared with lower doses, with optimal imaging occurring at three to six days after antibody administration. See, for example, Murray, J. Nuc. Med. 26: 3328 (1985) and Carraguillo et al., J. Nuc. Med. 26: 67 (1985). An exemplary radionuclide for direct labeling is ¹³¹I. Those skilled in the art will appreciate that non-radioactive conjugates may also be assembled depending on the selected agent to be conjugated.

In certain embodiments, the diagnostic effector moiety is a FRET (Fluorescence Resonance Energy Transfer) probe. FRET has been used for a variety of diagnostic applications including cancer diagnostics. A FRET probe may include a cleavable linker (enzyme sensitive or pH linker) connecting the donor and acceptor moieties of the FRET probe, wherein cleavage results in enhanced fluorescence (including near Infrared) (see, e.g., A. Cobos-Correa et. al. Membrane-bound FRET probe visualizes MMP 12 activity in pulmonary inflammation, Nature Chemical Biology, 5(9), 628-63 (2009); S. Gehrig et. al. Spatially Resolved Monitoring of Neutrophil Elastase Activity with Ratiometric Fluorescent Reporters Angew. Chem. Int. Ed., 51, 6258-6261 (2012)).

### c) Functionalized Effector Moieties

In certain embodiments, effector moieties may be functionalized to contain one or more additional groups in addition to the effector moiety itself. For example, the effector moiety may contain cleavable linkers which release the payload from the antibody under particular conditions. In exemplary embodiments, the effector moiety may include a linker that is cleavable by cellular enzymes and/or is pH sensitive. Additionally or alternatively, the effector moiety may contain a disulfide bond that cleaved by intracellular glutathione upon uptake into the cell.

In yet other embodiments, the effector moiety may include hydrophilic and biocompatible moieties such as poly(glycine), poly(oxazoline), and/or PEG moieties.

### Linkers

In general, any linker or linker technology known in the art or will be known in the art for use in ADCs may be used to create or construct an ADC of the present disclosure provide the linker comprises an alkyne group capable of forming a triazole with the azido group of the bisecting 6-azido-GlcNAc or GlcNAz residue of the bisected N-glycan. In certain embodiments, the linker is a cleavable linker and in other embodiments, the linker is a non-cleavable linker. The linker comprises a distal end and a proximal end, wherein the proximal end comprises an alkyne functional group capable of forming a triazole linkage with the azido group of the bisecting 6-azido-GlcNAc or GlcNAz residue of the bisected N-glycan and the distal end is directly or indirectly covalently linked to a payload. In particular embodiments, the linker comprises a distal end and a proximal end, wherein the proximal end comprises a strained cyclooctyne, and the distal end is directly or indirectly covalently linked to a therapeutic agent. In a further embodiment, the strained cyclooctyne comprises dibenzocyclooctyne (DBCO) amine. A representative linker payload comprising a DBCO functional group is represented by

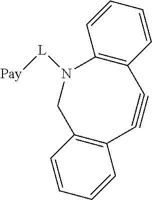

wherein Pay is a payload and L is a linker or bond. In particular embodiments, L may be represented by L¹ and L² in which each is independently a linker element or a bond.

The representative linker conjugated to a payload and the azido group of a bisecting GlcNAz residue of a bisected N-glycan of an antibody is represented by

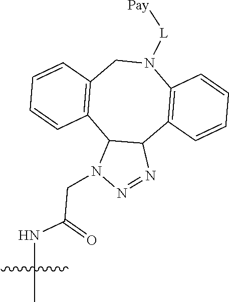

or its regioisomer

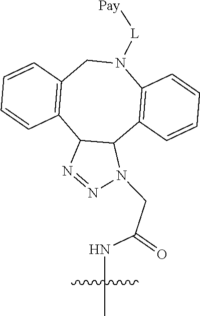

wherein Pay is a payload and L is a linker or bond. In particular embodiments, L may be represented by L¹ and L² in which each is independently a linker element or a bond, and the wavy line identifies the bond between the amine group of GlcNAz and the C2 carbon of the glucose comprising the GlcNAz wherein the GlcNAz is the bisecting sugar of a bisected N-glycan linked to the antibody.

The representative linker conjugated to a payload and the azido group of a bisecting 6-azido-GlcNAc residue of a bisected N-glycan of an antibody is represented by

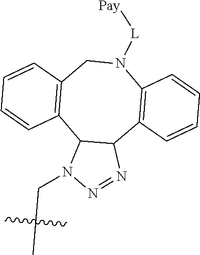

or its regioisomer

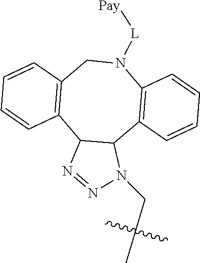

wherein Pay is a payload and L is a linker or bond. In particular embodiments, L may be represented by L¹ and L² in which each is independently a linker element or a bond, and the wavy line identifies the bond between the C5 and C6 carbons of the glucose comprising 6-azido-GlcNAc wherein the 6-azido-GlcNAc is the bisecting sugar of a bisected N-glycan linked to the antibody.

Exemplary linker elements that may be used in the context of the present disclosure include, a sequence of one or more amino acids, for example, dipeptide Val-Cit (valine-citrulline), Val-Ala (valine-alanine), dipeptide site in protease-cleavable linker, Ala-Phe (alanine-phenylalanine), Gly-Gly-Phe-Gly (glycine-glycine-phenylalanine-glycine), dipeptide site in protease-cleavable linker, PABC (p-aminobenzylcarbamate), and variants and combinations thereof.

Examples of linkers and linker-payloads that can be used in the context of the present disclosure include but are not limited to those disclosed in the following patent publications: WO2021055865; WO2021067861; WO2019195665; WO2019236954; WO2021207701; WO2020132655; WO2018201087; WO2018031690; WO2018112253; WO2017096311; WO2015123679; WO2015095755; WO2015057699; U.S. Pat. Nos. 9,504,756; 10,550,190; 10,869,929; WO2018025168; US20210138077; RE45272E; WO2016102679; WO2009099741; WO2010009124; WO2016040835; WO2016094455; WO2016127081; WO2017062271; WO2018234636; WO2018235024; WO2018237335; and WO2019122447, the contents of each patent publication with respect to linkers and linker-payloads are incorporated herein by reference in its entirety, with the proviso that the maleimide, succinimide, sulfone, or other functional group of the linker used for conjugating the linker to an SH or amino group of a protein is replaced by a functional group comprising an alkyne group, in particular embodiments, a strained cyclooctyne, which in a further embodiment may be DBCO, to render the linker capable of being conjugated to the bisecting 6-azido-GlcNAc or GlcNAz residue of the bisected N-glycan in the context of the present invention.

In certain embodiments, the linkers are stable in physiological conditions. In certain embodiments, the linkers are cleavable, for instance, able to release at least the payload portion in the presence of an enzyme or at a particular pH range or value. In some embodiments, a linker comprises an enzyme-cleavable moiety. Illustrative enzyme-cleavable moieties include, but are not limited to, peptide bonds, ester linkages, hydrazones, and disulfide linkages. In some embodiments, the linker comprises a cathepsin-cleavable linker.

In some embodiments, the linker comprises a non-cleavable moiety.

In some embodiments, the linker comprises one or more amino acids. Suitable amino acids include natural, non-natural, standard, non-standard, proteinogenic, non-proteinogenic, and L- or D-a-amino acids. In some embodiments, the linker comprises alanine, valine, glycine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or combination thereof. In certain embodiments, one or more side chains of the amino acids is linked to a side chain group, described below. In some embodiments, the linker comprises valine and citrulline. In some embodiments, the linker comprises lysine, valine, and citrulline. In some embodiments, the linker comprises lysine, valine, and alanine. In some embodiments, the linker comprises valine and alanine.

In some embodiments, the linker comprises a self-immolative group. The self-immolative group can be any such group known to those of skill. In particular embodiments, the self-immolative group is p-aminobenzyl (PAB), or a derivative thereof such as p-aminobenzyloxycarbonyl (PABC). Those of skill will recognize that a self-immolative group is capable of carrying out a chemical reaction that releases the remaining atoms of a linker from a payload.

In particular embodiments, the glycoprotein of the present invention comprises the following structure:

GP-[B-T-L-Pay]ₙ

wherein GP is a glycoprotein; B is a bisected N-glycan wherein the bisecting sugar residue is azido-GlcNAc (either 6-azido-GlcNAc or GlcNAz) having an azide functional group; L is a linker element comprising an alkyne functional group or a bond; Pay is a payload, which comprises an alkyne functional group when L is a bond; T is a triazole linkage formed between the linkage of the azide functional group and the alkyne functional group; and, n is an integer from 1-20. In particular embodiments, the glycoprotein is a Fab or scFv comprising at least one N-glycosylation site. In particular embodiments, the glycoprotein is a fusion protein comprising the Fc domain of an antibody fused to a heterologous protein or heterologous glycoprotein. In particular embodiments, the heterologous glycoprotein is a Fab or scFv comprising at least one N-glycosylation site.

In particular embodiments, L is a self-immolative linker. In particular embodiments, L has the formula L¹-L², wherein each of L¹ and L² is independently a linker element or a bond. In particular embodiments, the L comprises a distal end comprising an alkyne containing group linked to the azide of the 6-azido-GlcNAc or GlcNAz of bisected N-glycan B in triazole linkage T and a proximal end linked to Pay. In a further embodiment, the alkyne group is a strained cyclooctyne. In a further embodiment, the strained cyclooctyne is DBCO.

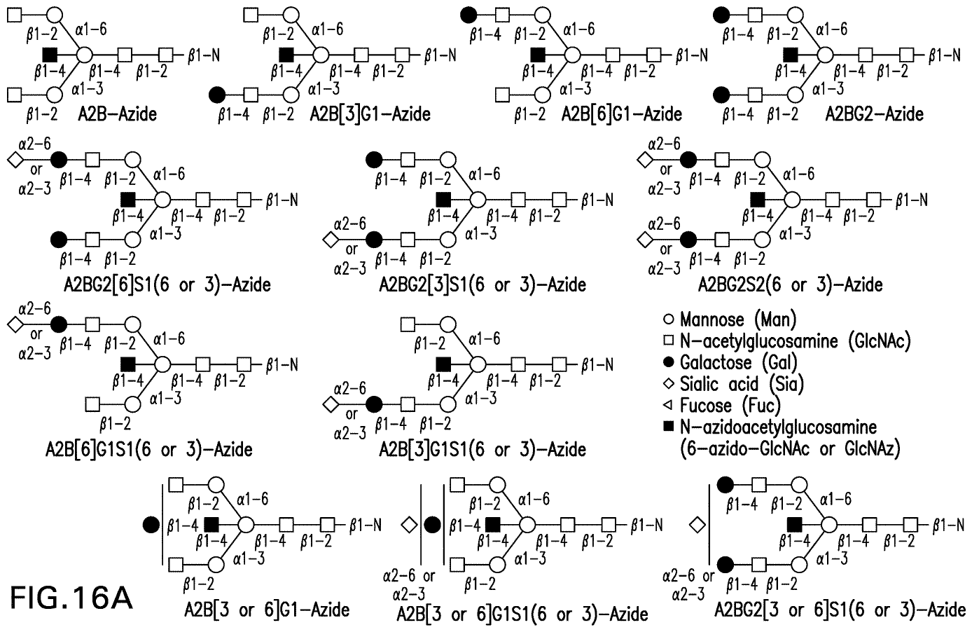

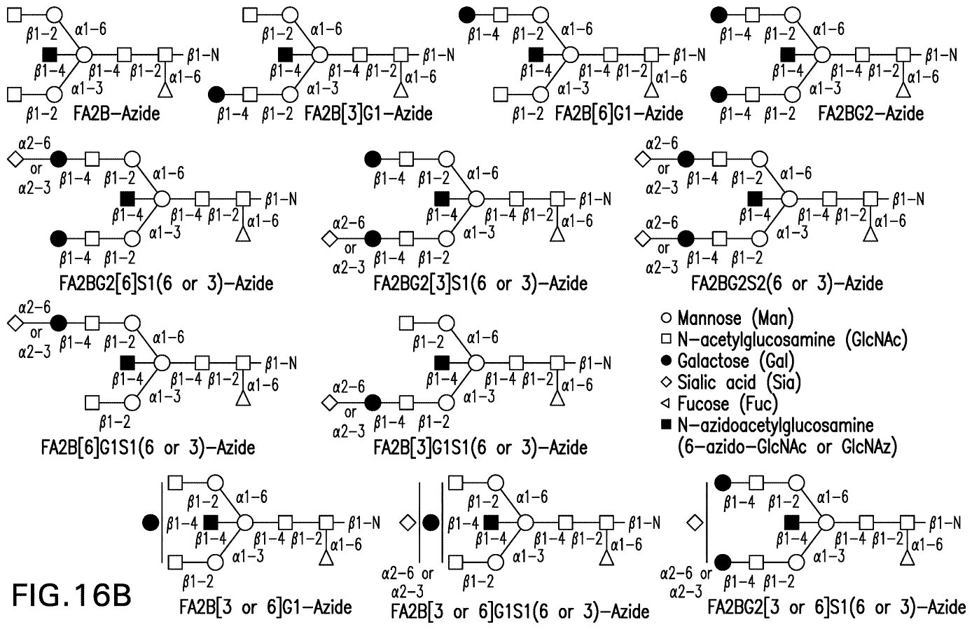

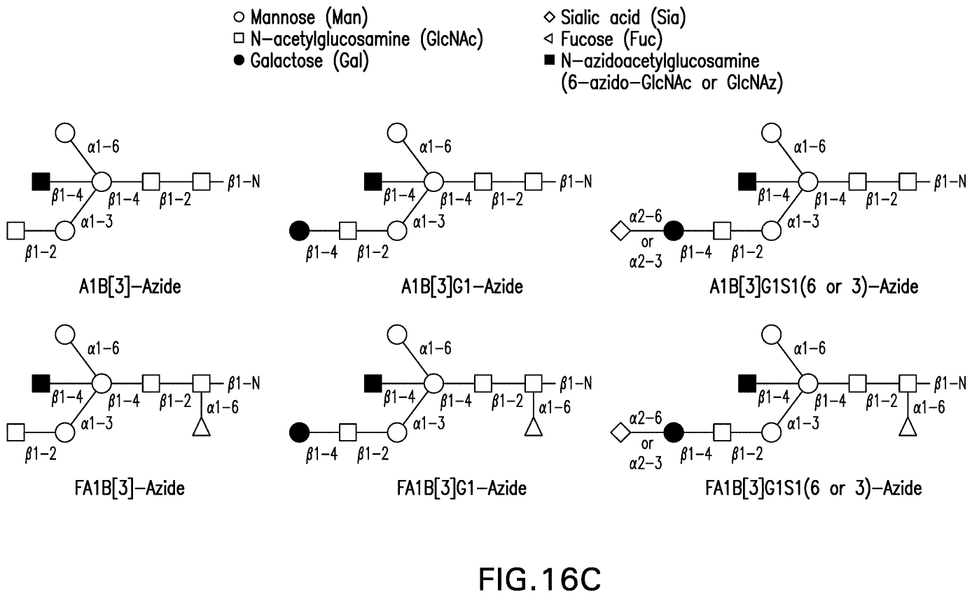

In particular embodiments, the bisected N-glycan B comprises a structure selected from those shown in FIG. 16A, 16B, or 16C, e.g., selected from A2B-azide; FA2B-azide; A2B[3]G1-azide; FA2B[3]G1-azide; A2B[6]G1-azide; FA2B[6]G1-azide; A2BG2-azide; FA2BG2-azide; A2B[3]G1S1(6 or 3)-azide; FA2B[3]G1S1(6 or 3)-azide; A2B[6]G1S1(6 or 3)-azide; FA2B[6]G1S1(6 or 3)-azide; A2BG2S2(6 or 3)-azide; FA2BG2S2(6 or 3)-azide; A1B[3]-azide; FA1B[3]-azide; A1B[3]G1-azide; FA1B[3]G1-azide; A1B[3]G1S1(6 or 3)-azide; and FA1B[3]G1S1(6 or 3)-azide, wherein the azide refers to the azide of 6-azido-GlcNAc or GlcNAz.

In a further embodiment, the triazole linkage has the structure

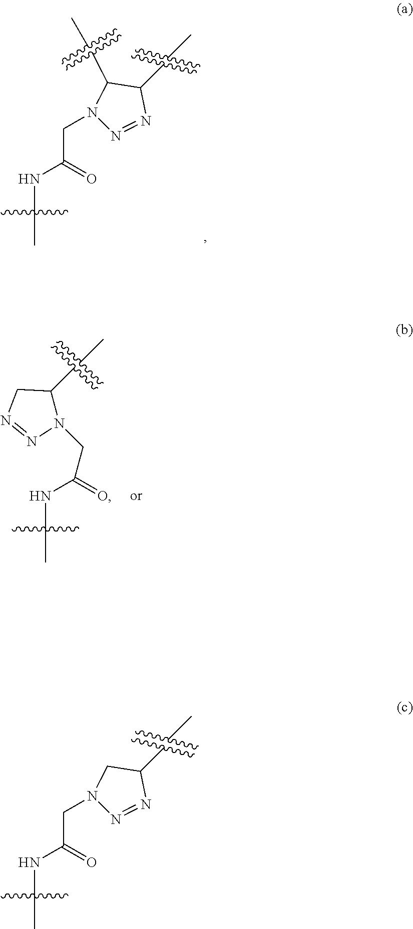

wherein the wavy line identifies the bond between the amine group of the GlcNAz residue and the C2 carbon of the glucose residue comprising the GlcNAz residue and the double wavy lines identify bonds between the alkyne functional group of L or Pay when L is a bond or the structure

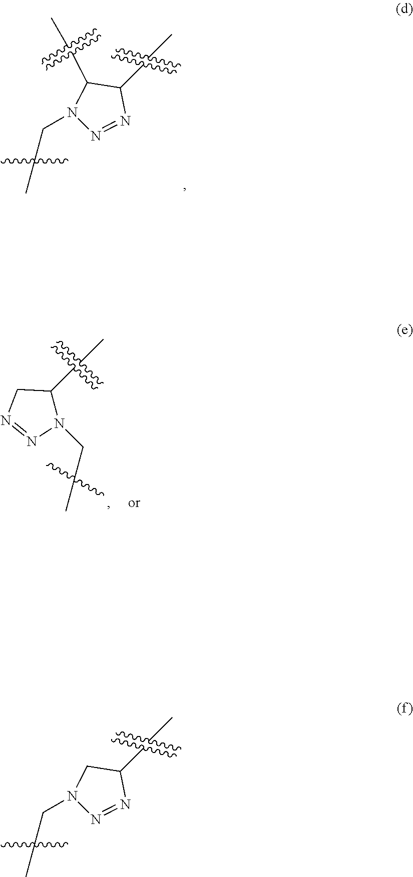

wherein the wavy line identifies the bond between the C5 and C6 carbons of the glucose residue comprising the 6-azido-GlcNAc residue and the double wavy lines identify bonds between the alkyne functional group of L or Pay when L is a bond.

The present invention further provides compositions comprising a plurality of glycoproteins and a pharmaceutically acceptable carrier, wherein each glycoprotein independently comprises the structure

GP-[B-T-L-Pay]ₙ

wherein GP is a glycoprotein; B is a bisected N-glycan wherein the bisecting sugar residue is 6-azido-GlcNAc or GlcNAz having an azide functional group; L is a linker element comprising an alkyne functional group or a bond; Pay is a payload, which comprises an alkyne functional group when L is a bond; T is a triazole linkage formed between the linkage of the azide functional group and the alkyne functional group; and, n is an integer from 1-20. In particular embodiments, the glycoprotein is a Fab or scFv comprising at least one N-glycosylation site. In particular embodiments, the glycoprotein is a fusion protein comprising the Fc domain of an antibody fused to a heterologous protein or heterologous glycoprotein. In particular embodiments, the heterologous glycoprotein is a Fab or scFv comprising at least one N-glycosylation site.

In particular embodiments, L is a self-immolative linker. In particular embodiments, L has the formula L¹-L², wherein each of L¹ and L² is independently a linker element or a bond. In particular embodiments, the L comprises a distal end comprising an alkyne containing group linked to the azide of the 6-azido-GlcNAc or GlcNAz of bisected N-glycan B in triazole linkage T and a proximal end linked to Pay. In a further embodiment, the alkyne group is a strained cyclooctyne. In a further embodiment, the strained cyclooctyne is DBCO.

In a further embodiment, the triazole linkage has the structure

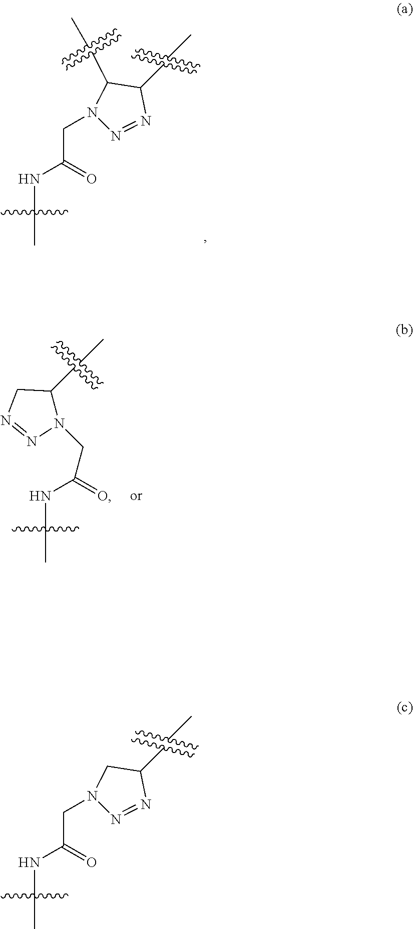

wherein the wavy line identifies the bond between the amine group of the GlcNAz residue and the C2 carbon of the glucose residue comprising the GlcNAz residue and the double wavy lines identify bonds between the alkyne functional group of L or Pay when L is a bond; or, the structure

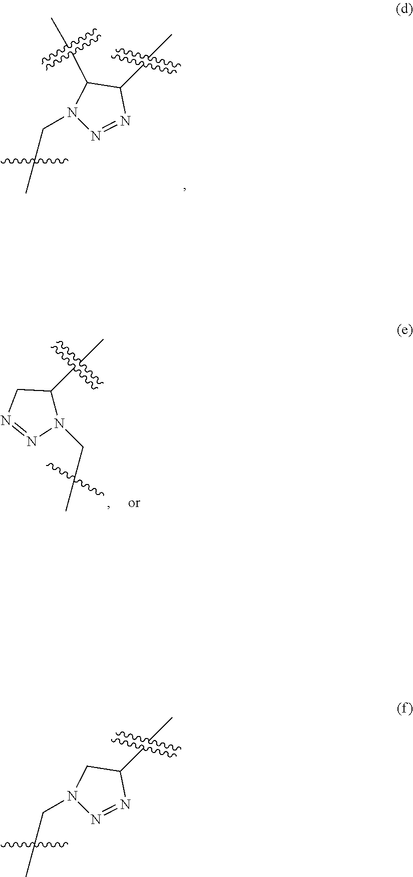

wherein the wavy line identifies the bond between the C5 and C6 carbons of the glucose residue comprising the 6-azido-GlcNAc residue and the double wavy lines identify bonds between the alkyne functional group of L or Pay when L is a bond.

In particular embodiments, the predominant bisected N-glycan B in the composition comprises a structure selected from A2B-azide; FA2B-azide; A2B[3]G1-azide; FA2 [3]BG1-azide; A2B[6]G1-azide; FA2B[6]G1-azide; A2BG2-azide; FA2BG2-azide; A2B[3]G1S1(6 or 3)-azide; FA2B[3]G1S1(6 or 3)-azide; A2B[6]G1S1(6 or 3)-azide; FA2B[6]G1S1(6 or 3)-azide; A2BG2S2(6 or 3)-azide; FA2BG2S2(6 or 3)-azide; A1B[3]-azide; FA1B[3]-azide; A1B[3]G1-azide; FA1B[3]G1-azide; A1B[3]G1S1(6 or 3)-azide; and FA1B[3]G1S1(6 or 3)-azide, wherein the azide refers to the azide of 6-azido-GlcNAc or GlcNAz, which forms a triazole linkage T with the alkyne functional group.

In particular embodiments of the composition, the predominant bisected N-glycan B in the composition comprises the mannose of the α1,3 arm linked at the non-reducing end to the reducing end of a GlcNAc residue. In particular embodiments of the composition, the predominant bisected N-glycan B in the composition comprises the mannose of the α1,3 arm linked at the non-reducing end to the reducing end of a GlcNAc residue and the mannose of the α1,6 arm is linked at the non-reducing end to the reducing end of a GlcNAc residue.

In particular embodiments of the composition, the predominant N-glycan B in the composition comprises the structure A2B-azide or FA2B-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B-azide or FA2B-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structures A2B-azide and FA2B-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2B-azide and FA2B-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structure A2B[3]G1-azide or FA2B[3]G1-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[3]G1-azide or FA2B[3]G1-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structures A2B[3]G1-azide and FA2B[3]G1-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2B[3]G1-azide and FA2B[3]G1-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structure A2B[6]G1-azide or FA2B[6]G1-azide, wherein the azide is part of the triazole linkage T. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[6]G1-azide or FA2B[6]G1-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structures A2B[6]G1-azide and FA2B[6]G1-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2B[6]G1-azide and FA2B[6]G1-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structure A2BG2-azide or FA2BG2-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2BG2S2-azide or FA2BG2S2-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structures A2BG2-azide and FA2BG2-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2BG2-azide and FA2BG2-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structure A2B[3]G1S1(6 or 3)-azide or FA2B[3]G1S1(6 or 3)-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[3]G1S1(6 or 3)-azide or FA2B[3]G1S1(6 or 3)-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structures A2B[3]G1S1(6 or 3)-azide and FA2B[3]G1S1(6 or 3)-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2B[3]G1S1(6 or 3)-azide and FA2B[3]G1S1(6 or 3)-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structure A2B[6]G1S1(6 or 3)-azide or FA2B[6]G1S1(6 or 3)-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[6]G1S1(6 or 3)-azide or FA2B[6]G21S1(6 or 3)-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structures A2B[6]G1S1(6 or 3)-azide and FA2B[6]G1S1(6 or 3)-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2B[6]G1S1(6 or 3)-azide and FA2B[6]G1S1(6 or 3)-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structures A2BG2S2(6 or 3)-azide or FA2BG2S2(6 or 3)-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2BG2S2(6 or 3)-azide or FA2BG2S2(6 or 3)-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structures A2BG2S2(6 or 3)-azide and FA2BG2S2(6 or 3)-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2BG2S2(6 or 3)-azide and FA2BG2S2(6 or 3)-azide, wherein the azide refers to the azide of 6-azido-GlcNAc or GlcNAz, which forms a triazole linkage T with the alkyne functional group.

In a further embodiment, the ADC of the present invention comprises the following structure:

Ab-[B-T-L-Pay]ₙ

wherein Ab is an antibody; B is a bisected N-glycan wherein the bisecting sugar residue is 6-azido-GlcNAc or GlcNAz having an azide functional group; L is a linker element comprising an alkyne functional group or a bond; Pay is a payload, which comprises an alkyne functional group when L is a bond; T is a triazole linkage formed between the linkage of the azide functional group and the alkyne functional group; and, n is an integer from 1-20.

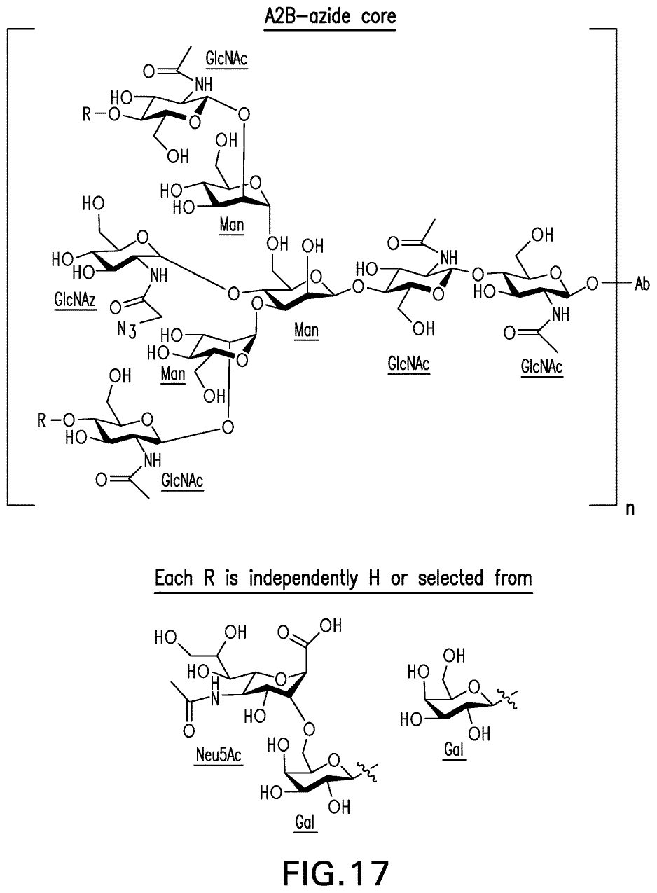

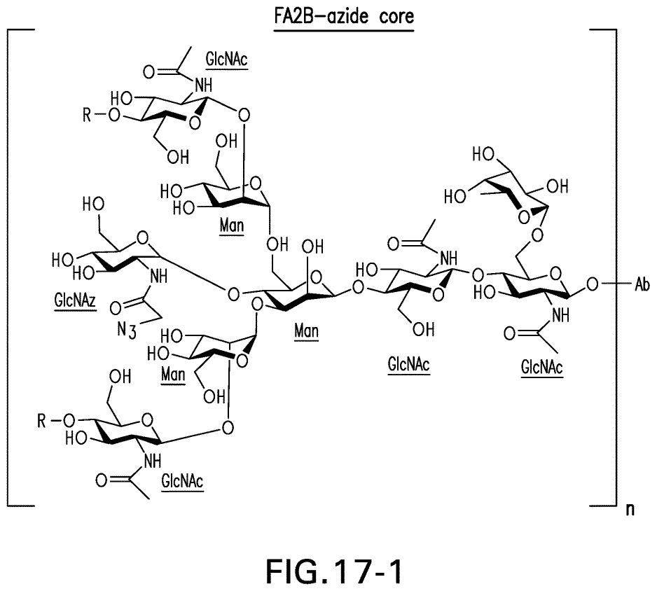

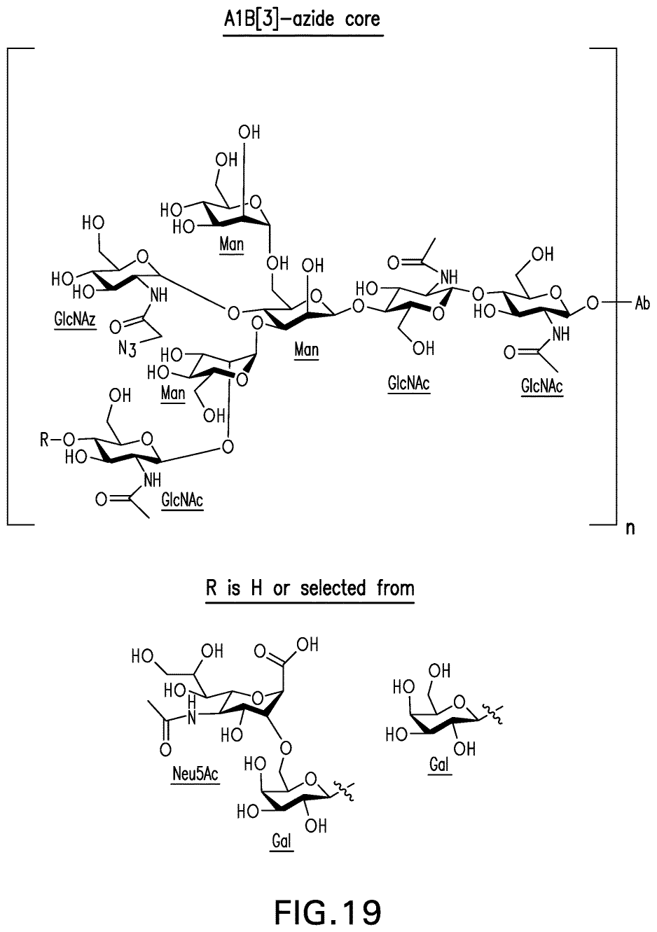

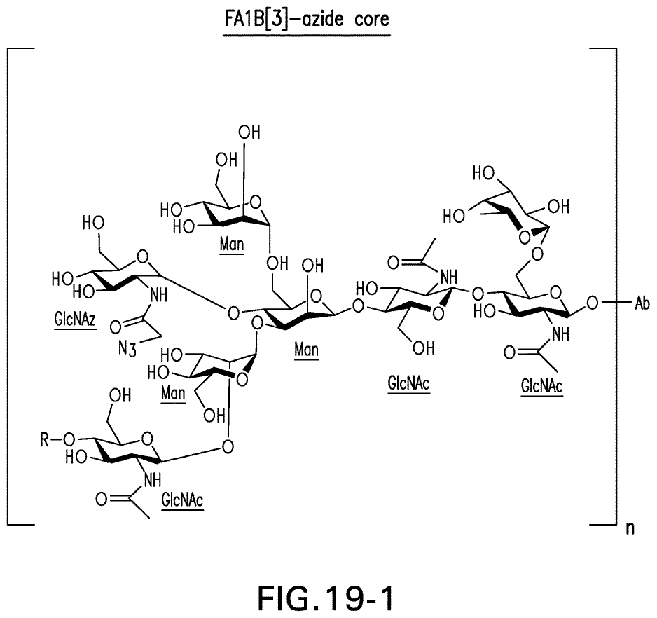

In particular embodiments, L is a self-immolative linker. In particular embodiments, L has the formula L¹-L², wherein each of L¹ and L² is independently a linker element or a bond. In particular embodiments, the L comprises a distal end comprising an alkyne containing group linked to the azide of the 6-azido-GlcNAc or GlcNAz of bisected N-glycan B in triazole linkage T and a proximal end linked to Pay. In a further embodiment, the alkyne group is a strained cyclooctyne. In a further embodiment, the strained cyclooctyne is DBCO. In particular embodiments, bisected N-glycan B comprises a structure selected from those shown in FIG. 16A, 16B, or 16C, e.g., selected from A2B-azide; FA2B-azide; A2B[3]G1-azide; FA2B[3]G1-azide; A2B[6]G1-azide; FA2B[6]G1-azide; A2BG2-azide; FA2BG2-azide; A2B[3]G1S1(6 or 3)-azide; FA2B[3]G1S1(6 or 3)-azide; A2B[6]G1S1(6 or 3)-azide; FA2B[6]G1S1(6 or 3)-azide; A2BG2S2(6 or 3)-azide; FA2BG2S2(6 or 3)-azide; A1B[3]-azide; FA1B[3]-azide; A1B[3]G1-azide; FA1B[3]G1-azide; A1B[3]G1S1(6 or 3)-azide; and FA1B[3]G1S1(6 or 3)-azide, wherein the azide refers to the azide of 6-azido-GlcNAc or GlcNAz, which forms a triazole linkage T with the alkyne functional group. Representative structures of bisected N-glycans comprising a bisecting 6-azido-GlcNAc or GlcNAz residue are shown in FIGS. 17 and 19.

In a further embodiment, the triazole linkage has the structure

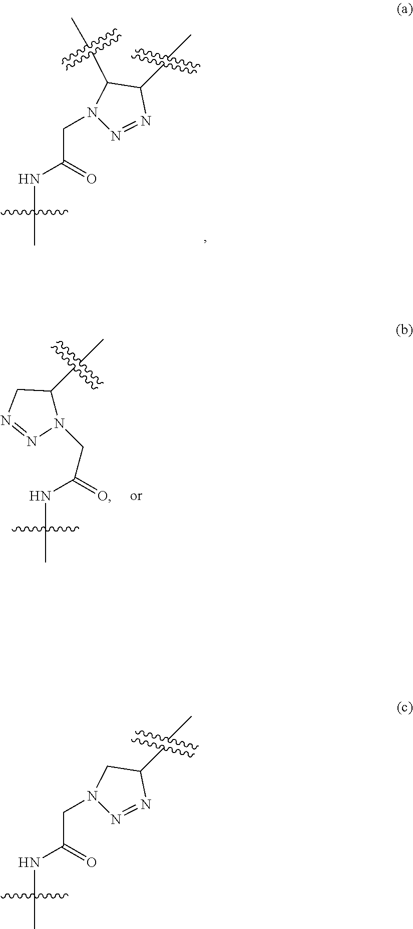

wherein the wavy line identifies the bond between the amine group of the GlcNAz residue and the C2 carbon of the glucose residue comprising the GlcNAz residue and the double wavy lines identify bonds between the alkyne functional group of L or Pay when L is a bond; or, the structure

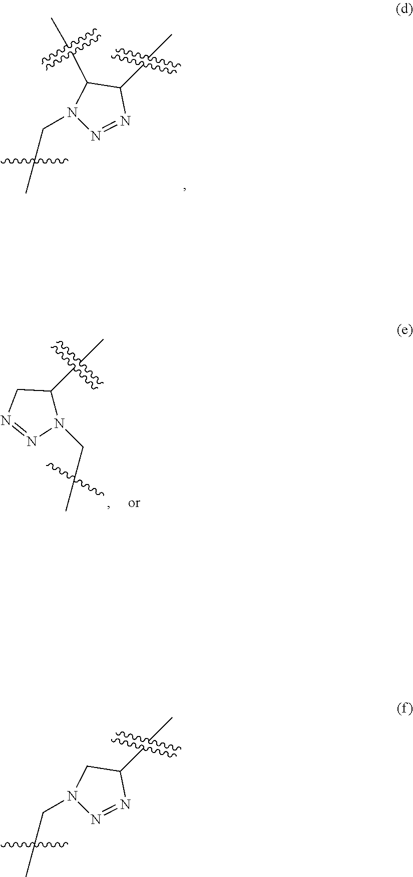

wherein the wavy line identifies the bond between the C5 and C6 carbons of the glucose residue comprising the 6-azido-GlcNAc residue and the double wavy lines identify bonds between the alkyne functional group of L or Pay when L is a bond.

In a further embodiment, the present invention further provides compositions comprising a plurality of ADCs and a pharmaceutically acceptable carrier, wherein each ADC independently comprises the structure

Ab-[B-T-L-Pay]ₙ

wherein Ab is an antibody; B is a bisected N-glycan wherein the bisecting sugar residue is 6-azido-GlcNAc or GlcNAz having an azide functional group; L is a linker element comprising an alkyne functional group or a bond; Pay is a payload, which comprises an alkyne functional group when L is a bond; T is a triazole linkage formed between the linkage of the azide functional group and the alkyne functional group; and, n is an integer from 1-20. In particular embodiments, L is a self-immolative linker. In particular embodiments, L has the formula L¹-L², wherein each of L¹ and L² is independently a linker element or a bond. In particular embodiments, the L comprises a distal end comprising an alkyne containing group linked to the azide of the 6-azido-GlcNAc or GlcNAz of bisected N-glycan B in triazole linkage T and a proximal end linked to Pay. In a further embodiment, the alkyne group is a strained cyclooctyne. In a further embodiment, the strained cyclooctyne is DBCO.

In a further embodiment, the triazole linkage has the structure

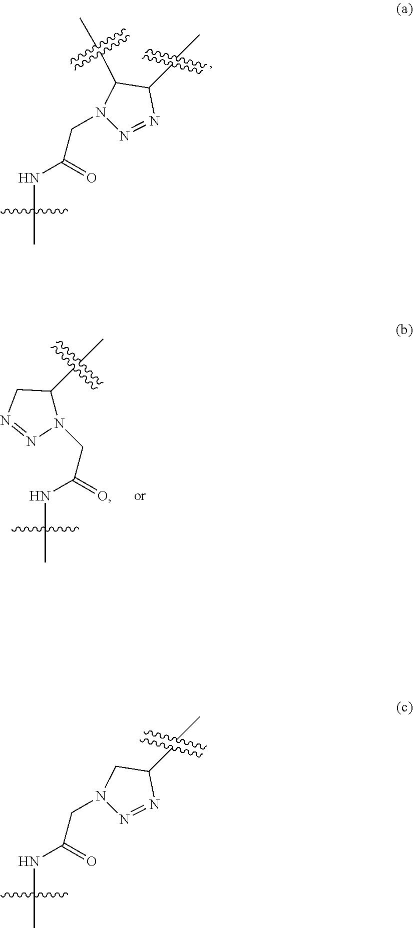

wherein the wavy line identifies the bond between the amine group of the GlcNAz residue and the C2 carbon of the glucose residue comprising the GlcNAz residue and the double wavy lines identify bonds between the alkyne functional group of L or Pay when L is a bond; or, the structure

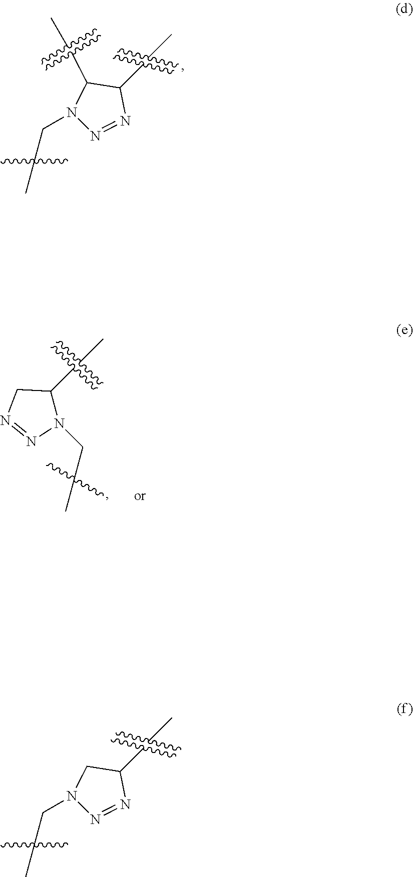

wherein the wavy line identifies the bond between the C5 and C6 carbons of the glucose residue comprising the 6-azido-GlcNAc residue and the double wavy lines identify bonds between the alkyne functional group of L or Pay when L is a bond.

In particular embodiments, the predominant bisected N-glycan B in the composition comprises a structure selected from A2B-azide; FA2B-azide; A2B[3]G1-azide; FA2 [3]BG1-azide; A2B[6]G1-azide; FA2B[6]G1-azide; A2BG2-azide; FA2BG2-azide; A2B[3]G1S1(6 or 3)-azide; FA2B[3]G1S1(6 or 3)-azide; A2B[6]G1S1(6 or 3)-azide; FA2B[6]G1S1(6 or 3)-azide; A2BG2S2(6 or 3)-azide; FA2BG2S2(6 or 3)-azide; A1B[3]-azide; FA1B[3]-azide; A1B[3]G1-azide; FA1B[3]G1-azide; A1B[3]G1S1(6 or 3)-azide; and FA1B[3]G1S1(6 or 3)-azide, wherein the azide refers to the azide of 6-azido-GlcNAc or GlcNAz, wherein the azide refers to the azide of 6-azido-GlcNAc or GlcNAz, which forms a triazole linkage T with the alkyne functional group.

In particular embodiments of the composition, the predominant bisected N-glycan B in the composition comprises the mannose of the α1,3 arm linked at the non-reducing end to the reducing end of a GlcNAc residue. In particular embodiments of the composition, the predominant bisected N-glycan B in the composition comprises the mannose of the α1,3 arm linked at the non-reducing end to the reducing end of a GlcNAc residue and the mannose of the α1,6 arm is linked at the non-reducing end to the reducing end of a GlcNAc residue. In particular embodiments of the composition, the predominant N-glycan B in the composition comprises the structure A2B-azide or FA2B-azide. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B-azide or FA2B-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structures A2B-azide and FA2B-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2B-azide and FA2B-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structure A2B[3]G1-azide or FA2B[3]G1-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[3]G1-azide or FA2B[3]G1-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structures A2B[3]G1-azide and FA2B[3]G1-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2B[3]G1-azide and FA2B[3]G1-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structure A2B[6]G1-azide or FA2B[6]G1-azide., wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[6]G1-azide or FA2B[6]G1-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structures A2B[6]G1-azide and FA2B[6]G1-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2B[6]G1-azide and FA2B[6]G1-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structure A2BG2-azide or FA2BG2-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2BG2-azide or FA2BG2-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structures A2BG2-azide and FA2BG2-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2BG2-azide and FA2BG2-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structure A2B[3]G1S1(6)-azide or FA2B[3]G1S1(6)-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[3]G1S1(6)-azide or FA2B[3]G1S1(6)-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structures A2B[3]G1S1(6)-azide and FA2B[3]G1S1(6)-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2B[3]G1S1(6)-azide and FA2B[3]G1S1(6)-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structure A2B[6]G1S1(6)-azide or FA2B[6]G1S1(6)-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[6]G1S1(6)-azide or FA2B[6]G21S1(6)-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structures A2B[6]G1S1(6)-azide and FA2B[6]G1S1(6)-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2B[6]G1S1(6)-azide and FA2B[6]G1S1(6)-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structures A2BG2S2(6)-azide or FA2BG2S2(6)-azide, wherein the azide refers to the azide of 6-azido-GlcNAc or GlcNAz, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2BG2S2(6)-azide or FA2BG2S2(6)-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structures A2BG2S2(6)-azide and FA2BG2S2(6)-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2BG2S2(6)-azide and FA2BG2S2(6)-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structure A2B[3]G1S1(3)-azide or FA2B[3]G1S1(3)-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[3]G1S1(3)-azide or FA2B[3]G1S1(3)-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structures A2B[3]G1S1(3)-azide and FA2B[3]G1S1(3)-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2B[3]G1S1(3)-azide and FA2B[3]G1S1(3)-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structure A2B[6]G1S1(3)-azide or FA2B[6]G1S1(3)-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[6]G1S1(3)-azide or FA2B[6]G21S1(3)-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structures A2B[6]G1S1(3)-azide and FA2B[6]G1S1(3)-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2B[6]G1S1(3)-azide and FA2B[6]G1S1(3)-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structures A2BG2S2(3)-azide or FA2BG2S2(3)-azide, wherein the wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2BG2S2(3)-azide or FA2BG2S2(3)-azide, wherein the azide refers to the azide of 6-azido-GlcNAc or GlcNAz, which forms a triazole linkage T with the alkyne functional group.

In particular embodiments of the composition, the predominant N-glycan B in the composition comprises the structures A2BG2S2(3)-azide and FA2BG2S2(3)-azide, wherein the azide refers to the azide of 6-azido-GlcNAc or GlcNAz, which forms a triazole linkage T with the alkyne functional group. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2BG2S2(3)-azide and FA2BG2S2(3)-azide, wherein the azide refers to the azide of 6-azido-GlcNAc or GlcNAz, which forms a triazole linkage T with the alkyne functional group.

In a further embodiment, the present invention further provides compositions comprising a plurality of ADCs and a pharmaceutically acceptable carrier, wherein each ADC independently comprises an antibody conjugated to a bisected N-glycan comprising a structure selected from A2B-triazole-L-payload; FA2B-triazole-L-payload; A2B[3]G1-triazole-L-payload; FA2 [3]BG1-triazole-L-payload; A2B[6]G1-triazole-L-payload; FA2B[6]G1-triazole-L-payload; A2BG2-triazole-L-payload; FA2BG2-triazole-L-payload; A2B[3]G1S1(6 or 3)-triazole-L-payload; FA2B[3]G1S1(6 or 3)-triazole-L-payload; A2B[6]G1S1(6 or 3)-triazole-L-payload; FA2B[6]G1S1(6 or 3)-triazole-L-payload; A2BG2S2(6 or 3)-triazole-L-payload; FA2BG2S2(6 or 3)-triazole-L-payload; A1B[3]-triazole-L-payload; FA1B[3]-triazole-L-payload; A1B[3]G1-triazole-L-payload; FA1B[3]G1-triazole-L-payload; A1B[3]G1S1(6 or 3)-triazole-L-payload; and FA1B[3]G1S1(6 or 3)-triazole-L-payload, wherein L is a linker or a bond.

In particular embodiments of the composition, the predominant bisected N-glycan in the composition comprises the structure A2B-triazole-L-payload or FA2B-triazole-L-payload. In particular embodiments of the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B-triazole-L-payload or FA2B-triazole-L-payload.

In particular embodiments of the composition, the predominant bisected N-glycan in the composition comprises the structures A2B-triazole-L-payload and FA2B-triazole-L-payload. In particular embodiments of the composition, the predominant bisected N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2B-triazole-L-payload and FA2B-triazole-L-payload.

In particular embodiments of the composition, the predominant bisected N-glycan in the composition comprises the structure A2B[3]G1-triazole-L-payload or FA2B[3]G1-triazole-L-payload. In particular embodiments of the composition, the predominant bisected N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[3]G1-triazole-L-payload or FA2B[3]G1-triazole-L-payload.

In particular embodiments of the composition, the predominant bisected N-glycan in the composition comprises the structures A2B[3]G1-triazole-L-payload and FA2B[3]G1-triazole-L-payload. In particular embodiments of the composition, the predominant bisected N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2B[3]G1-triazole-L-payload and FA2B[3]G1-triazole-L-payload.

In particular embodiments of the composition, the predominant bisected N-glycan in the composition comprises the structure A2B[6]G1-triazole-L-payload or FA2B[6]G1-triazole-L-payload. In particular embodiments of the composition, the predominant bisected N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[6]G1-triazole-L-payload or FA2B[6]G1-triazole-L-payload.

In particular embodiments of the composition, the predominant bisected N-glycan in the composition comprises the structures A2B[6]G1-triazole-L-payload and FA2B[6]G1-triazole-L-payload. In particular embodiments of the composition, the predominant bisected N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2B[6]G1-triazole-L-payload and FA2B[6]G1-triazole-L-payload.

In particular embodiments of the composition, the predominant bisected N-glycan in the composition comprises the structure A2BG2-triazole-L-payload or FA2BG2-triazole-L-payload. In particular embodiments of the composition, the predominant bisected N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2BG2-triazole-L-payload or FA2BG2-triazole-L-payload.

In particular embodiments of the composition, the predominant bisected N-glycan in the composition comprises the structures A2BG2-triazole-L-payload and FA2BG2-triazole-L-payload. In particular embodiments of the composition, the predominant bisected N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2BG2-triazole-L-payload and FA2BG2-triazole-L-payload.

In particular embodiments of the composition, the predominant bisected N-glycan in the composition comprises the structure A2B[3]G1S1(6)-triazole-L-payload or FA2B[3]G1S1(6)-triazole-L-payload. In particular embodiments of the composition, the predominant bisected N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[3]G1S1(6)-triazole-L-payload or FA2B[3]G1S1(6)-triazole-L-payload.

In particular embodiments of the composition, the predominant bisected N-glycan in the composition comprises the structures A2B[3]G1S1(6)-triazole-L-payload and FA2B[3]G1S1(6)-triazole-L-payload. In particular embodiments of the composition, the predominant bisected N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2B[3]G1S1(6)-triazole-L-payload and FA2B[3]G1S1(6)-triazole-L-payload.

In particular embodiments of the composition, the predominant bisected N-glycan in the composition comprises the structure A2B[6]G1S1(6)-triazole-L-payload or FA2B[6]G1S1(6)-triazole-L-payload. In particular embodiments of the composition, the predominant bisected N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[6]G1S1(6)-triazole-L-payload or FA2B[6]G21S1(6)-triazole-L-payload.

In particular embodiments of the composition, the predominant bisected N-glycan in the composition comprises the structures A2B[6]G1S1(6)-triazole-L-payload and FA2B[6]G1S1(6)-triazole-L-payload. In particular embodiments of the composition, the predominant bisected N-glycasn in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2B[6]G1S1(6)-triazole-L-payload and FA2B[6]G1S1(6)-triazole-L-payload.

In particular embodiments of the composition, the predominant bisected N-glycan in the composition comprises the structures A2BG2S2(6)-triazole-L-payload or FA2BG2S2(6)-triazole-L-payload. In particular embodiments of the composition, the predominant bisected N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2BG2S2(6)-triazole-L-payload or FA2BG2S2(6)-triazole-L-payload.

In particular embodiments of the composition, the predominant bisected N-glycan in the composition comprises the structures A2BG2S2(6)-triazole-L-payload and FA2BG2S2(6)-triazole-L-payload. In particular embodiments of the composition, the predominant bisected N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2BG2S2(6)-triazole-L-payload and FA2BG2S2(6)-triazole-L-payload.

In particular embodiments of the composition, the predominant bisected N-glycan in the composition comprises the structure A2B[3]G1S1(3)-triazole-L-payload or FA2B[3]G1S1(3)-triazole-L-payload. In particular embodiments of the composition, the predominant bisected N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[3]G1S1(3)-triazole-L-payload or FA2B[3]G1S1(3)-triazole-L-payload.

In particular embodiments of the composition, the predominant bisected N-glycan in the composition comprises the structures A2B[3]G1S1(3)-triazole-L-payload and FA2B[3]G1S1(3)-triazole-L-payload. In particular embodiments of the composition, the predominant bisected N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2B[3]G1S1(3)-triazole-L-payload and FA2B[3]G1S1(3)-triazole-L-payload.

In particular embodiments of the composition, the predominant bisected N-glycan in the composition comprises the structure A2B[6]G1S1(3)-triazole-L-payload or FA2B[6]G1S1(3)-triazole-L-payload. In particular embodiments of the composition, the predominant bisected N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[6]G1S1(3)-triazole-L-payload or FA2B[6]G21S1(3)-triazole-L-payload.

In particular embodiments of the composition, the predominant bisected N-glycan in the composition comprises the structures A2B[6]G1S1(3)-triazole-L-payload and FA2B[6]G1S1(3)-triazole-L-payload. In particular embodiments of the composition, the predominant bisected N-glycasn in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2B[6]G1S1(3)-triazole-L-payload and FA2B[6]G1S1(3)-triazole-L-payload.

In particular embodiments of the composition, the predominant bisected N-glycan in the composition comprises the structures A2BG2S2(3)-triazole-L-payload or FA2BG2S2(3)-triazole-L-payload. In particular embodiments of the composition, the predominant bisected N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2BG2S2(3)-triazole-L-payload or FA2BG2S2(3)-triazole-L-payload.

In particular embodiments of the composition, the predominant bisected N-glycan in the composition comprises the structures A2BG2S2(3)-triazole-L-payload and FA2BG2S2(3)-triazole-L-payload. In particular embodiments of the composition, the predominant bisected N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2BG2S2(3)-triazole-L-payload and FA2BG2S2(3)-triazole-L-payload.

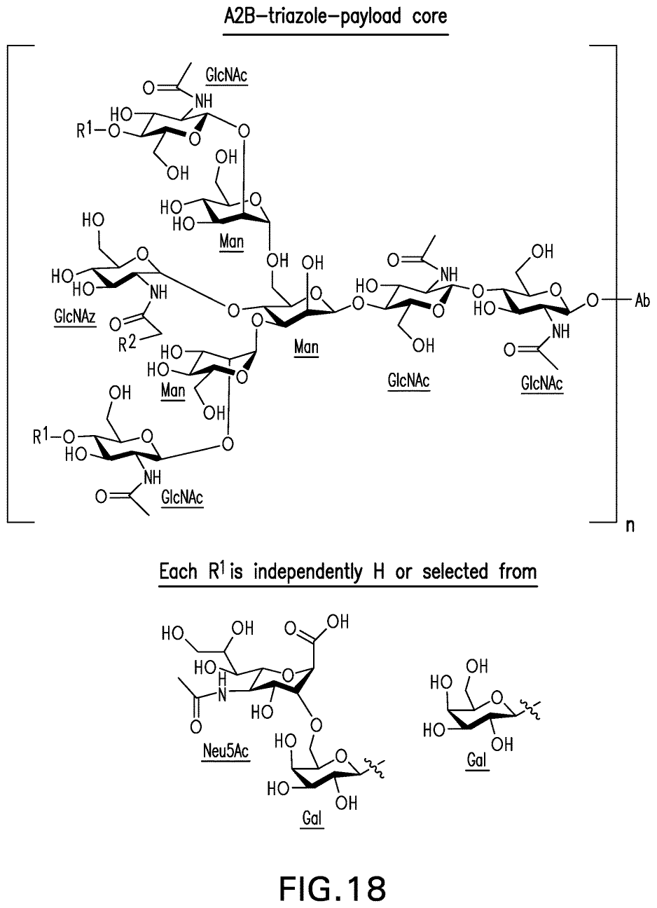

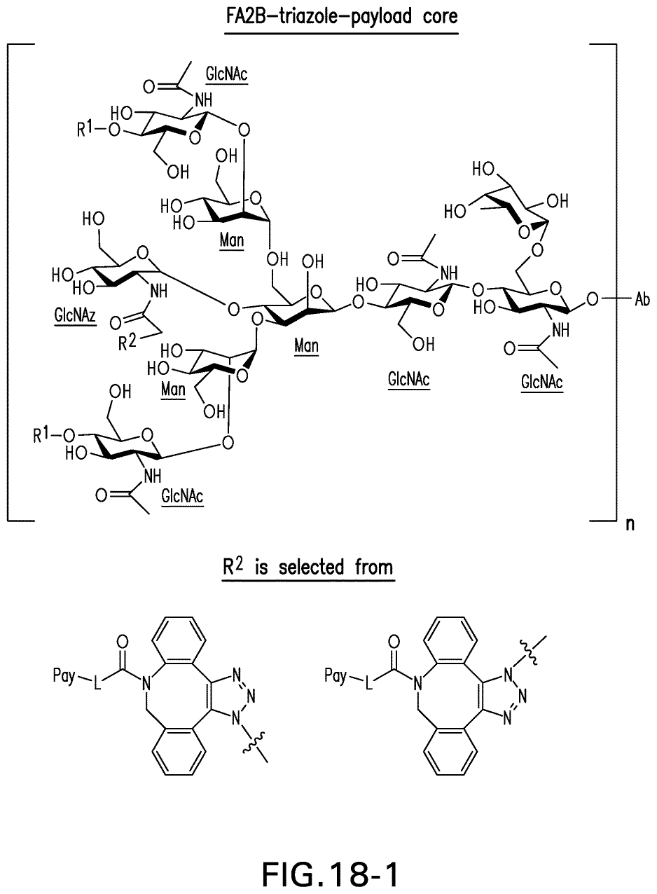

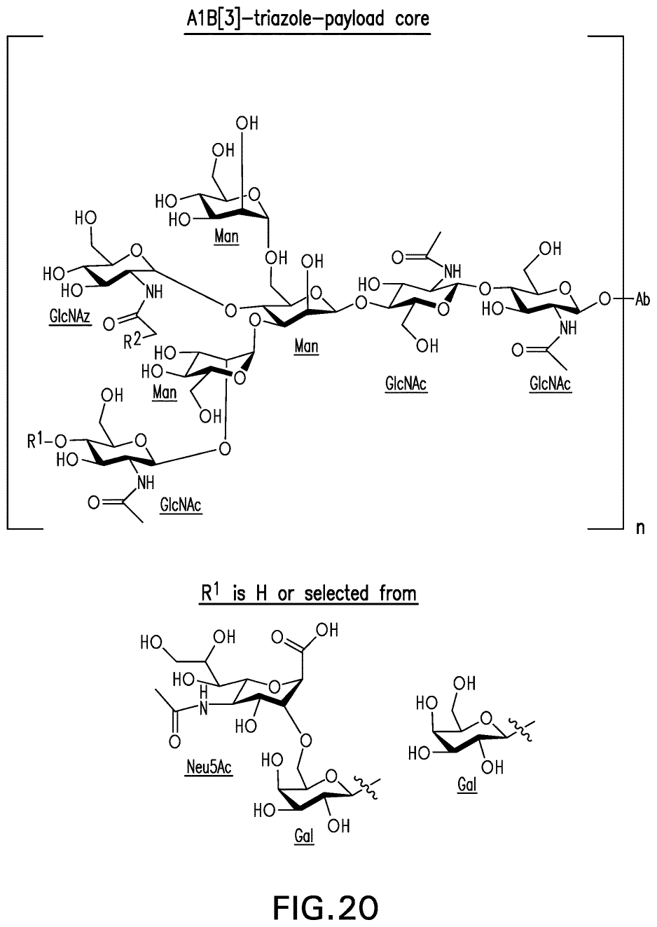

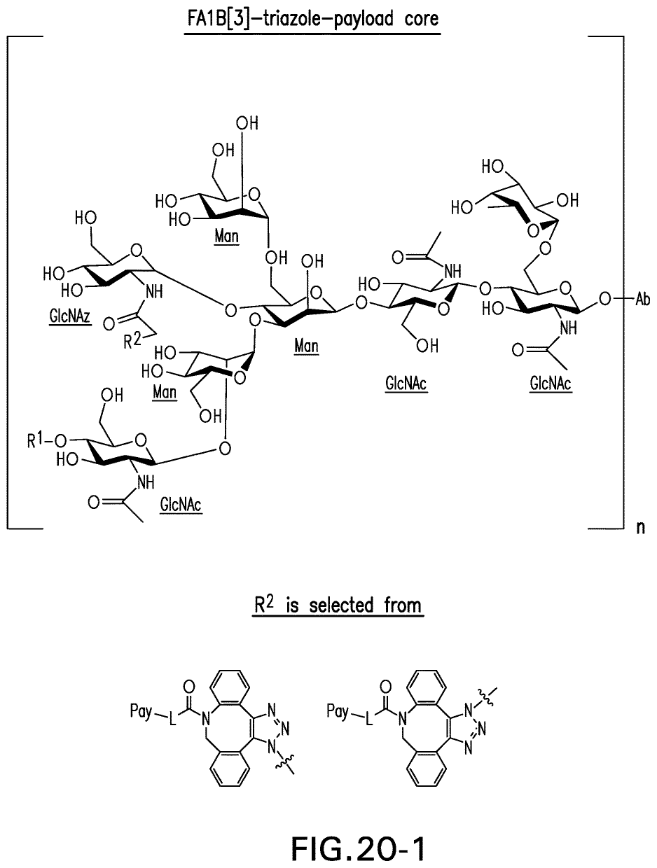

Representative structures of bisected N-glycans comprising a bisecting 6-azido-GlcNAc or GlcNAz residue conjugated to a payload via a triazole linkage are shown in FIGS. 18 and 20.

### Synthesis of Glycoproteins Comprising Bisected N-Glycans Wherein the Bisecting Sugar is GlcNAz

Mammalian and human complex N-glycans comprise GlcNAc, mannose, galactose, and sialic acid residues. Complex N-glycans comprise a paucimannose core represented by the formula Man₃GlcNAc₂ forming a Y-shaped molecule wherein the terminal mannose residues are on the distal end of the chitobiose core and the GlcNAc residue at the reducing end of the chitobiose core is linked to asparagine (Asn) in a β1-N linkage. The terminal mannose residues are linked by α1-6 and α1-3 linkages to a central mannose residue to provide an α1-6-linked mannose arm and an α1-3-linked mannose arm. The central mannose is in turn linked to the chitobiose core by a β1-4 linkage. Terminal GlcNAc residues, when present, are linked to the terminal mannose residue on the α1-3-linked mannose arm in a β1-2 linkage by GnT-I to produce hybrid N-glycans, and then to the terminal mannose residue on the a1-6 linked arm by GnT-II to produce bi-antennary N-glycans. An additional GlcNAc residue, when present, may be linked to the terminal mannose residue on the α1-3-linked mannose arm of bi-antennary N-glycans in a β1-4 linkage by GnT-IV or the terminal mannose residue on the α1-6-linked mannose arm of bi-antennary N-glycans in a β1-6 linkage by GnT-V to produce tri-antennary N-glycans. In a further aspect, a GlcNAc residue, when present, may be linked to the arm of the tri-antennary N-glycan that is linked to only one GlcNAc residue with the appropriate GnT to produce multi-antennary N-glycans in which each arm comprises two GlcNAc residues, the α1-3-linked mannose arm comprising GlcNAc residues linked in a β1-2 linkage and a β1-4 linkage and the α1-6-linked mannose arm comprising GlcNAc residues linked in a β1-2 linkage and a β1-6 linkage. In an alternate aspect, the central mannose of bi-antennary N-glycans may be linked to a GlcNAc residue by a β1-4 linkage by GnTIII to produce bisected N-glycans. Terminal galactose residues, when present, are linked to the terminal GlcNAc residues of bi-antennary, tri-antennary, or multi-antennary N-glycans in a β1-4 linkage by GalT or to the α1-6-linked mannose arm and an α1-3-linked mannose arm GlcNAc residues of bisected N-glycans in a β1-4 linkage by GalT. Terminal sialic acid residues, when present, are linked to one or more of the terminal galactose residues of any of the bi-antennary, tri-antennary, multi-antennary, or bisected N-glycans comprising terminal galactose residues in either an α2-3 or α2-6 linkage by α2,3-siaT or α2,6-siaT, respectively, to produce α2,3- or α2.6-sialylated N-glycans. In particular embodiments, the sialic acid is N-Acetylneuraminic acid (Neu5Ac or NANA), a predominant sialic acid found in human cells and many mammalian cells.

Thus, in particular embodiments of the present invention, the N-glycosylated glycoproteins obtained from cell culture comprising mammalian or human cells or yeast cells genetically engineered to produce complex N-glycans are sequentially reacted with one or more glycosidases selected from group consisting of neuraminidase, galactosidase, and N-acetylglucosaminidase, to remove one or more terminal sugar residues on the N-glycan to produce a core structure. For example, in a first step, the glycoproteins are reacted with one or more neuraminidases to remove the sialic acid residues from those N-glycans that comprise terminal sialic acid residues to provide a population of N-glycans comprising terminal galactose residues. In a second step, the glycoproteins may then be reacted with one or more galactosidases that remove the galactose residues from those N-glycans that comprise terminal galactose residues to provide a population of N-glycans comprising terminal GlcNAc residues. In a third step, the glycoproteins may then be reacted with one or more N-acetylglucosaminidases that remove the GlcNAc residues from those N-glycans that comprise terminal galactose residues to provide a population of paucimannose N-glycans (M3), each N-glycan comprising a central mannose residue linked to a chitobiose core in a β1-4 linkage and two terminal mannose residues, one linked to the central mannose in an α1,3 linkage and the other linked to the central mannose in an α1,6 linkage. After any one of the three steps, the glycoproteins may be further reacted with a fucosidase to remove fucose residues from those the N-glycans comprising a fucose linked to the GlcNAc residue at the reducing end of the chitobiose core in an α1,6 linkage.

In a further embodiment, following any one of the above three steps, the population of N-glycans may be reacted with a glycosyltransferase capable of adding a specific sugar to the terminal sugar residues of a particular population of N-glycans. Successive reactions, each utilizing a specific glycosyltransferase, may be used to add sugars to the termini of the N-glycans in a stepwise manner to produce a population of N-glycans having particular glycoforms. The particular glycosyltransferase that may be used in a particular reaction will depend on the N-glycan substrate to be reacted upon by the glycosyltransferase as each glycosyltransferase is specific for a particular substrate.

Thus, in one embodiment of the present invention, a population of glycoproteins is reacted with a neuraminidase, a galactosidase, and one or more N-acetylglucosaminidases to produce a population of glycoproteins comprising fucosylated and non-fucosylated M3 N-glycans. The fucosylated and non-fucosylated M3 N-glycans are reacted with an GnT-I in the presence of UDP-GlcNAc to produce a population of glycoproteins comprising predominantly A1[3] N-glycans and FA1[3] N-glycans in which the α1,3-linked mannose residue is linked to a GlcNAc residue by a β1-2 linkage. The population of glycoproteins may then be reacted with GnT-III in the presence of UDP-GlcNAz or UDP-6-azido-GlcNAc to produce a population of glycoproteins comprising predominantly A1B[3]-azide N-glycans and/or FA1B[3]-azide N-glycans.

The population of glycoproteins comprising predominantly A1B[3]-azide N-glycans and FA1B[3]-azide N-glycans may then be reacted with a GalT in the presence of UDP-galactose to produce a population of glycoproteins comprising predominantly A1B[3]G1-azide N-glycans and/or FA1B[3]G1-azide N-glycans in which galactose is linked to the GlcNAc residue on the α1,3 arm in a β1-4 linkage.

The population of glycoproteins comprising predominantly A1B[3]G1-azide and FA1B[3]G1-azide N-glycans may then be reacted with an α2,6- or α2,3-siaT in the presence of CMP-sialic acid to produce a population of glycoproteins comprising predominantly A1B[3]G1S1(6 or 3)-azide N-glycans and/or FA1B[3]G1S1(6 or 3)-azide N-glycans in which sialic acid is linked to the galactose residue in an α2-6 or α2-3 linkage, respectively.

In particular embodiments, any one of the above glycoprotein populations may be reacted with a payload comprising an alkyne functional group under metal-free click chemistry conditions to produce a glycoprotein conjugates in which the azide functional group of the 6-azido-GlcNAc or GlcNAz is linked to the alkyne functional group of the payload in a triazole linkage. Exemplary glycoprotein conjugates may comprise predominantly A1B[3]-triazole-payload N-glycans; FA1B[3]-triazole-payload N-glycans; A1B[3]G1-triazole-payload N-glycans; FA1B[3]G1-triazole-payload N-glycans; A1B[3]G1S1(6 or 3)-triazole-payload N-glycans; and/or FA1B[3]G1S1(6 or 3)-triazole-payload N-glycans. In particular embodiments, the glycoprotein population comprises predominantly a mixture of two or three of the aforementioned exemplary glycoprotein conjugates.

In particular embodiments, one or more of the above glycoprotein populations may be reacted with a fucosidase to produce a substantially afucosylated population of glycoproteins in which the N-glycans lack fucose or reacted with a fucosyltransferase in the presence of GDP-fucose to produce a substantially fucosylated population of glycoproteins in which the N-glycans comprise fucose. In particular embodiments, the fucosyltransferase is an al-6-fucosyltransferase, which transfers a fucose residue from GDP-fucose to the GlcNAc residue on the reducing end of the chitobiose core of the N-glycan in an α1-6 linkage.

In another embodiment of the present invention, a population of glycoproteins is reacted with a neuraminidase, and a galactosidase to produce a population of glycoproteins comprising predominantly fucosylated and non-fucosylated A2 N-glycans (A2 and/or FA2, respectively). The population of glycoproteins may then be reacted with GnT-III in the presence of UDP-6-azido-GlcNAc or UDP-GlcNAz to produce a population of glycoproteins comprising predominantly A2B-azide and/or FA2B-azide N-glycans.

The population of glycoproteins comprising predominantly A1B-azide N-glycans and/or FA1B-azide N-glycans may then be reacted with a GalT in the presence of UDP-galactose to produce a population of glycoproteins comprising predominantly A2B[3]G1-azide; FA2B[3]G1-azide, A2B[6]G1-azide N-glycans; FA2B[6]G1-azide N-glycans; A2BG2-azide N-glycans; and/or FA2BG2-azide N-glycans in which galactose is linked to the GlcNAc residue on the α1,3 arm in a β1-4 linkage and/or α1,6 arm in a β1-4 linkage.

The population of glycoproteins comprising predominantly A2B[3]G1-azide, FA2B[3]G1-azide N-glycans, A2B[6]G1-azide N-glycans, FA2B[6]G1-azide N-glycans, A2BG2-azide N-glycans, and/or FA2BG2-azide N-glycans may then be reacted with an α2,6- or α2,3-siaT in the presence of CMP-sialic acid to produce a population of glycoproteins comprising predominantly A2B[3]G1S1(6 or 3)-azide N-glycans; FA2B[3]G1S1(6 or 3)-azide N-glycans; A2B[6]G1S1(6 or 3)-azide N-glycans; FA2B[6]G1S1(6 or 3)-azide N-glycans; A2BG2S2(6 or 3)-azide N-glycans; and/or FA2BG2S2(6 or 3)-azide N-glycans in which sialic acid is linked to the galactose residue in an α2-6 or α2-3 linkage, respectively.

In particular embodiments, any one of the above glycoprotein populations may be reacted with a payload comprising an alkyne functional group under metal-free click chemistry conditions to produce a glycoprotein conjugates in which the azide functional group of the 6-azido-GlcNAc or GlcNAz is linked to the alkyne functional group of the payload in a triazole linkage. Exemplary glycoprotein conjugates may comprise predominantly A2B[3]G1-triazole-payload N-glycans; FA2B[3]G1-triazole-payload N-glycans, A2B[6]G1-triazole-payload N-glycans; FA2B[6]G1-triazole-payload N-glycans; A2BG2-triazole-payload N-glycans; and FA2BG2-triazole-payload N-glycans; A2B[3]G1-triazole-payload N-glycans; FA2B[3]G1-triazole-payload N-glycans; A2B[6]G1-triazole-payload N-glycans; FA2B[6]G1-triazole-payload N-glycans; A2BG2-triazole-payload N-glycans; FA2BG2-triazole-payload N-glycans; A2B[3]G1S1(6 or 3)-triazole-payload N-glycans; FA2B[3]G1S1(6 or 3)-triazole-payload N-glycans; A2B[6]G1S1(6 or 3)-triazole-payload N-glycans; FA2B[6]G1S1(6 or 3)-triazole-payload N-glycans; A2BG2S2(6 or 3)-azide N-glycans; and/or FA2BG2S2(6 or 3)-triazole-payload N-glycans. In particular embodiments, the glycoprotein population comprises predominantly a mixture of two or three of the aforementioned exemplary glycoprotein conjugates.

In particular embodiments, one or more of the above exemplary glycoprotein populations may be reacted with a fucosidase to produce a substantially afucosylated population of glycoproteins in which the N-glycans lack fucose or reacted with a fucosyltransferase in the presence of GDP-fucose to produce a substantially fucosylated population of glycoproteins in which the N-glycans comprise fucose. In particular embodiments, the fucosyltransferase is an α1-6-fucosyltransferase, which transfers a fucose residue from GDP-fucose to the GlcNAc residue on the reducing end of the chitobiose core of the N-glycan in an α1-6 linkage.

In particular embodiments, the glycoprotein is an antibody, a Fab, or scFv comprising at least one N-glycosylation site. In particular embodiments, the glycoprotein is a fusion protein comprising the Fc domain of an antibody fused to a heterologous protein or heterologous glycoprotein. In particular embodiments, the heterologous glycoprotein is a Fab or scFv comprising at least one N-glycosylation site.

Thus, in one embodiment of the present invention, a population of antibodies is reacted with a neuraminidase, a galactosidase, and one or more N-acetylglucosaminidases to produce a population of antibodies comprising fucosylated and non-fucosylated M3 N-glycans. The fucosylated and non-fucosylated M3 N-glycans are reacted with an GnT-I in the presence of UDP-GlcNAc to produce a population of antibodies comprising predominantly A1[3] N-glycans and FA1[3] N-glycans in which the α1,3-linked mannose residue is linked to a GlcNAc residue by a β1-2 linkage. The population of antibodies may then be reacted with GnT-III in the presence of UDP-6-azido-GlcNAc or UDP-GlcNAz to produce a population of antibodies comprising predominantly A1B[3]-azide N-glycans and/or FA1B[3]-azide N-glycans.

The population of antibodies comprising predominantly A1B[3]-azide N-glycans and FA1B[3]-azide N-glycans may then be reacted with a GalT in the presence of UDP-galactose to produce a population of antibodies comprising predominantly A1B[3]G1-azide N-glycans and/or FA1B[3]G1-azide N-glycans in which galactose is linked to the GlcNAc residue on the α1,3 arm in a β1-4 linkage.

The population of antibodies comprising predominantly A1B[3]G1-azide and FA1B[3]G1-azide N-glycans may then be reacted with an α2,6- or α2,3-siaT in the presence of CMP-sialic acid to produce a population of antibodies comprising predominantly A1B[3]G1S1(6 or 3)-azide N-glycans and/or FA1B[3]G1S1(6 or 3)-azide N-glycans in which sialic acid is linked to the galactose residue in an α2-6 or α2-3 linkage, respectively.

In particular embodiments, any one of the above antibody populations may be reacted with a payload comprising an alkyne functional group under metal-free click chemistry conditions to produce an antibody conjugates in which the azide functional group of the 6-azido-GlcNAc or GlcNAz is linked to the alkyne functional group of the payload in a triazole linkage. Exemplary antibody conjugates may comprise predominantly A1B[3]-triazole-payload N-glycans; FA1B[3]-triazole-payload N-glycans; A1B[3]G1-triazole-payload N-glycans; FA1B[3]G1-triazole-payload N-glycans; A1B[3]G1S1(6 or 3)-triazole-payload N-glycans; and/or FA1B[3]G1S1(6 or 3)-triazole-payload N-glycans. In particular embodiments, the antibody population comprises predominantly a mixture of two or three of the aforementioned exemplary antibody conjugates.

In particular embodiments, one or more of the above antibody populations may be reacted with a fucosidase to produce a substantially afucosylated population of antibodies in which the N-glycans lack fucose or reacted with a fucosyltransferase in the presence of GDP-fucose to produce a substantially fucosylated population of antibodies in which the N-glycans comprise fucose. In particular embodiments, the fucosyltransferase is an α1-6-fucosyltransferase, which transfers a fucose residue from GDP-fucose to the GlcNAc residue on the reducing end of the chitobiose core of the N-glycan in an α1-6 linkage.

In another embodiment of the present invention, a population of antibodies is reacted with a neuraminidase, and a galactosidase to produce a population of antibodies comprising predominantly fucosylated and non-fucosylated A2 N-glycans (A2 and/or FA2, respectively). The population of antibodies may then be reacted with GnT-III in the presence of UDP-6-azido-GlcNAc or UDP-GlcNAz to produce a population of antibodies comprising predominantly A2B-azide and/or FA2B-azide N-glycans.

The population of antibodies comprising predominantly A1B-azide N-glycans and/or FA1B-azide N-glycans may then be reacted with a GalT in the presence of UDP-galactose to produce a population of antibodies comprising predominantly A2B[3]G1-azide; FA2B[3]G1-azide, A2B[6]G1-azide N-glycans; FA2B[6]G1-azide N-glycans; A2BG2-azide N-glycans; and/or FA2BG2-azide N-glycans in which galactose is linked to the GlcNAc residue on the α1,3 arm in a β1-4 linkage and/or α1,6 arm in a β1-4 linkage.

The population of antibodies comprising predominantly A2B[3]G1-azide, FA2B[3]G1-azide N-glycans, A2B[6]G1-azide N-glycans, FA2B[6]G1-azide N-glycans, A2BG2-azide N-glycans, and/or FA2BG2-azide N-glycans may then be reacted with an α2,6- or α2,3-siaT in the presence of CMP-sialic acid to produce a population of antibodies comprising predominantly A2B[3]G1S1(6 or 3)-azide N-glycans; FA2B[3]G1S1(6 or 3)-azide N-glycans; A2B[6]G1S1(6 or 3)-azide N-glycans; FA2B[6]G1S1(6 or 3)-azide N-glycans; A2BG2S2(6 or 3)-azide N-glycans; and/or FA2BG2S2(6 or 3)-azide N-glycans in which sialic acid is linked to the galactose residue in an α2-6 or α2-3 linkage, respectively.

In particular embodiments, any one of the above antibody populations may be reacted with a payload comprising an alkyne functional group under metal-free click chemistry conditions to produce an antibody conjugates in which the azide functional group of the 6-azido-GlcNAc or GlcNAz is linked to the alkyne functional group of the payload in a triazole linkage. Exemplary antibody conjugates may comprise predominantly A2B[3]G1-triazole-payload N-glycans; FA2B[3]G1-triazole-payload N-glycans, A2B[6]G1-triazole-payload N-glycans; FA2B[6]G1-triazole-payload N-glycans; A2BG2-triazole-payload N-glycans; and FA2BG2-triazole-payload N-glycans; A2B[3]G1-triazole-payload N-glycans; FA2B[3]G1-triazole-payload N-glycans; A2B[6]G1-triazole-payload N-glycans; FA2B[6]G1-triazole-payload N-glycans; A2BG2-triazole-payload N-glycans; FA2BG2-triazole-payload N-glycans; A2B[3]G1S1(6 or 3)-triazole-payload N-glycans; FA2B[3]G1S1(6 or 3)-triazole-payload N-glycans; A2B[6]G1S1(6 or 3)-triazole-payload N-glycans; FA2B[6]G1S1(6 or 3)-triazole-payload N-glycans; A2BG2S2(6 or 3)-azide N-glycans; and/or FA2BG2S2(6 or 3)-triazole-payload N-glycans. In particular embodiments, the antibody population comprises predominantly a mixture of two or three of the aforementioned exemplary antibody conjugates.

In particular embodiments, one or more of the above exemplary antibody populations may be reacted with a fucosidase to produce a substantially afucosylated population of antibodies in which the N-glycans lack fucose or reacted with a fucosyltransferase in the presence of GDP-fucose to produce a substantially fucosylated population of antibodies in which the N-glycans comprise fucose. In particular embodiments, the fucosyltransferase is an α1-6-fucosyltransferase, which transfers a fucose residue from GDP-fucose to the GlcNAc residue on the reducing end of the chitobiose core of the N-glycan in an α1-6 linkage.

Remodeling of glycoprotein N-glycans may be found in patent publication WO2022221163, which is incorporated herein by reference in its entirety.

The following examples are intended to promote a further understanding of the present invention.

### General Methods and Materials

Anti-HER2 Human IgG1 Trastuzumab biosimilar (SIM0005), anti-HER2 mouse IgG2a (BE0277), isotype human IgG1 (BP0297), and isotype mouse IgG2a (BE00085) were purchased from BioXCell. α2-3/6/8 Neuraminidase (P0720) and β1-4 Galactosidase (P0745) were purchased from New England Biolabs (NEB). Human serum IgG (14506), α2-6 Sialyltransferase (SialyIT, SAE0090), and β1-4 Galactosyltransferase (GalT, SAE0093) were purchased from Sigma-Aldrich. Human N-acetylglucosaminyltransferase III (GnT-III, 7359-GT) was purchased from R&D Systems. Rapid™ PNGase F (P0710) was purchased from NEB. SKBR3 cell line was purchased from the American Type Culture Collection (ATCC). Mouse anti-human IgG Fc gamma-FITC (209-095-098) for cell binding assay and AffiniPure rat Anti-Mouse IgG (415-065-166) for serum stability assays were purchased from Jackson ImmunoResearch.

Tris-HCl (T-5941), sodium acetate (S2889) sodium chloride (C5670), magnesium chloride (M4880), manganese chloride solution (M1787), sodium chloride solution (S5150), CMP-NANA (C8271), UDP-Gal (U4500), UDP-GlcNAc (U4375), NH₂NH₂·AcOH (259748), Bis(2-cyanoethyl)-N,N-diisopropyl phosporamidite (766305), m-CPBA (273031), Tetrazole (88185), DEAE-Sepharose (DFF100), Triethylammonium bicarbonate buffer (T7408), 1,4-dithiothreitol (D9779), 1-[Bis(dimethylamino)methylene]-1H-1,2,3-triazolo[4,5-b]pyridinium 3-oxid hexafluorophosphate (HATU) (445460), Diispropylethylamine (D125806), Acetonitrile (900667), Discovery N-glycan SPE columns (55465-U), and empty solid phase extraction (SPE) columns and frits (57607-U) were purchased from Sigma-Aldrich.

Ac3-6AzGlcNAc (1258) was purchased from Click Chemistry Tools. Heat-inactivated fetal bovine serum (FBS) (16140-071), Dulbecco's phosphate-buffered saline (DPBS) (14190-144) and McCoy's 5A (16600-082) buffer were purchased from Gibco. Dibenzocyclooctyne (DBCO)-PEG8-acid (BP-24017) and DBCO-PEG4-Val-Cit-PAB-MMAE (BP-25659)), and NHS-PEG4-Val-Cit-PAB-MMAE (BP-25503) were purchased from Broadpharm.

UDP-azidoacetylglucosamine (UDP-GlcNAz or UDP-6-azido-GlcNAc) (ES104) was purchased from R&D Systems.

Hydrophilic interaction liquid chromatography (HILIC) columns for N-glycan chromatography (186004742), solid phase extraction (SPE) μPlate (186002780), μPlate extraction manifold (186001831), SPE vacuum manifold (WAT200607), RapiGest™ SF (186008090), Glycoworks® buffer (186008100), N-glycan standards (186008791), and Rapifluor™-MS (186008091) were purchased from Waters™.

10K molecular weight cutoff (MWCO) filters (UFC503096) were purchased from Millipore Sigma. Octet sensors (18-5101) and Incucyte FabFluor probes (4722) were purchased from Sartorius. MabSelect SuRe™ protein A resin (17543801) was obtained from Cytiva. Male rat plasma (Sprague Dowley rat plasma K2EDTA, RATPLEDTA2-M) was purchased from BioIVT. Reagents for CellTiter-Glo® luminescent cell viability assay were purchased from Progema (G7570).

The chromatography and mass spectrometry for glycan profiling and protein mass analysis was performed on an Agilent 1290 Infinity II Liquid Chromatography (LC) system in tandem with Agilent 6230 Series quadrupole time-of-flight MS system (LC-QToF-MS). Agilent MassHunter software was used for the analysis, including data acquisition, chromatogram/mass analysis, mass deconvolution, and drug-antibody ratio (DAR) calculation. Hydrophobic interactions chromatography (HIC) and size-exclusion chromatography (SEC) studies were performed on Agilent 1290 Infinity II LC systems (with ChemStation C.01.10). Small-molecule LC-MS analysis for payload synthesis was performed on Agilent 1290 Infinity II LC systems in tandem with Agilent 6130 Series quadrupole MS system (with ChemStation C.01.07 SR3). Small molecule purification was conducted using an Agilent preparative 1260 Infinity II LC system (with ChemStation C.01.08). The analysis of ADC recovery from rat plasma was performed on the Synapt G2-S(quantitative ToF-MS system) from Waters (with MaxEnt software for mass deconvolution). Cell internalization study was carried out using an Incucyte system from Sartorius. ADC cell binding experiments were performed on Guava® Flow cytometry system from Millipore Sigma.

Protein/antibody concentration was measured using NanoDrop™ OneC (Thermo Scientific). BCA (bicinchoninic acid) assay signal was read on Synergy™ Neo2 (BioTek) plate reader. Melting temperature (Tm) and aggregation temperature (Tagg) were performed on Uncle (all-in-one biologics stability screening platform) from Unchained Labs (Pleasanton, CA).

### Synthesis of UDP 6-Azido-GlcNAc:

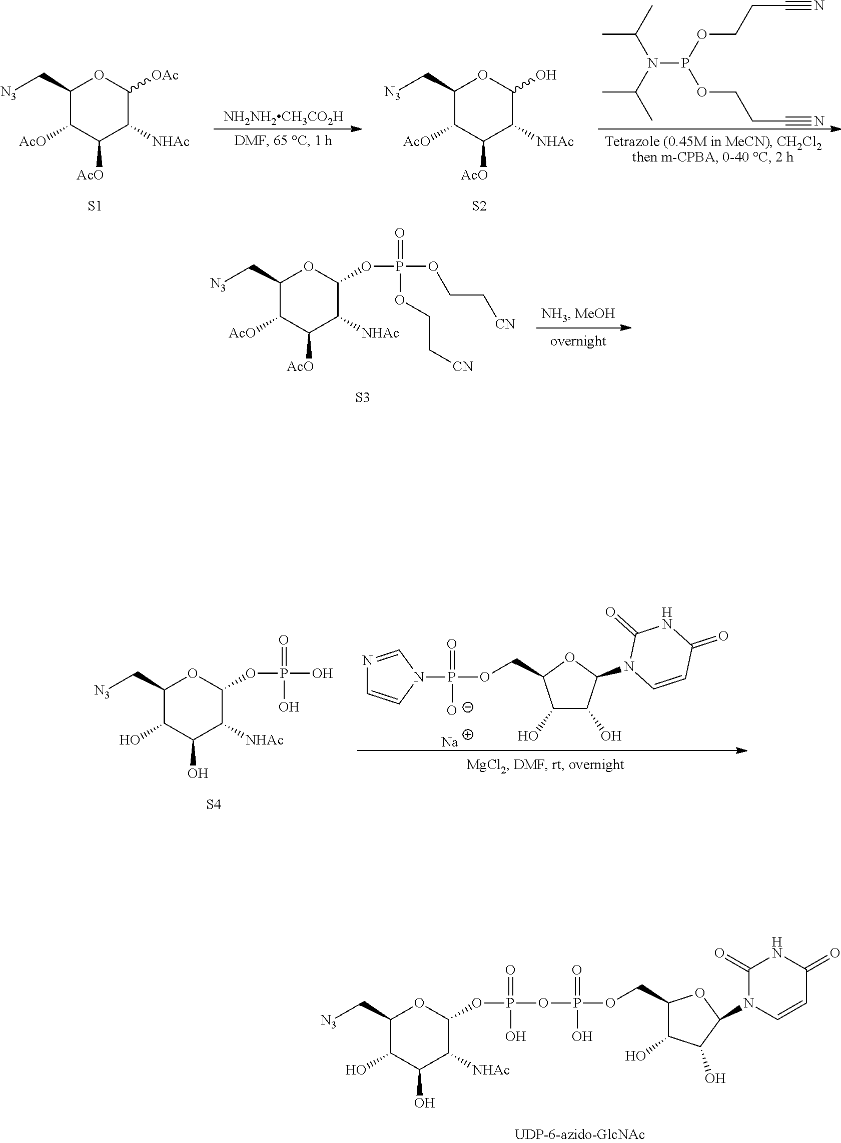

UDP-6-azido-GlcNAc was synthesized as described in literature with minor modifications (Morrison et al., Carbohydrate Research, 495: 108071 (2020); Zhao et al., Nature Protocols, 5: 636-646 (2010); Mayer et al., Bioorg Med Chem Lett, 21: 1199-2012011). Compound S1(200 mg, 0.53 mmol), NH₂NH₂·CH₃CO₂H (59.3 mg, 0.644 mmol) in dry dimethyl formamide (DMF) (3.0 mL) was stirred at 65 °C for 1 hour. After cooling to room temperature (RT), the reaction solution was diluted with 20 mL EtOAc and washed with water (2×20 mL), brine (20 mL), and the aqueous phases were back extracted with EtOAc (10 mL). The combined organic layer was dried over MgSO₄, filtered and concentrated, subjected to flash column chromatography using hexane:EtOAc (1:1) to afford S2 as viscous yellow oil (115 mg, 65% yield), which was used for next step without further purification.

To compound S2(100 mg, 0.30 mmol) in CH₂Cl₂ (1.5 mL) were added tetrazole (2.69 mL, 1.2 mmol, 0.45 M in MeCN) and a solution of bis(2-cyanoethyl)-N,N-diisopropyl phosporamidite (164 mg, 0.6 mmol) in CH₂Cl₂ (1 mL) in dropwise over 10 min under N₂atmosphere at 0 °C. The reaction was allowed to room temperature slowly and stirring was continued for a further 60 minutes. After cooling to −40 °C, m-CPBA (170 mg, 075 mmol, 77%) was added and the reaction mixture was allowed to warm slowly to RT and stirring continued for 30 minutes. The reaction was partitioned between CH₂Cl₂ (10 mL) and 10% aq. Na₂S₂O₃ solution (10 mL). The organic layer was washed with saturated NaHCO₃ (10 mL), brine (10 mL) and dried over MgSO₄, filtered and concentrated. Flash column chromatography of the crude residue (70-100% Ethyl acetate in hexane) afforded compound S3 (120 mg, 77%) as a viscous liquid and was carried for the next step.

Compound S3 (50 mg, 0.09 mmol) in MeOH (1 mL) was added aqueous NH₃ (7.5 mL, 4.8 mmol) and the flask was sealed, stirred at room temperature overnight. The mixture was partially concentrated to the aqueous fraction, then freeze-dried. The residue was triturated with acetone (2×10 mL) and dried under high vacuum to give compound S4 (13 mg, 41.2%) as fluffy solid. Spectral data was in well accordance with literature data (1H nuclear magnetic resonance (NMR), mass, liquid chromatography/mass spectroscopy (LC/MS)) (Chuh et al., Journal of the American Chemical Society, 136:12283-12295 (2014); Kruger et al., Chem Biol, 12:131-40 (2005)).

To sugar monophosphate S4 (25 mg, 0.07 mmol) in dry DMF (0.5 mL) was cooled in an ice bath and MgCl₂ (8.39 mg, 0.08 mmol) was added. The resulting mixture was stirred for 10 minutes, generating a fine suspension. The ice bath was removed, allowing the reaction to warm to room temperature. Next, UMP-Imidazole phosphate ester (Dabrowski-Tumanski et al., European Journal of Organic Chemistry, 11:2147-2154 (2013)) (34.9 mg, 0.08 mmol) was added in portion wise and stirred for 18 hours. The resulting mixture was then concentrated in vacuo and purified by using DEAE-Sepharose column (0 to 500 mM aqueous TEAB solution). The compound containing fractions were freeze-dried to afford UDP-6-azido-GlcNAc as a white foam (13.8 mg, 28.5% yield) and spectral data (¹H NMR, mass, LC/MS) agreed with previously reported literature (Morrison, et al., Carbohydrate Research, 495, 108071 (2020); Zhao et al., Nature Protocols, 5 (4), 636-646 (2010); Guan et al., Chemistry—A European Journal, 16, 13343-13345 (2010)).

### Synthesis of UDP-GlcNAc Derivatives:

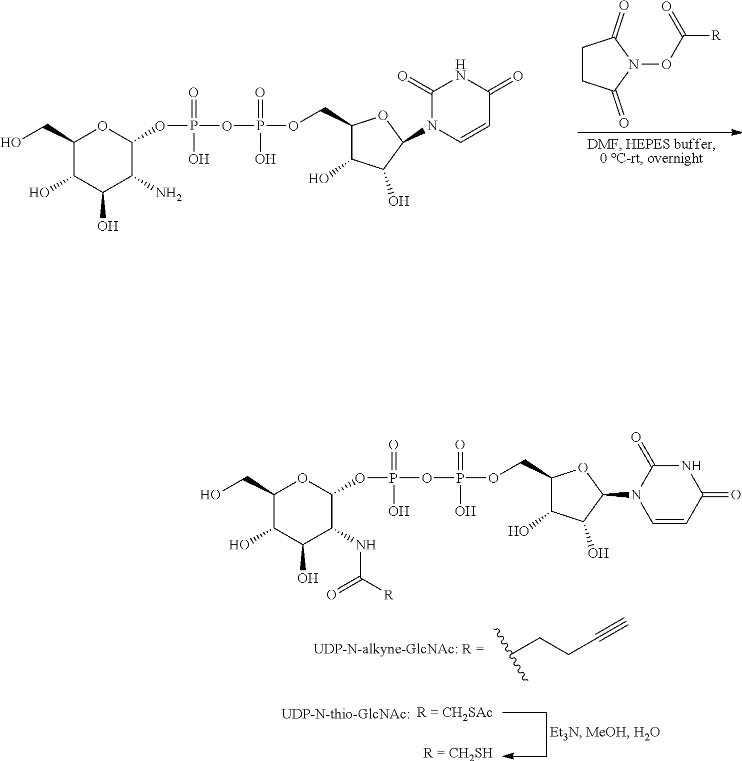

UDP-GlcNAc analogs chemically diversified at the C2-acylamide moiety were synthesized via active ester chemistry by reacting corresponding N-Hydroxysuccinimide (NHS) esters with UDP-Glucosamine (UDP-GlcNH₂). The synthesis of UDP-GlcNH₂ was carried out according to the previously reported procedure and structures were confirmed by 1H NMR (Craggs et al., Biochemistry, 57, 3387-3401 (2018); Hu et al., Nature Chemical Biology, 13, 1267-1273 (2017); Morais et al., Canadian J. Chem., 84, 587-596 (2006)). To a stirred solution of UDP-GlcNH₂ (10 μM) in HEPES (4-(2-hydroxyethyl)-1-piperazineethanesulfonic acid) buffer (2.5 mL, pH 8) was added 2.5 mL of corresponding N-hydroxy succinimide ester (10 mM in DMF) at 0 °C (Masuko et al., J. Org. Chem., 77, 1449-1456 (2012)). The reaction mixture was allowed to cool to room temperature over 2 hours and stirring continued for overnight. Reaction mixture was concentrated and crude mixtures were purified by reverse phase C18 column chromatography (4 g of C18 cartridge, ACN/aq. 10 mM TEAB) to afford UDP-GlcNAc analogs. In the UDP-N-thio-GlcNAc case, an additional step was performed to deprotect thio acyl functionality (Hanashima et al., Chemistry—A European Journal, 12, 3449-3462 (2006)). Crude UDP-N-SAc-GlcNAc was treated with MeOH/water/NEt₃ (5 mL, 5:2:1) for overnight and purified in the same way as described above to generate the final product. Spectral data of UDP-N-Alkyne-GlcNAc and UDP-N-thio-GlcNAc were in well agreement with literature data.

### Synthesis of DBCO-PEG8-MMAE

To a round-bottom flask was added DBCO-PEG8-COOH) (100 mg, 0.137 mmol), HATU (1-[Bis(dimethylamino)methylene]-1H-1,2,3-triazolo[4,5-b]pyridinium 3-oxid hexafluorophosphate; 2 eq., 104 mg), diisopropylethylamine (2 eq., 48 μL), and 4 ml N, N-dimethylformamide. The reaction was stirred at room temperature for 30 minutes, followed by the addition of MMAE (Monomethylauristatin E; 1.24 eq., 122 mg) and another incubation for 3 hours. The formation of the desired product was confirmed by Agilent 6130 Series quadrupole MS system (Capillary voltage: 3K, desolvation temperature: 350 °C). Observed [M+H]⁺ 1428.7; calculated [M+H]+ 1428.85. The product was purified using Agilent preparative HPLC systems (1260 Infinity II) equipped with Waters™ SunFire® C18 column (19×100 mm, 5 μm).

### N-Glycan Profiling Using HILIC

N-glycan profiling using HILIC was performed as follows. (A) Glycan Isolation: 7.5 μL antibody from 3 mg/ml stock solution (25 mM Tris-HCl pH 7.5) was mixed with 6 μL RapiGest™ SF (Waters, 50 mg/mL in 25 mM sodium acetate pH 5.5) and 15.3 μL water in a 1.5 mL microtube. The mixture was incubated at 95 °C for 5 minutes and then cooled down to room temperature for 2 minutes. 1.2 μL Rapid™ PNGase F was then added to the tube followed by another incubation of 10 minutes at 50 °C. After the PNGase F digestion, 12 μL RapidFluor™-MS solution (Waters, 70 mg/ml in DMF) was added, gently mixed, and incubated at room temperature for 20 minutes. After the incubation, the sample was diluted with 360 μL acetonitrile (leading to a 10:90 v/v water:acetonitrile mixture).

(B) Purification: Two purifications were used to ensure a good signal-to-noise ratio in glycan profile analysis using LC-MS. For the first purification, Oasis SPE (Solid Phase Extraction) μPlate from Waters (along with the use of μPlate extraction manifold) was washed with water (1 column volume) and conditioned with water-acetonitrile solution (10:90 v/v, 1 column volume). The glycan sample prepared above (in 90% acetonitrile solution) was then charged to the μPlate, followed by washing with washing buffer (formic acid/water/acetonitrile 1:9:90 v/v/v, 2 column volume). The N-glycans were eluted using 90 μL elution buffer (200 mM ammonium acetate in 5% acetonitrile). The collected sample was mixed with acetonitrile again to a 10:90 v/v ratio and was ready for the second purification using the Discovery SPE cartridge from Sigma-Aldrich (along with the use of a 20-wells SPE vacuum manifold). The protocols used in the first purification were repeated except for the decreased volume of elution buffer (70 μL). The collected glycan sample was transferred to HPLC vials and subjected to LC-MS analysis.

(C) Analysis: LC-MS analysis was performed on an Agilent 1290 Infinity II LC system in tandem with Agilent 6230 Series quadrupole time-of-flight MS system (LC-QtoF-MS). An ACQUITY BEH Glycan column (130 Å, 1.7 μm, 2.1×150 mm) was used for the analysis with the mobile phase A to be 50 mM ammonium formate in H₂O (pH 4.4) and phase B to be 100% acetonitrile. The analysis was carried out at 60 °C with the gradient shown below:

| Time (min) | Flow rate (mL/min) | % A | % B |
|---|---|---|---|
| 0 | 0.4 | 25 | 75 |
| 35 | 0.4 | 46 | 54 |
| 36.5 | 0.2 | 100 | 0 |
| 39.5 | 0.2 | 100 | 0 |
| 43.1 | 0.2 | 25 | 75 |
| 47.6 | 0.4 | 25 | 75 |
| 55 | 0.4 | 25 | 75 |

### N-Glycan Engineering to Create Substantially Homogeneous Azido Glycoforms

Recombinant human N-Acetylglucosaminyltransferase III (GnT-III) was used to introduce azidoacetylglucosamine (6-azido-GlcNAc or GlcNAz, R&D Systems) to the FA2 glycoform of antibody. To create homogeneous FA2 glycoform, 1 mg antibody, 50 μg β1-4 galactosidase (New England Biolabs), and 20 μg α2-3/6 neuraminidase were mixed in a 1.5 mL microtube, followed by buffer transfer into the reaction buffer (50 mM sodium acetate buffer pH 5.5 with 5 mM CaCl₂)) using 10K molecular-weight cutoff (MWCO) tubes. After buffer transfer, the volume of the reaction was adjusted to 200 μL to give a final antibody concentration of 5 mg/mL. The reaction was incubated at 37 °C for 16 hours. The complete conversion of glycoforms into FA2 was confirmed by LC-MS using the protocol described in the previous section. The reaction then was diluted with 2× (v/v) 25 mM phosphate buffered saline (PBS) pH 7.5 and the antibody was purified using protein A resin (Cytiva).

To install 6-azido-GlcNAc or GlcNAz, the glycoengineered antibody (FA2) was mixed with 5 weight percent (wt %) GnT-III in a 1.5 mL microtube, followed by buffer transfer into the GnT-III buffer (50 mM Tris pH 7 with 50 mM NaCl and 10 mM MnCl₂) using 10 K MWCO tubes. The sample volume was adjusted with the GnT-III buffer to give a final antibody concentration of 5 mg/mL. 10 equivalent UDP-6-azido-GlcNAc or UDP-GlcNAz (R&D Systems) was then added to the sample to initiate the reaction. The reaction was incubated at 30 °C with shaking until the reaction was complete (24-48 hours). Complete 6-azido-GlcNAc or GlcNAc installation (product: FA2B-Az) was confirmed by LC-MS using the protocol above.

To build the azido-glycoforms with terminal galactose or sialic acid, 2.5 wt % of β1-4 galactosyltransferase (Homo sapiens) and 4 wt % of α2-6 sialyltransferase (Homo sapiens) were introduced to the antibody bearing the FA2B-Az glycoform. The reaction was carried out in 25 mM Tris-HCl buffer containing 10 mM MgCl₂ and MnC12 at 37 °C for 48-72 hours. Complete glycoengineering of the FA2BG2-Az and FA2BG2S-Az glycoforms was confirmed by LC-MS using the protocol above.

### Payload Conjugation

Monomethylauristatin E (MMAE) derivatives (the payloads) containing a dibenzocyclooctyne (DBCO) group were used for the antibody conjugation through the copper-free click chemistry. The payloads were dissolved in dimethylsulfoxide (DMSO) to make a 100 mg/mL stock solution. The conjugation reactions were carried out by mixing the glycoengineered antibody prepared above with 30 equivalent payloads in 25 mM Tris 7.4 buffer with 100 mM NaCl. The reaction volume was adjusted to give a final antibody concentration between 4-5 mg/mL. Additional DMSO was added to the reaction to a final amount of 10% (v/v) DMSO to improve the payload solubility. The reaction was incubated at room temperature with shaking. The LC-QToF-MS was used to monitor the reaction until the reaction was complete. A longer incubation time is required for the reactions with antibodies having galactosylated and sialylated N-glycans. To purify the product, the samples were diluted with 2× (v/v) 25 mM Tris-HCl pH 7.5 buffer charged to a protein A (Cytiva) column. After washing with 5 column volume of the Tris-HCl buffer, the MMAE-ADCs were eluted from the column, buffer-exchanged into 50 mM sodium acetate buffer (pH 5.5), and stored at 4 °C before use. BCA assays were used to determine the concentration of ADCs.
Column: Agilent Poroshell EC-C18, 2.1×50 mm, 1.9 μm
Mobile phase A: Water with 0.1% (v/v) formic acid
Mobile phase B: Acetonitrile with 0.1% (v/v) formic acid
Temperature: 60 °C.

| Time (min) | Flow rate (mL/min) | % A | % B |
|---|---|---|---|
| 0 | 0.5 | 95 | 5 |
| 2 | 0.5 | 95 | 5 |
| 2.1 | 0.5 | 80 | 20 |
| 4 | 0.5 | 50 | 50 |
| 5 | 0.5 | 5 | 95 |
| 6 | 0.5 | 5 | 95 |
| 6.1 | 0.5 | 95 | 5 |
| 7 | 0.5 | 95 | 5 |

### Protein (Antibody) Mass Analysis Using ToF-UPLC

For intact antibody mass analysis, 2 μL antibody solution (from 3-4 mg/ml samples) was diluted with 40 μL water in a high performance liquid chromatography (HPLC) vial. For reduced antibody mass analysis, 1 μL antibody solution (from 3-4 mg/mL samples) was diluted with 40 μL 25 mM 1,4-dithiothreitol (DTT) solution (in water) in an HPLC vial, followed by an incubation of 30 minutes at 37 °C. The samples were subjected to the LC-QToF-MS system (Capillary voltage: 5K, desolvation temperature: 400 °C) equipped with an Agilent Poroshell EC-C18 column (2.1×50 mm, 1.9 μm) which enables the separation of the antibody from small molecules (e.g., payloads). Water with 0.1% (v/v) acetic acid and acetonitrile with 0.1% (v/v) acetic acid were used as the mobile phase A and B, respectively. The analysis was carried out at 40 °C with the gradient shown below:

| Time (min) | Flow rate (mL/min) | % A | % B |
|---|---|---|---|
| 0 | 0.5 | 95 | 5 |
| 2 | 0.5 | 95 | 5 |
| 2.1 | 0.5 | 80 | 20 |
| 4 | 0.5 | 50 | 50 |
| 5 | 0.5 | 5 | 95 |
| 6 | 0.5 | 5 | 95 |
| 6.1 | 0.5 | 95 | 5 |
| 7 | 0.5 | 95 | 5 |

The MS data from the analyses were imported to the Agilent MassHunter BioConfirm software (version B.08.00) for mass deconvolution. The deconvoluted mass data were analyzed using the MassHunter DAR Calculator to determine the drug-to-antibody ratio.

### Synthesis of DBCO-PEG8-MMAE

To a round-bottom flask was added DBCO-PEGg-COOH (100 mg, 0.137 mmol), HATU (2 eq., 104 mg), diisopropylethylamine (2 eq., 48 μL), and 4 mL N, N-dimethylformamide. The reaction was stirred at room temperature for 30 minutes, followed by the addition of MMAE (1.24 eq., 122 mg) and another incubation for 3 hours. The formation of the desired product was confirmed by Agilent 6130 Series quadrupole MS system (Capillary voltage: 3K, desolvation temperature: 350 °C). Observed [M+H]⁺ 1428.7; calculated [M+H]⁺ 1428.85. The product was purified using Agilent preparative HPLC systems (1260 Infinity II) equipped with Waters SunFire C18 column (19×100 mm, 5 μm).

### Hydrophobic Interactions Chromatography (HIC) Analysis

Samples were analyzed using TSKgel Butyl-NPR 2.5 μm 4.6×100 mm column (Tosoh) on an Agilent 1290 Infinity II HPLC system. 20 μg of each sample was injected and was monitored at 220 nm, 254 nm, and 280 nm. 12.5 mM sodium phosphate (pH 7.0) with 1 M ammonium sulfate was used as mobile phase A and 50 mM sodium phosphate (pH 7.0) with 20% isopropanol was used as mobile phase B. Gradient elution was performed with 5-95% B at 0.4 mL/min over 30 minutes at 25 °C.

### Size-Exclusion Chromatography (SEC) Analysis

SEC-UPLC was performed using an ACQUITY Protein BEH SEC 200 Å, 1.7 μm 4.6×150 mm column (Waters) on an Agilent 1290 Infinity II HPLC system, monitoring at 280 nm. 20 μg of each sample was injected and eluted with 50 mM PBS (pH 7.3) at 0.3 mL/min over 30 minutes at 25 °C.

### Binding Affinity Studies Using Biolayer Interferometry

The Octet® Red384 biolayer interferometry system (ForteBio, now Sartorius) was used for the measurement of the antibody/ADC-HER2 binding affinity. Ni-NTA Octet® sensors were hydrated in Octet buffer (25 mM Tris-HCl pH 7.4, 50 mM NaCl, 0.02% Tween 20) for 30 minutes before the experiments. To begin the measurement, the sensors were loaded with 0.5 μg/mL His-tagged FcγRs for 240 seconds, followed by baseline equilibration for 2 minutes. Association of recombinant human ACE2 was performed with the antibody or ADCs from 12.5 to 1000 nM for 600 seconds. Dissociation was measured for 600 seconds. The equilibrium dissociation constant (KD) values were calculated using a 1:1 global fit model in the Octet data analysis software. The assays were carried out at 30 °C and the Octet buffer was used as the solvent in all the steps.

For binding response analysis with differently glycoengineered ADCs, Nickel-Nitriloacetic acid (Ni-NTA) sensors were loaded with 0.5 μg/mL His-tagged FcγR-IIa or FcγR-IIIa for 240 seconds, followed by incubation with 5 μg/mL FcγR-IIa or 40 μg/mL FcγR-IIIa for 240 seconds. The binding response was monitored in real-time.

### Western Blotting for Studying HER2 Binding

Standard SDS-PAGE and Western detection protocols were used except the human anti-HER2-MMAE ADC was used as the primary antibody in the experiment. Briefly, 2 μg recombinant human HER2 was added to a NuPage™ 4-12% Bis-Tris gel (Invitrogen NP0323BOX), followed by electrophoresis at 200V for 50 minutes. Protein transfer from the gel to a Polyvinylidene fluoride (PVDF) membrane was carried out using iBlot™ 2 Gel Transfer Device (ThermoFisher) at 20 V for 7 minutes. Following the transfer, the membrane was blocked using 5% bovine serum albumen (BSA) for 2 hours at room temperature, then washed 3× with ris-buffered saline with 0.1% Tween® 20 (TBST), and incubated with the 10 μg/mL ADC at 4 °C overnight. The ADC was detected using a goat anti-human IgG antibody (1 μg/mL, LI-COR, 926-32232). The blot was then imaged using LI-COR, Odyssey® CLx.

### ADC Synthesis Using Lysine Conjugation

NHS-PEG4-Val-Cit-PAB-MMAE from Broadpharm (BP-25503) was used for the antibody conjugation through lysine residues. The payload was dissolved in DMSO to a final stock concentration of 25 mg/mL. The conjugation reactions were carried out by mixing the native anti-HER2 human IgG1 Trastuzumab biosimilar with 5 equivalent payloads in 1×PBS buffer. The reactions were adjusted to give a final antibody concentration of 4 mg/mL and 10% DMSO. The reaction was incubated at room temperature with shaking. The reactions were monitored by the LC-QToF-MS every 30 minutes until the reaction reached a DAR of 2, immediately followed by protein A purification. The concentration of the resulting ADCs was determined by bicinchoninic acid assays (BCAs).

### ADC Synthesis Using Engineered Cysteine Conjugation

Antibody conjugation through engineered cysteine was performed as described in previously reported methods (Adhikari et al., Methods Mol. Biol. 2078, 51-69 (2020)). Trastuzumab biosimilar and an isotype IgG1 antibody with site mutations at serine 375 (S375C; Eu numbering scheme) and the MMAE derivative with a Val-Cit-PAB-maleimide linker were used. The reaction was carried out by mixing the antibodies with 5 equivalent of the payload (25 mg/mL, DMSO stock solution) in 1×PBS. The reaction volume was adjusted to a final antibody concentration of 4 mg/mL and 10% DMSO. The reaction was incubated at room temperature with shaking until the DAR reached 2 (tracking by LC-MS). The concentration of the resulting ADCs was determined by BCA.

### Cell Internalization

SKBR3 cells (ATCC HTB-30) expressing HER2 (epidermal growth factor receptor 2) were seeded to clear bottom 96-well plates to a density of 9,000 cells per well (50 μL). The cells were allowed to adhere to the well bottom for 24 hours. Test antibodies and antibody conjugates were mixed with FabFluor (0.5 mg/mL) in a 1:3 ratio and the volume was adjusted using fresh culture media (McCoy's 5A with 10% fetal bovine serum) to give a final concentration of 4 μg/mL. After an incubation of 30 minutes at 37 °C, the mixture was added to the cell plates (50 μL each well). The test antibodies/conjugates internalization was monitored every 30 minutes by the fluorescence readout using Incucyte Internalization Assay (Sartorius). This assay was done in triplicate.

### Cell Binding Assay

SKBR3 cells from culture plates were harvested and suspended in ice-cold PBS. The cells were washed with PBS (ice-cold) once using a centrifuge and resuspended in 0.5 mL stain buffer (Dulbecco's phosphate-buffered saline (PBS), pH 7.4, 2% heat-inactivated fetal bovine serum (FBS), 0.09% sodium azide). The test antibodies or antibody conjugates were added to the cell solution (10 μg/mL), followed by incubation at 4 °C for 30 minutes. After another washing step using ice-cold PBS, the secondary mAb (mouse anti-human IgG Fc gamma-FITC) was added to the cell solution (stain buffer) and incubated for 30 minutes (4 °C). The cells were then washed with PBS, resuspended to the stain buffer, and subjected to a Guava® flow cytometry system (Millipore Sigma). About 1×10⁶ cells were used per staining condition.

### ADCs Stability Study in Rat Plasma

Anti-HER2 mouse IgG2a-MMAE ADC from 6 mg/mL stock solution was added to 30 μL male rat plasma (Sprague-Dawley K2EDTA) to a final concentration of 100 μg/mL. The samples were incubated at 37 °C for three days, followed by affinity purification using AffiniPure Rat Anti-Mouse IgG to recover the remaining ADCs in the plasma. The relative amount of the ADCs was quantitated using Waters Acquity I-class UPLC in tandem with Synapt G2-S quantitative ToF-MS system (Capillary voltage: 3K, Cone voltage: 2K, desolvation temperature: 450 °C.). POROS™ R2 reverse-phase columns (2.1×30 mm, 10 μm) from Applied Biosystems were used for the UPLC system. For the day 0 control experiments, the samples were frozen immediately at −80 °C after ADCs were added. Samples from all the time points were frozen, thawed, and processed together in one experiment.

### Plotting and Graphic

Data plotting and t-test analysis were done by GraphPad Prism 8. Figures were created by Adobe Illustrator.

### Statistics and Reproducibility

All the experiments were reproduced at least 2 times (N=3) unless otherwise specified. Analyses were performed using GraphPad Prism 8 with all the collected data points included.

### Example 1

### Antibody-Drug Conjugation Through N-Glycan Bisecting GlcNAc

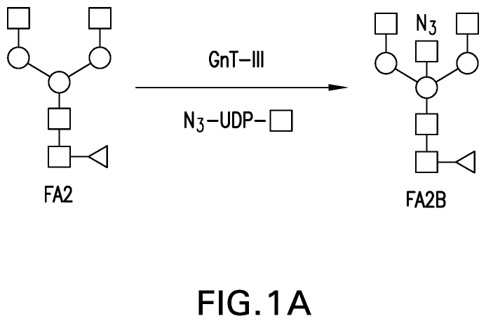

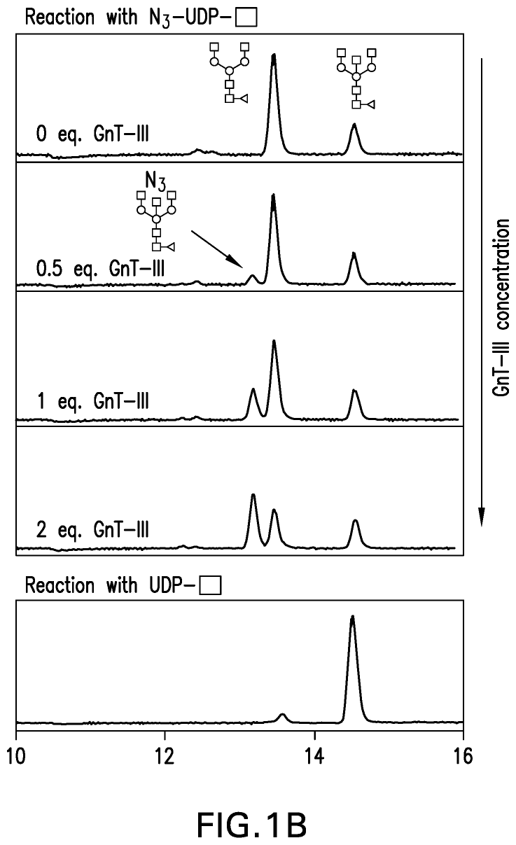

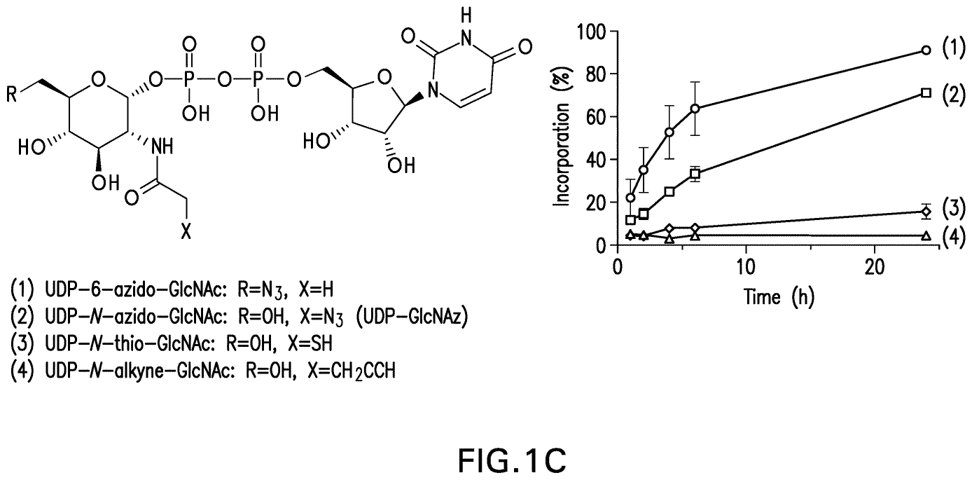

In recent decades, many glycoengineering enzymes are reported to have in vitro activities on intact proteins (Hsu et al., Communications Biology 5, 328 (2022); Thomann et al., PLOS ONE 10, e0134949, doi: 10.1371/journal.pone.0134949 (2015)). This provides an innovative approach to controlling glycan structures and also creating chemical handles for protein conjugation. Human GnT-III serves to install a GlcNAc residue through a β1-4 linkage to the bisecting-site mannose of the N-glycan pentasaccharide core.30 (See also FIG. 1A). We recently reported its in vitro activity on intact human serum IgGs bearing terminal GlcNAc residues, i.e., the FA2 glycoform (Oxford notation) (Hsu et al., Communications Biology 5, 328 (2022)). We further found that this enzyme tolerates the incorporation of azido-GlcNAc from the UDP-azido-GlcNAc donor to IgG glycans (See FIG. 1B). Having a clickable tag, the azide group, on the N-glycan bisecting site is appealing for ADC development because it is site-specific, gene editing-free, and potentially can preserve the natural features of IgG N-glycans. To test this, we used an anti-cancer mouse IgG2a (BioXCell BE0277) as a substrate, a monoclonal antibody (mAb) that binds to human epidermal growth factor receptor 2 (HER2), a Trastuzumab biosimilar.

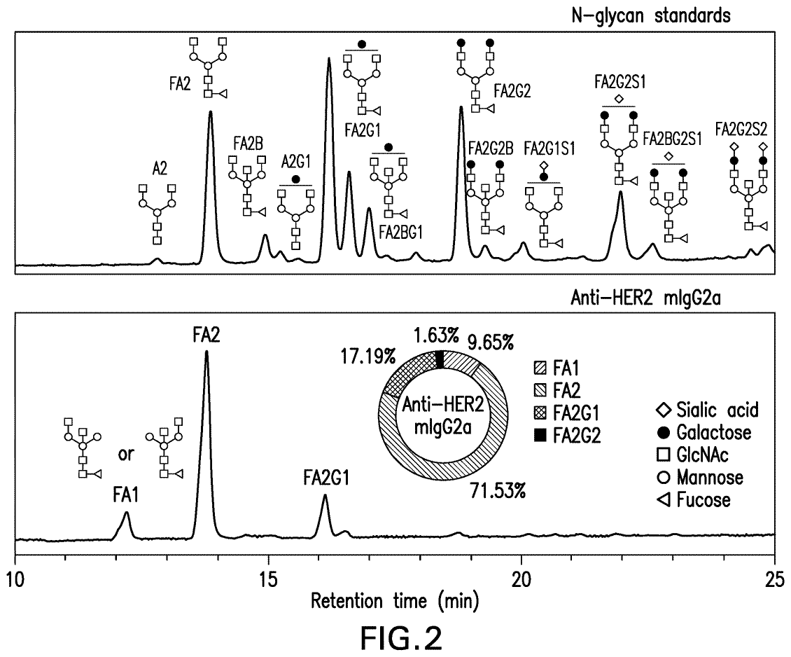

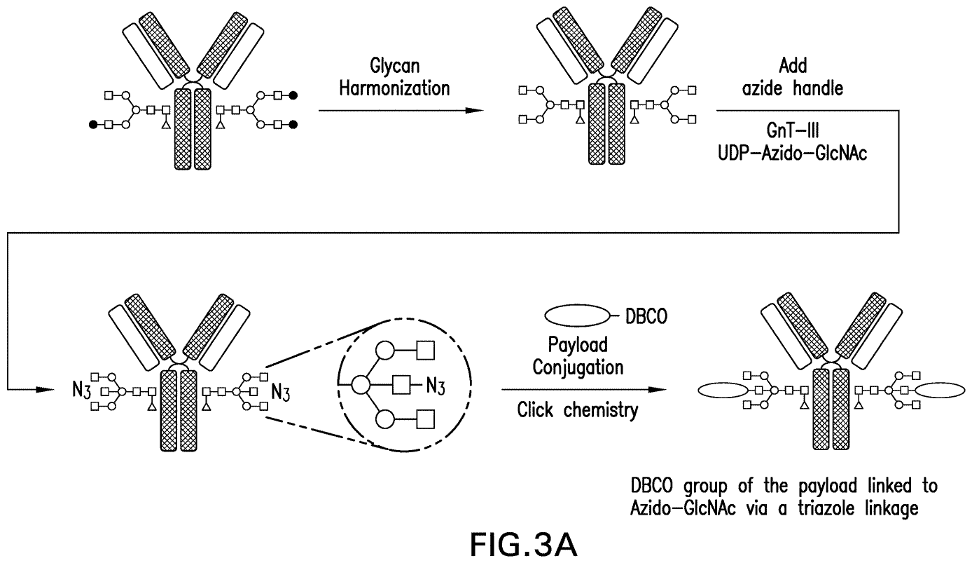

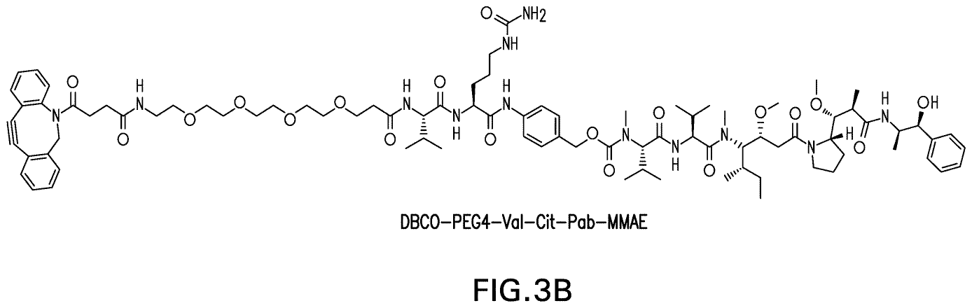

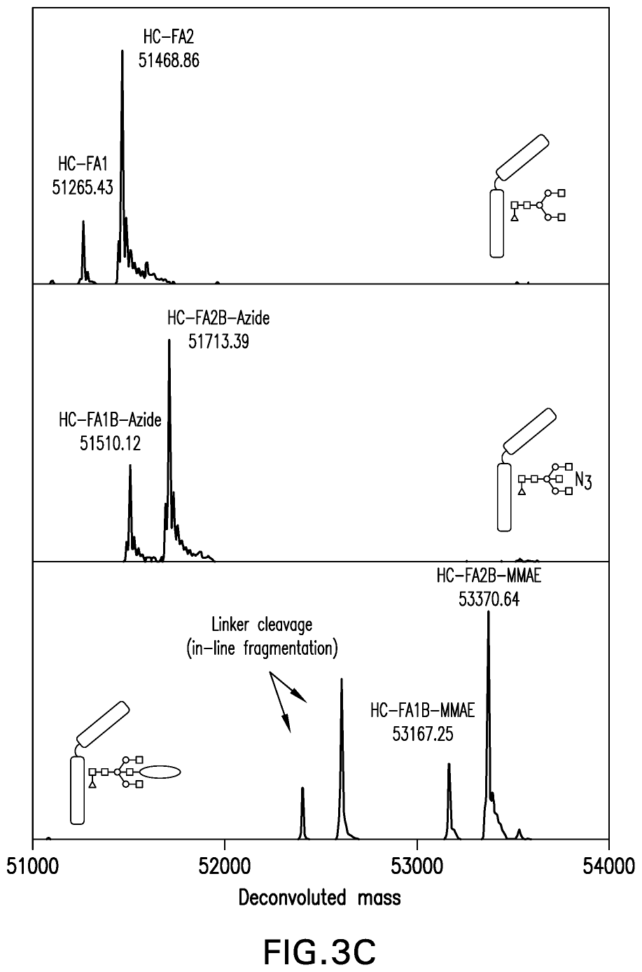

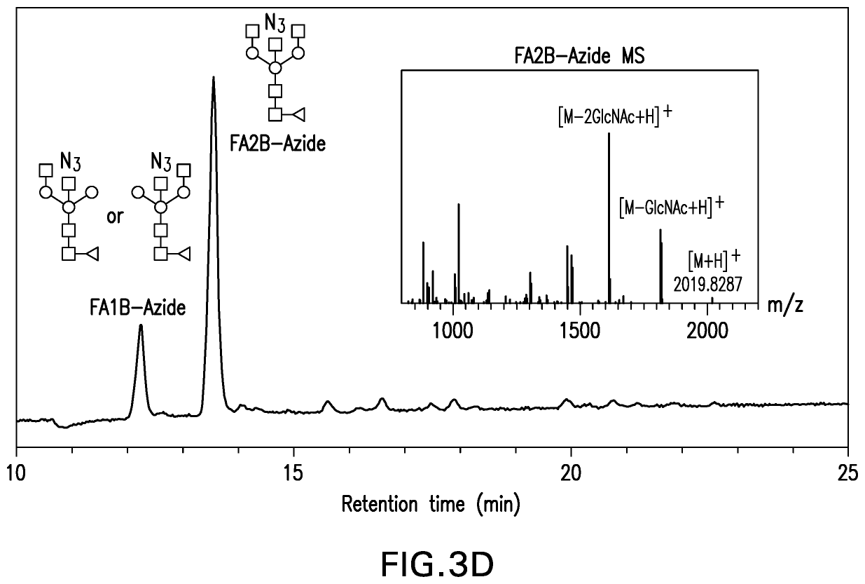

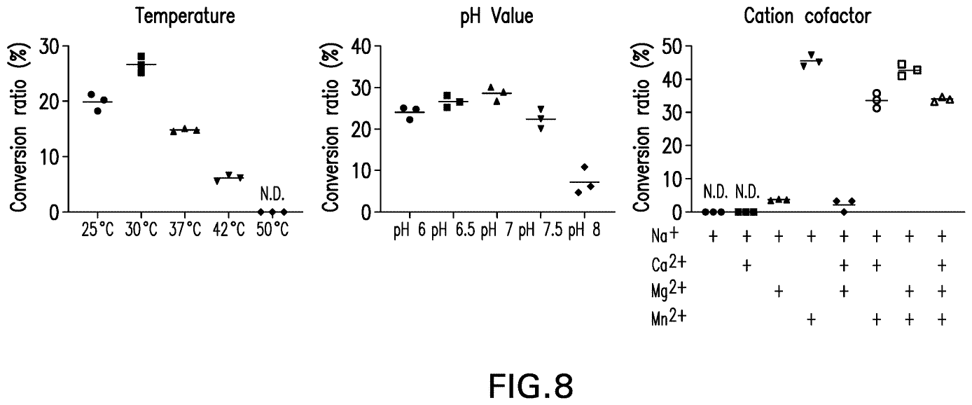

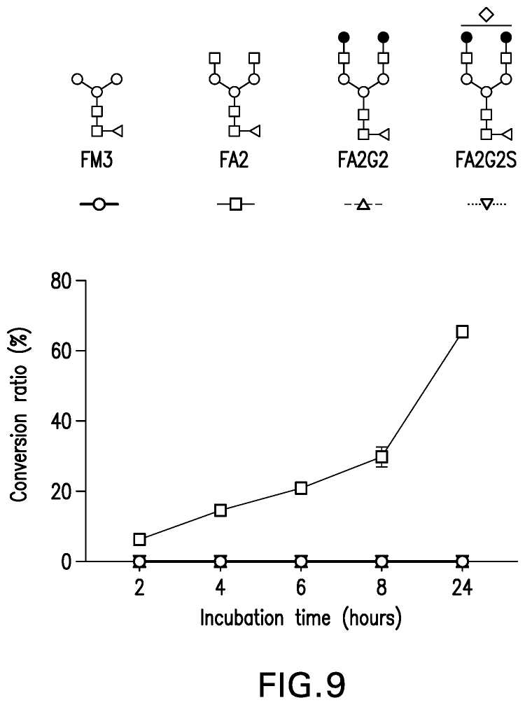

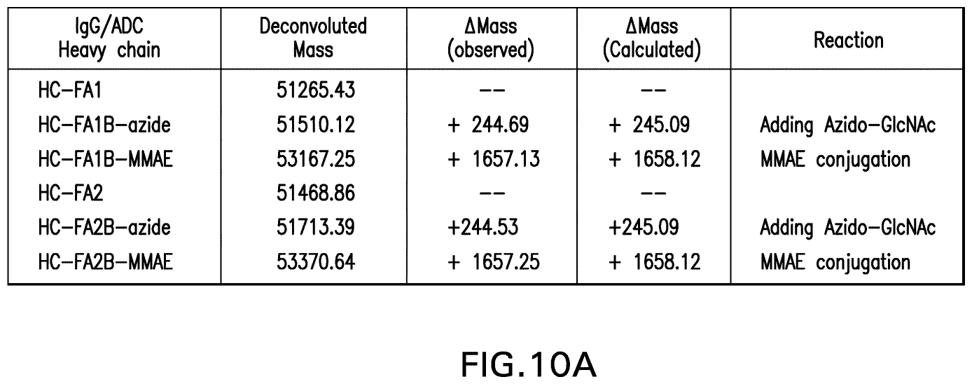

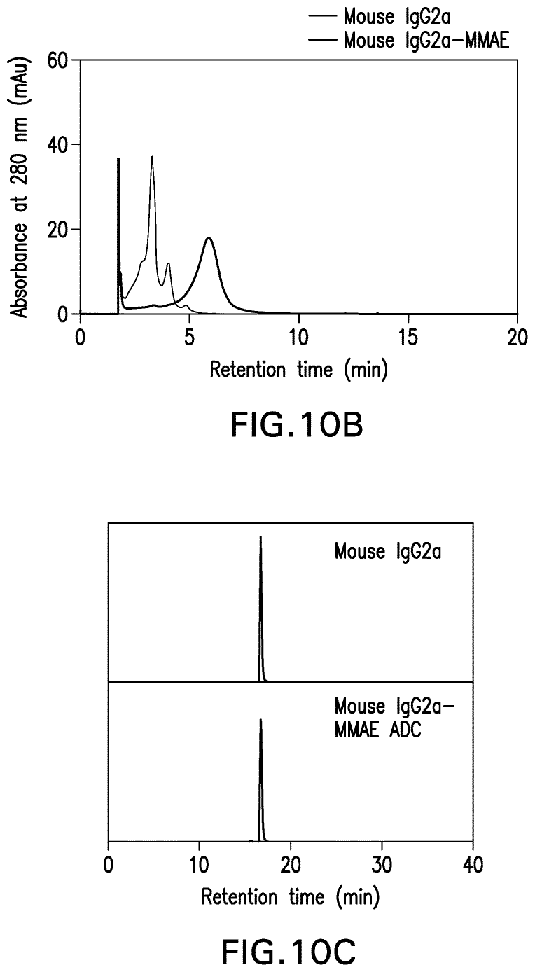

Liquid chromatograph-mass spectrometer (LC-MS) analyses showed that the native glycan profile of the substrate comprises 72% FA2, 10% FA1, 17% FA2G1, and a trace amount of FA2G2 N-glycan species (FIG. 2). To introduce azido-GlcNAc to the bisecting site, we used galactosidase to remove terminal galactose, followed by the treatment with GnT-III enzymes (FIG. 3A). The formation of azido-glycoforms (FA2B-Azide) was confirmed by deconvoluted protein mass spectrometry as well as the LC-MS analyses of the isolated glycans (FIG. 3B-3C). With the use of optimized working conditions, full conversion of the substrate glycans into azido-glycoforms can be achieved within 48 hours with the use of 0.1 equivalent of GnT-III to the substrate (FIG. 8). No GnTIII activity was observed when IgG glycan bears terminal galactose or sialic acid (FIG. 9). Next, we incubated the glycoengineered mouse IgG with MMAE having a protease-cleavable linker (Val-Cit-Pab) and a dibenzocyclooctyne (DBCO) group for click-chemistry. The formation of the mouse IgG-MMAE conjugate was confirmed by MS analyses with an observed drug-to-antibody ratio of two (DAR=2). (FIG. 3C, FIG. 10). The conjugate formation was also supported by the change of molecule hydrophilicity as observed in hydrophilic interaction chromatography (HIC). Size-exclusion chromatography (SEC) suggested no aggregation or degradation after MMAE conjugation (FIG. 10).

### Example 2

### Characterization of the Bisecting Glycan-Bridged Linker Cleavage and Stability

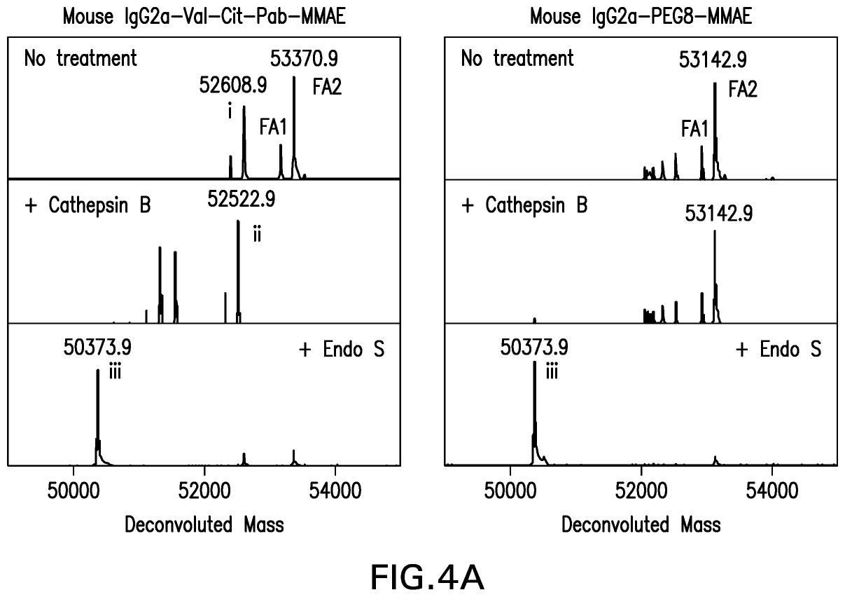

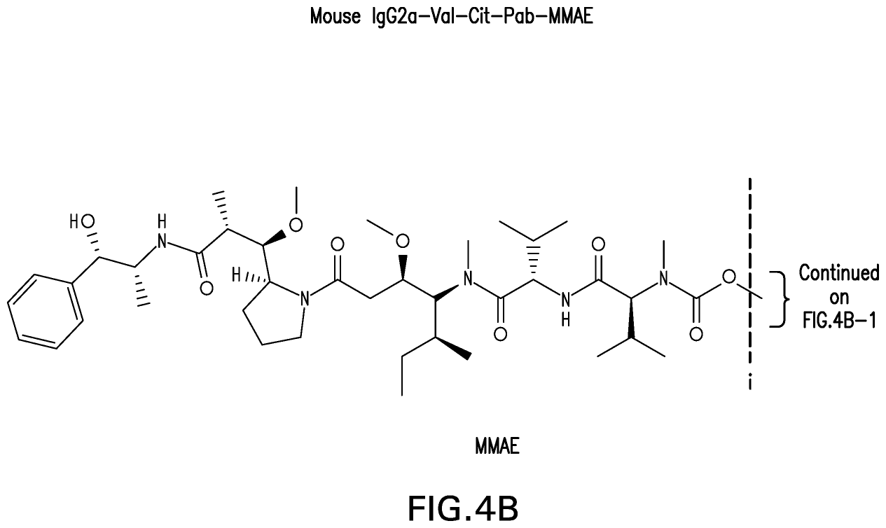

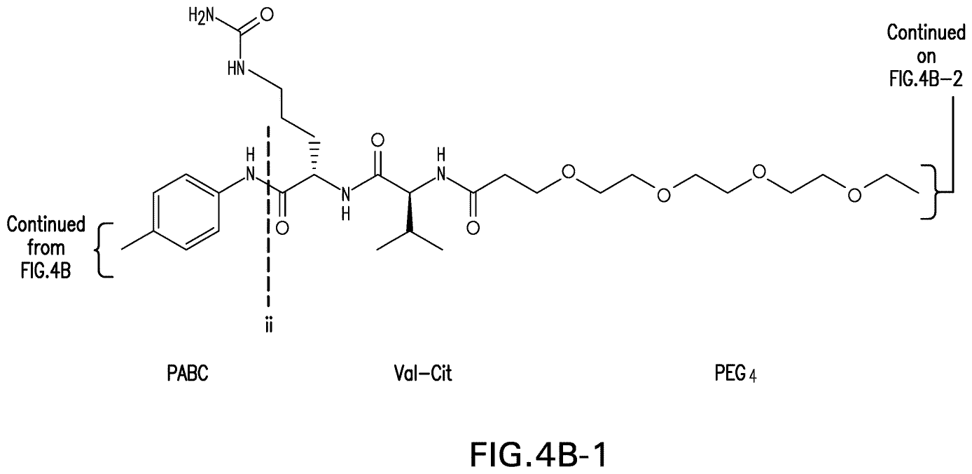

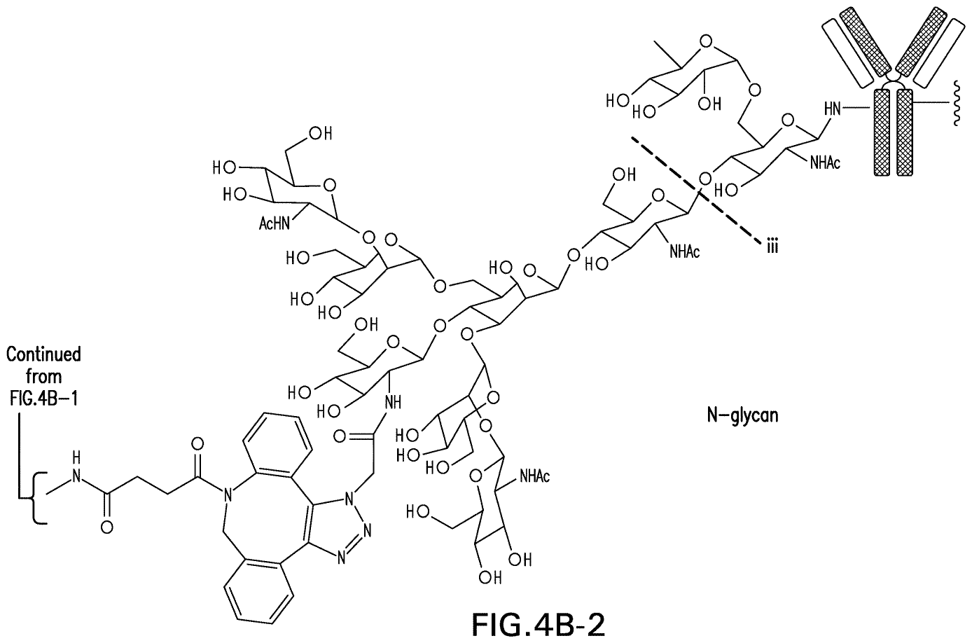

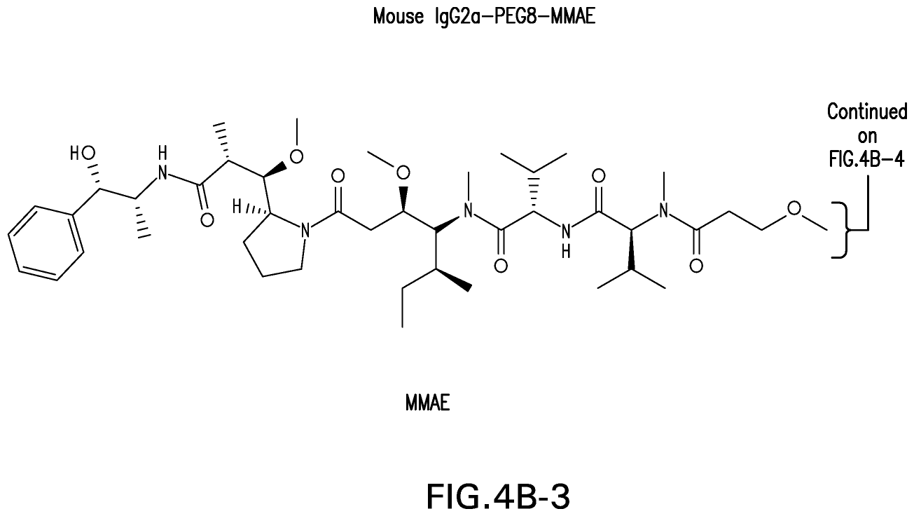

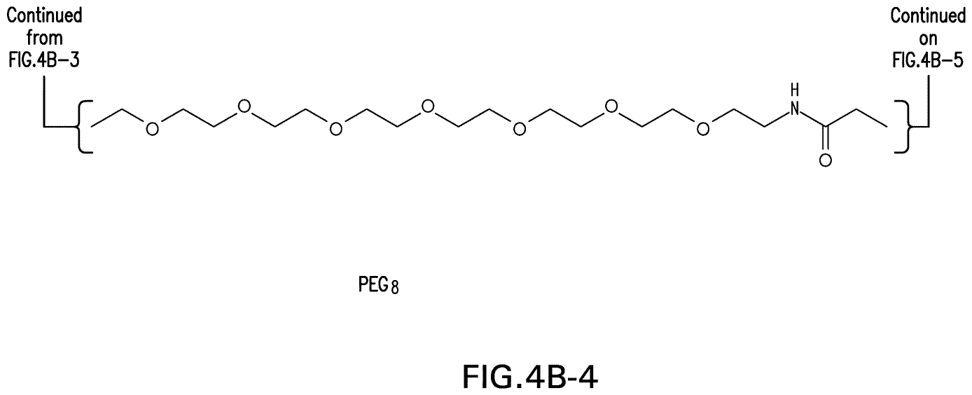

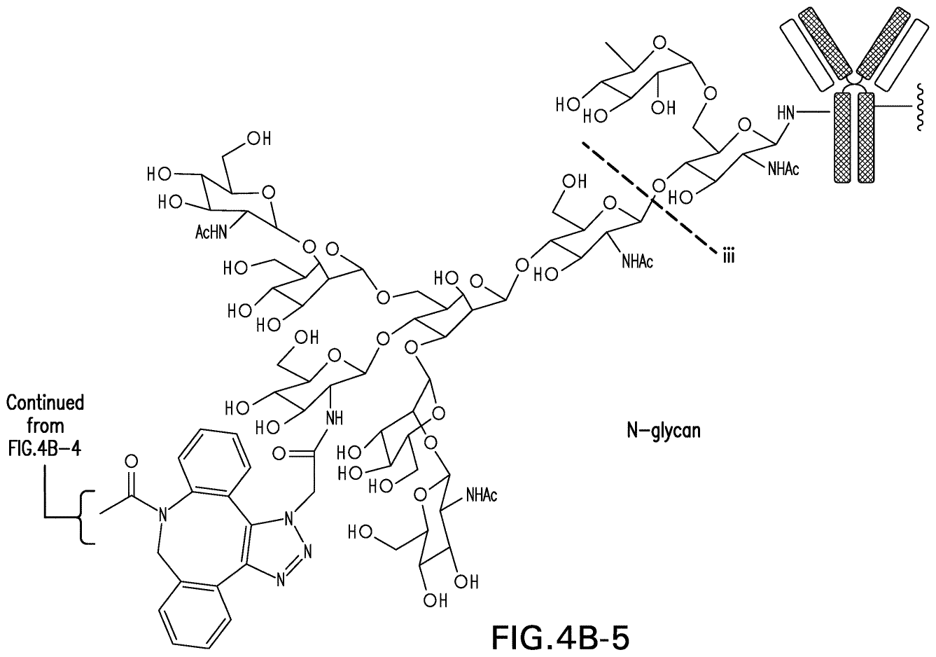

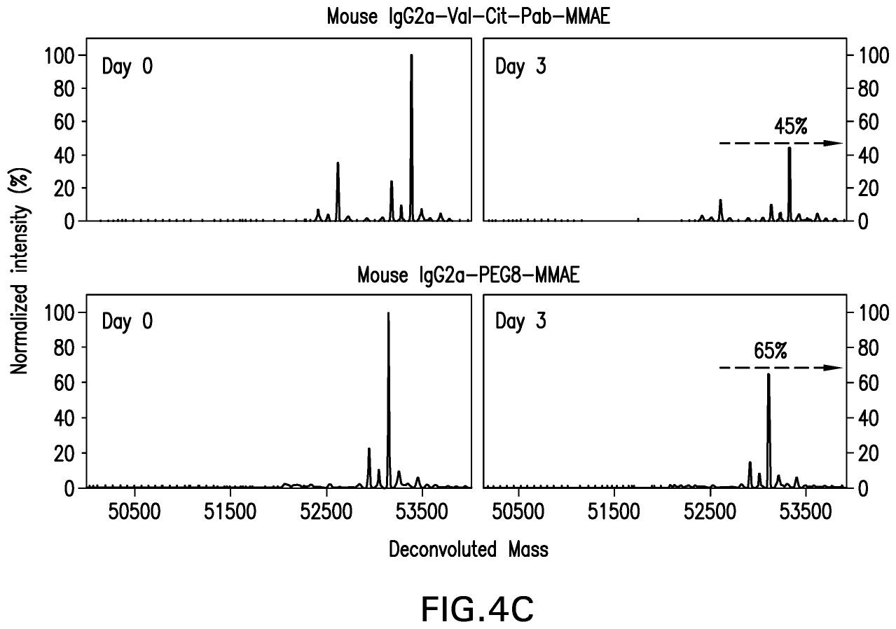

The proposed mechanism of action for anti-cancer ADCs includes 1) binding to the targeted antigens on the cancer cell surface, 2) internalization of the ADCs, and 3) the release of the cytotoxic payload to kill the cancer cells (Peters, & Brown, Biosci. Rep. 35, e00225, doi: 10.1042/BSR20150089 (2015); Chalouni. & Doll, J. Exper. & Clinical Cancer Res. 37, 20 (2018)). The linker that connects the antibody framework and the drug payload plays a critical role in the payload release mechanism (Su et al., Acta Pharmaceutica Sinica B 11, 3889-3907 (2021)). They are designed to be stable in the bloodstream but can be efficiently cleaved at the tumor site. Val-Cit-PABC (Valine-Citrulline-p-aminobenzylcarbamate) is a peptide linker widely used for ADC development as it has outstanding stability in plasma but remains labile to lysosomal enzymes, such as Cathepsin B. Glycan-bridged conjugation presents another possibility to design enzyme-mediated payload release through the activity of glycosidases. For example, the endoglycosidase from Streptococcus pyogenes (also known as Endo S) shaves off IgG glycans by cleaving the β1-4 linkage between the GlcNAc residues in the chitobiose core (Freeze & Kranz, Curr. Protoc. Mol. Biol. 17.13A.1, doi: 10.1002/0471142727.mb1713as89 (2010); Collin & Olsen, EMBO J. 20, 3046-3055 (2001)). It is a secreted enzyme that can efficiently deglycosylate humoral IgG, which contributes to the pathogenicity of the pathogen (Collin & Olsen, EMBO J. 20, 3046-3055 (2001)). Thus, this enzyme potentially releases payloads from our ADC. To test this, we incubated the mouse IgG-MMAE ADC with Endo S for two hours at 0.1 equivalent. We observed full removal of the N-glycan-payload moiety (FIG. 4A-4B). The treatment of Cathepsin B under the same buffer condition also released the MMAE payload from the Val-Cit-PABC cleavable site. In comparison, the ADC analog without the Val-Cit-PABC linker (mouse IgG-PEG8-MMAE) tolerates Cathepsin B treatment but not Endo S. These results suggested that it is possible to achieve glycosidase-dependent payload release using N-glycan-bridged conjugation strategies. It could be designed for anti-bacterial ADC development against species secreting endoglycosidase (e.g., S. pyogenes).

Next, we studied the linker stability and the ADCs degradation rate in the physiological environment by using a plasma stability assay. The mouse ADCs were incubated in rat plasma for three days at 37 °C. The remaining ADCs in the plasma were salvaged using an anti-mouse antibody and their amount was quantitated by LC-MS. As shown in FIG. 4C, about 45% of the ADC with the Val-Cit-PABC linker was recovered from the plasma; while the ADC without the cleavable linker showed an improved lifetime with 65% of the material recovered. In both cases, there was no linker/payload cleavage observed but the decreasing signal of the entire ADCs.

### Example 3

### Bisecting Glycan-Bridged Conjugation Applies to Human IgG and is Bio-Compatible

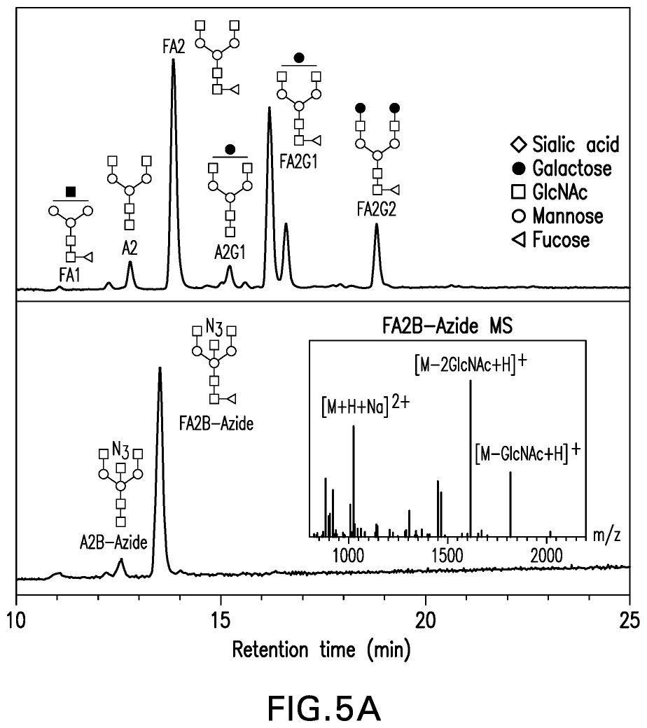

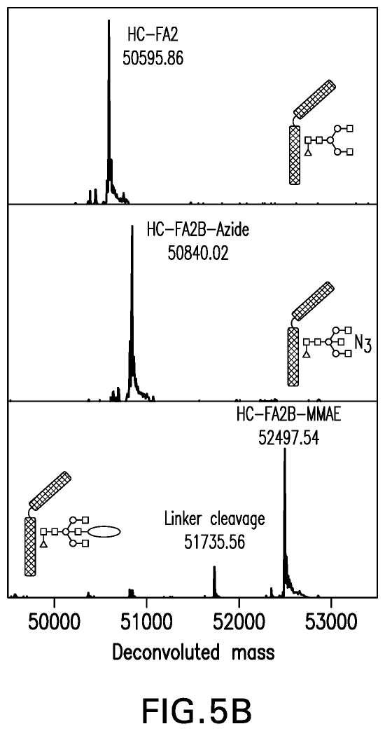

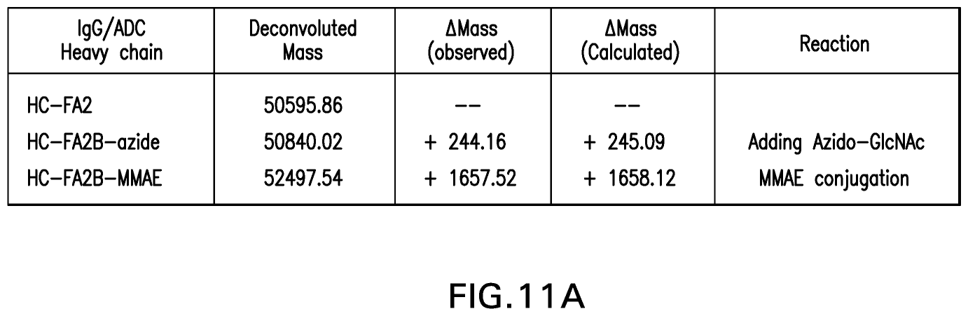

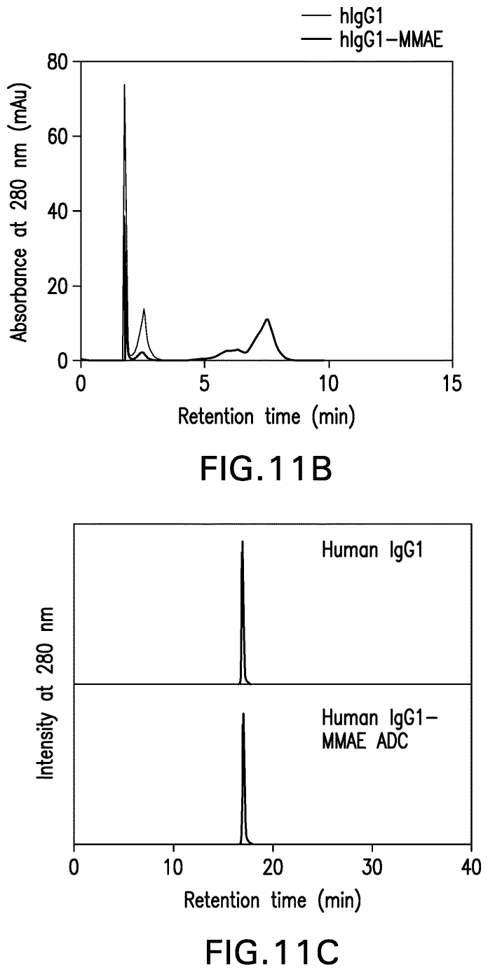

To study the impact of the ADC's biochemical properties, we applied this conjugation strategy to a human IgG1 Trastuzumab biosimilar antibody. Trastuzumab is a well-studied mAb for breast and stomach cancer drug development.40 Similar to the mouse IgG2a substrate, the native glycan profile of the Trastuzumab biosimilar comprises bi-antennary N-glycans with terminal galactose or GlcNAc (FIG. 5A). However, it also has a small amount (<10%) of afucosylated glycoforms that were not found in the mouse IgG. We removed terminal galactose and confirmed the addition of an azido-GlcNAc residue to the A2 and FA2 glycoforms using LC-MS as well as the downstream MMAE conjugation (FIG. 5B, FIG. 11).

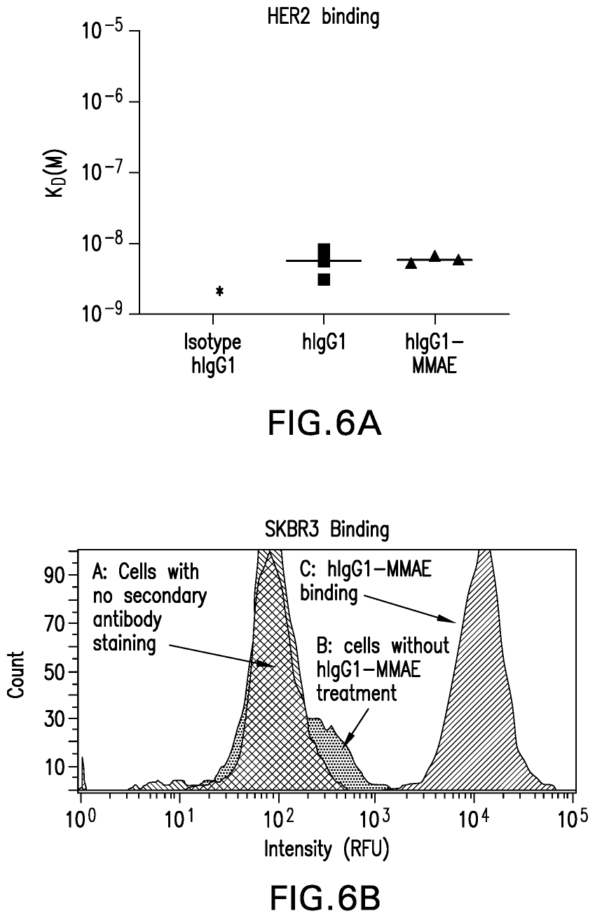

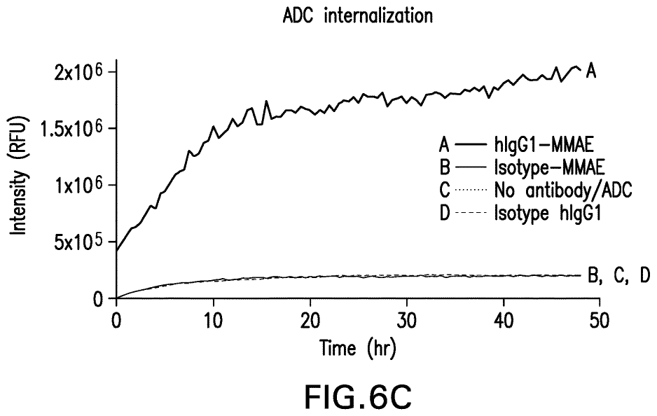

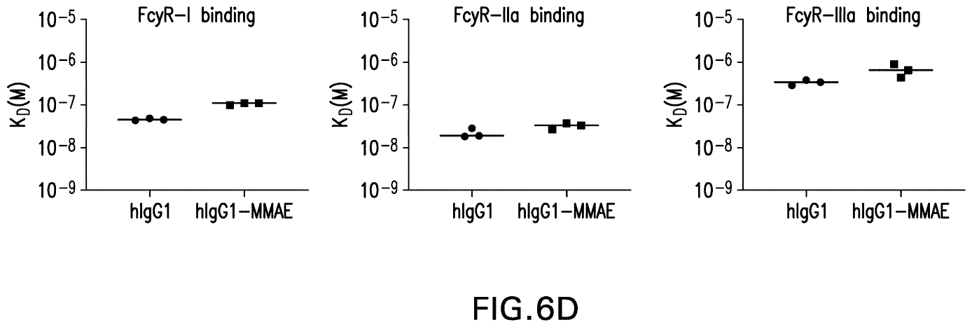

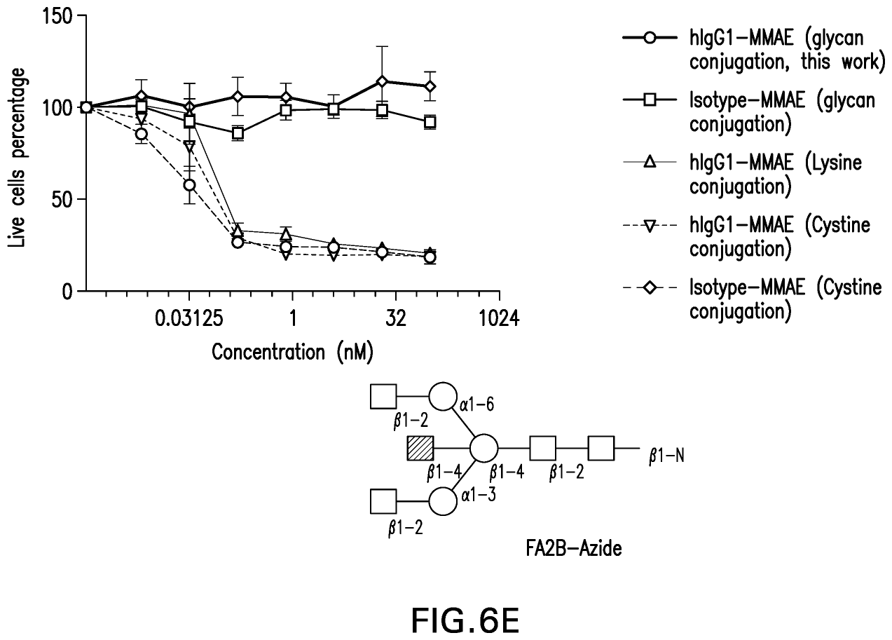

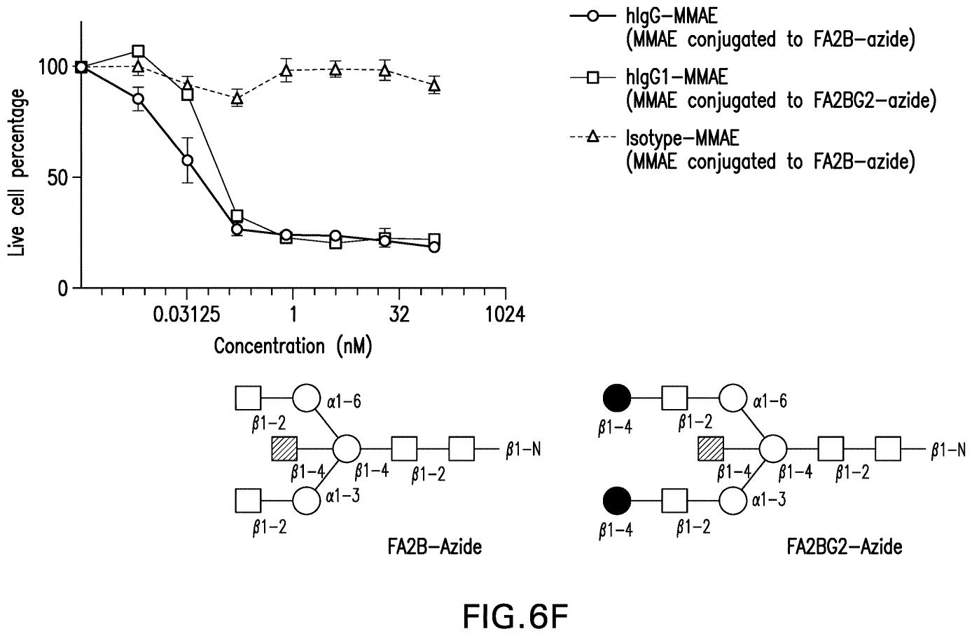

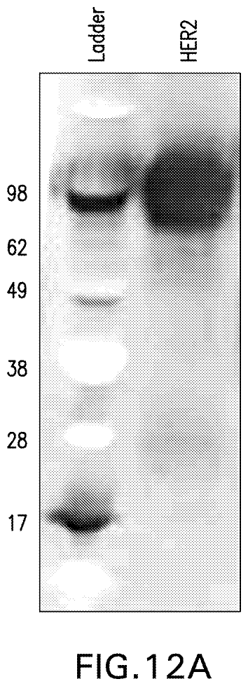

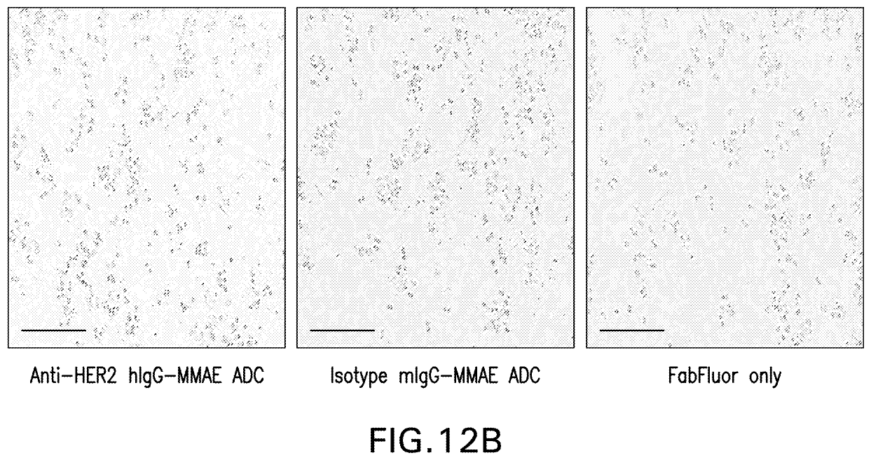

Next, we studied whether human IgG-MMAE conjugates (IgG1-azido-GlcNAc-MMAE ADC) preserve the binding ability to its target antigen. Antibodies bind to antigenic targets through the antigen-binding region (Fab) which is spatially separated from the glycosylation site (N297 in the Fc region, position number as determined by the EU numbering scheme). Dissociation constant (KD) measurement using biolayer interferometry (BLI) indicated that payload conjugation at N297 does not impact IgG binding affinity for recombinant human HER2 (FIG. 6A). This was supported by Western blotting using the ADC as the primary antibody, as well as cell-based experiments where the binding of the ADC to SKBR3 (HER2 expressing cell line) cell surface was observed (FIG. 6B, FIG. 12). Moreover, we did a cell internalization assay and showed that the ADC undergoes endocytosis, indicating successful delivery of the payload into the cells. (FIG. 6C, FIG. 12). To evaluate the ADC's potency in killing HER2-expressing cells, the viability of SKBR3 cells was measured after 72-hour incubation with the IgG-MMAE conjugates (FIG. 6B). A dose-dependent decrease in cell viability was observed with an EC₅₀ of about 0.1 nM. We further prepared anti-HER2-MMAE ADCs using other conjugation methods, including the random lysine conjugation through amine-NHS ester reaction and engineered cysteine conjugation (S375C) through cysteine-maleimide chemistry (Sussman et al., Protein Eng. Des. Sel. 31 47-54 (2018)). All IgG-MMAE ADCs showed good SKBR3-killing potency, with a slightly improved EC₅₀ found in the ADC using glycan conjugation. As negative controls, we used isotype IgG1-MMAE ADCs, which showed no activity in the cell viability assays. Our findings suggest that the IgG-MMAE conjugates using glycan conjugation are effective in targeting HER2-expressing cells and have the potential for further development as therapeutic agents.

On the other hand, the impact of conjugation is clearer on IgG-FcγR interactions. FcγR is a family of proteins found on the surface of immune cells. They bind to the hinge region and the CH2 domain of IgG, which covers the glycosylation site, and play versatile roles in antigen recognition and immune response regulation (de Taeye et al., Antibodies (Basel) 8, doi: 10.3390/antib8020030 (2019); Junker et al., Immunology 11, doi: 10.3389/fimmu.2020.01393 (2020); Nimmerjahn & Ravetch, Nature Reviews Immunol. 8, 34-47 (2008)). FcγR-I and FcγR-Ila are good IgG binders (Bruhns et al., Blood 113, 3716-3725, doi: 10.1182/blood-2008-09-179754 (2009)). Our data showed that the Trastuzumab biosimilar has a binding affinity (KD) in the nano-molar scale to FcγR-I and FcγR-IIa (FIG. 6D). Its MMAE conjugate to N297 showed a compatible binding affinity but slightly increased KD values (1.4-fold). Compared to FcγR-I and FcγR-IIa, FcγR-IIIa is known to have weak interaction with IgG (Bruhns et al., Blood 113, 3716-3725 (2009)). It showed a binding affinity in the micro-molar scale (KD) to the Trastuzumab biosimilar but an approximately 2-fold increase after payload conjugation through the glycans.

FIG. 6E cell viability studies demonstrate target-specific toxicity of the hIgG-MMAE was comparable to ADCs synthesized by using lysine conjugation or engineered cysteine conjugation (positive controls). FIG. 6F shows that HER2-expressing cell killing activity between hIgG-MMAE conjugated to FA2B-azide or FA2BG2-azide were comparable.

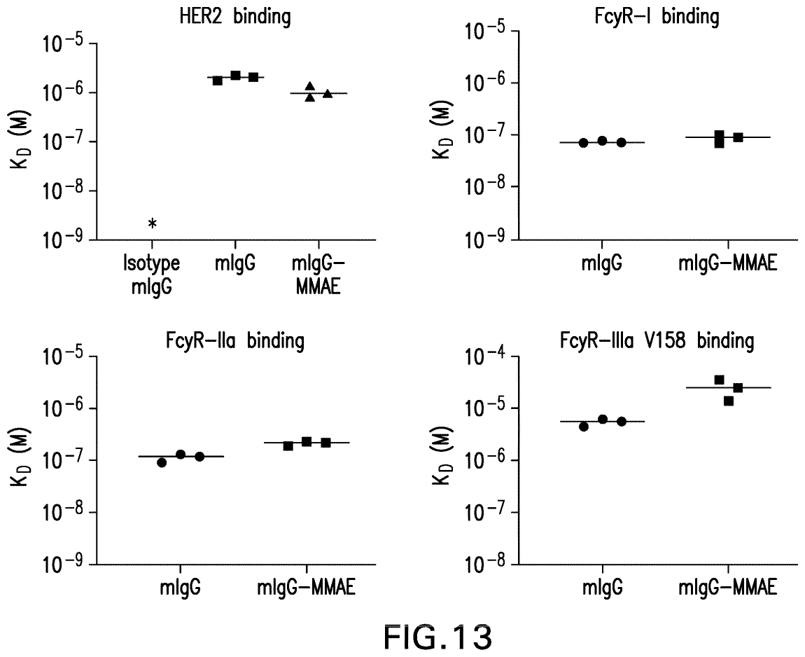

Mouse IgG2a is reported to have cross-binding affinities to certain types of human FcγRs, including FcγR-I, FcγR-IIa, and FcγR-IIIa V158 (Temming et al., Mol. Immunol 127, 79-86 (2020)). Similar to the human IgG1 conjugate, attaching MMAE to the mouse anti-HER2 IgG2a resulted in slightly increased KD values, as indicated by Biolayer Interferometry (BLI) assays (FIG. 13). Overall, the MMAE ADCs prepared in this study preserve binding abilities to the antigen target and FcγRs while a slight decrease in FcγRs binding affinity was observed (<10-fold KD change). Since payload molecules could have distinct chemical and physical properties, the effect of payload conjugation should be evaluated when different payloads are used, especially for weaker IgG-protein interactions, e.g., FcγR-IIIa.

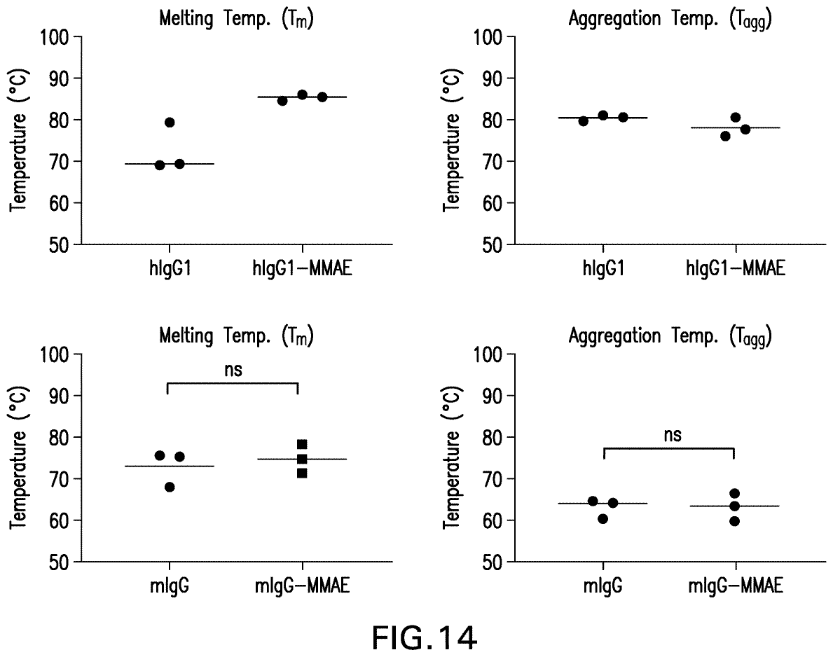

In addition to the binding ability, we also studied the protein conformation stability of the MMAE-conjugated ADCs by measuring their melting temperature (Tm) and aggregation temperature (Tagg) (FIG. 14). No detectable decrease in Tm and Tagg was observed after MMAE conjugation while, unexpectedly, an increase of Tm for approximately 10 °C was found in the human IgG-MMAE ADC. It has been reported that payload conjugation to the IgG CH2 domain could result in both positive and negative effects, depending on the payload properties and the conjugation sites.46 MMAE molecules have high hydrophobicity and likely adopt inward orientation in the Fc region to minimize the exposure to the hydrophilic surrounding. They could form non-polar interactions with each other, or with the hydrophobic IgG residues in the CH2 domain (e.g., F241, F243, V262, V264), which stabilizes the Fc conformation and results in the Tm increase (Chen et al., ACS Chem. Biol. 11, 1852-1861 (2016)).

### Example 4

### Tuning ADC Pharmacological Properties by Glycoengineering

It is widely accepted that IgG glycans are involved in FcγR interactions and thus play regulatory roles in effector cell function, such as antibody-dependent cellular cytotoxicity (ADCC) (Thomann et al., PLOS ONE 10, e0134949, doi: 10.1371/journal.pone.0134949 (2015); Li et al., Proc. National Acad. Sci. (USA) 114, 3485-3490 (2017); Gudelj et al., Cell. Immunol. 333, 65-79 (2018); Cambay et al., Curr. Res. Immunol. 1, 23-37 (2020)). In addition, the lifetime and physical properties of IgG can be affected by the glycan structures. For example, sialylation of IgG glycans was reported to increase IgG longevity by either facilitating the evasion from serum circulation mediated by asialoglycoprotein receptors, or by enhancing IgG interaction with neonatal Fc gamma receptors (FcRn) (Ashwell & Harford Annu. Rev. Biochem.51, 531-554, doi:10.1146/annurev.bi.51.070182.002531 (1982); Bas et al., J. Immunol. 202, 1582 (2019); Morell et al., J. Biol. Chem.246, 1461-1467 (1971)). On the other hand, IgG galactosylation is known to promote C1q binding and downstream complement activity (van Osch et al., J. Immunol., ji2100399, doi: 10.4049/jimmunol.2100399 (2021); Dekkers et al., Front. Immunol. 8, doi: 10.3389/fimmu.2017.00877 (2017); Peschke et al., Front. Immunol. 8, 646 (2017)). IgG glycans, therefore, serve as versatile handles to fine-tune the immune response and pharmacokinetic properties of antibodies, and potentially as for ADCs.

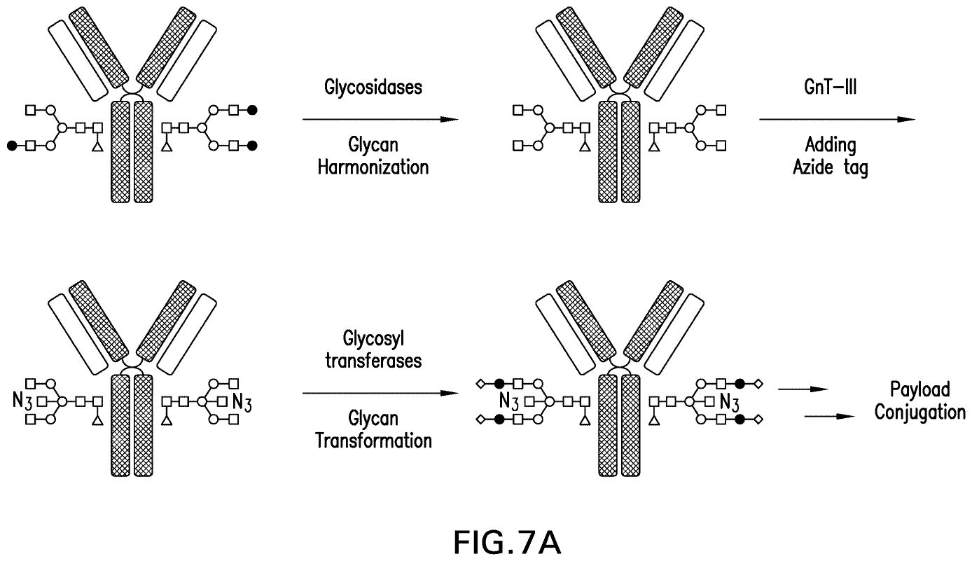

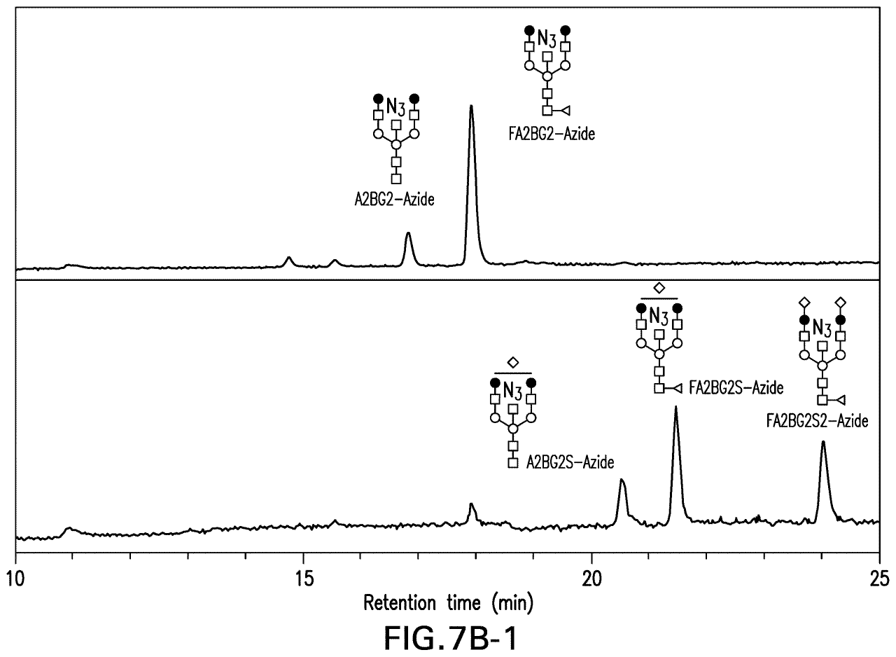

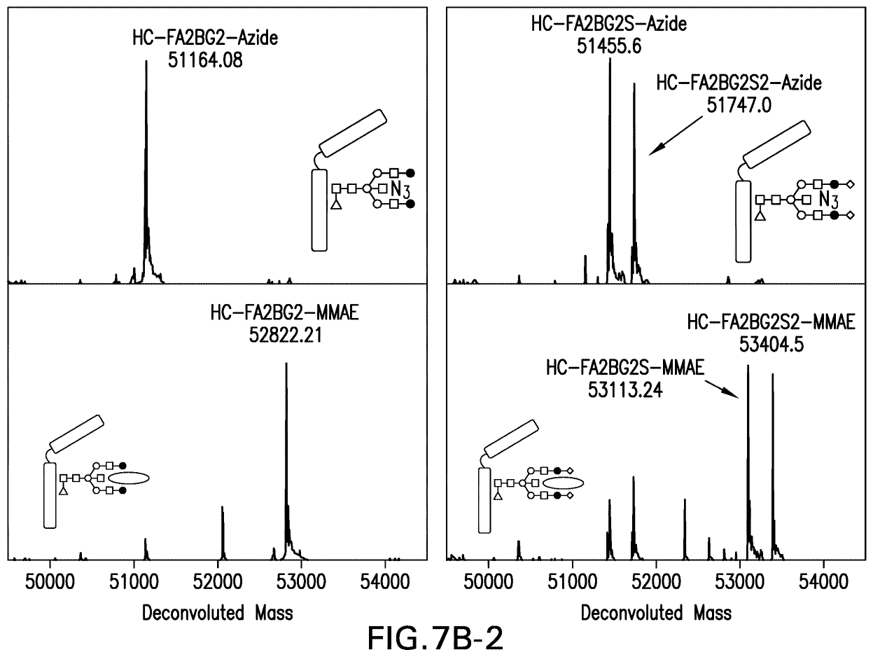

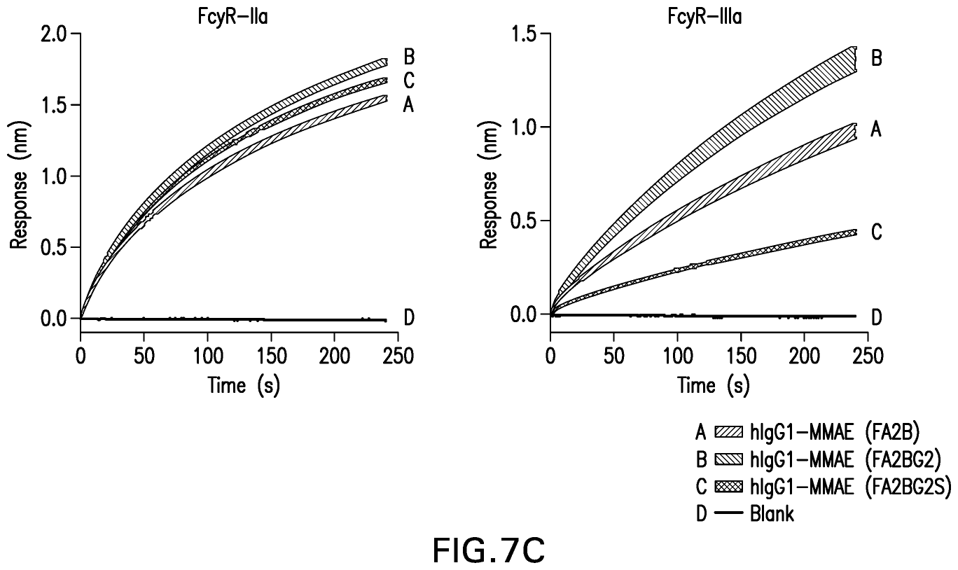

Payload conjugation through the bisecting GlcNAc residues preserves the opportunity of engineering the bi-antennary arms of the N-glycan. This can be achieved by introducing glycosyltransferases to IgG with the azido-glycoforms (e.g., FA2B-Azide) to rebuild the terminal galactose or sialic acid residues (FIG. 7A). Here, we use the human IgG1 Trastuzumab biosimilar as an example. Treating the substrate with human β1-4 galactosyltransferase (Homo sapiens) following the GnT-III reaction gave the di-galactosylated glycoform (FA2BG2-Azide). Further addition of α2-6 sialyltransferase (Homo sapiens) led to the formation of both mono- and bisialylated N-glycans (FA2BG2S-Azide and FA2BG2S2-Azide) (FIG. 7B). These successive glycan remodeling steps can be sped up by using a solid-phase glycan remodeling approach, as we reported earlier (Hsu et al., Comm. Biol. 5, 328 (2022)). After the incubation with the MMAE payload, the formation of the corresponding ADCs was confirmed by LC-MS analyses with over 95% conversion observed within 48 to 72 hours.

Given that glycans regulate IgG-FcγR interactions, we tested whether similar effects could be found on ADCs. We used BLI to measure the binding response of FcγR-IIa and FcγR-IIIa to the human IgG1-MMAE ADCs with different glycoforms, as shown in FIG. 7C. Compared to the ADC with the FA2B glycoform, galactosylation and sialylation slightly improved the ADC binding affinity to FcγR-IIa. This result is highly consistent with the reported IgG1-FcγR-IIa binding data (Thomann et al., PLOS ONE 10, e0134949, doi: 10.1371/journal.pone.0134949 (2015)). In comparison, more substantial impacts of the ADC glycoforms were observed on FcγR-IIIa binding. A 40% increase in BLI response was found when the MMAE ADC is galactosylated; while sialylation led to a dramatic decrease of 55% response in the assay. Our results demonstrate the present invention provides the ability to manipulate effector activity of ADCs through glycoengineering. Furthermore, this approach provides a handle to control the function of ADC to ensure optimal drug potency and safety.

### CITATIONS

1. Tong, J. T. W., Harris, P. W. R., Brimble, M. A. & Kavianinia, I. An Insight into FDA Approved Antibody-Drug Conjugates for Cancer Therapy. Molecules 26, 5847 (2021).
2. Leung, D. et al. Antibody Conjugates-Recent Advances and Future Innovations. Antibodies (Basel) 9, doi: 10.3390/antib9010002 (2020).
3 Lehar, S. M. et al. Novel antibody-antibiotic conjugate eliminates intracellular S. aureus. Nature 527, 323-328 (2015).
4. McPherson, M. J. & Hobson, A. D. Pushing the Envelope: Advancement of ADCs Outside of Oncology. Methods Mol Biol 2078, 23-36 (2020).
5. Banik, S. M. et al. Lysosome-targeting chimaeras for degradation of extracellular proteins. Nature 584, 291-297 (2020).
6. Alves, N. J. Antibody conjugation and formulation. Antib Ther 2, 33-39 (2019).
7. Chari, R. V. J., Miller, M. L. & Widdison, W. C. Antibody-Drug Conjugates: An Emerging Concept in Cancer Therapy. Angewandte Chemie International Edition 53, 3796-3827 (2014).
8. Junutula, J. R. et al. Site-specific conjugation of a cytotoxic drug to an antibody improves the therapeutic index. Nature Biotechnology 26, 925-932 (2008).
9. Junutula, J. R. et al. Rapid identification of reactive cysteine residues for site-specific labeling of antibody-Fabs. J Immunol Methods 332, 41-52 (2008).
10. Shinmi, D. et al. One-Step Conjugation Method for Site-Specific Antibody-Drug Conjugates through Reactive Cysteine-Engineered Antibodies. Bioconjugate Chemistry 27, 1324-1331 (2016).
11. Beerli, R. R., Hell, T., Merkel, A. S. & Grawunder, U. Sortase Enzyme-Mediated Generation of Site-Specifically Conjugated Antibody Drug Conjugates with High In Vitro and In Vivo Potency. PLOS ONE 10, e0131177 (2015).
12. Chen, L. et al. Improved variants of SrtA for site-specific conjugation on antibodies and proteins with high efficiency. Scientific Reports 6, 31899 (2016).
13. Kishimoto, S. et al. Site-Specific Chemical Conjugation of Antibodies by Using Affinity Peptide for the Development of Therapeutic Antibody Format. Bioconjug Chem 30, 698-702 (2019).
14. Dennler, P. et al. Transglutaminase-Based Chemo-Enzymatic Conjugation Approach Yields Homogeneous Antibody-Drug Conjugates. Bioconjugate Chemistry 25, 569-578 (2014).
15. Agnew, B. et al. Site-specific glycan-conjugated NISTmAb antibody drug conjugate mimetics: synthesis, characterization, and utility. Anal Bioanal Chem 413, 4989-5001 (2021).
16. Li, X., Fang, T. & Boons, G. J. Preparation of well-defined antibody-drug conjugates through glycan remodeling and strain-promoted azide-alkyne cycloadditions. Angew Chem Int Ed Engl 53, 7179-7182 (2014).
17. Okeley, N. M. et al. Metabolic engineering of monoclonal antibody carbohydrates for antibody-drug conjugation. Bioconjug Chem 24, 1650-1655 (2013).
18. Zhou, Q. et al. Site-Specific Antibody-Drug Conjugation through Glycoengineering. Bioconjugate Chemistry 25, 510-520 (2014).
19. Zhu, Z. et al. Site-specific antibody-drug conjugation through an engineered glycotransferase and a chemically reactive sugar. MAbs 6, 1190-1200 (2014).
20. Higel, F., Seidl, A., Sörgel, F. & Friess, W. N-glycosylation heterogeneity and the influence on structure, function and pharmacokinetics of monoclonal antibodies and Fc fusion proteins. Eur J Pharm Biopharm 100, 94-100 (2016).
21. Pucić, M. et al. High throughput isolation and glycosylation analysis of IgG-variability and heritability of the IgG glycome in three isolated human populations. Mol Cell Proteomics 10, doi: 10.1074/mcp.M111.010090 (2011).
22. Walls, A. C. et al. Glycan shield and epitope masking of a coronavirus spike protein observed by cryo-electron microscopy. Nature Structural & Molecular Biology 23, 899-905 (2016).
23. van Geel, R. et al. Chemoenzymatic Conjugation of Toxic Payloads to the Globally Conserved N-Glycan of Native mAbs Provides Homogeneous and Highly Efficacious Antibody-Drug Conjugates. Bioconjugate Chemistry 26, 2233-2242 (2015).
24. Krapp, S., Mimura, Y., Jefferis, R., Huber, R. & Sondermann, P. Structural analysis of human IgG-Fc glycoforms reveals a correlation between glycosylation and structural integrity. J Mol Biol 325, 979-989 (2003).
25. Mimura, Y. et al. The influence of glycosylation on the thermal stability and effector function expression of human IgG1-Fc: properties of a series of truncated glycoforms. Mol Immunol 37, 697-706 (2000).
26. Mimura, Y. et al. Glycosylation engineering of therapeutic IgG antibodies: challenges for the safety, functionality and efficacy. Protein Cell 9, 47-62 (2018).
27. Goetze, A. M. et al. High-mannose glycans on the Fc region of therapeutic IgG antibodies increase serum clearance in humans. Glycobiology 21, 949-959 (2011).
28. Chiang, A. W. et al. Modulating carbohydrate-protein interactions through glycoengineering of monoclonal antibodies to impact cancer physiology. Curr Opin Struct Biol 40, 104-111 (2016).
29. Huang, W., Giddens, J., Fan, S.-Q., Toonstra, C. & Wang, L.-X. Chemoenzymatic Glycoengineering of Intact IgG Antibodies for Gain of Functions. Journal of the American Chemical Society 134, 12308-12318 (2012).
30. Narasimhan, S. Control of glycoprotein synthesis. UDP-GlcNAc: glycopeptide beta 4-N-acetylglucosaminyltransferase III, an enzyme in hen oviduct which adds GlcNAc in beta 1-4 linkage to the beta-linked mannose of the trimannosyl core of N-glycosyl oligosaccharides. J Biol Chem 257, 10235-10242 (1982)
31. Horisawa, K. Specific and quantitative labeling of biomolecules using click chemistry. Frontiers in Physiology 5, doi: 10.3389/fphys.2014.00457 (2014).
32. Bai, R., Petit, G. R. & Hamel, E. Dolastatin 10, a powerful cytostatic peptide derived from a marine animal: Inhibition of tubulin polymerization mediated through the vinca alkaloid binding domain. Biochemical Pharmacology 39, 1941-1949 (1990).
33. Hsu, Y.-P. et al. Successive remodeling of IgG glycans using a solid-phase enzymatic platform. Communications Biology 5, 328 (2022).
34. Thomann, M. et al. In Vitro Glycoengineering of IgG1 and Its Effect on Fc Receptor Binding and ADCC Activity. PLOS ONE 10, e0134949, doi: 10.1371/journal.pone.0134949 (2015).
35. Peters, C. & Brown, S. Antibody-drug conjugates as novel anti-cancer chemotherapeutics. Biosci Rep 35, e00225, doi: 10.1042/BSR20150089 (2015).
36. Chalouni, C. & Doll, S. Fate of Antibody-Drug Conjugates in Cancer Cells. Journal of Experimental & Clinical Cancer Research 37, 20 (2018).
37 Su, Z. et al. Antibody-drug conjugates: Recent advances in linker chemistry. Acta Pharmaceutica Sinica B 11, 3889-3907 (2021).
38. Freeze, H. H. & Kranz, C. Endoglycosidase and glycoamidase release of N-linked glycans. Curr Protoc Mol Biol 17.13A.1, doi: 10.1002/0471142727.mb1713as89 (2010).
39. Collin, M. & Olsén, A. EndoS, a novel secreted protein from Streptococcus pyogenes with endoglycosidase activity on human IgG. EMBO J 20, 3046-3055 (2001).
40. Hudis, C. A. Trastuzumab—Mechanism of Action and Use in Clinical Practice. New England Journal of Medicine 357, 39-51 (2007).
41. de Taeye, S. W., Rispens, T. & Vidarsson, G. The Ligands for Human IgG and Their Effector Functions. Antibodies (Basel) 8, doi: 10.3390/antib8020030 (2019).
42. Junker, F., Gordon, J. & Qureshi, O. Fc Gamma Receptors and Their Role in Antigen Uptake, Presentation, and T Cell Activation. Frontiers in Immunology 11, doi: 10.3389/fimmu.2020.01393 (2020).
43. Nimmerjahn, F. & Ravetch, J. V. Fcγ receptors as regulators of immune responses. Nature Reviews Immunology 8, 34-47 (2008).
44. Bruhns, P. et al. Specificity and affinity of human Fcγ receptors and their polymorphic variants for human IgG subclasses. Blood 113, 3716-3725 (2009).
45. Temming, A. R. et al. Cross-reactivity of mouse IgG subclasses to human Fc gamma receptors: Antibody deglycosylation only eliminates IgG2b binding. Mol Immunol 127, 79-86 (2020).
46. Yamazoe, S. et al. Impact of Drug Conjugation on Thermal and Metabolic Stabilities of Aglycosylated and N-Glycosylated Antibodies. Bioconjugate Chemistry 33, 576-585 (2022).
47. Chen, W. et al. Stabilizing the CH2 Domain of an Antibody by Engineering in an Enhanced Aromatic Sequon. ACS Chem Biol 11, 1852-1861 (2016).
48. Li, T. et al. Modulating IgG effector function by Fc glycan engineering. Proceedings of the National Academy of Sciences 114, 3485-3490 (2017).
49. Gudelj, I., Lauc, G. & Pezer, M. Immunoglobulin G glycosylation in aging and diseases. Cell Immunol 333, 65-79 (2018).
50. Cambay, F. et al. Impact of IgG1 N-glycosylation on their interaction with Fc gamma receptors. Current Research in Immunology 1, 23-37 (2020).
51. Ashwell, G. & Harford, J. Carbohydrate-specific receptors of the liver. Annu Rev Biochem 51, 531-554, doi: 10.1146/annurev.bi.51.070182.002531 (1982).
52. Bas, M. et al. Fc Sialylation Prolongs Serum Half-Life of Therapeutic Antibodies. The Journal of Immunology 202, 1582 (2019).
53. Morell, A. G., Gregoriadis, G., Scheinberg, I. H., Hickman, J. & Ashwell, G. The Role of Sialic Acid in Determining the Survival of Glycoproteins in the Circulation. Journal of Biological Chemistry 246, 1461-1467 (1971).
54. van Osch, T. L. J. et al. Fc Galactosylation Promotes Hexamerization of Human IgG1, Leading to Enhanced Classical Complement Activation. The Journal of Immunology, ji2100399, doi: 10.4049/jimmunol.2100399 (2021).
55. Dekkers, G. et al. Decoding the Human Immunoglobulin G-Glycan Repertoire Reveals a Spectrum of Fc-Receptor- and Complement-Mediated-Effector Activities. Frontiers in Immunology 8, doi: 10.3389/fimmu.2017.00877 (2017).
56. Peschke, B., Keller, C. W., Weber, P., Quast, I. & Lünemann, J. D. Fc-Galactosylation of Human Immunoglobulin Gamma Isotypes Improves C1q Binding and Enhances Complement-Dependent Cytotoxicity. Front Immunol 8, 646 (2017).
57. Lu, R.-M. et al. Development of therapeutic antibodies for the treatment of diseases. Journal of Biomedical Science 27, 1 (2020).
58. Czajkowsky, D. M., Hu, J., Shao, Z. & Pleass, R. J. Fc-fusion proteins: new developments and future perspectives. EMBO Mol Med 4, 1015-1028 (2012).
59. Lu, D. et al. Fab-scFv fusion protein: an efficient approach to production of bispecific antibody fragments. Journal of Immunological Methods 267, 213-226 (2002).

### EXEMPLARY EMBODIMENTS OF THE INVENTION

Embodiment 1. A glycoprotein comprising a protein conjugated to at least one bisected N-glycan in which the nonreducing end of the β1,4 linked mannose residue of the trimannosyl core (M3) of the bisected N-glycan is linked to the reducing end of an azido-N-acetyl glucosamine residue in a β1,4 linkage,
wherein M3 is represented by the formula comprising Manα1-3(Manα1-6)Manβ1-4GlcNAcβ1-4GlcNAcβ1-Asn;
wherein the azido-N-acetyl glucosamine is selected from the group consisting of 6-azido-GlcNAc comprising the formula:

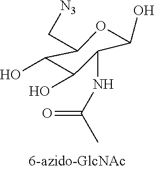 [SMILES: CC(=O)NC1C(O)OC(CN=[N+]=[N-])C(O)C1O]

and GlcNAz comprising the formula:

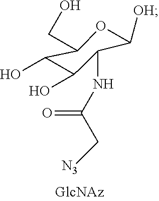 [SMILES: [N-]=[N+]=NCC(=O)NC1C(O)OC(CO)C(O)C1O]

and
wherein the nonreducing end of at least one of the α1,6-linked or α1,3-linked mannose residues is linked to the reducing end of an N-acetyl glucosamine (GlcNAc) residue by a β1,2 linkage.
   Embodiment 2. The glycoprotein of embodiment 1, wherein the nonreducing end of the GlcNAc residue is linked to the reducing end of a galactose residue by a β1,4 linkage.
   Embodiment 3. The glycoprotein of embodiment 2, wherein the nonreducing end of the galactose residue is linked to the reducing end of a sialic acid residue by an α2,6 linkage.
   Embodiment 4. The glycoprotein of embodiment 3, wherein the nonreducing end of the galactose residue is linked to the reducing end of a sialic acid residue by an α2,3 linkage.
   Embodiment 5. The glycoprotein of embodiment 1, wherein the nonreducing end of the α1,6-linked mannose residue is linked to the reducing end of a first GlcNAc residue by a β1,2 linkage and the nonreducing end of the α1,3-linked mannose residue is linked to the reducing end of a second GlcNAc residue by a β1,2 linkage.
   Embodiment 6. The glycoprotein of embodiment 5, wherein (i) the nonreducing end of the first GlcNAc residue is linked to the reducing end of a galactose residue by a β1,4 linkage; (ii) the nonreducing end of the second GlcNAc residue is linked to the reducing end of a galactose residue by a β1,4 linkage; or (iii) the nonreducing end of the first GlcNAc residue is linked to the reducing end of a first galactose residue by a β1,4 linkage and the nonreducing end of the second GlcNAc residue is linked to the reducing end of a second galactose residue by a β1,4 linkage.
   Embodiment 7. The glycoprotein of embodiment 6, wherein the nonreducing end of the galactose residue of (i) or (ii) or the nonreducing end of the first and second galactose residues of (iii) each linked to the reducing end of a sialic acid residue by an α2,3 linkage.
   Embodiment 8. The glycoprotein of embodiment 6, wherein the nonreducing end of the galactose residue of (i) or (ii) or the nonreducing end of the first and second galactose residues of (iii) each linked to the reducing end of a sialic acid residue by an α2,6 linkage.
   Embodiment 9. The glycoprotein of embodiment 1, wherein the azido group of the azido-N-acetyl glucosamine is conjugated to a payload comprising an alkyne functional group in a triazole linkage.
   Embodiment 10. The glycoprotein of embodiment 9, wherein the payload is a therapeutic agent, a diagnostic agent, radionuclide, or protecting group.
   Embodiment 11. The glycoprotein of embodiment 10, wherein the therapeutic agent is a cytotoxic agent, an anti-inflammatory agent, a peptide, an immune agonist, or a nucleic acid molecule or nucleic acid analog.
   Embodiment 12. The glycoprotein of embodiment 11, wherein the anti-inflammatory agent is a glucocorticoid receptor agonist.
   Embodiment 13. The glycoprotein of embodiment 12, wherein the anti-inflammatory agent is Cortisol, cortisone acetate, beclometasone, prednisone, prednisolone, methylprednisolone, betamethasone, trimcinolone, budesonide, dexamethasone, fluticasone, or mometasone.
   Embodiment 14. The glycoprotein of embodiment 11, wherein the cytotoxic agent is selected from the group consisting of taxanes, anthracyclines, camptothecins, epothilones, mytomycins, combretastatins, vinca alkaloids, maytansinoids, enediynes, duocarmycins, tubulysins, amatoxins, bleomycins, dolastatins and auristatins, pyrrolobenzodiazepine dimers, indolinobenzodiazepine dimers, and radioisotopes.
   Embodiment 15. The glycoprotein of embodiment 1, wherein the protein comprises the fragment crystallizable (Fc) domain of an antibody.
   Embodiment 16. The glycoprotein of embodiment 1, wherein the protein is an antibody having a heavy chain and a light chain.
   Embodiment 17. An antibody conjugated to at least one bisected N-glycan in which the nonreducing end of the β1,4 linked mannose residue of the trimannosyl core (M3) of the bisected N-glycan is linked to the reducing end of an azido-N-acetyl glucosamine residue in a β1,4 linkage,
wherein M3 is represented by the formula comprising Manα1-3(Manα1-6)Manβ1-4GlcNAcβ1-4GlcNAcβ1-Asn;
wherein the azido-N-acetyl glucosamine is selected from the group consisting of 6-azido-GlcNAc comprising the formula:

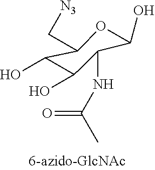 [SMILES: CC(=O)NC1C(O)OC(CN=[N+]=[N-])C(O)C1O]

and GlcNAz comprising the formula:

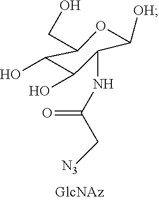 [SMILES: [N-]=[N+]=NCC(=O)NC1C(O)OC(CO)C(O)C1O]

and
wherein the nonreducing end of at least one of the α1,6-linked or α1,3-linked mannose residues is linked to the reducing end of an N-acetyl glucosamine (GlcNAc) residue by a β1,2 linkage.
   Embodiment 18. The antibody of embodiment 17, wherein the nonreducing end of the GlcNAc residue is linked to the reducing end of a galactose residue by a β1,4 linkage.
   Embodiment 19. The antibody of embodiment 18, wherein the nonreducing end of the galactose residue is linked to the reducing end of a sialic acid residue by an α2,6 linkage.
   Embodiment 20. The antibody of embodiment 18, wherein the nonreducing end of the galactose residue is linked to the reducing end of a sialic acid residue by an α2,3 linkage.
   Embodiment 21. The antibody of embodiment 17, wherein the nonreducing end of the α1,6-linked mannose residue is linked to the reducing end of a first GlcNAc residue by a β1,2 linkage and the nonreducing end of the α1,3-linked mannose residue is linked to the reducing end of a second GlcNAc residue by a β1,2 linkage.
   Embodiment 22. The antibody of embodiment 21, wherein (i) the nonreducing end of the first GlcNAc residue is linked to the reducing end of a galactose residue by a β1,4 linkage; (ii) the nonreducing end of the second GlcNAc residue is linked to the reducing end of a galactose residue by a β1,4 linkage; or (iii) the nonreducing end of the first GlcNAc residue is linked to the reducing end of a first galactose residue by a β1,4 linkage and the nonreducing end of the second GlcNAc residue is linked to the reducing end of a second galactose residue by a β1,4 linkage.
   Embodiment 23. The antibody of embodiment 22, wherein the nonreducing end of the galactose residue of (i) or (ii) or the nonreducing end of the first and second galactose residues of (iii) each linked to the reducing end of a sialic acid residue by an α2,3 linkage.
   Embodiment 24. The antibody of embodiment 221, wherein the nonreducing end of the galactose residue of (i) or (ii) or the nonreducing end of the first and second galactose residues of (iii) each linked to the reducing end of a sialic acid residue by an α2,6 linkage.
   Embodiment 25. The antibody of embodiment 17, wherein the azido group of the azido-N-acetyl glucosamine is conjugated to a payload comprising an alkyne functional group in a triazole linkage.
   Embodiment 26. The antibody of embodiment 25, wherein the payload is a therapeutic agent, a diagnostic agent, radionuclide, or protecting group.
   Embodiment 27. The antibody of embodiment 26, wherein the therapeutic agent is a cytotoxic agent, an anti-inflammatory agent, a peptide, an immune agonist, or a nucleic acid molecule or nucleic acid analog.
   Embodiment 28. The antibody of embodiment 27, wherein the anti-inflammatory agent is a glucocorticoid receptor agonist.
   Embodiment 29. The antibody of embodiment 28, wherein the anti-inflammatory agent is Cortisol, cortisone acetate, beclometasone, prednisone, prednisolone, methylprednisolone, betamethasone, trimcinolone, budesonide, dexamethasone, fluticasone, or mometasone.
   Embodiment 30. The antibody of embodiment 27, wherein the cytotoxic agent is selected from the group consisting of taxanes, anthracyclines, camptothecins, epothilones, mytomycins, combretastatins, vinca alkaloids, maytansinoids, enediynes, duocarmycins, tubulysins, amatoxins, bleomycins, dolastatins and auristatins, pyrrolobenzodiazepine dimers, indolinobenzodiazepine dimers, and radioisotopes.
   Embodiment 31. The antibody of embodiment 17, wherein the bisected N-glycan is conjugated to an asparagine residue at position 297 of the constant domain, wherein the position is according to Eu numbering.
   Embodiment 32. The antibody of embodiment 17, wherein the bisected N-glycan is conjugated to an asparagine residue of a N-glycosylation site comprising the amino acid sequence asparagine-X-serine/threonine wherein X is any amino acid except proline.
   Embodiment 33. An antibody conjugate comprising the structure:

Ab-[B-T-L-Pay]ₙ

wherein Ab is an antibody; B is a bisected N-glycan wherein the bisecting sugar residue is azido-GlcNAc or GlcNAz having an azide functional group; L is a linker element comprising an alkyne functional group or a bond; Pay is a payload, which comprises an alkyne functional group when L is a bond; T is a triazole linkage formed between the linkage of the azide functional group and the alkyne functional group; and, n is an integer from 1-20.
Embodiment 34. The antibody of embodiment 33, wherein L is a self-immolative linker.
Embodiment 35. The antibody of embodiment 33, wherein L has the formula L¹-L², wherein each of L¹ and L² is independently a linker element or a bond.
Embodiment 36. The antibody of embodiment 33, wherein L comprises a distal end comprising an alkyne containing group linked to the azide of the azido-GlcNAc or GlcNAz of bisected N-glycan B in triazole linkage T and a proximal end linked to Pay.
Embodiment 37. The antibody of embodiment 36, wherein the alkyne group is a strained cyclooctyne.
Embodiment 38. The antibody of embodiment 37, wherein the strained cyclooctyne is DBCO.
Embodiment 39. The antibody of embodiment 33, wherein the bisected N-glycan B comprises a structure selected from the group consisting of A2B-azide; FA2B-azide; A2B[3]G1-azide; FA2 [3]BG1-azide; A2B[6]G1-azide; FA2B[6]G1-azide; A2BG2-azide; FA2BG2-azide; A2B[3]G1S1(6)-azide; FA2B[3]G1S1(6)-azide; A2B[6]G1S1(6)-azide; FA2B[6]G1S1(6)-azide; A2BG2S2(6)-azide; FA2BG2S2(6)-azide; A2B[3]G1S1(3)-azide; FA2B[3]G1S1(3)-azide; A2B[6]G1S1(3)-azide; FA2B[6]G1S1(3)-azide; A2BG2S2(3)-azide; FA2BG2S2(3)-azide; A1B[3]-azide; FA1B[3]-azide; A1B[3]G1-azide; FA1B[3]G1-azide; A1B[3]G1S1(6)-azide; A1B[3]G1S1(3)-azide; FA1B[3]G1S1(6)-azide; and, FA1B[3]G1S1(3)-azide, wherein the azide refers to the azide of 6-azido-GlcNAc or GlcNAz and which reacts with the alkyne to form the triazole linkage T.
Embodiment 40. The antibody of embodiment 33, wherein the bisecting sugar residue is GlcNAz and the triazole linkage has the structure:

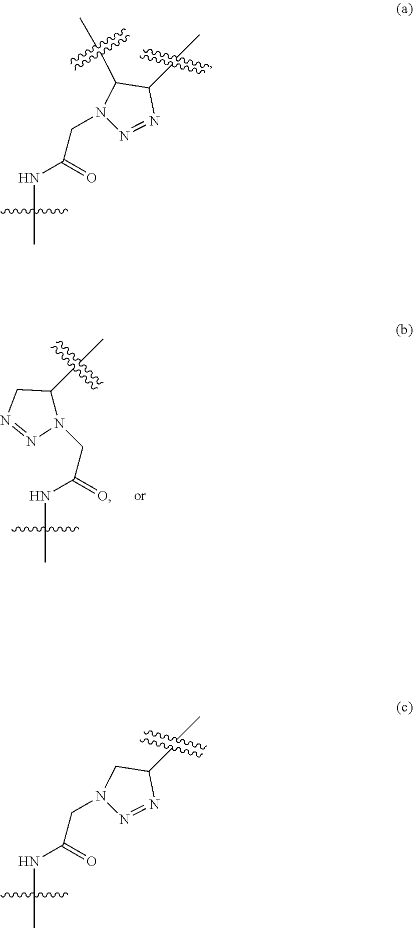

wherein the wavy line identifies the bond between the amine group of the GlcNAz residue and the C2 carbon of the glucose residue comprising the GlcNAz residue and the double wavy lines identify bonds between the alkyne functional group of L or Pay when L is a bond; or, the bisecting sugar residue is 6-azido-GlcNAc and the triazole linkage has the structure:

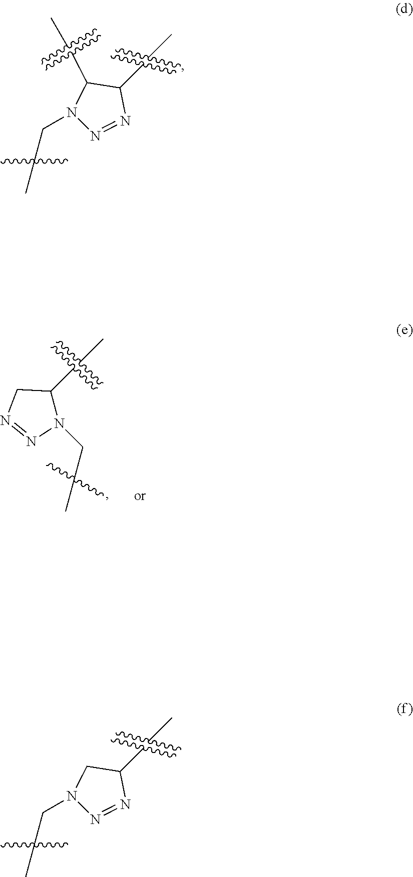

wherein the wavy line identifies the bond between the C5 and C6 carbons of the glucose residue comprising the 6-azido-GlcNAc residue and the double wavy lines identify bonds between the alkyne functional group of L or Pay when L is a bond.
Embodiment 41. A composition comprising a plurality of antibody-drug conjugates (ADCs) and a pharmaceutically acceptable carrier, wherein each ADC independently comprises the structure

Ab-[B-T-L-Pay]ₙ

wherein Ab is an antibody; B is a bisected N-glycan wherein the bisecting sugar residue is azido-GlcNAc or GlcNAz residue having an azide functional group; L is a linker element comprising an alkyne group or a bond; Pay is a payload, which comprises an alkyne group when L is a bond; T is a triazole linkage formed between the linkage of the azide group and the alkyne group; and, n is an integer from 1-20.
Embodiment 42. The composition of embodiment 41, wherein L is a self-immolative linker.
Embodiment 43. The composition of embodiment 41, wherein L has the formula L¹-L², wherein each of L¹ and L² is independently a linker element or a bond.
Embodiment 44. The composition of embodiment 41, wherein L comprises a distal end comprising an alkyne containing group linked to the azide of the azido-GlcNAc or GlcNAz of bisected N-glycan B in triazole linkage T and a proximal end linked to Pay.
Embodiment 45. The composition of embodiment 44, wherein the alkyne group is a strained cyclooctyne.
Embodiment 46. The composition of embodiment 45, wherein the strained cyclooctyne is DBCO.
Embodiment 47. The composition of embodiment 41, wherein the bisected N-glycan B comprises a structure selected from the group consisting of A2B-azide; FA2B-azide; A2B[3]G1-azide; FA2 [3]BG1-azide; A2B[6]G1-azide; FA2B[6]G1-azide; A2BG2-azide; FA2BG2-azide; A2B[3]G1S1(6)-azide; FA2B[3]G1S1(6)-azide; A2B[6]G1S1(6)-azide; FA2B[6]G1S1(6)-azide; A2BG2S2(6)-azide; FA2BG2S2(6)-azide; A2B[3]G1S1(3)-azide; FA2B[3]G1S1(3)-azide; A2B[6]G1S1(3)-azide; FA2B[6]G1S1(3)-azide; A2BG2S2(3)-azide; FA2BG2S2(3)-azide; A1B[3]-azide; FA1B[3]-azide; A1B[3]G1-azide; FA1B[3]G1-Azide; A1B[3]G1S1(6)-azide; A1B[3]G1S1(3)-azide; FA1B[3]G1S1(6)-azide; and, FA1B[3]G1S1(3)-azide, wherein the azide refers to the azide of 6-azido-GlcNAc or GlcNAz and which reacts with the alkyne to form the triazole linkage T.
Embodiment 48. The composition of embodiment 41, wherein the bisecting sugar residue is GlcNAz and the triazole linkage has the structure:

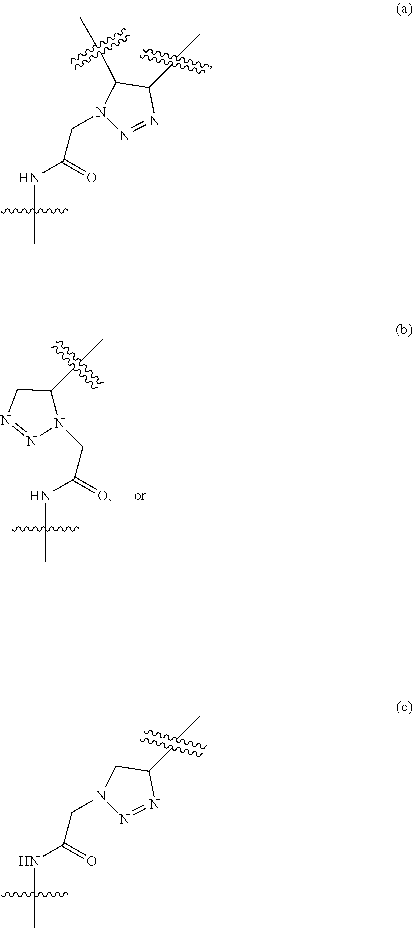

wherein the wavy line identifies the bond between the amine group of the GlcNAz residue and the C2 carbon of the glucose residue comprising the GlcNAz residue and the double wavy lines identify bonds between the alkyne group of L or Pay when L is a bond; or, the bisecting sugar residue is 6-azido-GlcNAc and the triazole linkage has the structure:

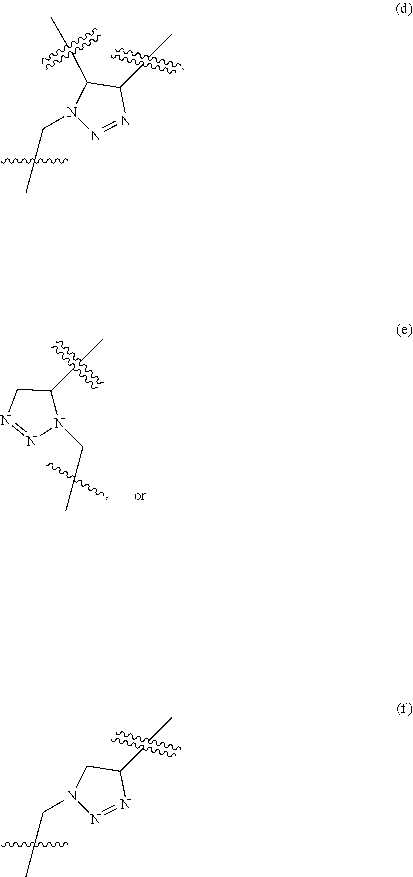

wherein the wavy line identifies the bond between the C5 and C6 carbons of the glucose residue comprising the 6-azido-GlcNAc residue and the double wavy lines identify bonds between the alkyne group of L or Pay when L is a bond.
Embodiment 49. The composition of embodiment 41, wherein the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B-azide or FA2B-azide or both A2B-azide and FA2B-azide, wherein the azide is part of the triazole linkage T.
Embodiment 50. The composition of embodiment 41, wherein the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[3]G1-azide or FA2B[3]G1-azide or both A2B[3]G1-azide and FA2B[3]G1-azide, wherein the azide is part of the triazole linkage T.
Embodiment 51. The composition of embodiment 41, wherein the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[6]G1-azide or FA2B[6]G1-azide or both A2B[6]G1-azide and FA2B[6]G1-azide, wherein the azide is part of the triazole linkage T.
Embodiment 52. The composition of embodiment 41, wherein the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2BG2S2-azide or FA2BG2S2-azide or both A2BG2-azide and FA2BG2-azide, wherein the azide is part of the triazole linkage T.
Embodiment 53. The composition of embodiment 401, wherein the composition, the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[3]G1S1(6 or 3)-azide or FA2B[3]G1S1(6 or 3)-azide or both A2B[3]G1S1(6 or 3)-azide and FA2B[3]G1S1(6 or 3)-azide, wherein the azide is part of the triazole linkage T.
Embodiment 54. The composition of embodiment 41, wherein the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structure A2B[6]G1S1(6 or 3)-azide or FA2B[6]G21S1(6 or 3)-azide or both A2B[6]G1S1(6 or 3)-azide and FA2B[6]G1S1(6 or 3)-azide, wherein the azide is part of the triazole linkage T.
Embodiment 55. The composition of embodiment 41, wherein the predominant N-glycans in the composition comprise greater than 50 mole %, 60 mole %, 70 mole %, 80 mole %, 90 mole %, 95 mole %, 96 mole %, 97 mole %, 98 mole %, 99 mole %, or 100 mole % of the structures A2BG2S2(6 or 3)-azide or FA2BG2S2(6 or 3)-azide or both A2BG2S2(6 or 3)-azide and FA2BG2S2(6 or 3)-azide, wherein the azide is part of the triazole linkage T.
Embodiment 56. An antibody comprising one or more N-glycans selected from the group consisting of A2B-azide; FA2B-azide; A2B[3]G1-azide; FA2 [3]BG1-azide; A2B[6]G1-azide; FA2B[6]G1-azide; A2BG2-azide; FA2BG2-azide; A2B[3]G1S1(6)-azide; FA2B[3]G1S1(6)-azide; A2B[6]G1S1(6)-azide; FA2B[6]G1S1(6)-azide; A2BG2S2(6)-azide; FA2BG2S2(6)-azide; A2B[3]G1S1(3)-azide; FA2B[3]G1S1(3)-azide; A2B[6]G1S1(3)-azide; FA2B[6]G1S1(3)-azide; A2BG2S2(3)-azide; FA2BG2S2(3)-azide; A1B[3]-azide; FA1B[3]-azide; A1B[3]G1-azide; FA1B[3]G1-Azide; A1B[3]G1S1(6)-azide; A1B[3]G1S1(3)-azide; FA1B[3]G1S1(6)-azide; and, FA1B[3]G1S1(3)-azide, wherein the azide refers to the azide of 6-azido-GlcNAc or GlcNAz.
Embodiment 57. An antibody comprising one or more N-glycans selected from the group consisting of A2B-azide; FA2B-azide; A2B[3]G1-azide; FA2 [3]BG1-azide; A2B[6]G1-azide; FA2B[6]G1-azide; A2BG2-azide; FA2BG2-azide; A2B[3]G1S1(6)-azide; FA2B[3]G1S1(6)-azide; A2B[6]G1S1(6)-azide; FA2B[6]G1S1(6)-azide; A2BG2S2(6)-azide; FA2BG2S2(6)-azide; A2B[3]G1S1(3)-azide; FA2B[3]G1S1(3)-azide; A2B[6]G1S1(3)-azide; FA2B[6]G1S1(3)-azide; A2BG2S2(3)-azide; FA2BG2S2(3)-azide; A1B[3]-azide; FA1B[3]-azide; A1B[3]G1-azide; FA1B[3]G1-Azide; A1B[3]G1S1(6)-azide; A1B[3]G1S1(3)-azide; FA1B[3]G1S1(6)-azide; and, FA1B[3]G1S1(3)-azide, wherein the azide refers to the azide of 6-azido-GlcNAc.
Embodiment 58. An antibody comprising one or more N-glycans selected from the group consisting of A2B-azide; FA2B-azide; A2B[3]G1-azide; FA2 [3]BG1-azide; A2B[6]G1-azide; FA2B[6]G1-azide; A2BG2-azide; FA2BG2-azide; A2B[3]G1S1(6)-azide; FA2B[3]G1S1(6)-azide; A2B[6]G1S1(6)-azide; FA2B[6]G1S1(6)-azide; A2BG2S2(6)-azide; FA2BG2S2(6)-azide; A2B[3]G1S1(3)-azide; FA2B[3]G1S1(3)-azide; A2B[6]G1S1(3)-azide; FA2B[6]G1S1(3)-azide; A2BG2S2(3)-azide; FA2BG2S2(3)-azide; A1B[3]-azide; FA1B[3]-azide; A1B[3]G1-azide; FA1B[3]G1-Azide; A1B[3]G1S1(6)-azide; A1B[3]G1S1(3)-azide; FA1B[3]G1S1(6)-azide; and, FA1B[3]G1S1(3)-azide, wherein the azide refers to the azide of GlcNAz.
Embodiment 59. An antibody-drug conjugate (ADC) comprising
an antibody of embodiment 57 or 58 conjugated to a payload comprising an alkyne group.
   Embodiment 60. The ADC of embodiment 59, wherein the payload is a cytotoxic agent, an anti-inflammatory agent, a radionuclide, or diagnostic agent.
   Embodiment 61. The composition of embodiment 41, wherein the predominant N-glycans in the composition comprise greater than 50 mole % of the structure A2B[3]G1-azide or FA2B[3]G1-azide or both A2B[3]G1-azide and FA2B[3]G1-azide, wherein the azide is part of the triazole linkage T.
   Embodiment 62. The composition of embodiment 41, wherein the predominant N-glycans in the composition comprise greater than 50 mole % of the structure A2B[6]G1-azide or FA2B[6]G1-azide or both A2B[6]G1-azide and FA2B[6]G1-azide, wherein the azide is part of the triazole linkage T.
   Embodiment 63. The composition of embodiment 41, wherein the predominant N-glycans in the composition comprise greater than 50 mole % of the structure A2BG2S2-azide or FA2BG2S2-azide or both A2BG2-azide and FA2BG2-azide, wherein the azide is part of the triazole linkage T.
   Embodiment 64. The composition of embodiment 41, wherein the composition, the predominant N-glycans in the composition comprise greater than 50 mole % of the structure A2B[3]G1S1(6 or 3)-azide or FA2B[3]G1S1(6 or 3)-azide or both A2B[3]G1S1(6 or 3)-azide and FA2B[3]G1S1(6 or 3)-azide, wherein the azide is part of the triazole linkage T.
   Embodiment 65. The composition of embodiment 41, wherein the predominant N-glycans in the composition comprise greater than 50 mole % of the structure A2B[6]G1S1(6 or 3)-azide or FA2B[6]G21S1(6 or 3)-azide or both A2B[6]G1S1(6 or 3)-azide and FA2B[6]G1S1(6 or 3)-azide, wherein the azide is part of the triazole linkage T.
   Embodiment 66. The composition of embodiment 41, wherein the predominant N-glycans in the composition comprise greater than 50 mole % of the structures A2BG2S2(6 or 3)-azide or FA2BG2S2(6 or 3)-azide or both A2BG2S2(6 or 3)-azide and FA2BG2S2(6 or 3)-azide, wherein the azide is part of the triazole linkage T.
   Embodiment 67. The composition of embodiment 41, wherein the predominant N-glycans in the composition comprise greater than 60 mole %, 70 mole %, 80 mole %, 90 mole %, 95 mole %, 96 mole %, 97 mole %, 98 mole %, 99 mole %, or 100 mole % of the structure A2B[3]G1-azide or FA2B[3]G1-azide or both A2B[3]G1-azide and FA2B[3]G1-azide, wherein the azide is part of the triazole linkage T.
   Embodiment 68. The composition of embodiment 41, wherein the predominant N-glycans in the composition comprise greater than 60 mole %, 70 mole %, 80 mole %, 90 mole %, 95 mole %, 96 mole %, 97 mole %, 98 mole %, 99 mole %, or 100 mole % of the structure A2B[6]G1-azide or FA2B[6]G1-azide or both A2B[6]G1-azide and FA2B[6]G1-azide, wherein the azide is part of the triazole linkage T.
   Embodiment 69. The composition of embodiment 41, wherein the predominant N-glycans in the composition comprise greater than 60 mole %, 70 mole %, 80 mole %, 90 mole %, 95 mole %, 96 mole %, 97 mole %, 98 mole %, 99 mole %, or 100 mole % of the structure A2BG2S2-azide or FA2BG2S2-azide or both A2BG2-azide and FA2BG2-azide, wherein the azide is part of the triazole linkage T.
   Embodiment 70. The composition of embodiment 41, wherein the composition, the predominant N-glycans in the composition comprise greater than 60 mole %, 70 mole %, 80 mole %, 90 mole %, 95 mole %, 96 mole %, 97 mole %, 98 mole %, 99 mole %, or 100 mole % of the structure A2B[3]G1S1(6 or 3)-azide or FA2B[3]G1S1(6 or 3)-azide or both A2B[3]G1S1(6 or 3)-azide and FA2B[3]G1S1(6 or 3)-azide, wherein the azide is part of the triazole linkage T.
   Embodiment 71. The composition of embodiment 41, wherein the predominant N-glycans in the composition comprise greater than 60 mole %, 70 mole %, 80 mole %, 90 mole %, 95 mole %, 96 mole %, 97 mole %, 98 mole %, 99 mole %, or 100 mole % of the structure A2B[6]G1S1(6 or 3)-azide or FA2B[6]G21S1(6 or 3)-azide or both A2B[6]G1S1(6 or 3)-azide and FA2B[6]G1S1(6 or 3)-azide, wherein the azide is part of the triazole linkage T.
   Embodiment 72. The composition of embodiment 41, wherein the predominant N-glycans in the composition comprise greater than 60 mole %, 70 mole %, 80 mole %, 90 mole %, 95 mole %, 96 mole %, 97 mole %, 98 mole %, 99 mole %, or 100 mole % of the structures A2BG2S2(6 or 3)-azide or FA2BG2S2(6 or 3)-azide or both A2BG2S2(6 or 3)-azide and FA2BG2S2(6 or 3)-azide, wherein the azide is part of the triazole linkage T.
   Embodiment 73. The composition of embodiment 41, wherein the predominant N-glycans in the composition comprise greater than 70 mole % of the structure A2B[3]G1-azide or FA2B[3]G1-azide or both A2B[3]G1-azide and FA2B[3]G1-azide, wherein the azide is part of the triazole linkage T.
   Embodiment 74. The composition of embodiment 41, wherein the predominant N-glycans in the composition comprise greater than 70 mole % of the structure A2B[6]G1-azide or FA2B[6]G1-azide or both A2B[6]G1-azide and FA2B[6]G1-azide, wherein the azide is part of the triazole linkage T.
   Embodiment 75. The composition of embodiment 41, wherein the predominant N-glycans in the composition comprise greater than 70 mole % of the structure A2BG2S2-azide or FA2BG2S2-azide or both A2BG2-azide and FA2BG2-azide, wherein the azide is part of the triazole linkage T.
   Embodiment 76. The composition of embodiment 41, wherein the composition, the predominant N-glycans in the composition comprise greater than 70 mole % of the structure A2B[3]G1S1(6 or 3)-azide or FA2B[3]G1S1(6 or 3)-azide or both A2B[3]G1S1(6 or 3)-azide and FA2B[3]G1S1(6 or 3)-azide, wherein the azide is part of the triazole linkage T.
   Embodiment 77. The composition of embodiment 41, wherein the predominant N-glycans in the composition comprise greater than 70 mole % of the structure A2B[6]G1S1(6 or 3)-azide or FA2B[6]G21S1(6 or 3)-azide or both A2B[6]G1S1(6 or 3)-azide and FA2B[6]G1S1(6 or 3)-azide, wherein the azide is part of the triazole linkage T.
   Embodiment 78. The composition of embodiment 41, wherein the predominant N-glycans in the composition comprise greater than 70 mole % of the structures A2BG2S2(6 or 3)-azide or FA2BG2S2(6 or 3)-azide or both A2BG2S2(6 or 3)-azide and FA2BG2S2(6 or 3)-azide, wherein the azide is part of the triazole linkage T.
   Embodiment 79. The composition of embodiment 41, wherein the predominant N-glycans in the composition comprise greater than 80 mole %, 90 mole %, 95 mole %, 96 mole %, 97 mole %, 98 mole %, 99 mole %, or 100 mole % of the structure A2B[3]G1-azide or FA2B[3]G1-azide or both A2B[3]G1-azide and FA2B[3]G1-azide, wherein the azide is part of the triazole linkage T.
   Embodiment 80. The composition of embodiment 41, wherein the predominant N-glycans in the composition comprise greater than 80 mole % of the structure A2B[6]G1-azide or FA2B[6]G1-azide or both A2B[6]G1-azide and FA2B[6]G1-azide, wherein the azide is part of the triazole linkage T.
   Embodiment 81. The composition of embodiment 41, wherein the predominant N-glycans in the composition comprise greater than 80 mole % of the structure A2BG2S2-azide or FA2BG2S2-azide or both A2BG2-azide and FA2BG2-azide, wherein the azide is part of the triazole linkage T.
   Embodiment 82. The composition of embodiment 41, wherein the composition, the predominant N-glycans in the composition comprise greater than 80 mole % of the structure A2B[3]G1S1(6 or 3)-azide or FA2B[3]G1S1(6 or 3)-azide or both A2B[3]G1S1(6 or 3)-azide and FA2B[3]G1S1(6 or 3)-azide, wherein the azide is part of the triazole linkage T.
   Embodiment 83. The composition of embodiment 41, wherein the predominant N-glycans in the composition comprise greater than 80 mole % of the structure A2B[6]G1S1(6 or 3)-azide or FA2B[6]G21S1(6 or 3)-azide or both A2B[6]G1S1(6 or 3)-azide and FA2B[6]G1S1(6 or 3)-azide, wherein the azide is part of the triazole linkage T.
   Embodiment 84. The composition of embodiment 41, wherein the predominant N-glycans in the composition comprise greater than 80 mole % of the structures A2BG2S2(6 or 3)-azide or FA2BG2S2(6 or 3)-azide or both A2BG2S2(6 or 3)-azide and FA2BG2S2(6 or 3)-azide, wherein the azide is part of the triazole linkage T.
   Embodiment 85. The composition of embodiment 41, wherein the predominant N-glycans in the composition comprise greater than 90 mole % of the structure A2B[3]G1-azide or FA2B[3]G1-azide or both A2B[3]G1-azide and FA2B[3]G1-azide, wherein the azide is part of the triazole linkage T.
   Embodiment 86. The composition of embodiment 41, wherein the predominant N-glycans in the composition comprise greater than 90 mole % of the structure A2B[6]G1-azide or FA2B[6]G1-azide or both A2B[6]G1-azide and FA2B[6]G1-azide, wherein the azide is part of the triazole linkage T.
   Embodiment 87. The composition of embodiment 41, wherein the predominant N-glycans in the composition comprise greater than 90 mole % of the structure A2BG2S2-azide or FA2BG2S2-azide or both A2BG2-azide and FA2BG2-azide, wherein the azide is part of the triazole linkage T.
   Embodiment 88. The composition of embodiment 41, wherein the composition, the predominant N-glycans in the composition comprise greater than 90 mole % of the structure A2B[3]G1S1(6 or 3)-azide or FA2B[3]G1S1(6 or 3)-azide or both A2B[3]G1S1(6 or 3)-azide and FA2B[3]G1S1(6 or 3)-azide, wherein the azide is part of the triazole linkage T.
   Embodiment 89. The composition of embodiment 41, wherein the predominant N-glycans in the composition comprise greater than 90 mole % of the structure A2B[6]G1S1(6 or 3)-azide or FA2B[6]G21S1(6 or 3)-azide or both A2B[6]G1S1(6 or 3)-azide and FA2B[6]G1S1(6 or 3)-azide, wherein the azide is part of the triazole linkage T.
   Embodiment 90. The composition of embodiment 41, wherein the predominant N-glycans in the composition comprise greater than 90 mole % of the structures A2BG2S2(6 or 3)-azide or FA2BG2S2(6 or 3)-azide or both A2BG2S2(6 or 3)-azide and FA2BG2S2(6 or 3)-azide, wherein the azide is part of the triazole linkage T.
   Embodiment 91. The composition of embodiment 41, wherein the predominant N-glycans in the composition comprise greater than 95 mole % of the structure A2B[3]G1-azide or FA2B[3]G1-azide or both A2B[3]G1-azide and FA2B[3]G1-azide, wherein the azide is part of the triazole linkage T.
   Embodiment 92. The composition of embodiment 41, wherein the predominant N-glycans in the composition comprise greater than 95 mole % of the structure A2B[6]G1-azide or FA2B[6]G1-azide or both A2B[6]G1-azide and FA2B[6]G1-azide, wherein the azide is part of the triazole linkage T.
   Embodiment 93. The composition of embodiment 41, wherein the predominant N-glycans in the composition comprise greater than 95 mole % of the structure A2BG2S2-azide or FA2BG2S2-azide or both A2BG2-azide and FA2BG2-azide, wherein the azide is part of the triazole linkage T.
   Embodiment 94. The composition of embodiment 41, wherein the composition, the predominant N-glycans in the composition comprise greater than 95 mole % of the structure A2B[3]G1S1(6 or 3)-azide or FA2B[3]G1S1(6 or 3)-azide or both A2B[3]G1S1(6 or 3)-azide and FA2B[3]G1S1(6 or 3)-azide, wherein the azide is part of the triazole linkage T.
   Embodiment 95. The composition of embodiment 41, wherein the predominant N-glycans in the composition comprise greater than 95 mole % of the structure A2B[6]G1S1(6 or 3)-azide or FA2B[6]G21S1(6 or 3)-azide or both A2B[6]G1S1(6 or 3)-azide and FA2B[6]G1S1(6 or 3)-azide, wherein the azide is part of the triazole linkage T.
   Embodiment 96. The composition of embodiment 41, wherein the predominant N-glycans in the composition comprise greater than 95 mole % of the structures A2BG2S2(6 or 3)-azide or FA2BG2S2(6 or 3)-azide or both A2BG2S2(6 or 3)-azide and FA2BG2S2(6 or 3)-azide, wherein the azide is part of the triazole linkage T.
   Embodiment 97. The composition of embodiment 41, wherein the predominant N-glycans in the composition comprise greater than 96 mole % of the structure A2B[3]G1-azide or FA2B[3]G1-azide or both A2B[3]G1-azide and FA2B[3]G1-azide, wherein the azide is part of the triazole linkage T.
   Embodiment 98. The composition of embodiment 41, wherein the predominant N-glycans in the composition comprise greater than 96 mole % of the structure A2B[6]G1-azide or FA2B[6]G1-azide or both A2B[6]G1-azide and FA2B[6]G1-azide, wherein the azide is part of the triazole linkage T.
   Embodiment 99. The composition of embodiment 41, wherein the predominant N-glycans in the composition comprise greater than 96 mole % of the structure A2BG2S2-azide or FA2BG2S2-azide or both A2BG2-azide and FA2BG2-azide, wherein the azide is part of the triazole linkage T.
   Embodiment 100 The composition of embodiment 41, wherein the composition, the predominant N-glycans in the composition comprise greater than 96 mole % of the structure A2B[3]G1S1(6 or 3)-azide or FA2B[3]G1S1(6 or 3)-azide or both A2B[3]G1S1(6 or 3)-azide and FA2B[3]G1S1(6 or 3)-azide, wherein the azide is part of the triazole linkage T.
   Embodiment 101. The composition of embodiment 41, wherein the predominant N-glycans in the composition comprise greater than 96 mole % of the structure A2B[6]G1S1(6 or 3)-azide or FA2B[6]G21S1(6 or 3)-azide or both A2B[6]G1S1(6 or 3)-azide and FA2B[6]G1S1(6 or 3)-azide, wherein the azide is part of the triazole linkage T.
   Embodiment 102. The composition of embodiment 41, wherein the predominant N-glycans in the composition comprise greater than 96 mole % of the structures A2BG2S2(6 or 3)-azide or FA2BG2S2(6 or 3)-azide or both A2BG2S2(6 or 3)-azide and FA2BG2S2(6 or 3)-azide, wherein the azide is part of the triazole linkage T.
   Embodiment 103. The composition of embodiment 41, wherein the predominant N-glycans in the composition comprise greater than 97 mole % of the structure A2B[3]G1-azide or FA2B[3]G1-azide or both A2B[3]G1-azide and FA2B[3]G1-azide, wherein the azide is part of the triazole linkage T.
   Embodiment 104. The composition of embodiment 41, wherein the predominant N-glycans in the composition comprise greater than 97 mole % of the structure A2B[6]G1-azide or FA2B[6]G1-azide or both A2B[6]G1-azide and FA2B[6]G1-azide, wherein the azide is part of the triazole linkage T.
   Embodiment 105. The composition of embodiment 41, wherein the predominant N-glycans in the composition comprise greater than 97 mole % of the structure A2BG2S2-azide or FA2BG2S2-azide or both A2BG2-azide and FA2BG2-azide, wherein the azide is part of the triazole linkage T.
   Embodiment 106. The composition of embodiment 41, wherein the composition, the predominant N-glycans in the composition comprise greater than 97 mole % of the structure A2B[3]G1S1(6 or 3)-azide or FA2B[3]G1S1(6 or 3)-azide or both A2B[3]G1S1(6 or 3)-azide and FA2B[3]G1S1(6 or 3)-azide, wherein the azide is part of the triazole linkage T.
   Embodiment 107. The composition of embodiment 41, wherein the predominant N-glycans in the composition comprise greater than 97 mole % of the structure A2B[6]G1S1(6 or 3)-azide or FA2B[6]G21S1(6 or 3)-azide or both A2B[6]G1S1(6 or 3)-azide and FA2B[6]G1S1(6 or 3)-azide, wherein the azide is part of the triazole linkage T.
   Embodiment 108. The composition of embodiment 41, wherein the predominant N-glycans in the composition comprise greater than 97 mole % of the structures A2BG2S2(6 or 3)-azide or FA2BG2S2(6 or 3)-azide or both A2BG2S2(6 or 3)-azide and FA2BG2S2(6 or 3)-azide, wherein the azide is part of the triazole linkage T.
   Embodiment 109. The composition of embodiment 41, wherein the predominant N-glycans in the composition comprise greater than 98 mole % of the structure A2B[3]G1-azide or FA2B[3]G1-azide or both A2B[3]G1-azide and FA2B[3]G1-azide, wherein the azide is part of the triazole linkage T.
   Embodiment 110. The composition of embodiment 41, wherein the predominant N-glycans in the composition comprise greater than 98 mole % of the structure A2B[6]G1-azide or FA2B[6]G1-azide or both A2B[6]G1-azide and FA2B[6]G1-azide, wherein the azide is part of the triazole linkage T.
   Embodiment 111. The composition of embodiment 41, wherein the predominant N-glycans in the composition comprise greater than 98 mole % of the structure A2BG2S2-azide or FA2BG2S2-azide or both A2BG2-azide and FA2BG2-azide, wherein the azide is part of the triazole linkage T.
   Embodiment 112. The composition of embodiment 41, wherein the composition, the predominant N-glycans in the composition comprise greater than 98 mole % of the structure A2B[3]G1S1(6 or 3)-azide or FA2B[3]G1S1(6 or 3)-azide or both A2B[3]G1S1(6 or 3)-azide and FA2B[3]G1S1(6 or 3)-azide, wherein the azide is part of the triazole linkage T.
   Embodiment 113. The composition of embodiment 41, wherein the predominant N-glycans in the composition comprise greater than 98 mole % of the structure A2B[6]G1S1(6 or 3)-azide or FA2B[6]G21S1(6 or 3)-azide or both A2B[6]G1S1(6 or 3)-azide and FA2B[6]G1S1(6 or 3)-azide, wherein the azide is part of the triazole linkage T.
   Embodiment 114. The composition of embodiment 41, wherein the predominant N-glycans in the composition comprise greater than 98 mole % of the structures A2BG2S2(6 or 3)-azide or FA2BG2S2(6 or 3)-azide or both A2BG2S2(6 or 3)-azide and FA2BG2S2(6 or 3)-azide, wherein the azide is part of the triazole linkage T.
   Embodiment 115. The composition of embodiment 41, wherein the predominant N-glycans in the composition comprise greater than 99 mole % of the structure A2B[3]G1-azide or FA2B[3]G1-azide or both A2B[3]G1-azide and FA2B[3]G1-azide, wherein the azide is part of the triazole linkage T.
   Embodiment 116. The composition of embodiment 41, wherein the predominant N-glycans in the composition comprise greater than 99 mole % of the structure A2B[6]G1-azide or FA2B[6]G1-azide or both A2B[6]G1-azide and FA2B[6]G1-azide, wherein the azide is part of the triazole linkage T.
   Embodiment 117. The composition of embodiment 41, wherein the predominant N-glycans in the composition comprise greater than 99 mole % of the structure A2BG2S2-azide or FA2BG2S2-azide or both A2BG2-azide and FA2BG2-azide, wherein the azide is part of the triazole linkage T.
   Embodiment 118. The composition of embodiment 41, wherein the composition, the predominant N-glycans in the composition comprise greater than 99 mole % of the structure A2B[3]G1S1(6 or 3)-azide or FA2B[3]G1S1(6 or 3)-azide or both A2B[3]G1S1(6 or 3)-azide and FA2B[3]G1S1(6 or 3)-azide, wherein the azide is part of the triazole linkage T.
   Embodiment 119. The composition of embodiment 41, wherein the predominant N-glycans in the composition comprise greater than 99 mole % of the structure A2B[6]G1S1(6 or 3)-azide or FA2B[6]G21S1(6 or 3)-azide or both A2B[6]G1S1(6 or 3)-azide and FA2B[6]G1S1(6 or 3)-azide, wherein the azide is part of the triazole linkage T.
   Embodiment 120. The composition of embodiment 41, wherein the predominant N-glycans in the composition comprise greater than 99 mole % of the structures A2BG2S2(6 or 3)-azide or FA2BG2S2(6 or 3)-azide or both A2BG2S2(6 or 3)-azide and FA2BG2S2(6 or 3)-azide, wherein the azide is part of the triazole linkage T.
   Embodiment 121. The composition of embodiment 41, wherein the predominant N-glycans in the composition comprise greater than 100 mole % of the structure A2B[3]G1-azide or FA2B[3]G1-azide or both A2B[3]G1-azide and FA2B[3]G1-azide, wherein the azide is part of the triazole linkage T.
   Embodiment 122. The composition of embodiment 41, wherein the predominant N-glycans in the composition comprise greater than 100 mole % of the structure A2B[6]G1-azide or FA2B[6]G1-azide or both A2B[6]G1-azide and FA2B[6]G1-azide, wherein the azide is part of the triazole linkage T.
   Embodiment 123. The composition of embodiment 41, wherein the predominant N-glycans in the composition comprise greater than 100 mole % of the structure A2BG2S2-azide or FA2BG2S2-azide or both A2BG2-azide and FA2BG2-azide, wherein the azide is part of the triazole linkage T.
   Embodiment 124. The composition of embodiment 41, wherein the composition, the predominant N-glycans in the composition comprise greater than 100 mole % of the structure A2B[3]G1S1(6 or 3)-azide or FA2B[3]G1S1(6 or 3)-azide or both A2B[3]G1S1(6 or 3)-azide and FA2B[3]G1S1(6 or 3)-azide, wherein the azide is part of the triazole linkage T.
   Embodiment 125. The composition of embodiment 41, wherein the predominant N-glycans in the composition comprise greater than 100 mole % of the structure A2B[6]G1S1(6 or 3)-azide or FA2B[6]G21S1(6 or 3)-azide or both A2B[6]G1S1(6 or 3)-azide and FA2B[6]G1S1(6 or 3)-azide, wherein the azide is part of the triazole linkage T.
   Embodiment 126. The composition of embodiment 41, wherein the predominant N-glycans in the composition comprise greater than 100 mole % of the structures A2BG2S2(6 or 3)-azide or FA2BG2S2(6 or 3)-azide or both A2BG2S2(6 or 3)-azide and FA2BG2S2(6 or 3)-azide, wherein the azide is part of the triazole linkage T.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the specification and the claims attached herein.

1. A glycoprotein comprising a protein conjugated to at least one bisected N-glycan in which the nonreducing end of the β1,4 linked mannose residue of the trimannosyl core (M3) of the bisected N-glycan is linked to the reducing end of an azido-N-acetyl glucosamine residue in a β1,4 linkage,
wherein M3 is represented by the formula comprising Manα1-3(Manα1-6)Manβ1-4GlcNAcβ1-4GlcNAcβ1-Asn;
wherein the azido-N-acetyl glucosamine is selected from the group consisting of 6-azido-GlcNAc comprising the formula: 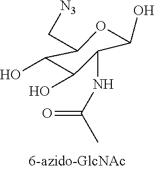 [SMILES: CC(=O)NC1C(O)OC(CN=[N+]=[N-])C(O)C1O]
and GlcNAz comprising the formula: 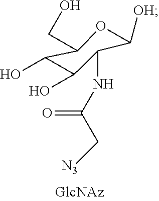 [SMILES: [N-]=[N+]=NCC(=O)NC1C(O)OC(CO)C(O)C1O]
and
wherein the nonreducing end of at least one of the α1,6-linked or α1,3-linked mannose residues is linked to the reducing end of an N-acetyl glucosamine (GlcNAc) residue by a β1,2 linkage.

2. The glycoprotein of claim 1, wherein the nonreducing end of the GlcNAc residue is linked to the reducing end of a galactose residue by a β1,4 linkage.

3. The glycoprotein of claim 2, wherein the nonreducing end of the galactose residue is linked to the reducing end of a sialic acid residue by an α2,6 linkage.

4. The glycoprotein of claim 3, wherein the nonreducing end of the galactose residue is linked to the reducing end of a sialic acid residue by an α2,3 linkage.

5. The glycoprotein of claim 1, wherein the nonreducing end of the α1,6-linked mannose residue is linked to the reducing end of a first GlcNAc residue by a β1,2 linkage and the nonreducing end of the α1,3-linked mannose residue is linked to the reducing end of a second GlcNAc residue by a β1,2 linkage.

6. The glycoprotein of claim 5, wherein (i) the nonreducing end of the first GlcNAc residue is linked to the reducing end of a galactose residue by a β1,4 linkage; (ii) the nonreducing end of the second GlcNAc residue is linked to the reducing end of a galactose residue by a β1,4 linkage; or (iii) the nonreducing end of the first GlcNAc residue is linked to the reducing end of a first galactose residue by a β1,4 linkage and the nonreducing end of the second GlcNAc residue is linked to the reducing end of a second galactose residue by a β1,4 linkage.

7. The glycoprotein of claim 6, wherein the nonreducing end of the galactose residue of (i) or (ii) or the nonreducing end of the first and second galactose residues of (iii) each linked to the reducing end of a sialic acid residue by an α2,3 linkage.

8. The glycoprotein of claim 6, wherein the nonreducing end of the galactose residue of (i) or (ii) or the nonreducing end of the first and second galactose residues of (iii) each linked to the reducing end of a sialic acid residue by an α2,6 linkage.

9. The glycoprotein of claim 1, wherein the azido group of the azido-N-acetyl glucosamine is conjugated to a payload comprising an alkyne function group in a triazole linkage.

10.-14. (canceled)

15. The glycoprotein of claim 1, wherein the protein comprises the fragment crystallizable (Fc) domain of an antibody.

16. The glycoprotein of claim 1, wherein the protein is an antibody having a heavy chain and a light chain.

17. An antibody conjugated to at least one bisected N-glycan in which the nonreducing end of the β1,4 linked mannose residue of the trimannosyl core (M3) of the bisected N-glycan is linked to the reducing end of an azido-N-acetyl glucosamine residue in a β1,4 linkage,
wherein M3 is represented by the formula comprising Manα1-3(Manα1-6)Manβ1-4GlcNAcβ1-4GlcNAcβ1-Asn;
wherein the azido-N-acetyl glucosamine is selected from the group consisting of 6-azido-GlcNAc comprising the formula: 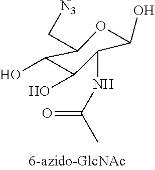 [SMILES: CC(=O)NC1C(O)OC(CN=[N+]=[N-])C(O)C1O]
and GlcNAz comprising the formula: 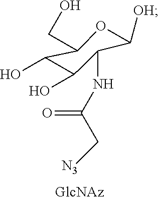 [SMILES: [N-]=[N+]=NCC(=O)NC1C(O)OC(CO)C(O)C1O]
and
wherein the nonreducing end of at least one of the α1,6-linked or α1,3-linked mannose residues is linked to the reducing end of an N-acetyl glucosamine (GlcNAc) residue by a β1,2 linkage.

18-24. (canceled)

25. The antibody of claim 17, wherein the azido group of the azido-N-acetyl glucosamine is conjugated to a payload comprising an alkyne function group in a triazole linkage.

26-32. (canceled)

33. An antibody conjugate comprising the structure:
Ab-[B-T-L-Pay]ₙ
wherein Ab is an antibody; B is a bisected N-glycan wherein the bisecting sugar residue is azido-GlcNAc or GlcNAz having an azide group; L is a linker element comprising an alkyne group or a bond; Pay is a payload, which comprises an alkyne group when L is a bond; T is a triazole linkage formed between the linkage of the azide group and the alkyne group; and, n is an integer from 1-20.

34. The antibody of claim 33, wherein L is a self-immolative linker.

35. The antibody of claim 33, wherein L has the formula L¹-L², wherein each of L¹ and L² is independently a linker element or a bond.

36. The antibody of claim 33, wherein L comprises a distal end comprising an alkyne containing group linked to the azide of the azido-GlcNAc or GlcNAz of bisected N-glycan B in triazole linkage T and a proximal end linked to Pay.

37-40. (canceled)

41. A composition comprising the antibody conjugate of claim 33.

42-55. (canceled)

56. An antibody comprising one or more N-glycans selected from the group consisting of A2B-azide; FA2B-azide; A2B[3]G1-azide; FA2 [3]BG1-azide; A2B[6]G1-azide; FA2B[6]G1-azide; A2BG2-azide; FA2BG2-azide; A2B[3]G1S1(6)-azide; FA2B[3]G1S1(6)-azide; A2B[6]G1S1(6)-azide; FA2B[6]G1S1(6)-azide; A2BG2S2(6)-azide; FA2BG2S2(6)-azide; A2B[3]G1S1(3)-azide; FA2B[3]G1S1(3)-azide; A2B[6]G1S1(3)-azide; FA2B[6]G1S1(3)-azide; A2BG2S2(3)-azide; FA2BG2S2(3)-azide; A1B[3]-azide; FA1B[3]-azide; A1B[3]G1-azide; FA1B[3]G1-Azide; A1B[3]G1S1(6)-azide; A1B[3]G1S1(3)-azide; FA1B[3]G1S1(6)-azide; and, FA1B[3]G1S1(3)-azide, wherein the azide refers to the azide of 6-azido-GlcNAc or GlcNAz.

57-58. (canceled)

59. An antibody-drug conjugate (ADC) comprising an antibody of claim 56 conjugated to a payload comprising an alkyne group.

60. (canceled)
